(19) United States
(12) Patent Application Publication
(10) Pub. No.: US 2017/0354655 A1
(43) Pub. Date: Dec. 14, 2017

(54) TLR INHIBITOR AND BRUTON'S TYROSINE KINASE INHIBITOR COMBINATIONS

(71) Applicant: Pharmacyclics LLC, Sunnyvale, CA (US)

(72) Inventors: Darrin Beaupre, Campbell, CA (US); Betty Y. Chang, Cupertino, CA (US); Hsu-Ping Kuo, Sunnyvale, CA (US)

(21) Appl. No.: 15/527,443

(22) PCT Filed: Nov. 17, 2015
(86) PCT No.: PCT/US15/61091

§ 371 Date: May 17, 2017

Related U.S. Application Data
(60) Provisional application No. 62/080,921, filed on Nov. 17, 2014.
(60) Provisional application No. 62/127,740, filed on Mar. 3, 2015.

(51) Int. Cl. A61K 31/517; A61K 31/519; A61K 45/06

(52) U.S. Cl. CPC A61K 31/517; A61K 45/06; A61K 31/519

(57) ABSTRACT

Provided are compositions for and methods of treating a B-cell malignancy in a subject in need thereof, by administering to the subject a therapeutically effective amount of a combination comprising an inhibitor of a BTK inhibitor and a TLR inhibitor.

### RELATED APPLICATIONS

The present application claims the benefit of priority from U.S. Provisional Patent Application No. 62/080,921, filed on Nov. 17, 2014; and U.S. Provisional Patent Application No. 62/127,740, filed on Mar. 3, 2015, the contents of each of which are herein incorporated by reference in their entireties.

### BACKGROUND OF THE INVENTION

Bruton's tyrosine kinase (BTK), a member of the Tec family of non-receptor tyrosine kinases, is a key signaling enzyme expressed in all hematopoietic cells types except T lymphocytes and natural killer cells. BTK plays an essential role in the B-cell signaling pathway linking cell surface B-cell receptor (BCR) stimulation to downstream intracellular responses.

### SUMMARY OF THE INVENTION

In some embodiments, methods of treating a B-cell malignancy are provided. The methods include the steps of administering to the subject a therapeutically effective amount of a combination comprising a BTK inhibitor and a TLR9 inhibitor selected from the group consisting of a non-specific TLR inhibitor; a TLR6/7/8/9 antagonist; and a TLR9 antagonist, wherein the TLR9 antagonist is selected from the group consisting of chloroquine, quinacrine, monesin, bafilomycin A1, wortmannin, iODN, (+)-morphinans, 9-aminoacridine, 4-aminoquinoline, 4-aminoquinolines, 7,8,9,10-tetrahydro-6H-cyclohepta[b]quinolin-11-ylamine; 1-methyl-2,3-dihydro-1H-pyrrolo[2,3-b]quinolin-4-ylamine; 1,6-dimethyl-2,3-dihydro-1H-pyrrolo[2,3-b]quinolin-4-ylamine; 6-bromo-1-methyl-2,3-dihydro-1H-pyrrolo[2,3-b]quinolin-4-ylamine; 1-methyl-2,3,4,5-tetrahydro-1H-azepino[2,3-b]quinolin-6-ylamine; 3,3-dimethyl-3,4-dihydro-acridin-9-ylamine; 1-benzyl-2,3-dihydro-H-pyrrolo[2,3-b]quinolin-4-ylamine; 6-methyl-1-phenyl-2,3-dihydro-1H-pyrrolo[2,3-b]quinolin-4-ylamine; N*2*,N*2*-Dimethyl-quinoline-2,4-diamine, 2,7-Dimethyl-dibenzo[b,g][1,8]naphthyridin-11-ylamine; 2,4-Dimethyl-benzo[b][1,8]naphthyridin-5-ylamine; 7-Fluoro-2,4-dimethyl-benzo[b][1,8]naphthyridin-5-ylamine; 1,2,3,4-Tetrahydro-acridin-9-ylamine Tacrine hydrochloridehydrate; 2,3-Dihydro-1H-cyclopenta[b]quinolin-9-ylamine; 2,4,9-Trimethyl-benzo[b][1,8]naphthyridin-5-ylamine; 9-Amino-3,3-dimethyl-1,2,3,4-tetrahydro-acridin-1-ol and 7-Ethoxy-N*3*-furan-2-ylmethyl-acridine-3,9-diamine; quinazolines, N,N-dimethyl-N′-{2-[4-(4-methyl-piperazin-1-yl)-phenyl]-3,4-dihydro-quinazoline-4-yl}-ethane-1,2,-diamine; N′-[6,7-Dimethoxy-2-(4-phenyl-piperazin-1-yl)-quinazolin-4-yl]-N,N-dimethyl-ethane-1,2-diamine; N′-[6,7-Dimethoxy-2-(4-methyl-piperazin-1-yl)-quinazolin-4-yl]-N,N-dimethyl-ethane-1,2-diamine; N,N-Dimethyl-N′-(2-phenyl-quinazolin-4-yl)-ethane-1,2-diamine; Dimethyl-(2-{2-[4-(4-methyl-piperazin-1-yl)-phenyl]-quinazolin-4-yloxy}-ethyl)-amine; N′-(2-Biphenyl-4-yl-quinazolin-4-yl)-N,N-dimethyl-ethane-1,2-diamine and Dimethyl-[2-(2-phenyl-quinazolin-4-yloxy)-ethyl]-amine; ODN 2088, ODN with a TTAGGG sequence, G-ODN, statins, atorvastatin, IMO-2125 (Idera Pharmaceuticals), IRS 869, CMZ 203-84, CMZ 203-85, CMZ 203-88, CMZ 203-88-1, CMZ 203-89, CMZ 203-91, INH-ODN 2114, ODN A151, ODN INH-1, ODN INH-18, ODN 4084, ODN 4084-F, and ODN INH-47.

In some embodiments, methods of treating a diffuse large B-cell lymphoma (DLBCL) or a marginal zone lymphoma (MZL) are provided. The methods include the step of administering to a subject in need thereof a therapeutically effective amount of a combination comprising a BTK inhibitor and a TLR inhibitor, wherein the TLR inhibitor is a non-specific TLR inhibitor, a TLR6/7/8/9 antagonist, or a TLR9 antagonist selected from the group consisting of chloroquine, quinacrine, monesin, bafilomycin A1, wortmannin, iODN, (+)-morphinans, 9-aminoacridine, 4-aminoquinoline, 4-aminoquinolines, 7,8,9,10-tetrahydro-6H-cyclohepta[b]quinolin-11-ylamine; 1-methyl-2,3-dihydro-1H-pyrrolo[2,3-b]quinolin-4-ylamine; 1,6-dimethyl-2,3-dihydro-H-pyrrolo[2,3-b]quinolin-4-ylamine; 6-bromo-1-methyl-2,3-dihydro-1H-pyrrolo[2,3-b]quinolin-4-ylamine; 1-methyl-2,3,4,5-tetrahydro-1H-azepino[2,3-b]quinolin-6-ylamine; 3,3-dimethyl-3,4-dihydro-acridin-9-ylamine; 1-benzyl-2,3-dihydro-1H-pyrrolo[2,3-b]quinolin-4-ylamine; 6-methyl-1-phenyl-2,3-dihydro-1H-pyrrolo[2,3-b]quinolin-4-ylamine; N*2*,N*2*-Dimethyl-quinoline-2,4-diamine, 2,7-Dimethyl-dibenzo[b,g][1,8]naphthyridin-11-ylamine; 2,4-Dimethyl-benzo[b][1,8]naphthyridin-5-ylamine; 7-Fluoro-2,4-dimethyl-benzo[b][1,8]naphthyridin-5-ylamine; 1,2,3,4-Tetrahydro-acridin-9-ylamine Tacrine hydrochloridehydrate; 2,3-Dihydro-1H-cyclopenta[b]quinolin-9-ylamine; 2,4,9-Trimethyl-benzo[b][1,8]naphthyridin-5-ylamine; 9-Amino-3,3-dimethyl-1,2,3,4-tetrahydro-acridin-1-ol and 7-Ethoxy-N*3*-furan-2-ylmethyl-acridine-3,9-diamine; quinazolines, N,N-dimethyl-N′-{2-[4-(4-methyl-piperazin-1-yl)-phenyl]-3,4-dihydro-quinazoline-4-yl}-ethane-1,2,-diamine; N′-[6,7-Dimethoxy-2-(4-phenyl-piperazin-1-yl)-quinazolin-4-yl]-N,N-dimethyl-ethane-1,2-diamine; N′-[6,7-Dimethoxy-2-(4-methyl-piperazin-1-yl)-quinazolin-4-yl]-N,N-dimethyl-ethane-1,2-diamine; N,N-Dimethyl-N′-(2-phenyl-quinazolin-4-yl)-ethane-1,2-diamine; Dimethyl-(2-{2-[4-(4-methyl-piperazin-1-yl)-phenyl]-quinazolin-4-yloxy}-ethyl)-amine; N′-(2-Biphenyl-4-yl-quinazolin-4-yl)-N,N-dimethyl-ethane-1,2-diamine and Dimethyl-[2-(2-phenyl-quinazolin-4-yloxy)-ethyl]-amine; ODN 2088, ODN with a TTAGGG sequence, G-ODN, statins, atorvastatin, IMO-2125 (Idera Pharmaceuticals), IRS 869, CMZ 203-84, CMZ 203-85, CMZ 203-88, CMZ 203-88-1, CMZ 203-89, CMZ 203-91, INH-ODN 2114, ODN A151, ODN INH-1, ODN INH-18, ODN 4084, ODN 4084-F, and ODN INH-47.

In some embodiments, methods of treating a B-cell malignancy associated with over-activated TLR signaling are provided. The methods include detecting the presence of absence of a mutation in MYD88 in a sample from an individual; and administering to the individual a therapeutically effective amount of a combination comprising a BTK inhibitor and a TLR inhibitor if the individual has a mutation in MYD88, wherein the TLR inhibitor is selected from the group consisting of a non-specific TLR inhibitor; a TLR6/7/8/9 antagonist; and a TLR9 antagonist, wherein the TLR9 antagonist is selected from the group consisting of chloroquine, quinacrine, monesin, bafilomycin A1, wortmannin, iODN, (+)-morphinans, 9-aminoacridine, 4-aminoquinoline, 4-aminoquinolines, 7,8,9,10-tetrahydro-6H-cyclohepta[b]quinolin-11-ylamine; 1-methyl-2,3-dihydro-1H-pyrrolo[2,3-b]quinolin-4-ylamine; 1,6-dimethyl-2,3-dihydro-H-pyrrolo[2,3-b]quinolin-4-ylamine; 6-bromo-1-methyl-2,3-dihydro-1H-pyrrolo[2,3-b]quinolin-4-ylamine; 1-methyl-2,3,4,5-tetrahydro-1H-azepino[2,3-b]quinolin-6-ylamine; 3,3-dimethyl-3,4-dihydro-acridin-9-ylamine; 1-benzyl-2,3-dihydro-1H-pyrrolo[2,3-b]quinolin-4-ylamine; 6-methyl-1-phenyl-2,3-dihydro-1H-pyrrolo[2,3-b]quinolin-4-ylamine; N*2*,N*2*-Dimethyl-quinoline-2,4-diamine, 2,7-Dimethyl-dibenzo[b,g][1,8]naphthyridin-11-ylamine; 2,4-Dimethyl-benzo[b][1,8]naphthyridin-5-ylamine; 7-Fluoro-2,4-dimethyl-benzo[b][1,8]naphthyridin-5-ylamine; 1,2,3,4-Tetrahydro-acridin-9-ylamine Tacrine hydrochloridehydrate; 2,3-Dihydro-1H-cyclopenta[b]quinolin-9-ylamine; 2,4,9-Trimethyl-benzo[b][1,8]naphthyridin-5-ylamine; 9-Amino-3,3-dimethyl-1,2,3,4-tetrahydro-acridin-1-ol and 7-Ethoxy-N*3*-furan-2-ylmethyl-acridine-3,9-diamine; quinazolines, N,N-dimethyl-N′-{2-[4-(4-methyl-piperazin-1-yl)-phenyl]-3,4-dihydro-quinazoline-4-yl}-ethane-1,2,-diamine; N′-[6,7-Dimethoxy-2-(4-phenyl-piperazin-1-yl)-quinazolin-4-yl]-N,N-dimethyl-ethane-1,2-diamine; N′-[6,7-Dimethoxy-2-(4-methyl-piperazin-1-yl)-quinazolin-4-yl]-N,N-dimethyl-ethane-1,2-diamine; N,N-Dimethyl-N′-(2-phenyl-quinazolin-4-yl)-ethane-1,2-diamine; Dimethyl-(2-{2-[4-(4-methyl-piperazin-1-yl)-phenyl]-quinazolin-4-yloxy}-ethyl)-amine; N′-(2-Biphenyl-4-yl-quinazolin-4-yl)-N,N-dimethyl-ethane-1,2-diamine and Dimethyl-[2-(2-phenyl-quinazolin-4-yloxy)-ethyl]-amine; ODN 2088, ODN with a TTAGGG sequence, G-ODN, statins, atorvastatin, IMO-2125 (Idera Pharmaceuticals), IRS 869, CMZ 203-84, CMZ 203-85, CMZ 203-88, CMZ 203-88-1, CMZ 203-89, CMZ 203-91, INH-ODN 2114, ODN A151, ODN INH-1, ODN INH-18, ODN 4084, ODN 4084-F, and ODN INH-47.

In some embodiments, methods of selecting an individual having a B-cell malignancy for therapy with a combination comprising a BTK inhibitor and a TLR inhibitor, wherein the TLR inhibitor is selected from the group consisting of a non-specific TLR inhibitor; a TLR6/7/8/9 antagonist; and a TLR9 antagonist, wherein the TLR9 antagonist is selected from the group consisting of chloroquine, quinacrine, monesin, bafilomycin A1, wortmannin, iODN, (+)-morphinans, 9-aminoacridine, 4-aminoquinoline, 4-aminoquinolines, 7,8,9,10-tetrahydro-6H-cyclohepta[b]quinolin-11-ylamine; 1-methyl-2,3-dihydro-H-pyrrolo[2,3-b]quinolin-4-ylamine; 1,6-dimethyl-2,3-dihydro-1H-pyrrolo[2,3-b]quinolin-4-ylamine; 6-bromo-1-methyl-2,3-dihydro-1H-pyrrolo[2,3-b]quinolin-4-ylamine; 1-methyl-2,3,4,5-tetrahydro-1H-azepino[2,3-b]quinolin-6-ylamine; 3,3-dimethyl-3,4-dihydro-acridin-9-ylamine; 1-benzyl-2,3-dihydro-1H-pyrrolo[2,3-b]quinolin-4-ylamine; 6-methyl-1-phenyl-2,3-dihydro-1H-pyrrolo[2,3-b]quinolin-4-ylamine; N*2*,N*2*-Dimethyl-quinoline-2,4-diamine, 2,7-Dimethyl-dibenzo[b,g][1,8]naphthyridin-11-ylamine; 2,4-Dimethyl-benzo[b][1,8]naphthyridin-5-ylamine; 7-Fluoro-2,4-dimethyl-benzo[b][1,8]naphthyridin-5-ylamine; 1,2,3,4-Tetrahydro-acridin-9-ylamine Tacrine hydrochloridehydrate; 2,3-Dihydro-1H-cyclopenta[b]quinolin-9-ylamine; 2,4,9-Trimethyl-benzo[b][1,8]naphthyridin-5-ylamine; 9-Amino-3,3-dimethyl-1,2,3,4-tetrahydro-acridin-1-ol and 7-Ethoxy-N*3*-furan-2-ylmethyl-acridine-3,9-diamine; quinazolines, N,N-dimethyl-N′-{2-[4-(4-methyl-piperazin-1-yl)-phenyl]-3,4-dihydro-quinazoline-4-yl}-ethane-1,2,-diamine; N′-[6,7-Dimethoxy-2-(4-phenyl-piperazin-1-yl)-quinazolin-4-yl]-N,N-dimethyl-ethane-1,2-diamine; N′-[6,7-Dimethoxy-2-(4-methyl-piperazin-1-yl)-quinazolin-4-yl]-N,N-dimethyl-ethane-1,2-diamine; N,N-Dimethyl-N′-(2-phenyl-quinazolin-4-yl)-ethane-1,2-diamine; Dimethyl-(2-{2-[4-(4-methyl-piperazin-1-yl)-phenyl]-quinazolin-4-yloxy}-ethyl)-amine; N′-(2-Biphenyl-4-yl-quinazolin-4-yl)-N,N-dimethyl-ethane-1,2-diamine and Dimethyl-[2-(2-phenyl-quinazolin-4-yloxy)-ethyl]-amine; ODN 2088, ODN with a TTAGGG sequence, G-ODN, statins, atorvastatin, IMO-2125 (Idera Pharmaceuticals), IRS 869, CMZ 203-84, CMZ 203-85, CMZ 203-88, CMZ 203-88-1, CMZ 203-89, CMZ 203-91, INH-ODN 2114, ODN A151, ODN INH-1, ODN INH-18, ODN 4084, ODN 4084-F, and ODN INH-47, comprising: detecting the presence of absence of a mutation in MYD88 in a sample from an individual; and characterizing the individual as a candidate for therapy with the combination comprising a BTK inhibitor and a TLR inhibitor if the individual has a mutation in MYD88.

In some embodiments, a pharmaceutical composition is provided. The pharmaceutical composition comprises a BTK inhibitor and a TLR inhibitor, wherein the TLR inhibitor is selected from the group consisting of a non-specific TLR inhibitor; a TLR7/8/9 antagonist; and a TLR9 antagonist, wherein the TLR9 antagonist is selected from the group consisting of is selected from the group consisting of a non-specific TLR inhibitor; a TLR6/7/8/9 antagonist; and a TLR9 antagonist, wherein the TLR9 antagonist is selected from the group consisting of chloroquine, quinacrine, monesin, bafilomycin A1, wortmannin, iODN, (+)-morphinans, 9-aminoacridine, 4-aminoquinoline, 4-aminoquinolines, 7,8,9,10-tetrahydro-6H-cyclohepta[b]quinolin-11-ylamine; 1-methyl-2,3-dihydro-1H-pyrrolo[2,3-b]quinolin-4-ylamine; 1,6-dimethyl-2,3-dihydro-1H-pyrrolo[2,3-b]quinolin-4-ylamine; 6-bromo-1-methyl-2,3-dihydro-1H-pyrrolo[2,3-b]quinolin-4-ylamine; 1-methyl-2,3,4,5-tetrahydro-1H-azepino[2,3-b]quinolin-6-ylamine; 3,3-dimethyl-3,4-dihydro-acridin-9-ylamine; 1-benzyl-2,3-dihydro-1H-pyrrolo[2,3-b]quinolin-4-ylamine; 6-methyl-1-phenyl-2,3-dihydro-1H-pyrrolo[2,3-b]quinolin-4-ylamine; N*2*,N*2*-Dimethyl-quinoline-2,4-diamine, 2,7-Dimethyl-dibenzo[b,g][1,8]naphthyridin-11-ylamine; 2,4-Dimethyl-benzo[b][1,8]naphthyridin-5-ylamine; 7-Fluoro-2,4-dimethyl-benzo[b][1,8]naphthyridin-5-ylamine; 1,2,3,4-Tetrahydro-acridin-9-ylamine Tacrine hydrochloridehydrate; 2,3-Dihydro-1H-cyclopenta[b]quinolin-9-ylamine; 2,4,9-Trimethyl-benzo[b][1,8]naphthyridin-5-ylamine; 9-Amino-3,3-dimethyl-1,2,3,4-tetrahydro-acridin-1-ol and 7-Ethoxy-N*3*-furan-2-ylmethyl-acridine-3,9-diamine; quinazolines, N,N-dimethyl-N′-{2-[4-(4-methyl-piperazin-1-yl)-phenyl]-3,4-dihydro-quinazoline-4-yl}-ethane-1,2,-diamine; N′-[6,7-Dimethoxy-2-(4-phenyl-piperazin-1-yl)-quinazolin-4-yl]-N,N-dimethyl-ethane-1,2-diamine; N′-[6,7-Dimethoxy-2-(4-methyl-piperazin-1-yl)-quinazolin-4-yl]-N,N-dimethyl-ethane-1,2-diamine; N,N-Dimethyl-N′-(2-phenyl-quinazolin-4-yl)-ethane-1,2-diamine; Dimethyl-(2-{2-[4-(4-methyl-piperazin-1-yl)-phenyl]-quinazolin-4-yloxy}-ethyl)-amine; N′-(2-Biphenyl-4-yl-quinazolin-4-yl)-N,N-dimethyl-ethane-1,2-diamine and Dimethyl-[2-(2-phenyl-quinazolin-4-yloxy)-ethyl]-amine; ODN 2088, ODN with a TTAGGG sequence, G-ODN, statins, atorvastatin, IMO-2125 (Idera Pharmaceuticals), IRS 869, CMZ 203-84, CMZ 203-85, CMZ 203-88, CMZ 203-88-1, CMZ 203-89, CMZ 203-91, INH-ODN 2114, ODN A151, ODN INH-1, ODN INH-18, ODN 4084, ODN 4084-F, and ODN INH-47.

Disclosed herein, in certain embodiments, are methods of treating a B-cell malignancy in a subject in need thereof, comprising administering to the subject a therapeutically effective amount of a combination comprising a BTK inhibitor and a TLR inhibitor. In some embodiments, the combination provides a synergistic therapeutic effect compared to administration of the BTK inhibitor or the TLR inhibitor alone. In some embodiments, the TLR inhibitor is selected from a non-specific TLR inhibitor, a TLR7/8/9 antagonist, and a TLR9 antagonist. In some embodiments, the non-specific TLR inhibitor is selected from the group consisting of chloroquine and bafilomycin A. In some embodiments, the TLR7/8/9 antagonist is selected from the group consisting of CPG52364, IMO 8400, and IMO-9200. In some embodiments, the TLR9 antagonist is selected from the group consisting of chloroquine, quinacrine, monesin, bafilomycin A1, wortmannin, iODN, (+)-morphinans, 9-aminoacridine, 4-aminoquinoline, 4-aminoquinolines, 7,8,9,10-tetrahydro-6H-cyclohepta[b]quinolin-11-ylamine; 1-methyl-2,3-dihydro-1H-pyrrolo[2,3-b]quinolin-4-ylamine; 1,6-dimethyl-2,3-dihydro-H-pyrrolo[2,3-b]quinolin-4-ylamine; 6-bromo-1-methyl-2,3-dihydro-1H-pyrrolo[2,3-b]quinolin-4-ylamine; 1-methyl-2,3,4,5-tetrahydro-1H-azepino[2,3-b]quinolin-6-ylamine; 3,3-dimethyl-3,4-dihydro-acridin-9-ylamine; 1-benzyl-2,3-dihydro-1H-pyrrolo[2,3-b]quinolin-4-ylamine; 6-methyl-1-phenyl-2,3-dihydro-1H-pyrrolo[2,3-b]quinolin-4-ylamine; N*2*,N*2*-Dimethyl-quinoline-2,4-diamine, 2,7-Dimethyl-dibenzo[b,g][1,8]naphthyridin-11-ylamine; 2,4-Dimethyl-benzo[b][1,8]naphthyridin-5-ylamine; 7-Fluoro-2,4-dimethyl-benzo[b][1,8]naphthyridin-5-ylamine; 1,2,3,4-Tetrahydro-acridin-9-ylamine Tacrine hydrochloridehydrate; 2,3-Dihydro-1H-cyclopenta[b]quinolin-9-ylamine; 2,4,9-Trimethyl-benzo[b][1,8]naphthyridin-5-ylamine; 9-Amino-3,3-dimethyl-1,2,3,4-tetrahydro-acridin-1-ol and 7-Ethoxy-N*3*-furan-2-ylmethyl-acridine-3,9-diamine; quinazolines, N,N-dimethyl-N′-{2-[4-(4-methyl-piperazin-1-yl)-phenyl]-3,4-dihydro-quinazoline-4-yl}-ethane-1,2,-diamine; N′-[6,7-Dimethoxy-2-(4-phenyl-piperazin-1-yl)-quinazolin-4-yl]-N,N-dimethyl-ethane-1,2-diamine; N′-[6,7-Dimethoxy-2-(4-methyl-piperazin-1-yl)-quinazolin-4-yl]-N,N-dimethyl-ethane-1,2-diamine; N,N-Dimethyl-N′-(2-phenyl-quinazolin-4-yl)-ethane-1,2-diamine; Dimethyl-(2-{2-[4-(4-methyl-piperazin-1-yl)-phenyl]-quinazolin-4-yloxy}-ethyl)-amine; N′-(2-Biphenyl-4-yl-quinazolin-4-yl)-N,N-dimethyl-ethane-1,2-diamine and Dimethyl-[2-(2-phenyl-quinazolin-4-yloxy)-ethyl]-amine; ODN 2088, ODN with a TTAGGG sequence, G-ODN, statins, atorvastatin, IMO-2125 (Idera Pharmaceuticals), IRS 869, CMZ 203-84, CMZ 203-85, CMZ 203-88, CMZ 203-88-1, CMZ 203-89, CMZ 203-91, INH-ODN 2114, ODN A151, ODN INH-1, ODN INH-18, ODN 4084, ODN 4084-F, and ODN INH-47. In some embodiments, the BTK inhibitor is a compound of Formula (D)

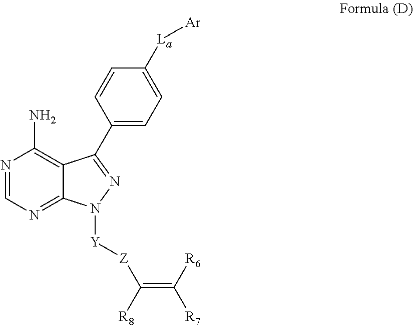

wherein

La is CH₂, O, NH or S;

Ar is an optionally substituted aromatic carbocycle or an aromatic heterocycle;

Y is an optionally substituted alkyl, heteroalkyl, carbocycle, heterocycle, or combination thereof;

Z is C(O), OC(O), NHC(O), C(S), S(O)ₓ, OS(O)ₓ, NHS(O)ₓ, where x is 1 or 2; and

R₆, R₇, and R₈ are independently selected from H, alkyl, heteroalkyl, carbocycle, heterocycle, or combinations thereof.

In some embodiments, the BTK inhibitor is ibrutinib. In some embodiments, the BTK inhibitor is ibrutinib and the TLR inhibitor is chloroquine. In some embodiments the B-cell malignancy is diffuse large B-cell lymphoma (DLBCL), marginal zone lymphoma (MZL), acute lymphoblastic leukemia (ALL), acute myelogenous leukemia (AML), chronic myelogenous leukemia (CML), acute monocytic leukemia (AMoL), chronic lymphocytic leukemia (CLL), small lymphocytic lymphoma (SLL), high-risk small lymphocytic lymphoma (SLL), follicular lymphoma (FL), mantle cell lymphoma (MCL), Waldenstrom's macroglobulinemia, multiple myeloma, extranodal marginal zone B cell lymphoma, nodal marginal zone B cell lymphoma, Burkitt's lymphoma, non-Burkitt high grade B cell lymphoma, primary mediastinal B-cell lymphoma (PMBL), immunoblastic large cell lymphoma, precursor B-lymphoblastic lymphoma, B cell prolymphocytic leukemia, lymphoplasmacytic lymphoma, splenic marginal zone lymphoma, plasma cell myeloma, plasmacytoma, mediastinal (thymic) large B cell lymphoma, intravascular large B cell lymphoma, primary effusion lymphoma, or lymphomatoid granulomatosis. In some embodiments, the B-cell malignancy is relapsed or refractory. In some embodiments, the B-cell malignancy is a non-Hodgkin's lymphoma. In some embodiments, the B-cell malignancy is diffuse large B-cell lymphoma (DLBCL). In some embodiments, the DLBCL is activated B-cell diffuse large B-cell lymphoma (ABC-DLBCL). In some embodiments, the ABC-DLBCL is characterized by a mutation in MYD88. In some embodiments, the mutation is at position 265 of MYD88. In some embodiments, the mutation is an L265P mutation. In some embodiments, the B-cell malignancy marginal zone lymphoma (MZL). In some embodiments, the BTK inhibitor is administered once a day, two times per day, three times per day, four times per day, or five times per day. In some embodiments, the BTK inhibitor is administered at a dosage of about 40 mg/day to about 1000 mg/day. In some embodiments, the BTK inhibitor is administered orally. In some embodiments, the BTK inhibitor and the TLR inhibitor are administered simultaneously, sequentially or intermittently. In some embodiments, the method further comprises administering a third therapeutic agent. In some embodiments, the third therapeutic agent is selected from among a chemotherapeutic agent or radiation therapeutic agent. In some embodiments, the chemotherapeutic agent is selected from among chlorambucil, ifosfamide, doxorubicin, mesalazine, thalidomide, lenalidomide, temsirolimus, everolimus, fludarabine, fostamatinib, paclitaxel, docetaxel, ofatumumab, rituximab, dexamethasone, prednisone, CAL-101, ibritumomab, tositumomab, bortezomib, pentostatin, endostatin, or a combination thereof.

Disclosed herein, in certain embodiments, are methods of treating a diffuse large B-cell lymphoma (DLBCL) or a marginal zone lymphoma (MZL) comprising administering to a subject in need thereof a therapeutically effective amount of a combination comprising a BTK inhibitor and a TLR inhibitor. In some embodiments, the combination provides a synergistic therapeutic effect compared to administration of the BTK inhibitor or the TLR inhibitor alone. In some embodiments, the TLR inhibitor is selected from a non-specific TLR inhibitor, a TLR7/8/9 antagonist, and a TLR9 antagonist. In some embodiments, the non-specific TLR inhibitor is selected from the group consisting of chloroquine and bafilomycin A. In some embodiments, the TLR7/8/9 antagonist is selected from the group consisting of CPG52364, IMO 8400, and IMO-9200. In some embodiments, the TLR9 antagonist is selected from the group consisting of chloroquine, quinacrine, monesin, bafilomycin A1, wortmannin, iODN, (+)-morphinans, 9-aminoacridine, 4-aminoquinoline, 4-aminoquinolines, 7,8,9,10-tetrahydro-6H-cyclohepta[b]quinolin-11-ylamine; 1-methyl-2,3-dihydro-1H-pyrrolo[2,3-b]quinolin-4-ylamine; 1,6-dimethyl-2,3-dihydro-1H-pyrrolo[2,3-b]quinolin-4-ylamine; 6-bromo-1-methyl-2,3-dihydro-1H-pyrrolo[2,3-b]quinolin-4-ylamine; 1-methyl-2,3,4,5-tetrahydro-1H-azepino[2,3-b]quinolin-6-ylamine; 3,3-dimethyl-3,4-dihydro-acridin-9-ylamine; 1-benzyl-2,3-dihydro-1H-pyrrolo[2,3-b]quinolin-4-ylamine; 6-methyl-1-phenyl-2,3-dihydro-1H-pyrrolo[2,3-b]quinolin-4-ylamine; N*2*,N*2*-Dimethyl-quinoline-2,4-diamine, 2,7-Dimethyl-dibenzo[b,g][1,8]naphthyridin-11-ylamine; 2,4-Dimethyl-benzo[b][1,8]naphthyridin-5-ylamine; 7-Fluoro-2,4-dimethyl-benzo[b][1,8]naphthyridin-5-ylamine; 1,2,3,4-Tetrahydro-acridin-9-ylamine Tacrine hydrochloridehydrate; 2,3-Dihydro-1H-cyclopenta[b]quinolin-9-ylamine; 2,4,9-Trimethyl-benzo[b][1,8]naphthyridin-5-ylamine; 9-Amino-3,3-dimethyl-1,2,3,4-tetrahydro-acridin-1-ol and 7-Ethoxy-N*3*-furan-2-ylmethyl-acridine-3,9-diamine; quinazolines, N,N-dimethyl-N′-{2-[4-(4-methyl-piperazin-1-yl)-phenyl]-3,4-dihydro-quinazoline-4-yl}-ethane-1,2,-diamine; N′-[6,7-Dimethoxy-2-(4-phenyl-piperazin-1-yl)-quinazolin-4-yl]-N,N-dimethyl-ethane-1,2-diamine; N′-[6,7-Dimethoxy-2-(4-methyl-piperazin-1-yl)-quinazolin-4-yl]-N,N-dimethyl-ethane-1,2-diamine; N,N-Dimethyl-N′-(2-phenyl-quinazolin-4-yl)-ethane-1,2-diamine; Dimethyl-(2-{2-[4-(4-methyl-piperazin-1-yl)-phenyl]-quinazolin-4-yloxy}-ethyl)-amine; N′-(2-Biphenyl-4-yl-quinazolin-4-yl)-N,N-dimethyl-ethane-1,2-diamine and Dimethyl-[2-(2-phenyl-quinazolin-4-yloxy)-ethyl]-amine; ODN 2088, ODN with a TTAGGG sequence, G-ODN, statins, atorvastatin, IMO-2125 (Idera Pharmaceuticals), IRS 869, CMZ 203-84, CMZ 203-85, CMZ 203-88, CMZ 203-88-1, CMZ 203-89, CMZ 203-91, INH-ODN 2114, ODN A151, ODN INH-1, ODN INH-18, ODN 4084, ODN 4084-F, and ODN INH-47. In some embodiments, the BTK inhibitor is a compound of Formula (D)

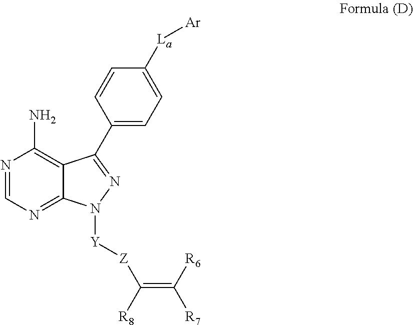

wherein

Lₐ is CH₂, O, NH or S;

Ar is an optionally substituted aromatic carbocycle or an aromatic heterocycle;

Y is an optionally substituted alkyl, heteroalkyl, carbocycle, heterocycle, or combination thereof;

Z is C(O), OC(O), NHC(O), C(S), S(O)ₓ, OS(O)ₓ, NHS(O)ₓ, where x is 1 or 2; and

R₆, R₇, and R₈ are independently selected from H, alkyl, heteroalkyl, carbocycle, heterocycle, or combinations thereof.

In some embodiments, the BTK inhibitor is ibrutinib. In some embodiments, the BTK inhibitor is ibrutinib and the TLR inhibitor is chloroquine. In some embodiments, the DLBCL is activated B-cell diffuse large B-cell lymphoma (ABC-DLBCL). In some embodiments, the ABC-DLBCL is characterized by a mutation in MYD88. In some embodiments, the mutation is at position 265 of MYD88. In some embodiments, the mutation is an L265P mutation. In some embodiments, the BTK inhibitor is administered once a day, two times per day, three times per day, four times per day, or five times per day. In some embodiments, the BTK inhibitor is administered at a dosage of about 40 mg/day to about 1000 mg/day. In some embodiments, the BTK inhibitor is administered orally. In some embodiments, the BTK inhibitor and the TLR inhibitor are administered simultaneously, sequentially or intermittently. In some embodiments, the method further comprises administering a third therapeutic agent. In some embodiments, the third therapeutic agent is selected from among a chemotherapeutic agent or radiation therapeutic agent. In some embodiments, the chemotherapeutic agent is selected from among chlorambucil, ifosfamide, doxorubicin, mesalazine, thalidomide, lenalidomide, temsirolimus, everolimus, fludarabine, fostamatinib, paclitaxel, docetaxel, ofatumumab, rituximab, dexamethasone, prednisone, CAL-101, ibritumomab, tositumomab, bortezomib, pentostatin, endostatin, or a combination thereof.

Disclosed herein, in certain embodiments, are methods of treating a B-cell malignancy associated with over-activated TLR signaling, comprising: (a) detecting the presence of absence of a mutation in MYD88 in a sample from an individual; and (b) administering to the individual a therapeutically effective amount of a combination comprising a BTK inhibitor and a TLR inhibitor if the individual has a mutation in MYD88. In some embodiments, the mutation is at amino acid position 198 or 265 of MYD88. In some embodiments, the mutation at amino acid position 198 of MYD88 is S198N. In some embodiments, the mutation at amino acid position 265 of MYD88 is L265P. In some embodiments, wherein sample is a nucleic acid molecule containing sample encoding MYD88 from the individual, and the detecting comprises testing the nucleic acid molecule containing sample to determine whether the nucleic acid molecules encoding MYD88 contain the mutation. In some embodiments, the nucleic acid molecule is RNA or DNA. In some embodiments, the DNA is genomic DNA. In some embodiments, the testing comprises amplifying the nucleic acid molecules encoding MYD88. In some embodiments, the amplification is by isothermal amplification or polymerase chain reaction (PCR). In some embodiments, the amplification is by PCR. In some embodiments, the testing comprises contacting nucleic acids with sequence specific nucleic acid probes, wherein the sequence specific nucleic acid probes bind to nucleic acids encoding MYD88 having a mutation and do not bind to nucleic acid encoding wild-type MYD88. In some embodiments, the testing comprises PCR amplification using the sequence specific nucleic acid probes. In some embodiments, the sample comprises one or more tumor cells. In some embodiments, the combination provides a synergistic therapeutic effect compared to administration of the BTK inhibitor or the TLR inhibitor alone. In some embodiments, the TLR inhibitor is selected from a non-specific TLR inhibitor, a TLR7/8/9 antagonist, and a TLR9 antagonist. In some embodiments, the non-specific TLR inhibitor is selected from the group consisting of chloroquine and bafilomycin A. In some embodiments, the TLR7/8/9 antagonist is selected from the group consisting of CPG52364, IMO 8400, and IMO-9200. In some embodiments, the TLR9 antagonist is selected from the group consisting of chloroquine, quinacrine, monesin, bafilomycin A1, wortmannin, iODN, (+)-morphinans, 9-aminoacridine, 4-aminoquinoline, 4-aminoquinolines, 7,8,9,10-tetrahydro-6H-cyclohepta[b]quinolin-11-ylamine; 1-methyl-2,3-dihydro-1H-pyrrolo[2,3-b]quinolin-4-ylamine; 1,6-dimethyl-2,3-dihydro-1H-pyrrolo[2,3-b]quinolin-4-ylamine; 6-bromo-1-methyl-2,3-dihydro-1H-pyrrolo[2,3-b]quinolin-4-ylamine; 1-methyl-2,3,4,5-tetrahydro-1H-azepino[2,3-b]quinolin-6-ylamine; 3,3-dimethyl-3,4-dihydro-acridin-9-ylamine; 1-benzyl-2,3-dihydro-H-pyrrolo[2,3-b]quinolin-4-ylamine; 6-methyl-1-phenyl-2,3-dihydro-1H-pyrrolo[2,3-b]quinolin-4-ylamine; N*2*,N*2*-Dimethyl-quinoline-2,4-diamine, 2,7-Dimethyl-dibenzo[b,g][1,8]naphthyridin-11-ylamine; 2,4-Dimethyl-benzo[b][1,8]naphthyridin-5-ylamine; 7-Fluoro-2,4-dimethyl-benzo[b][1,8]naphthyridin-5-ylamine; 1,2,3,4-Tetrahydro-acridin-9-ylamine Tacrine hydrochloridehydrate; 2,3-Dihydro-1H-cyclopenta[b]quinolin-9-ylamine; 2,4,9-Trimethyl-benzo[b][1,8]naphthyridin-5-ylamine; 9-Amino-3,3-dimethyl-1,2,3,4-tetrahydro-acridin-1-ol and 7-Ethoxy-N*3*-furan-2-ylmethyl-acridine-3,9-diamine; quinazolines, N,N-dimethyl-N′-{2-[4-(4-methyl-piperazin-1-yl)-phenyl]-3,4-dihydro-quinazoline-4-yl}-ethane-1,2,-diamine; N′-[6,7-Dimethoxy-2-(4-phenyl-piperazin-1-yl)-quinazolin-4-yl]-N,N-dimethyl-ethane-1,2-diamine; N′-[6,7-Dimethoxy-2-(4-methyl-piperazin-1-yl)-quinazolin-4-yl]-N,N-dimethyl-ethane-1,2-diamine; N,N-Dimethyl-N′-(2-phenyl-quinazolin-4-yl)-ethane-1,2-diamine; Dimethyl-(2-{2-[4-(4-methyl-piperazin-1-yl)-phenyl]-quinazolin-4-yloxy}-ethyl)-amine; N′-(2-Biphenyl-4-yl-quinazolin-4-yl)-N,N-dimethyl-ethane-1,2-diamine and Dimethyl-[2-(2-phenyl-quinazolin-4-yloxy)-ethyl]-amine; ODN 2088, ODN with a TTAGGG sequence, G-ODN, statins, atorvastatin, IMO-2125 (Idera Pharmaceuticals), IRS 869, CMZ 203-84, CMZ 203-85, CMZ 203-88, CMZ 203-88-1, CMZ 203-89, CMZ 203-91, INH-ODN 2114, ODN A151, ODN INH-1, ODN INH-18, ODN 4084, ODN 4084-F, and ODN INH-47. In some embodiments, the BTK inhibitor is a compound of Formula (D)

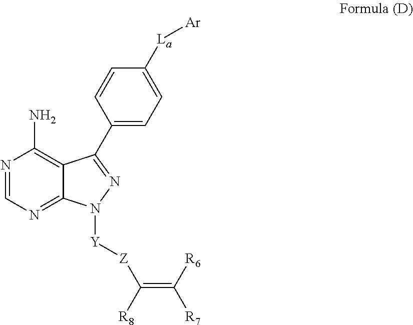

wherein

Lₐ is CH₂, O, NH or S;

Ar is an optionally substituted aromatic carbocycle or an aromatic heterocycle;

Y is an optionally substituted alkyl, heteroalkyl, carbocycle, heterocycle, or combination thereof;

Z is C(O), OC(O), NHC(O), C(S), S(O)ₓ, OS(O)ₓ, NHS(O)ₓ, where x is 1 or 2; and

R₆, R₇, and R₈ are independently selected from H, alkyl, heteroalkyl, carbocycle, heterocycle, or combinations thereof.

In some embodiments, the BTK inhibitor is ibrutinib. In some embodiments, the BTK inhibitor is ibrutinib and the TLR inhibitor is chloroquine. In some embodiments, the B-cell malignancy is a non-Hodgkin's lymphoma. In some embodiments the B-cell malignancy is diffuse large B-cell lymphoma (DLBCL), marginal zone lymphoma (MZL), acute lymphoblastic leukemia (ALL), acute myelogenous leukemia (AML), chronic myelogenous leukemia (CML), acute monocytic leukemia (AMoL), chronic lymphocytic leukemia (CLL), small lymphocytic lymphoma (SLL), high-risk small lymphocytic lymphoma (SLL), follicular lymphoma (FL), mantle cell lymphoma (MCL), Waldenstrom's macroglobulinemia, multiple myeloma, extranodal marginal zone B cell lymphoma, nodal marginal zone B cell lymphoma, Burkitt's lymphoma, non-Burkitt high grade B cell lymphoma, primary mediastinal B-cell lymphoma (PMBL), immunoblastic large cell lymphoma, precursor B-lymphoblastic lymphoma, B cell prolymphocytic leukemia, lymphoplasmacytic lymphoma, splenic marginal zone lymphoma, plasma cell myeloma, plasmacytoma, mediastinal (thymic) large B cell lymphoma, intravascular large B cell lymphoma, primary effusion lymphoma, or lymphomatoid granulomatosis. In some embodiments, the B-cell malignancy is relapsed or refractory. In some embodiments, the B-cell malignancy is diffuse large B-cell lymphoma (DLBCL). In some embodiments, the DLBCL is activated B-cell diffuse large B-cell lymphoma (ABC-DLBCL). In some embodiments, the ABC-DLBCL is characterized by a mutation in MYD88. In some embodiments, the mutation is at position 265 of MYD88. In some embodiments, the mutation is an L265P mutation. In some embodiments, the B-cell malignancy marginal zone lymphoma (MZL). In some embodiments, the BTK inhibitor is administered once a day, two times per day, three times per day, four times per day, or five times per day. In some embodiments, the BTK inhibitor is administered at a dosage of about 40 mg/day to about 1000 mg/day. In some embodiments, the BTK inhibitor is administered orally. In some embodiments, the BTK inhibitor and the TLR inhibitor are administered simultaneously, sequentially or intermittently. In some embodiments, the method further comprises administering a third therapeutic agent. In some embodiments, the third therapeutic agent is selected from among a chemotherapeutic agent or radiation therapeutic agent. In some embodiments, the chemotherapeutic agent is selected from among chlorambucil, ifosfamide, doxorubicin, mesalazine, thalidomide, lenalidomide, temsirolimus, everolimus, fludarabine, fostamatinib, paclitaxel, docetaxel, ofatumumab, rituximab, dexamethasone, prednisone, CAL-101, ibritumomab, tositumomab, bortezomib, pentostatin, endostatin, or a combination thereof.

Disclosed herein, in certain embodiments, are methods of selecting an individual having a B-cell malignancy for therapy with a combination comprising a BTK inhibitor and a TLR inhibitor, comprising: (a) detecting the presence of absence of a mutation in MYD88 in a sample from an individual; and (b) characterizing the individual as a candidate for therapy with the combination comprising a BTK inhibitor and a TLR inhibitor if the individual has a mutation in MYD88. In some embodiments, the mutation is at amino acid position 198 or 265 of MYD88. In some embodiments, the mutation at amino acid position 198 of MYD88 is S198N. In some embodiments, the mutation at amino acid position 265 of MYD88 is L265P. In some embodiments, wherein sample is a nucleic acid molecule containing sample encoding MYD88 from the individual, and the detecting comprises testing the nucleic acid molecule containing sample to determine whether the nucleic acid molecules encoding MYD88 contain the mutation. In some embodiments, the nucleic acid molecule is RNA or DNA. In some embodiments, the DNA is genomic DNA. In some embodiments, the testing comprises amplifying the nucleic acid molecules encoding MYD88. In some embodiments, the amplification is by isothermal amplification or polymerase chain reaction (PCR). In some embodiments, the amplification is by PCR. In some embodiments, the testing comprises contacting nucleic acids with sequence specific nucleic acid probes, wherein the sequence specific nucleic acid probes bind to nucleic acids encoding MYD88 having a mutation and do not bind to nucleic acid encoding wild-type MYD88. In some embodiments, the testing comprises PCR amplification using the sequence specific nucleic acid probes. In some embodiments, the sample comprises one or more tumor cells. In some embodiments, the combination provides a synergistic therapeutic effect compared to administration of the BTK inhibitor or the TLR inhibitor alone. In some embodiments, the TLR inhibitor is selected from a non-specific TLR inhibitor, a TLR7/8/9 antagonist, and a TLR9 antagonist. In some embodiments, the non-specific TLR inhibitor is selected from the group consisting of chloroquine and bafilomycin A. In some embodiments, the TLR7/8/9 antagonist is selected from the group consisting of CPG52364, IMO 8400, and IMO-9200. In some embodiments, the TLR9 antagonist is selected from the group consisting of chloroquine, quinacrine, monesin, bafilomycin A1, wortmannin, iODN, (+)-morphinans, 9-aminoacridine, 4-aminoquinoline, 4-aminoquinolines, 7,8,9,10-tetrahydro-6H-cyclohepta[b]quinolin-11-ylamine; 1-methyl-2,3-dihydro-1H-pyrrolo[2,3-b]quinolin-4-ylamine; 1,6-dimethyl-2,3-dihydro-1H-pyrrolo[2,3-b]quinolin-4-ylamine; 6-bromo-1-methyl-2,3-dihydro-1H-pyrrolo[2,3-b]quinolin-4-ylamine; 1-methyl-2,3,4,5-tetrahydro-1H-azepino[2,3-b]quinolin-6-ylamine; 3,3-dimethyl-3,4-dihydro-acridin-9-ylamine; 1-benzyl-2,3-dihydro-H-pyrrolo[2,3-b]quinolin-4-ylamine; 6-methyl-1-phenyl-2,3-dihydro-1H-pyrrolo[2,3-b]quinolin-4-ylamine; N*2*,N*2*-Dimethyl-quinoline-2,4-diamine, 2,7-Dimethyl-dibenzo[b,g][1,8]naphthyridin-11-ylamine; 2,4-Dimethyl-benzo[b][1,8]naphthyridin-5-ylamine; 7-Fluoro-2,4-dimethyl-benzo[b][1,8]naphthyridin-5-ylamine; 1,2,3,4-Tetrahydro-acridin-9-ylamine Tacrine hydrochloridehydrate; 2,3-Dihydro-1H-cyclopenta[b]quinolin-9-ylamine; 2,4,9-Trimethyl-benzo[b][1,8]naphthyridin-5-ylamine; 9-Amino-3,3-dimethyl-1,2,3,4-tetrahydro-acridin-1-ol and 7-Ethoxy-N*3*-furan-2-ylmethyl-acridine-3,9-diamine; quinazolines, N,N-dimethyl-N′-{2-[4-(4-methyl-piperazin-1-yl)-phenyl]-3,4-dihydro-quinazoline-4-yl}-ethane-1,2,-diamine; N′-[6,7-Dimethoxy-2-(4-phenyl-piperazin-1-yl)-quinazolin-4-yl]-N,N-dimethyl-ethane-1,2-diamine; N′-[6,7-Dimethoxy-2-(4-methyl-piperazin-1-yl)-quinazolin-4-yl]-N,N-dimethyl-ethane-1,2-diamine; N,N-Dimethyl-N′-(2-phenyl-quinazolin-4-yl)-ethane-1,2-diamine; Dimethyl-(2-{2-[4-(4-methyl-piperazin-1-yl)-phenyl]-quinazolin-4-yloxy}-ethyl)-amine; N′-(2-Biphenyl-4-yl-quinazolin-4-yl)-N,N-dimethyl-ethane-1,2-diamine and Dimethyl-[2-(2-phenyl-quinazolin-4-yloxy)-ethyl]-amine; ODN 2088, ODN with a TTAGGG sequence, G-ODN, statins, atorvastatin, IMO-2125 (Idera Pharmaceuticals), IRS 869, CMZ 203-84, CMZ 203-85, CMZ 203-88, CMZ 203-88-1, CMZ 203-89, CMZ 203-91, INH-ODN 2114, ODN A151, ODN INH-1, ODN INH-18, ODN 4084, ODN 4084-F, and ODN INH-47. In some embodiments, the BTK inhibitor is a compound of Formula (D)

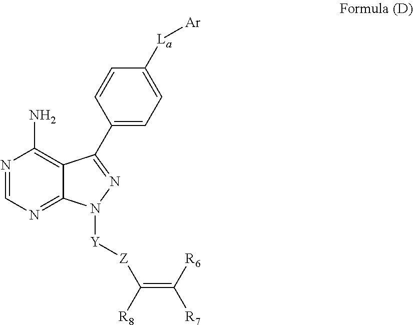

wherein

Lₐ is CH₂, O, NH or S;

Ar is an optionally substituted aromatic carbocycle or an aromatic heterocycle;

Y is an optionally substituted alkyl, heteroalkyl, carbocycle, heterocycle, or combination thereof;

Z is C(O), OC(O), NHC(O), C(S), S(O)ₓ, OS(O)ₓ, NHS(O)ₓ, where x is 1 or 2; and

R₆, R₇, and R₈ are independently selected from H, alkyl, heteroalkyl, carbocycle, heterocycle, or combinations thereof.

In some embodiments, the BTK inhibitor is ibrutinib. In some embodiments, the BTK inhibitor is ibrutinib and the TLR inhibitor is chloroquine. In some embodiments the B-cell malignancy is diffuse large B-cell lymphoma (DLBCL), marginal zone lymphoma (MZL), acute lymphoblastic leukemia (ALL), acute myelogenous leukemia (AML), chronic myelogenous leukemia (CML), acute monocytic leukemia (AMoL), chronic lymphocytic leukemia (CLL), small lymphocytic lymphoma (SLL), high-risk small lymphocytic lymphoma (SLL), follicular lymphoma (FL), mantle cell lymphoma (MCL), Waldenstrom's macroglobulinemia, multiple myeloma, extranodal marginal zone B cell lymphoma, nodal marginal zone B cell lymphoma, Burkitt's lymphoma, non-Burkitt high grade B cell lymphoma, primary mediastinal B-cell lymphoma (PMBL), immunoblastic large cell lymphoma, precursor B-lymphoblastic lymphoma, B cell prolymphocytic leukemia, lymphoplasmacytic lymphoma, splenic marginal zone lymphoma, plasma cell myeloma, plasmacytoma, mediastinal (thymic) large B cell lymphoma, intravascular large B cell lymphoma, primary effusion lymphoma, or lymphomatoid granulomatosis. In some embodiments, the B-cell malignancy is relapsed or refractory. In some embodiments, the B-cell malignancy is a non-Hodgkin's lymphoma. In some embodiments, the B-cell malignancy is diffuse large B-cell lymphoma (DLBCL). In some embodiments, the DLBCL is activated B-cell diffuse large B-cell lymphoma (ABC-DLBCL). In some embodiments, the ABC-DLBCL is characterized by a mutation in MYD88. In some embodiments, the mutation is at position 265 of MYD88. In some embodiments, the mutation is an L265P mutation. In some embodiments, the B-cell malignancy marginal zone lymphoma (MZL). In some embodiments, the method further includes administering the combination of BTK inhibitor and TLR inhibitor. In some embodiments, the BTK inhibitor is administered once a day, two times per day, three times per day, four times per day, or five times per day. In some embodiments, the BTK inhibitor is administered at a dosage of about 40 mg/day to about 1000 mg/day. In some embodiments, the BTK inhibitor is administered orally. In some embodiments, the BTK inhibitor and the TLR inhibitor are administered simultaneously, sequentially or intermittently. In some embodiments, the method further comprises administering a third therapeutic agent. In some embodiments, the third therapeutic agent is selected from among a chemotherapeutic agent or radiation therapeutic agent. In some embodiments, the chemotherapeutic agent is selected from among chlorambucil, ifosfamide, doxorubicin, mesalazine, thalidomide, lenalidomide, temsirolimus, everolimus, fludarabine, fostamatinib, paclitaxel, docetaxel, ofatumumab, rituximab, dexamethasone, prednisone, CAL-101, ibritumomab, tositumomab, bortezomib, pentostatin, endostatin, or a combination thereof.

Disclosed herein, in certain embodiments, are methods of treating a B-cell malignancy in a subject in need thereof, comprising administering to the subject a therapeutically effective amount of a combination comprising a BTK inhibitor and a TAK1 inhibitor. In some embodiments, the combination provides a synergistic therapeutic effect compared to administration of the BTK inhibitor or the TAK1 inhibitor alone. In some embodiments, the TAK1 inhibitor is selected from the group consisting of 5Z-7-oxozeaenol, LYTAK1, NG-25, celastrol, epoxyquinol B (EPQB), nemo-like kinase (NLK), USP18, VopZ, diterpene triepoxide, triptolide, 7-aminofuro[2,3-c]pyridines, naphthalimide derivatives, and oxindole derivatives. In some embodiments, the TAK1 inhibitor is 5Z-7-oxozeaenol. In some embodiments, the BTK inhibitor is a compound of Formula (D)

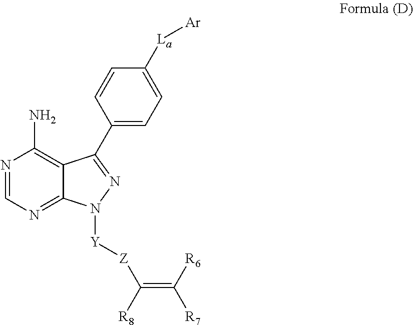

wherein

Lₐ is CH₂, O, NH or S;

Ar is an optionally substituted aromatic carbocycle or an aromatic heterocycle;

Y is an optionally substituted alkyl, heteroalkyl, carbocycle, heterocycle, or combination thereof;

Z is C(O), OC(O), NHC(O), C(S), S(O)ₓ, OS(O)ₓ, NHS(O)ₓ, where x is 1 or 2; and

R₆, R₇, and R₈ are independently selected from H, alkyl, heteroalkyl, carbocycle, heterocycle, or combinations thereof.

In some embodiments, the BTK inhibitor is ibrutinib. In some embodiments, the BTK inhibitor is ibrutinib and the TAK1 inhibitor is 5Z-7-oxozeaenol. In some embodiments the B-cell malignancy is diffuse large B-cell lymphoma (DLBCL), marginal zone lymphoma (MZL), acute lymphoblastic leukemia (ALL), acute myelogenous leukemia (AML), chronic myelogenous leukemia (CML), acute monocytic leukemia (AMoL), chronic lymphocytic leukemia (CLL), small lymphocytic lymphoma (SLL), high-risk small lymphocytic lymphoma (SLL), follicular lymphoma (FL), mantle cell lymphoma (MCL), Waldenstrom's macroglobulinemia, multiple myeloma, extranodal marginal zone B cell lymphoma, nodal marginal zone B cell lymphoma, Burkitt's lymphoma, non-Burkitt high grade B cell lymphoma, primary mediastinal B-cell lymphoma (PMBL), immunoblastic large cell lymphoma, precursor B-lymphoblastic lymphoma, B cell prolymphocytic leukemia, lymphoplasmacytic lymphoma, splenic marginal zone lymphoma, plasma cell myeloma, plasmacytoma, mediastinal (thymic) large B cell lymphoma, intravascular large B cell lymphoma, primary effusion lymphoma, or lymphomatoid granulomatosis. In some embodiments, the B-cell malignancy is relapsed or refractory. In some embodiments, the B-cell malignancy is a non-Hodgkin's lymphoma. In some embodiments, the B-cell malignancy is diffuse large B-cell lymphoma (DLBCL). In some embodiments, the DLBCL is activated B-cell diffuse large B-cell lymphoma (ABC-DLBCL). In some embodiments, the ABC-DLBCL is characterized by a mutation in MYD88. In some embodiments, the mutation is at position 265 of MYD88. In some embodiments, the mutation is an L265P mutation. In some embodiments, the B-cell malignancy marginal zone lymphoma (MZL). In some embodiments, the BTK inhibitor is administered once a day, two times per day, three times per day, four times per day, or five times per day. In some embodiments, the BTK inhibitor is administered at a dosage of about 40 mg/day to about 1000 mg/day. In some embodiments, the BTK inhibitor is administered orally. In some embodiments, the BTK inhibitor and the TAK1 inhibitor are administered simultaneously, sequentially or intermittently. In some embodiments, the method further comprises administering a third therapeutic agent. In some embodiments, the third therapeutic agent is selected from among a chemotherapeutic agent or radiation therapeutic agent. In some embodiments, the chemotherapeutic agent is selected from among chlorambucil, ifosfamide, doxorubicin, mesalazine, thalidomide, lenalidomide, temsirolimus, everolimus, fludarabine, fostamatinib, paclitaxel, docetaxel, ofatumumab, rituximab, dexamethasone, prednisone, CAL-101, ibritumomab, tositumomab, bortezomib, pentostatin, endostatin, or a combination thereof.

Disclosed herein, in certain embodiments, are pharmaceutical combinations comprising a BTK inhibitor and a TLR inhibitor. In some embodiments, the combination further comprises a pharmaceutically-acceptable excipient. In some embodiments, the combination provides a synergistic therapeutic effect compared to administration of the BTK inhibitor or the TLR inhibitor alone. In some embodiments, the TLR inhibitor is selected from a non-specific TLR inhibitor, a TLR7/8/9 antagonist, and a TLR9 antagonist. In some embodiments, the non-specific TLR inhibitor is selected from the group consisting of chloroquine and bafilomycin A. In some embodiments, the TLR7/8/9 antagonist is selected from the group consisting of CPG52364, IMO 8400, and IMO-9200. In some embodiments, the TLR9 antagonist is selected from the group consisting of chloroquine, quinacrine, monesin, bafilomycin A1, wortmannin, iODN, (+)-morphinans, 9-aminoacridine, 4-aminoquinoline, 4-aminoquinolines, 7,8,9,10-tetrahydro-6H-cyclohepta[b]quinolin-11-ylamine; 1-methyl-2,3-dihydro-1H-pyrrolo[2,3-b]quinolin-4-ylamine; 1,6-dimethyl-2,3-dihydro-1H-pyrrolo[2,3-b]quinolin-4-ylamine; 6-bromo-1-methyl-2,3-dihydro-1H-pyrrolo[2,3-b]quinolin-4-ylamine; 1-methyl-2,3,4,5-tetrahydro-1H-azepino[2,3-b]quinolin-6-ylamine; 3,3-dimethyl-3,4-dihydro-acridin-9-ylamine; 1-benzyl-2,3-dihydro-H-pyrrolo[2,3-b]quinolin-4-ylamine; 6-methyl-1-phenyl-2,3-dihydro-1H-pyrrolo[2,3-b]quinolin-4-ylamine; N*2*,N*2*-Dimethyl-quinoline-2,4-diamine, 2,7-Dimethyl-dibenzo[b,g][1,8]naphthyridin-11-ylamine; 2,4-Dimethyl-benzo[b][1,8]naphthyridin-5-ylamine; 7-Fluoro-2,4-dimethyl-benzo[b][1,8]naphthyridin-5-ylamine; 1,2,3,4-Tetrahydro-acridin-9-ylamine Tacrine hydrochloridehydrate; 2,3-Dihydro-1H-cyclopenta[b]quinolin-9-ylamine; 2,4,9-Trimethyl-benzo[b][1,8]naphthyridin-5-ylamine; 9-Amino-3,3-dimethyl-1,2,3,4-tetrahydro-acridin-1-ol and 7-Ethoxy-N*3*-furan-2-ylmethyl-acridine-3,9-diamine; quinazolines, N,N-dimethyl-N′-{2-[4-(4-methyl-piperazin-1-yl)-phenyl]-3,4-dihydro-quinazoline-4-yl}-ethane-1,2,-diamine; N′-[6,7-Dimethoxy-2-(4-phenyl-piperazin-1-yl)-quinazolin-4-yl]-N,N-dimethyl-ethane-1,2-diamine; N′-[6,7-Dimethoxy-2-(4-methyl-piperazin-1-yl)-quinazolin-4-yl]-N,N-dimethyl-ethane-1,2-diamine; N,N-Dimethyl-N′-(2-phenyl-quinazolin-4-yl)-ethane-1,2-diamine; Dimethyl-(2-{2-[4-(4-methyl-piperazin-1-yl)-phenyl]-quinazolin-4-yloxy}-ethyl)-amine; N′-(2-Biphenyl-4-yl-quinazolin-4-yl)-N,N-dimethyl-ethane-1,2-diamine and Dimethyl-[2-(2-phenyl-quinazolin-4-yloxy)-ethyl]-amine; ODN 2088, ODN with a TTAGGG sequence, G-ODN, statins, atorvastatin, IMO-2125 (Idera Pharmaceuticals), IRS 869, CMZ 203-84, CMZ 203-85, CMZ 203-88, CMZ 203-88-1, CMZ 203-89, CMZ 203-91, INH-ODN 2114, ODN A151, ODN INH-1, ODN INH-18, ODN 4084, ODN 4084-F, and ODN INH-47. In some embodiments, the BTK inhibitor is a compound of Formula (D)

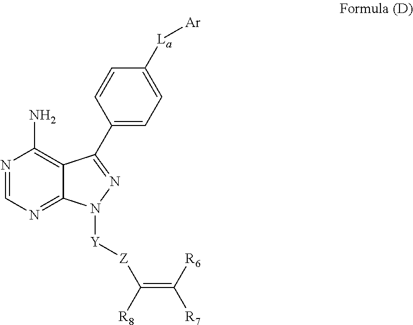

wherein

Lₐ is CH₂, O, NH or S;

Ar is an optionally substituted aromatic carbocycle or an aromatic heterocycle;

Y is an optionally substituted alkyl, heteroalkyl, carbocycle, heterocycle, or combination thereof;

Z is C(O), OC(O), NHC(O), C(S), S(O)ₓ, OS(O)ₓ, NHS(O)ₓ, where x is 1 or 2; and

R₆, R₇, and R₈ are independently selected from H, alkyl, heteroalkyl, carbocycle, heterocycle, or combinations thereof. In some embodiments, the BTK inhibitor is ibrutinib.

In some embodiments, the BTK inhibitor is ibrutinib and the TLR inhibitor is chloroquine. In some embodiments, the combination is in a combined dosage form. In some embodiments, the combination is in separate dosage forms.

Disclosed herein, in certain embodiments, are pharmaceutical combinations comprising a BTK inhibitor and a TAK1 inhibitor. In some embodiments, the combination further comprises a pharmaceutically-acceptable excipient. In some embodiments, the combination provides a synergistic therapeutic effect compared to administration of the BTK inhibitor or the TAK1 inhibitor alone. In some embodiments, the TAK1 inhibitor is selected from the group consisting of 5Z-7-oxozeaenol, LYTAK1, NG-25, celastrol, epoxyquinol B (EPQB), nemo-like kinase (NLK), USP18, VopZ, diterpene triepoxide, triptolide, 7-aminofuro[2,3-c]pyridines, naphthalimide derivatives, and oxindole derivatives. In some embodiments, the TAK1 inhibitor is 5Z-7-oxozeaenol. In some embodiments, the BTK inhibitor is a compound of Formula (D)

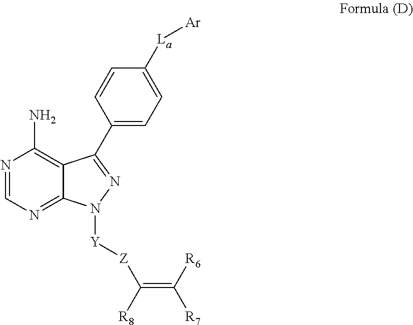

wherein

Lₐ is CH₂, O, NH or S;

Ar is an optionally substituted aromatic carbocycle or an aromatic heterocycle;

Y is an optionally substituted alkyl, heteroalkyl, carbocycle, heterocycle, or combination thereof;

Z is C(O), OC(O), NHC(O), C(S), S(O)ₓ, OS(O)ₓ, NHS(O)ₓ, where x is 1 or 2; and

R₆, R₇, and R₈ are independently selected from H, alkyl, heteroalkyl, carbocycle, heterocycle, or combinations thereof.

In some embodiments, the BTK inhibitor is ibrutinib. In some embodiments, the BTK inhibitor is ibrutinib and the TAK1 inhibitor is 5Z-7-oxozeaenol. In some embodiments, the combination is in a combined dosage form. In some embodiments, the combination is in separate dosage forms.

Disclosed herein, in certain embodiments, is a method of treating a non-Hodgkin's lymphoma in a subject in need thereof, comprising administering to the subject a therapeutically effective amount of a combination comprising a BTK inhibitor and a TLR inhibitor. In some embodiments, the combination provides a synergistic therapeutic effect compared to administration of the BTK inhibitor or the TLR inhibitor alone. In some embodiments, the TLR inhibitor is selected from a non-specific TLR inhibitor, a TLR6/7/8/9 antagonist, and a TLR9 antagonist. In some embodiments, the non-specific TLR inhibitor is selected from the group consisting of chloroquine and bafilomycin A. In some embodiments, the TLR7/8/9 antagonist is selected from the group consisting of CPG52364, IMO 8400, and IMO-9200. In some embodiments, the TLR9 antagonist is selected from the group consisting of chloroquine, quinacrine, monesin, bafilomycin A1, wortmannin, iODN, (+)-morphinans, 9-aminoacridine, 4-aminoquinoline, 4-aminoquinolines, 7,8,9,10-tetrahydro-6H-cyclohepta[b]quinolin-11-ylamine; 1-methyl-2,3-dihydro-1H-pyrrolo[2,3-b]quinolin-4-ylamine; 1,6-dimethyl-2,3-dihydro-H-pyrrolo[2,3-b]quinolin-4-ylamine; 6-bromo-1-methyl-2,3-dihydro-1H-pyrrolo[2,3-b]quinolin-4-ylamine; 1-methyl-2,3,4,5-tetrahydro-1H-azepino[2,3-b]quinolin-6-ylamine; 3,3-dimethyl-3,4-dihydro-acridin-9-ylamine; 1-benzyl-2,3-dihydro-1H-pyrrolo[2,3-b]quinolin-4-ylamine; 6-methyl-1-phenyl-2,3-dihydro-1H-pyrrolo[2,3-b]quinolin-4-ylamine; N*2*,N*2*-Dimethyl-quinoline-2,4-diamine, 2,7-Dimethyl-dibenzo[b,g][1,8]naphthyridin-11-ylamine; 2,4-Dimethyl-benzo[b][1,8]naphthyridin-5-ylamine; 7-Fluoro-2,4-dimethyl-benzo[b][1,8]naphthyridin-5-ylamine; 1,2,3,4-Tetrahydro-acridin-9-ylamine Tacrine hydrochloridehydrate; 2,3-Dihydro-1H-cyclopenta[b]quinolin-9-ylamine; 2,4,9-Trimethyl-benzo[b][1,8]naphthyridin-5-ylamine; 9-Amino-3,3-dimethyl-1,2,3,4-tetrahydro-acridin-1-ol and 7-Ethoxy-N*3*-furan-2-ylmethyl-acridine-3,9-diamine; quinazolines, N,N-dimethyl-N′-{2-[4-(4-methyl-piperazin-1-yl)-phenyl]-3,4-dihydro-quinazoline-4-yl}-ethane-1,2,-diamine; N′-[6,7-Dimethoxy-2-(4-phenyl-piperazin-1-yl)-quinazolin-4-yl]-N,N-dimethyl-ethane-1,2-diamine; N′-[6,7-Dimethoxy-2-(4-methyl-piperazin-1-yl)-quinazolin-4-yl]-N,N-dimethyl-ethane-1,2-diamine; N,N-Dimethyl-N′-(2-phenyl-quinazolin-4-yl)-ethane-1,2-diamine; Dimethyl-(2-{2-[4-(4-methyl-piperazin-1-yl)-phenyl]-quinazolin-4-yloxy}-ethyl)-amine; N′-(2-Biphenyl-4-yl-quinazolin-4-yl)-N,N-dimethyl-ethane-1,2-diamine and Dimethyl-[2-(2-phenyl-quinazolin-4-yloxy)-ethyl]-amine; ODN 2088, ODN with a TTAGGG sequence, G-ODN, statins, atorvastatin, IMO-2125 (Idera Pharmaceuticals), IRS 869, CMZ 203-84, CMZ 203-85, CMZ 203-88, CMZ 203-88-1, CMZ 203-89, CMZ 203-91, INH-ODN 2114, ODN A151, ODN INH-1, ODN INH-18, ODN 4084, ODN 4084-F, and ODN INH-47. In some embodiments, the BTK inhibitor is ibrutinib. In some embodiments, the non-Hodgkin's lymphoma is marginal zone lymphoma (MZL), extranodal marginal zone B-cell lymphoma (also known as mucosa-associated lymphoid tissue (MALT) lymphomas), nodal marginal zone B-cell lymphoma, splenic marginal zone B-cell lymphoma, lymphoplasmacytic lymphoma (Waldenstrom's macroglobulinemia), hairy cell leukemia, primary central nervous system (CNS) lymphoma, Burkitt lymphoma, chronic lymphocytic leukemia/small lymphocytic lymphoma (CLL/SLL), diffuse large B-cell lymphoma (DLBCL), primary mediastinal B-cell lymphoma, Intravascular large B-cell lymphoma, follicular lymphoma, immunoblastic large cell lymphoma, precursor B-lymphoblastic lymphoma, or mantle cell lymphoma. In some embodiments, the non-Hodgkin's lymphoma is DLBCL. In some embodiments, DLBCL is activated B-cell diffuse large B-cell lymphoma (ABC-DLBCL). In some embodiments, the ABC-DLBCL is characterized by a mutation in MYD88. In some embodiments, the mutation is at position 265 of MYD88. In some embodiments, the mutation is an L265P mutation. In some embodiments, the non-Hodgkin's lymphoma is MZL. In some embodiments, the non-Hodgkin's lymphoma is a relapsed or refractory non-Hodgkin's lymphoma. In some embodiments, the non-Hodgkin's lymphoma is an ibrutinib-resistant non-Hodgkin's lymphoma.

Disclosed herein, in certain embodiments, is a method of treating an ibrutinib-resistant non-Hodgkin's lymphoma in a subject in need thereof, comprising administering to the subject a therapeutically effective amount of a combination comprising ibrutinib and a TLR inhibitor. In some embodiments, the combination provides a synergistic therapeutic effect compared to administration of ibrutinib or the TLR inhibitor alone. In some embodiments, the TLR inhibitor is selected from a non-specific TLR inhibitor, a TLR6/7/8/9 antagonist, and a TLR9 antagonist. In some embodiments, the non-specific TLR inhibitor is selected from the group consisting of chloroquine and bafilomycin A. In some embodiments, the TLR7/8/9 antagonist is selected from the group consisting of CPG52364, IMO 8400, and IMO-9200. In some embodiments, the TLR9 antagonist is selected from the group consisting of chloroquine, quinacrine, monesin, bafilomycin A1, wortmannin, iODN, (+)-morphinans, 9-aminoacridine, 4-aminoquinoline, 4-aminoquinolines, 7,8,9,10-tetrahydro-6H-cyclohepta[b]quinolin-11-ylamine; 1-methyl-2,3-dihydro-1H-pyrrolo[2,3-b]quinolin-4-ylamine; 1,6-dimethyl-2,3-dihydro-1H-pyrrolo[2,3-b]quinolin-4-ylamine; 6-bromo-1-methyl-2,3-dihydro-1H-pyrrolo[2,3-b]quinolin-4-ylamine; 1-methyl-2,3,4,5-tetrahydro-1H-azepino[2,3-b]quinolin-6-ylamine; 3,3-dimethyl-3,4-dihydro-acridin-9-ylamine; 1-benzyl-2,3-dihydro-1H-pyrrolo[2,3-b]quinolin-4-ylamine; 6-methyl-1-phenyl-2,3-dihydro-1H-pyrrolo[2,3-b]quinolin-4-ylamine; N*2*,N*2*-Dimethyl-quinoline-2,4-diamine, 2,7-Dimethyl-dibenzo[b,g][1,8]naphthyridin-11-ylamine; 2,4-Dimethyl-benzo[b][1,8]naphthyridin-5-ylamine; 7-Fluoro-2,4-dimethyl-benzo[b][1,8]naphthyridin-5-ylamine; 1,2,3,4-Tetrahydro-acridin-9-ylamine Tacrine hydrochloridehydrate; 2,3-Dihydro-1H-cyclopenta[b]quinolin-9-ylamine; 2,4,9-Trimethyl-benzo[b][1,8]naphthyridin-5-ylamine; 9-Amino-3,3-dimethyl-1,2,3,4-tetrahydro-acridin-1-ol and 7-Ethoxy-N*3*-furan-2-ylmethyl-acridine-3,9-diamine; quinazolines, N,N-dimethyl-N′-{2-[4-(4-methyl-piperazin-1-yl)-phenyl]-3,4-dihydro-quinazoline-4-yl}-ethane-1,2,-diamine; N′-[6,7-Dimethoxy-2-(4-phenyl-piperazin-1-yl)-quinazolin-4-yl]-N,N-dimethyl-ethane-1,2-diamine; N′-[6,7-Dimethoxy-2-(4-methyl-piperazin-1-yl)-quinazolin-4-yl]-N,N-dimethyl-ethane-1,2-diamine; N,N-Dimethyl-N′-(2-phenyl-quinazolin-4-yl)-ethane-1,2-diamine; Dimethyl-(2-{2-[4-(4-methyl-piperazin-1-yl)-phenyl]-quinazolin-4-yloxy}-ethyl)-amine; N′-(2-Biphenyl-4-yl-quinazolin-4-yl)-N,N-dimethyl-ethane-1,2-diamine and Dimethyl-[2-(2-phenyl-quinazolin-4-yloxy)-ethyl]-amine; ODN 2088, ODN with a TTAGGG sequence, G-ODN, statins, atorvastatin, IMO-2125 (Idera Pharmaceuticals), IRS 869, CMZ 203-84, CMZ 203-85, CMZ 203-88, CMZ 203-88-1, CMZ 203-89, CMZ 203-91, INH-ODN 2114, ODN A151, ODN INH-1, ODN INH-18, ODN 4084, ODN 4084-F, and ODN INH-47. In some embodiments, the ibrutinib-resistant non-Hodgkin's lymphoma is marginal zone lymphoma (MZL), extranodal marginal zone B-cell lymphoma (also known as mucosa-associated lymphoid tissue (MALT) lymphomas), nodal marginal zone B-cell lymphoma, splenic marginal zone B-cell lymphoma, lymphoplasmacytic lymphoma (Waldenstrom macroglobulinemia), hairy cell leukemia, primary central nervous system (CNS) lymphoma, Burkitt lymphoma, chronic lymphocytic leukemia/small lymphocytic lymphoma (CLL/SLL), diffuse large B-cell lymphoma (DLBCL), primary mediastinal B-cell lymphoma, Intravascular large B-cell lymphoma, follicular lymphoma, immunoblastic large cell lymphoma, precursor B-lymphoblastic lymphoma, or mantle cell lymphoma. In some embodiments, the ibrutinib-resistant non-Hodgkin's lymphoma is ibrutinib-resistant DLBCL. In some embodiments, the ibrutinib-resistant DLBCL is ibrutinib-resistant activated B-cell diffuse large B-cell lymphoma (ABC-DLBCL). In some embodiments, the ibrutinib-resistant ABC-DLBCL is characterized by a mutation in MYD88. In some embodiments, the mutation is at position 265 of MYD88. In some embodiments, the mutation is an L265P mutation. In some embodiments, the ibrutinib-resistant non-Hodgkin's lymphoma is ibrutinib-resistant MZL.

Disclosed herein, in certain embodiments, is a method of selecting a subject having a non-Hodgkin's lymphoma for treatment with a combination of a BTK inhibitor and a TLR inhibitor, comprising: (a) determining the expression level of a TLR biomarker or a TLR-related biomarker; and (b) administering to the individual a therapeutically effective amount of a combination of a BTK inhibitor and a TLR inhibitor if there is no increase in the expression level of the TLR biomarker or the TLR-related biomarker relative to a control. Also disclosed herein, in certain embodiments, is a method of monitoring the disease progression in a subject having a non-Hodgkin's lymphoma, comprising: (a) determining the expression level of a TLR biomarker or a TLR-related biomarker; and (b) characterizing the subject as developed a resistance to a BTK inhibitor if the subject shows an increase in expression level of the TLR biomarker or the TLR-related biomarker relative to a control. In some embodiments, the expression level of the TLR biomarker or the TLR-related biomarker increases by 0.5-fold, 1-fold, 1.5-fold, 2-fold, 2.5-fold, 3-fold, 3.5-fold, 4-fold, 4.5-fold, 5-fold, 5.5-fold, 6-fold, 6.5-fold, 7-fold, 7.5-fold, 8-fold, 8.5-fold, 9-fold, 9.5-fold, 10-fold, 15-fold, 20-fold, 50-fold, or more compared to the control. In some embodiments, the control is the expression levels of the TLR biomarker or the TLR-related biomarker in an individual who is not insensitive toward the BTK inhibitor. In some embodiments, the control is the expression levels of the TLR biomarker or the TLR-related biomarker in an individual who has not been treated with the BTK inhibitor. In some embodiments, the TLR biomarker comprises TLR2, TLR3, TLR4, TLR5, or TLR9. In some embodiments, the TLR-related biomarker comprises a TLR interacting molecule, a TLR downstream effector, or a TLR-related cytokine or chemokine. In some embodiments, the TLR interacting molecule comprises CD14, HSPA1A, LY96, JIP3, RIPK2, or TIRAP. In some embodiments, the TLR downstream effector comprises CASP8, CHUK, EIF2AK2, IKBKB, IRAK2, IRF1, MAP2K4, NFKB2, NFKBIL1, NFRKB, PPARA, PTGS2, RELA, TAB1, or TRAF6. In some embodiments, the TLR related cytokine or chemokine comprises CCL2, CSF2, CSF3, CXCL10, IFNA1, IFNB1, IFNG, IL12A, IL1A, IL1B, IL2, IL6, IL8, or LTA. In some embodiments, the TLR inhibitor is selected from a non-specific TLR inhibitor, a TLR6/7/8/9 antagonist, and a TLR9 antagonist. In some embodiments, the non-specific TLR inhibitor is selected from the group consisting of chloroquine and bafilomycin A. In some embodiments, the TLR7/8/9 antagonist is selected from the group consisting of CPG52364, IMO 8400, and IMO-9200. In some embodiments, the TLR9 antagonist is selected from the group consisting of chloroquine, quinacrine, monesin, bafilomycin A1, wortmannin, iODN, (+)-morphinans, 9-aminoacridine, 4-aminoquinoline, 4-aminoquinolines, 7,8,9,10-tetrahydro-6H-cyclohepta[b]quinolin-11-ylamine; 1-methyl-2,3-dihydro-1H-pyrrolo[2,3-b]quinolin-4-ylamine; 1,6-dimethyl-2,3-dihydro-1H-pyrrolo[2,3-b]quinolin-4-ylamine; 6-bromo-1-methyl-2,3-dihydro-1H-pyrrolo[2,3-b]quinolin-4-ylamine; 1-methyl-2,3,4,5-tetrahydro-1H-azepino[2,3-b]quinolin-6-ylamine; 3,3-dimethyl-3,4-dihydro-acridin-9-ylamine; 1-benzyl-2,3-dihydro-1H-pyrrolo[2,3-b]quinolin-4-ylamine; 6-methyl-1-phenyl-2,3-dihydro-1H-pyrrolo[2,3-b]quinolin-4-ylamine; N*2*,N*2*-Dimethyl-quinoline-2,4-diamine, 2,7-Dimethyl-dibenzo[b,g][1,8]naphthyridin-11-ylamine; 2,4-Dimethyl-benzo[b][1,8]naphthyridin-5-ylamine; 7-Fluoro-2,4-dimethyl-benzo[b][1,8]naphthyridin-5-ylamine; 1,2,3,4-Tetrahydro-acridin-9-ylamine Tacrine hydrochloridehydrate; 2,3-Dihydro-1H-cyclopenta[b]quinolin-9-ylamine; 2,4,9-Trimethyl-benzo[b][1,8]naphthyridin-5-ylamine; 9-Amino-3,3-dimethyl-1,2,3,4-tetrahydro-acridin-1-ol and 7-Ethoxy-N*3*-furan-2-ylmethyl-acridine-3,9-diamine; quinazolines, N,N-dimethyl-N′-{2-[4-(4-methyl-piperazin-1-yl)-phenyl]-3,4-dihydro-quinazoline-4-yl}-ethane-1,2,-diamine; N′-[6,7-Dimethoxy-2-(4-phenyl-piperazin-1-yl)-quinazolin-4-yl]-N,N-dimethyl-ethane-1,2-diamine; N′-[6,7-Dimethoxy-2-(4-methyl-piperazin-1-yl)-quinazolin-4-yl]-N,N-dimethyl-ethane-1,2-diamine; N,N-Dimethyl-N′-(2-phenyl-quinazolin-4-yl)-ethane-1,2-diamine; Dimethyl-(2-{2-[4-(4-methyl-piperazin-1-yl)-phenyl]-quinazolin-4-yloxy}-ethyl)-amine; N′-(2-Biphenyl-4-yl-quinazolin-4-yl)-N,N-dimethyl-ethane-1,2-diamine and Dimethyl-[2-(2-phenyl-quinazolin-4-yloxy)-ethyl]-amine; ODN 2088, ODN with a TTAGGG sequence, G-ODN, statins, atorvastatin, IMO-2125 (Idera Pharmaceuticals), IRS 869, CMZ 203-84, CMZ 203-85, CMZ 203-88, CMZ 203-88-1, CMZ 203-89, CMZ 203-91, INH-ODN 2114, ODN A151, ODN INH-1, ODN INH-18, ODN 4084, ODN 4084-F, and ODN INH-47. In some embodiments, the BTK inhibitor is ibrutinib. In some embodiments, the non-Hodgkin's lymphoma is marginal zone lymphoma (MZL), extranodal marginal zone B-cell lymphoma (also known as mucosa-associated lymphoid tissue (MALT) lymphomas), nodal marginal zone B-cell lymphoma, splenic marginal zone B-cell lymphoma, lymphoplasmacytic lymphoma (Waldenstrom macroglobulinemia), hairy cell leukemia, primary central nervous system (CNS) lymphoma, Burkitt lymphoma, chronic lymphocytic leukemia/small lymphocytic lymphoma (CLL/SLL), diffuse large B-cell lymphoma (DLBCL), primary mediastinal B-cell lymphoma, Intravascular large B-cell lymphoma, follicular lymphoma, immunoblastic large cell lymphoma, precursor B-lymphoblastic lymphoma, or mantle cell lymphoma. In some embodiments, the non-Hodgkin's lymphoma is DLBCL. In some embodiments, DLBCL is activated B-cell diffuse large B-cell lymphoma (ABC-DLBCL). In some embodiments, the ABC-DLBCL is characterized by a mutation in MYD88. In some embodiments, the mutation is at position 265 of MYD88. In some embodiments, the mutation is an L265P mutation. In some embodiments, the non-Hodgkin's lymphoma is MZL. In some embodiments, the non-Hodgkin's lymphoma is a relapsed or refractory non-Hodgkin's lymphoma. In some embodiments, the non-Hodgkin's lymphoma is an ibrutinib-resistant non-Hodgkin's lymphoma.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1A-FIG. 1D illustrate the combination of chloroquine with ibrutinib in the presence or absence (no stimulation) of TLR9 agonists (ODN 2006, ODN 2216, or ODN 2395), as compared to neutral ODN with ibrutinib, in TMD8 cells.

FIG. 2A-FIG. 2C illustrate the combination of TLR9 antagonist (ODN TTAGGG) with ibrutinib in the presence or absence (no stimulation) of TLR9 agonists (ODN 2216 or ODN 2395), as compared to neutral ODN with ibrutinib, in TMD8 cells.

FIG. 3 illustrates combinations of different TLR9 antagonists with ibrutinib in the presence of TLR9 agonist (ODN 2116), as compared to neutral ODN with ibrutinib, in TMD8 cells.

FIG. 4A-FIG. 4D illustrate the combination of chloroquine with ibrutinib in the presence or absence of TLR9 agonist (ODN 2116), as compared to ibrutinib in vehicle, in HBL1 and LY10 cells.

FIG. 5 illustrates the combination of TLR9 antagonist (ODN INH-1) with ibrutinib, as compared to neutral ODN with ibrutinib, in HBL1 cells.

FIG. 6 illustrates the combination of TAK1 inhibitor (5Z-7-oxozeaenol) with ibrutinib in TMD8 cells.

FIG. 7A-FIG. 7D illustrate the synergistic growth suppression effect of ibrutinib and TLR inhibitor in ABC-DLBCL cells. FIG. 7A shows the combination index (C.I.) of ibrutinib combination with TLR inhibitor at indicated concentrations in TMD8 cells. FIG. 7B shows the drug dose matrix data of TMD8 cell line. The numbers indicate the percentage of growth inhibition of cells treated for 3 days with the corresponding compound combination relative to vehicle control-treated cells. The data were visualized over matrix using a color scale. FIG. 7C exemplifies an isobologram analysis of the data in FIG. 7B. The analysis indicates strong synergy for the combination of ibrutinib and TLR inhibitor. FIG. 7D shows the synergy scores of ibrutinib combined with TLR inhibitor in ABC-DLBCL cell lines with or without the stimulation of TLR9 agonist ODN 2216.

FIG. 8 illustrates increased ibrutinib sensitivity in TMD8 cells by TLR9 antagonists in the presence or absence of TLR9 agonist stimulation. TMD8 cells were treated with indicated concentrations of ibrutinib combined with TLR9 antagonists (ODN 4084-F, ODN INH-1, ODN INH-18, or ODN TTAGGG) or neutral ODN control in the absence (A) or presence of TLR9 agonists ODN 2216 (B) or ODN 2395 (C) for 3 days and the drug effect on cell growth was determined by CellTiter-Glo® luminescent cell viability assay.

FIG. 9 exemplifies increased ibrutinib sensitivity in TMD8 cells by TAK1 inhibitor. In panel A, TMD8 cells were treated with indicated concentrations of ibrutinib combined with TAK1 inhibitor (100 nM) or vehicle control for 3 days and the drug effect on cell growth was determined by CellTiter-Glo® luminescent cell viability assay. Panel B shows the combination index (C.I.) and synergy score of ibrutinib combined with TAK1 inhibitor in TMD8 cells.

FIG. 10 illustrates the combination of ibrutinib and TLR inhibitor in increased autophagic cell death in TMD8 cells. In panel A, TMD8 cells were treated for 2 days with ibrutinib (100 nM), TLR inhibitor (40 μM), or a combination, and analyzed for annexin-V binding and for PI uptake. The percentage of cells as annexin V positive, PI positive or double positive for both annexin V and PI are indicated. In panel B, the autophagic marker LC3B-II analysis by Western Blot was performed 1 or 2 days after indicated drug treatment. B-actin was used as a loading control.

FIG. 11 shows the combination of ibrutinib and TLR inhibitor on colony formation in HBL-1 cells. The combination reduces colony formation. HBL-1 cells were plated in 0.9% MethoCult (1000 cells/well) with indicated drug treatment and colony formation was scored after 7 days. Each graph represents quantification of 3 wells, expressed as mean±SD.

FIG. 12 exemplifies ibrutinib sensitivity in ABC-DLBCL cell lines in the presence of TLR9 agonist ODN2216. ODN2216 reduces ibrutinib sensitivity. ABC-DLBCL cell lines (A) TMD-8, (B) HBL-1, and (C) OCI-LY10 were treated with indicated concentrations of ibrutinib with or without the stimulation of TLR9 agonist ODN 2216 (1 μM) for 3 days and the drug effect on cell growth was determined by CellTiter-Glo® luminescent cell viability assay.

FIG. 13 shows the TLR gene expression in ibrutinib-resistant ABC-DLBCL cells. The gene expressions panels are illustrated as TLRs (A), TLR interacting molecules (B), TLR downstream effectors (C), and TLR related cytokines/chemokines (D) in TMD8 and HBL-1 cells. The gene expressions were measured by qPCR. Expression data were normalized to microglobulin, GAPDH, and HPRT1 reference genes. All data were presented as gene expression fold change of ibrutinib-resistant samples relative to wild-type (WT) control samples.

FIG. 14 (A)-FIG. 14(D) shows the effect of PIM1 muations on the upstream regulators of NF-kB signaling. TLR4, TLR7, IL1R1, TNFSF15, FASLG, TNF, TNFRSF10A, TNFRSF10B, TNFSF1A, CD40, and LTBR all exhibited higher relative gene expression compared to other genes iterated on the figure.

FIG. 15(A)-FIG. 15(B) shows the enrichment of genes associated with TLR and ILIA signalling pathways in PIM1 mutant cells. Graphs show upregulation of TLR and ILIA signalling pathways in PIM1 mutant cells.

FIG. 16(A)-FIG. 16(B) shows the relative expression of TLR4 and IL1R1 in different subpopulations of patients. More specifically, patients with progressive and stable disease have a significantly higher expression of TLR4 when compared to patients with complete response or partial response to theatment. Similarly, patients with progressive and stable disease have a significantly higher expression of ILR1 when compared to patients with complete response or partial response to treatment.

### DETAILED DESCRIPTION OF THE INVENTION

### Certain Terminology

Unless defined otherwise, all technical and scientific terms used herein have the same meaning as is commonly understood by one of skill in the art to which the claimed subject matter belongs. It is to be understood that the foregoing general description and the following detailed description are exemplary and explanatory only and are not restrictive of any subject matter claimed. In this application, the use of the singular includes the plural unless specifically stated otherwise. It must be noted that, as used in the specification and the appended claims, the singular forms “a,” “an” and “the” include plural referents unless the context clearly dictates otherwise. In this application, the use of “or” means “and/or” unless stated otherwise. Furthermore, use of the term “including” as well as other forms, such as “include”, “includes,” and “included,” is not limiting.

As used herein, ranges and amounts can be expressed as “about” a particular value or range. About also includes the exact amount. Hence “about 5 μL” means “about 5 μL” and also “5 μL.” Generally, the term “about” includes an amount that would be expected to be within experimental error.

The section headings used herein are for organizational purposes only and are not to be construed as limiting the subject matter described.

Bruton's Tyrosine Kinase (BTK) and TLR Overview

BTK is a key regulator of B-cell development, activation, signaling, and survival (Kurosaki, Curr Op Imm, 2000, 276-281; Schaeffer and Schwartzberg, Curr Op Imm 2000, 282-288). It plays a role in a number of other hematopoietic cell signaling pathways, e.g., Toll like receptor (TLR) and cytokine receptor-mediated TNF-α production in macrophages, IgE receptor (FcεRI) signaling in Mast cells, inhibition of Fas/APO-1 apoptotic signaling in B-lineage lymphoid cells, and collagen-stimulated platelet aggregation. See, e.g., C. A. Jeffries, et al., (2003), Journal of Biological Chemistry 278:26258-26264; N. J. Horwood, et al., (2003), The Journal of Experimental Medicine 197:1603-1611; Iwaki et al. (2005), Journal of Biological Chemistry 280(48):40261-40270; Vassilev et al. (1999), Journal of Biological Chemistry 274(3):1646-1656, and Quek et al. (1998), Current Biology 8(20):1137-1140.

Ibrutinib (PCI-32765) is an irreversible covalent inhibitor of BTK, inhibits proliferation, induces apoptosis, and has been shown to inhibit BTK in animal models. Further, clinical trials have demonstrated efficacy across several hematological malignancies (e.g. chronic lymphocytic leukemia (CLL) and diffuse large B-cell lymphoma (DLBCL)) including relapsed/refractory hematological malignancies. Indeed, about 70% of chronic lymphocytic leukemia (CLL) patient have demonstrated an objective complete or partial response in a clinical trial and an additional 15 to 20% of patients have a partial response with persistent lymphocytosis. At 26 months, the estimated progression-free survival rate among patients treated with ibrutinib is about 75%. For patients who have the activated B-cell like (ABC) subtype of DLBCL, the overall response rate is 41% and the overall survival is 9.7 month.

Toll-like receptors (TLRs) are a class of proteins that play a key role in the innate immune system. The TLRs include TLR1, TLR2, TLR3, TLR4, TLR5, TLR6, TLR7, TLR8, TLR9, TLR10, TLR11, TLR12, and TLR13. They are single, membrane-spanning, non-catalytic receptors usually expressed in sentinel cells such as macrophages and dendritic cells, that recognize structurally conserved molecules derived from microbes. Different TLRs can recognize different antigens, for example, TLR-6 recognizes bacterial lipoprotein TLR-7 and TLR-8 recognize single stranded RNA, and TLR-9 recognizes CpG DNA.

TLR signaling is divided into two distinct signaling pathways, one of which is the MyD88-dependent pathway. The MyD88-dependent response occurs on dimerization of the TLR receptor, and is utilized by every TLR except TLR3. Its primary effect is activation of NFκB and Mitogen-activated protein kinase. Mutation in MYD88 at position 265 leading to a change from leucine to proline have been identified in human lymphomas including ABC subtype of diffuse large B-cell lymphoma and Waldenstrom's macroglobulinemia.

TEC Family Kinase Inhibitors

BTK is a member of the Tyrosine-protein kinase (TEC) family of kinases. In some embodiments, the TEC family comprises BTK, ITK, TEC, RLK and BMX. In some embodiments, a covalent TEC family kinase inhibitor inhibits the kinase activity of BTK, ITK, TEC, RLK and BMX. In some embodiments, a covalent TEC family kinase inhibitor is a BTK inhibitor. In some embodiments, a covalent TEC family kinase inhibitor is an ITK inhibitor. In some embodiments, a covalent TEC family kinase inhibitor is a TEC inhibitor. In some embodiments, a covalent TEC family kinase inhibitor is a RLK inhibitor. In some embodiments, a covalent TEC family kinase inhibitor is a BMK inhibitor.

BTK Inhibitor Compounds Including Ibrutinib, and Pharmaceutically Acceptable Salts Thereof

The BTK inhibitor compounds described herein are selective for BTK and kinases having a cysteine residue in an amino acid sequence position of the tyrosine kinase that is homologous to the amino acid sequence position of cysteine 481 in BTK. The BTK inhibitor compound can form a covalent bond with Cys 481 of BTK (e.g., via a Michael reaction).

In some embodiments, the BTK inhibitor is a compound of Formula (A) having the structure:

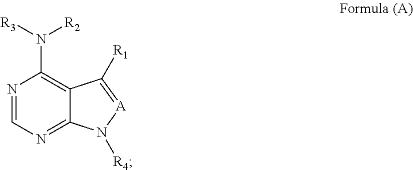

wherein:
A is N;
R₁ is phenyl-O-phenyl or phenyl-S-phenyl;
R₂ and R₃ are independently H;
R₄ is L₃-X-L₄-G, wherein,
L₃ is optional, and when present is a bond, optionally substituted or unsubstituted alkyl, optionally substituted or unsubstituted cycloalkyl, optionally substituted or unsubstituted alkenyl, optionally substituted or unsubstituted alkynyl;
X is optional, and when present is a bond, —O—, —C(═O)—, —S—, —S(═O)—, —S(═O)₂—, —NH—, —NR₉—, —NHC(O)—, —C(O)NH—, —NR₉C(O)—, —C(O)NR₉—, —S(═O)₂NH—, —NHS(═O)₂—, —S(═O)₂NR₉—, —NR₉S(═O)₂—, —OC(O)NH—, —NHC(O)O—, —OC(O)NR₉—, —NR₉C(O)O—, —CH═NO—, —ON═CH—, —NR₁₀C(O)NR₁₀—, heteroaryl-, aryl-, —NR₁₀C(═NR₁₁)NR₁₀—, —NR₁₀C(═NR₁₁)—, —C(═NR₁₁)NR₁₀—, —OC(═NR₁₁)—, or —C(═NR₁₁)O—;
L₄ is optional, and when present is a bond, substituted or unsubstituted alkyl, substituted or unsubstituted cycloalkyl, substituted or unsubstituted alkenyl, substituted or unsubstituted alkynyl, substituted or unsubstituted aryl, substituted or unsubstituted heteroaryl, substituted or unsubstituted heterocycle;
or L₃, X and L₄ taken together form a nitrogen containing heterocyclic ring;
G is

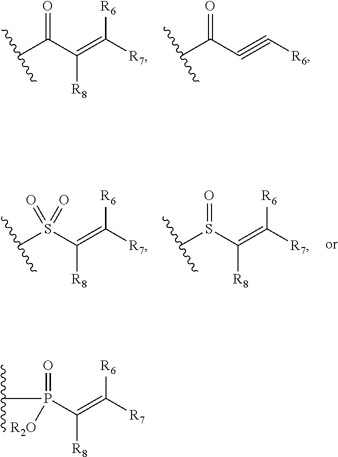

wherein,
R₆, R₇ and R₈ are independently selected from among H, halogen, CN, OH, substituted or unsubstituted alkyl or substituted or unsubstituted heteroalkyl or substituted or unsubstituted cycloalkyl, substituted or unsubstituted heterocycloalkyl, substituted or unsubstituted aryl, substituted or unsubstituted heteroaryl;
each R₉ is independently selected from among H, substituted or unsubstituted lower alkyl, and substituted or unsubstituted lower cycloalkyl;
each R₁₀ is independently H, substituted or unsubstituted lower alkyl, or substituted or unsubstituted lower cycloalkyl; or
two R₁₀ groups can together form a 5-, 6-, 7-, or 8-membered heterocyclic ring; or
R₁₀ and R₁₁ can together form a 5-, 6-, 7-, or 8-membered heterocyclic ring; or each R₁₁ is independently selected from H or substituted or unsubstituted alkyl; or a pharmaceutically acceptable salt thereof. In some embodiments, L₃, X and L₄ taken together form a nitrogen containing heterocyclic ring. In some embodiments, the nitrogen containing heterocyclic ring is a piperidine group. In some embodiments, G is

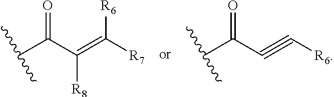

In some embodiments, the compound of Formula (A) is 1-[(3R)-3-[4-amino-3-(4-phenoxyphenyl)pyrazolo[3,4-d]pyrimidin-1-yl]piperidin-1-yl]prop-2-en-1-one.

In some embodiments, the BTK inhibitor compound of Formula (A) has the following structure of Formula (B):

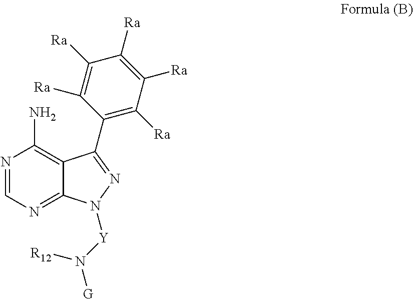

wherein:

Y is alkyl or substituted alkyl, or a 4-, 5-, or 6-membered cycloalkyl ring;

each Rₐ is independently H, halogen, —CF₃, —CN, —NO₂, OH, NH₂, -Lₐ-(substituted or unsubstituted alkyl), -Lₐ-(substituted or unsubstituted alkenyl), -Lₐ-(substituted or unsubstituted heteroaryl), or -Lₐ-(substituted or unsubstituted aryl), wherein Lₐ is a bond, O, S, —S(═O), —S(═O)₂, NH, C(O), CH₂, —NHC(O)O, —NHC(O), or —C(O)NH;

G is

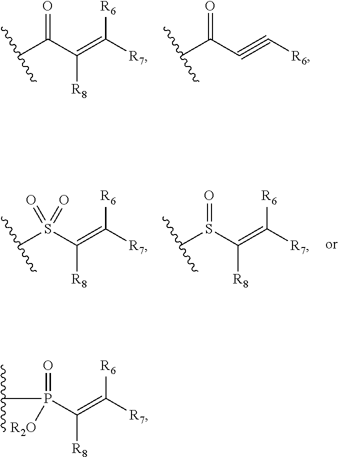

wherein,

R₆, R₇ and R₈ are independently selected from among H, lower alkyl or substituted lower alkyl, lower heteroalkyl or substituted lower heteroalkyl, substituted or unsubstituted lower cycloalkyl, and substituted or unsubstituted lower heterocycloalkyl;

R₁₂ is H or lower alkyl; or

Y and R₁₂ taken together form a 4-, 5-, or 6-membered heterocyclic ring; and

pharmaceutically acceptable active metabolites, pharmaceutically acceptable solvates, pharmaceutically acceptable salts, or pharmaceutically acceptable prodrugs thereof.

In some embodiments, G is selected from among

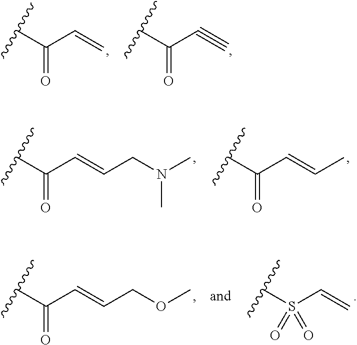

In some embodiments,

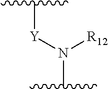

is selected from among

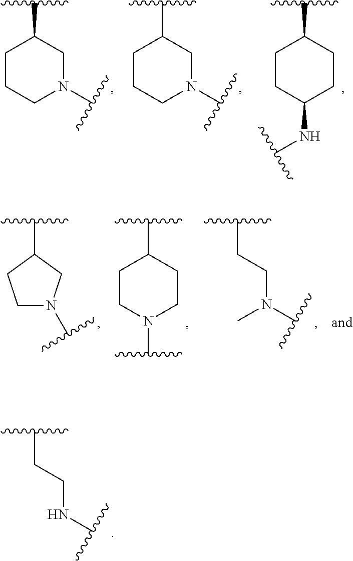

In some embodiments, the BTK inhibitor compound of Formula (B) has the following structure of Formula (C):

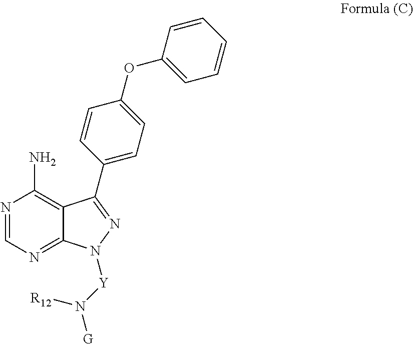

Y is alkyl or substituted alkyl, or a 4-, 5-, or 6-membered cycloalkyl ring;

R₁₂ is H or lower alkyl; or

Y and R₁₂ taken together form a 4-, 5-, or 6-membered heterocyclic ring;

G is

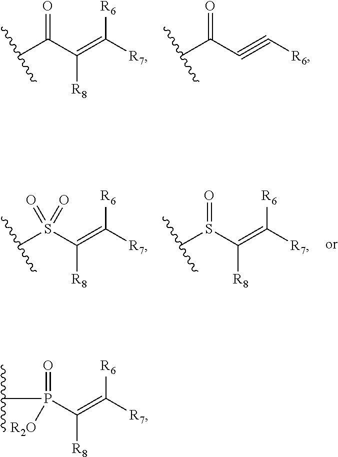

wherein,

R₆, R₇ and R₈ are independently selected from among H, lower alkyl or substituted lower alkyl, lower heteroalkyl or substituted lower heteroalkyl, substituted or unsubstituted lower cycloalkyl, and substituted or unsubstituted lower heterocycloalkyl; and

pharmaceutically acceptable active metabolites, pharmaceutically acceptable solvates, pharmaceutically acceptable salts, or pharmaceutically acceptable prodrugs thereof.

In some embodiments, the “G” group of any of Formula (A), Formula (B), or Formula (C) is any group that is used to tailor the physical and biological properties of the molecule. Such tailoring/modifications are achieved using groups which modulate Michael acceptor chemical reactivity, acidity, basicity, lipophilicity, solubility and other physical properties of the molecule. The physical and biological properties modulated by such modifications to G include, by way of example only, enhancing chemical reactivity of Michael acceptor group, solubility, in vivo absorption, and in vivo metabolism. In addition, in vivo metabolism may include, by way of example only, controlling in vivo PK properties, off-target activities, potential toxicities associated with cypP450 interactions, drug-drug interactions, and the like. Further, modifications to G allow for the tailoring of the in vivo efficacy of the compound through the modulation of, by way of example, specific and non-specific protein binding to plasma proteins and lipids and tissue distribution in vivo.

In some embodiments, the BTK inhibitor has the structure of Formula (D):

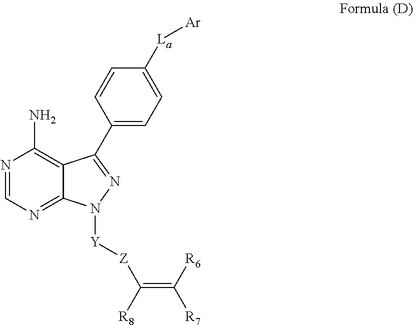

wherein

Lₐ is CH₂, O, NH or S;

Ar is an optionally substituted aromatic carbocycle or an aromatic heterocycle;

Y is an optionally substituted alkyl, heteroalkyl, carbocycle, heterocycle, or combination thereof;

Z is C(O), OC(O), NHC(O), C(S), S(O)ₓ, OS(O)ₓ, NHS(O)ₓ, where x is 1 or 2; and

R₆, R₇, and R₈ are independently selected from H, alkyl, heteroalkyl, carbocycle, heterocycle, or combinations thereof.

In some embodiments, Lₐ is O.

In some embodiments, Ar is phenyl.

In some embodiments, Z is C(O).

In some embodiments, each of R₁, R₂, and R₃ is H.

In some embodiments, provided herein is a compound of Formula (D). Formula (D) is as follows:

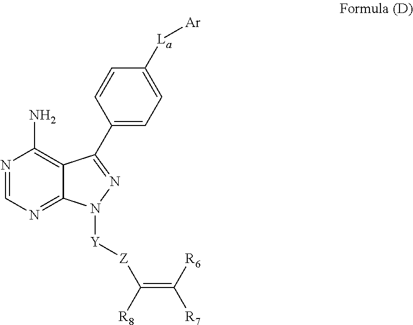

wherein:
Lₐ is CH₂, O, NH or S;
Ar is a substituted or unsubstituted aryl, or a substituted or unsubstituted heteroaryl;
Y is an optionally substituted group selected from among alkyl, heteroalkyl, cycloalkyl, heterocycloalkyl, aryl, and heteroaryl;
Z is C(═O), OC(═O), NHC(═O), C(═S), S(═O)ₓ, OS(═O)ₓ, NHS(═O)ₓ, where x is 1 or 2;
R₇ and R₈ are independently selected from among H, unsubstituted C₁-C₄alkyl, substituted C₁-C₄alkyl, unsubstituted C₁-C₄heteroalkyl, substituted C₁-C₄heteroalkyl, unsubstituted C₃-C₆cycloalkyl, substituted C₃-C₆cycloalkyl, unsubstituted C₂-C₆heterocycloalkyl, and substituted C₂-C₆heterocycloalkyl; or
R₇ and R₈ taken together form a bond;
R₆ is H, substituted or unsubstituted C₁-C₄alkyl, substituted or unsubstituted C₁-C₄heteroalkyl, C₁-C₆alkoxyalkyl, C₁-C₈alkylaminoalkyl, substituted or unsubstituted C₃-C₆cycloalkyl, substituted or unsubstituted aryl, substituted or unsubstituted C₂-C₈heterocycloalkyl, substituted or unsubstituted heteroaryl, C₁-C₄alkyl(aryl), C₁-C₄alkyl(heteroaryl), C₁-C₄alkyl(C₃-C₈cycloalkyl), or C₁-C₄alkyl(C₂-C₈heterocycloalkyl); and

pharmaceutically active metabolites, or pharmaceutically acceptable solvates, pharmaceutically acceptable salts, or pharmaceutically acceptable prodrugs thereof.

For any and all of the embodiments, substituents can be selected from among from a subset of the listed alternatives. For example, in some embodiments, Lₐ is CH₂, O, or NH. In other embodiments, Lₐ is O or NH. In yet other embodiments, Lₐ is O.

In some embodiments, Ar is a substituted or unsubstituted aryl. In yet other embodiments, Ar is a 6-membered aryl. In some other embodiments, Ar is phenyl.

In some embodiments, x is 2. In yet other embodiments, Z is C(═O), OC(═O), NHC(═O), S(═O)ₓ, OS(═O)ₓ, or NHS(═O)ₓ. In some other embodiments, Z is C(═O), NHC(═O), or S(═O)₂.

In some embodiments, R₇ and R₈ are independently selected from among H, unsubstituted C₁-C₄ alkyl, substituted C₁-C₄alkyl, unsubstituted C₁-C₄heteroalkyl, and substituted C₁-C₄heteroalkyl; or R₇ and R₈ taken together form a bond. In yet other embodiments, each of R₇ and R₈ is H; or R₇ and R₈ taken together form a bond.

In some embodiments, R₆ is H, substituted or unsubstituted C₁-C₄alkyl, substituted or unsubstituted C₁-C₄heteroalkyl, C₁-C₆alkoxyalkyl, C₁-C₂alkyl-N(C₁-C₃alkyl)₂, substituted or unsubstituted aryl, substituted or unsubstituted heteroaryl, C₁-C₄alkyl(aryl), C₁-C₄alkyl(heteroaryl), C₁-C₄alkyl(C₃-C₈cycloalkyl), or C₁-C₄alkyl(C₂-C₈heterocycloalkyl). In some other embodiments, R₆ is H, substituted or unsubstituted C₁-C₄alkyl, substituted or unsubstituted C₁-C₄heteroalkyl, C₁-C₆alkoxyalkyl, C₁-C₂alkyl-N(C₁-C₃alkyl)₂, C₁-C₄alkyl(aryl), C₁-C₄alkyl(heteroaryl), C₁-C₄alkyl(C₃-C₈cycloalkyl), or C₁-C₄alkyl(C₂-C₈heterocycloalkyl). In yet other embodiments, R₆ is H, substituted or unsubstituted C₁-C₄alkyl, —CH₂—O—(C₁-C₃alkyl), —CH₂—N(C₁-C₃alkyl)₂, C₁-C₄alkyl(phenyl), or C₁-C₄alkyl(5- or 6-membered heteroaryl). In some embodiments, R₆ is H, substituted or unsubstituted C₁-C₄alkyl, —CH₂—O—(C₁-C₃alkyl), —CH₂—N(C₁-C₃alkyl)₂, C₁-C₄alkyl(phenyl), or C₁-C₄alkyl(5- or 6-membered heteroaryl containing 1 or 2 N atoms), or C₁-C₄alkyl(5- or 6-membered heterocycloalkyl containing 1 or 2 N atoms).

In some embodiments, Y is an optionally substituted group selected from among alkyl, heteroalkyl, cycloalkyl, and heterocycloalkyl. In other embodiments, Y is an optionally substituted group selected from among C₁-C₆alkyl, C₁-C₆heteroalkyl, 4-, 5-, 6- or 7-membered cycloalkyl, and 4-, 5-, 6- or 7-membered heterocycloalkyl. In yet other embodiments, Y is an optionally substituted group selected from among C₁-C₆alkyl, C₁-C₆heteroalkyl, 5-, or 6-membered cycloalkyl, and 5-, or 6-membered heterocycloalkyl containing 1 or 2 N atoms. In some other embodiments, Y is a 5-, or 6-membered cycloalkyl, or a 5-, or 6-membered heterocycloalkyl containing 1 or 2 N atoms.

Any combination of the groups described above for the various variables is contemplated herein. It is understood that substituents and substitution patterns on the compounds provided herein can be selected by one of ordinary skill in the art to provide compounds that are chemically stable and that can be synthesized by techniques known in the art, as well as those set forth herein.

In some embodiments the BTK inhibitor compounds of Formula (A), Formula (B), Formula (C), Formula (D), include, but are not limited to, compounds selected from the group consisting of:

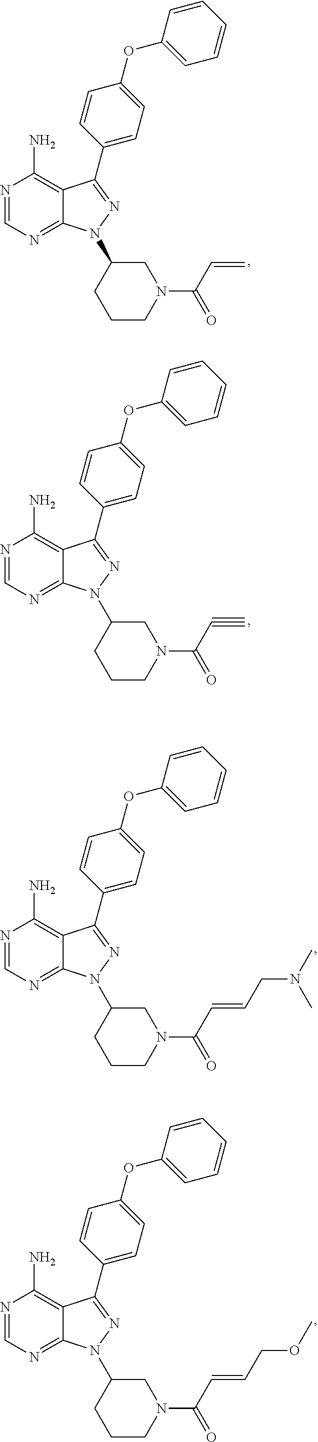

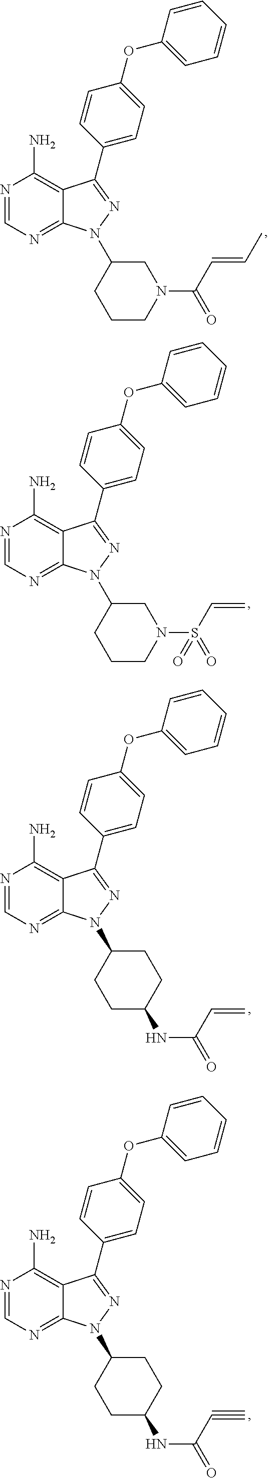

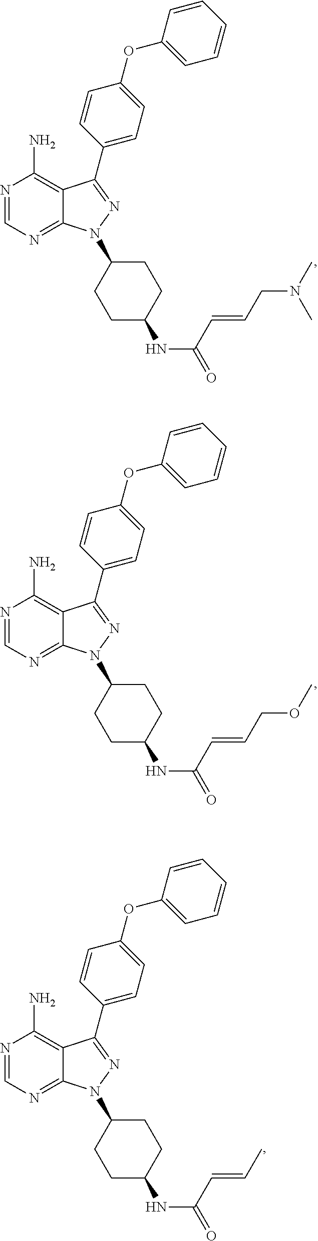

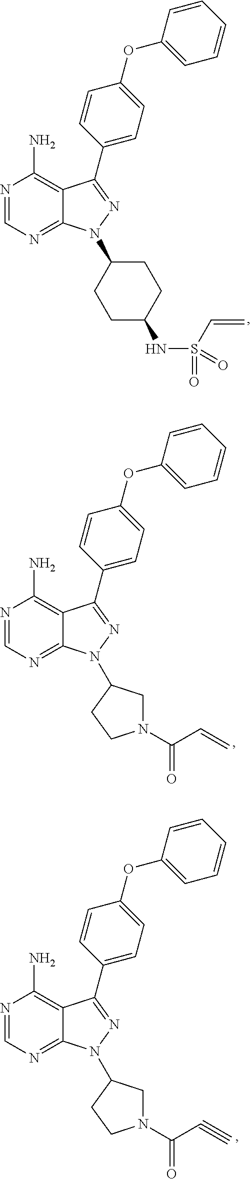

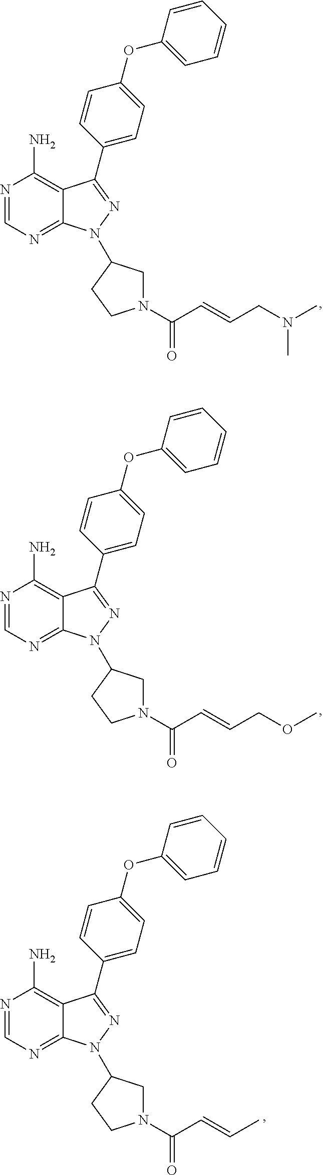 [SMILES: CN(C)C/C=C/C(=O)N1CCC(n2nc(-c3ccc(Oc4ccccc4)cc3)c3c(N)ncnc32)C1 ; COC/C=C/C(=O)N1CCC(n2nc(-c3ccc(Oc4ccccc4)cc3)c3c(N)ncnc32)C1 ; C/C=C/C(=O)N1CCC(n2nc(-c3ccc(Oc4ccccc4)cc3)c3c(N)ncnc32)C1]

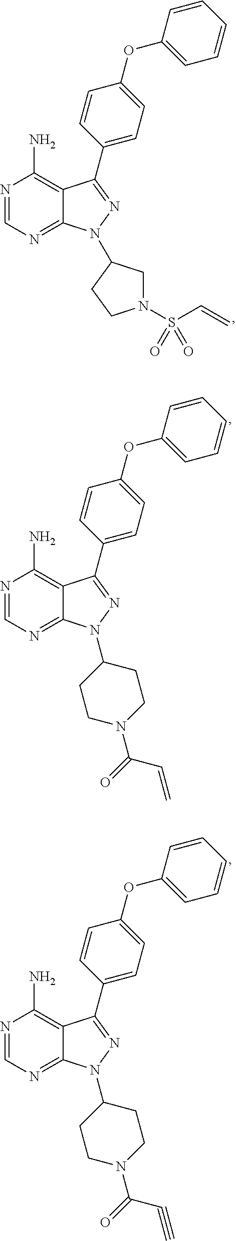 [SMILES: C=CS(=O)(=O)N1CCC(n2nc(-c3ccc(Oc4ccccc4)cc3)c3c(N)ncnc32)C1 ; C=CC(=O)N1CCC(n2nc(-c3ccc(Oc4ccccc4)cc3)c3c(N)ncnc32)CC1 ; C#CC(=O)N1CCC(n2nc(-c3ccc(Oc4ccccc4)cc3)c3c(N)ncnc32)CC1]

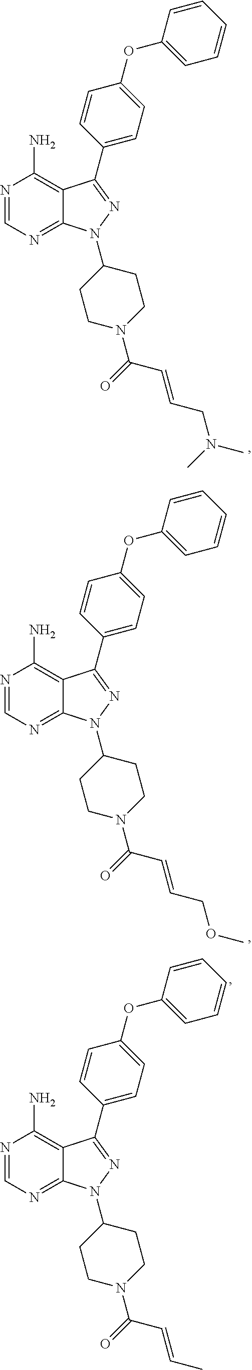

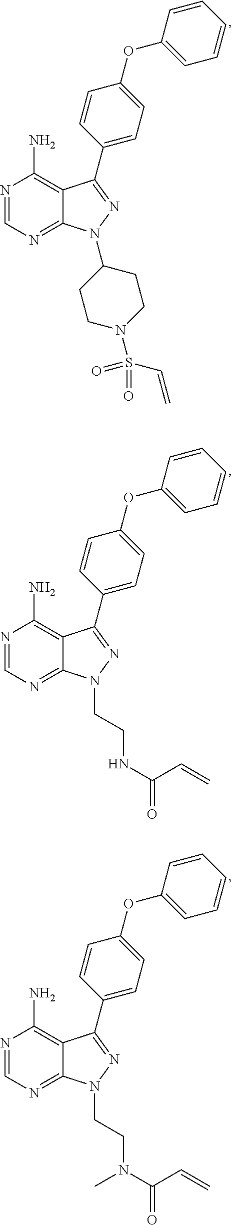 [SMILES: C=CS(=O)(=O)N1CCC(n2nc(-c3ccc(Oc4ccccc4)cc3)c3c(N)ncnc32)CC1 ; C=CC(=O)NCCn1nc(-c2ccc(Oc3ccccc3)cc2)c2c(N)ncnc21 ; C=CC(=O)N(C)CCn1nc(-c2ccc(Oc3ccccc3)cc2)c2c(N)ncnc21]

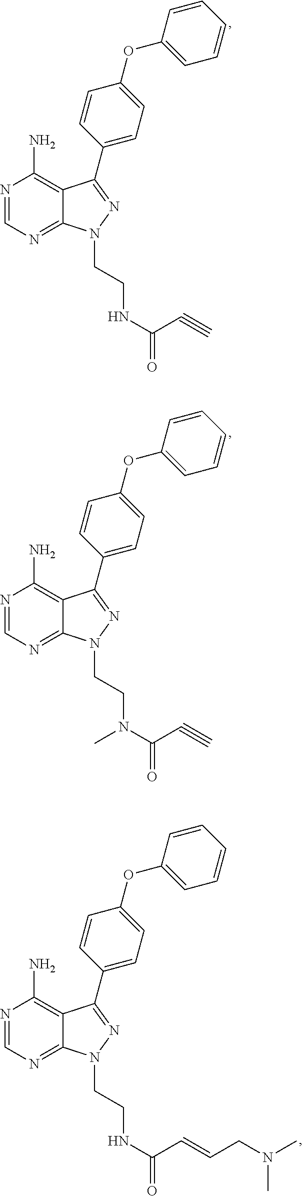 [SMILES: C#CC(=O)NCCn1nc(-c2ccc(Oc3ccccc3)cc2)c2c(N)ncnc21 ; C#CC(=O)N(C)CCn1nc(-c2ccc(Oc3ccccc3)cc2)c2c(N)ncnc21 ; CN(C)C/C=C/C(=O)NCCn1nc(-c2ccc(Oc3ccccc3)cc2)c2c(N)ncnc21]

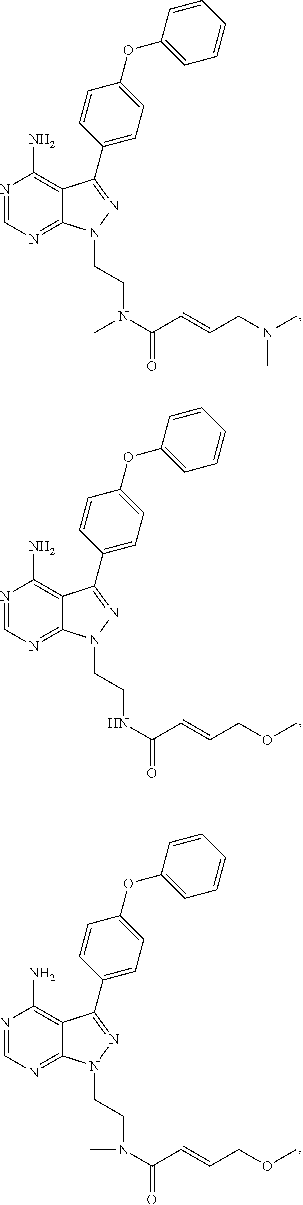 [SMILES: CN(C)C/C=C/C(=O)N(C)CCn1nc(-c2ccc(Oc3ccccc3)cc2)c2c(N)ncnc21 ; COC/C=C/C(=O)NCCn1nc(-c2ccc(Oc3ccccc3)cc2)c2c(N)ncnc21 ; COC/C=C/C(=O)N(C)CCn1nc(-c2ccc(Oc3ccccc3)cc2)c2c(N)ncnc21]

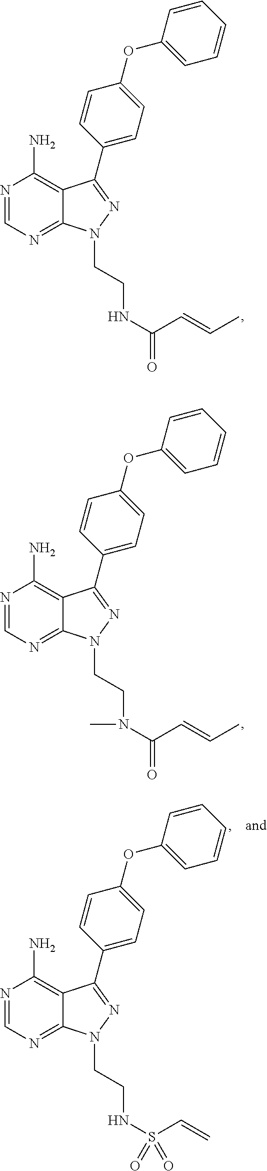

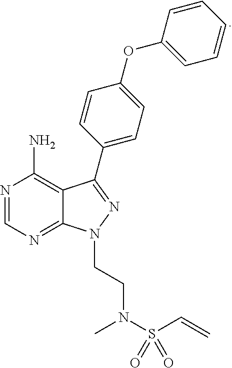 [SMILES: C=CS(=O)(=O)N(C)CCn1nc(-c2ccc(Oc3ccccc3)cc2)c2c(N)ncnc21]

In some embodiments, the BTK inhibitor compounds are selected from the group consisting of:

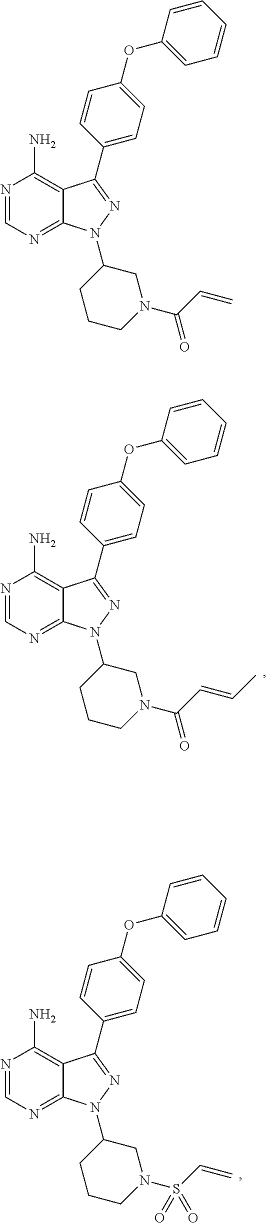 [SMILES: C=CC(=O)N1CCCC(n2nc(-c3ccc(Oc4ccccc4)cc3)c3c(N)ncnc32)C1 ; C/C=C/C(=O)N1CCCC(n2nc(-c3ccc(Oc4ccccc4)cc3)c3c(N)ncnc32)C1 ; C=CS(=O)(=O)N1CCCC(n2nc(-c3ccc(Oc4ccccc4)cc3)c3c(N)ncnc32)C1]

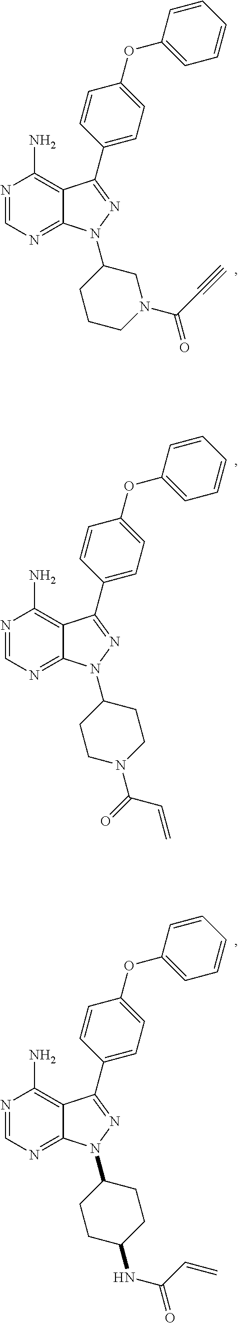

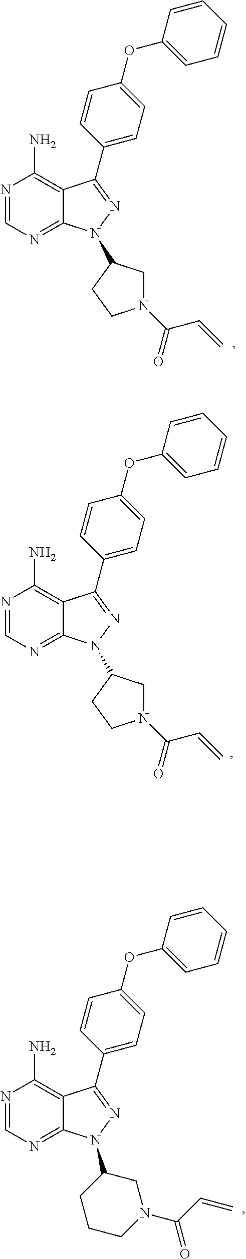

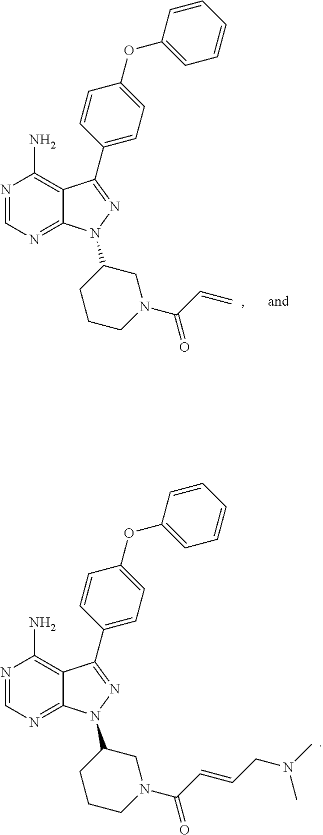

In some embodiments, the BTK inhibitor compounds are selected from the group consisting of:

1-(3-(4-amino-3-(4-phenoxyphenyl)-1H-pyrazolo[3,4-d]pyrimidin-1-yl)piperidin-1-yl)prop-2-en-1-one (Compound 4); (E)-1-(3-(4-amino-3-(4-phenoxyphenyl)-1H-pyrazolo[3,4-d]pyrimidin-1-yl)piperidin-1-yl)but-2-en-1-one (Compound 5); 1-(3-(4-amino-3-(4-phenoxyphenyl)-1H-pyrazolo[3,4-d]pyrimidin-1-yl)piperidin-1-yl)sulfonylethene (Compound 6); 1-(3-(4-amino-3-(4-phenoxyphenyl)-1H-pyrazolo[3,4-d]pyrimidin-1-yl)piperidin-1-yl)prop-2-yn-1-one (Compound 8); 1-(4-(4-amino-3-(4-phenoxyphenyl)-1H-pyrazolo[3,4-d]pyrimidin-1-yl)piperidin-1-yl)prop-2-en-1-one (Compound 9); N-((1s,4s)-4-(4-amino-3-(4-phenoxyphenyl)-1H-pyrazolo[3,4-d]pyrimidin-1-yl)cyclohexyl)acrylamide (Compound 10); 1-((R)-3-(4-amino-3-(4-phenoxyphenyl)-1H-pyrazolo[3,4-d]pyrimidin-1-yl)pyrrolidin-1-yl)prop-2-en-1-one (Compound 11); 1-((S)-3-(4-amino-3-(4-phenoxyphenyl)-1H-pyrazolo[3,4-d]pyrimidin-1-yl)pyrrolidin-1-yl)prop-2-en-1-one (Compound 12); 1-((R)-3-(4-amino-3-(4-phenoxyphenyl)-1H-pyrazolo[3,4-d]pyrimidin-1-yl)piperidin-1-yl)prop-2-en-1-one (Compound 13); 1-((S)-3-(4-amino-3-(4-phenoxyphenyl)-1H-pyrazolo[3,4-d]pyrimidin-1-yl)piperidin-1-yl)prop-2-en-1-one (Compound 14); and (E)-1-(3-(4-amino-3-(4-phenoxyphenyl)-1H-pyrazolo[3,4-d]pyrimidin-1-yl)piperidin-1-yl)-4-(dimethylamino)but-2-en-1-one (Compound 15).

Throughout the specification, groups and substituents thereof can be chosen by one skilled in the field to provide stable moieties and compounds.

The compounds of any of Formula (A), or Formula (B), or Formula (C), or Formula (D) can irreversibly inhibit Btk and may be used to treat patients suffering from Bruton's tyrosine kinase-dependent or Bruton's tyrosine kinase mediated conditions or diseases, including, but not limited to, cancer, autoimmune and other inflammatory diseases.

“Ibrutinib” or “1-((R)-3-(4-amino-3-(4-phenoxyphenyl)-1H-pyrazolo[3,4-d]pyrimidin-1-yl)piperidin-1-yl)prop-2-en-1-one” or “1-{(3R)-3-[4-amino-3-(4-phenoxyphenyl)-1H-pyrazolo[3,4-d]pyrimidin-1-yl]piperidin-1-yl}prop-2-en-1-one” or “2-Propen-1-one, 1-[(3R)-3-[4-amino-3-(4-phenoxyphenyl)-1H-pyrazolo[3,4-d]pyrimidin-1-yl]-1-piperidinyl-” or ibrutinib or any other suitable name refers to the compound with the following structure:

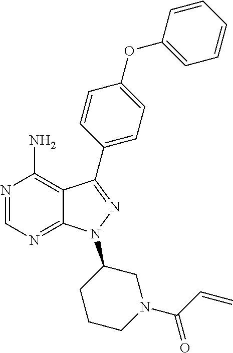 [SMILES: C=CC(=O)N1CCC[C@@H](n2nc(-c3ccc(Oc4ccccc4)cc3)c3c(N)ncnc32)C1]

A wide variety of pharmaceutically acceptable salts is formed from Ibrutinib and includes:
acid addition salts formed by reacting ibrutinib with an organic acid, which includes aliphatic mono- and dicarboxylic acids, phenyl-substituted alkanoic acids, hydroxyl alkanoic acids, alkanedioic acids, aromatic acids, aliphatic and aromatic sulfonic acids, amino acids, etc. and include, for example, acetic acid, trifluoroacetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid, and the like;
acid addition salts formed by reacting ibrutinib with an inorganic acid, which includes hydrochloric acid, hydrobromic acid, sulfuric acid, nitric acid, phosphoric acid, hydroiodic acid, hydrofluoric acid, phosphorous acid, and the like.

The term “pharmaceutically acceptable salts” in reference to ibrutinib refers to a salt of ibrutinib, which does not cause significant irritation to a mammal to which it is administered and does not substantially abrogate the biological activity and properties of the compound.

It should be understood that a reference to a pharmaceutically acceptable salt includes the solvent addition forms (solvates). Solvates contain either stoichiometric or non-stoichiometric amounts of a solvent, and are formed during the process of product formation or isolation with pharmaceutically acceptable solvents such as water, ethanol, methanol, methyl tert-butyl ether (MTBE), diisopropyl ether (DIPE), ethyl acetate, isopropyl acetate, isopropyl alcohol, methyl isobutyl ketone (MIBK), methyl ethyl ketone (MEK), acetone, nitromethane, tetrahydrofuran (THF), dichloromethane (DCM), dioxane, heptanes, toluene, anisole, acetonitrile, and the like. In one aspect, solvates are formed using, but limited to, Class 3 solvent(s). Categories of solvents are defined in, for example, the International Conference on Harmonization of Technical Requirements for Registration of Pharmaceuticals for Human Use (ICH), “Impurities: Guidelines for Residual Solvents, Q3C(R3), (November 2005). Hydrates are formed when the solvent is water, or alcoholates are formed when the solvent is alcohol. In some embodiments, solvates of ibrutinib, or pharmaceutically acceptable salts thereof, are conveniently prepared or formed during the processes described herein. In some embodiments, solvates of ibrutinib are anhydrous. In some embodiments, ibrutinib, or pharmaceutically acceptable salts thereof, exist in unsolvated form. In some embodiments, ibrutinib, or pharmaceutically acceptable salts thereof, exist in unsolvated form and are anhydrous.

In yet other embodiments, ibrutinib, or a pharmaceutically acceptable salt thereof, is prepared in various forms, including but not limited to, amorphous phase, crystalline forms, milled forms and nano-particulate forms. In some embodiments, ibrutinib, or a pharmaceutically acceptable salt thereof, is amorphous. In some embodiments, ibrutinib, or a pharmaceutically acceptable salt thereof, is amorphous and anhydrous. In some embodiments, ibrutinib, or a pharmaceutically acceptable salt thereof, is crystalline. In some embodiments, ibrutinib, or a pharmaceutically acceptable salt thereof, is crystalline and anhydrous.

In some embodiments, ibrutinib is prepared as outlined in U.S. Pat. No. 7,514,444.

In some embodiments, the Btk inhibitor is PCI-45292, PCI-45466, AVL-101/CC-101 (Avila Therapeutics/Celgene Corporation), AVL-263/CC-263 (Avila Therapeutics/Celgene Corporation), AVL-292/CC-292 (Avila Therapeutics/Celgene Corporation), AVL-291/CC-291 (Avila Therapeutics/Celgene Corporation), CNX 774 (Avila Therapeutics), BMS-488516 (Bristol-Myers Squibb), BMS-509744 (Bristol-Myers Squibb), CGI-1746 (CGI Pharma/Gilead Sciences), CGI-560 (CGI Pharma/Gilead Sciences), CTA-056, GDC-0834 (Genentech), HY-11066 (also, CTK4I7891, HMS3265G21, HMS3265G22, HMS3265H21, HMS3265H22, 439574-61-5, AG-F-54930), ONO-4059 (Ono Pharmaceutical Co., Ltd.), ONO-WG37 (Ono Pharmaceutical Co., Ltd.), PLS-123 (Peking University), RN486 (Hoffmann-La Roche), HM71224 (Hanmi Pharmaceutical Company Limited), LFM-A13, BGB-3111 (Beigene), KBP-7536 (KBP BioSciences), ACP-196 (Acerta Pharma), JTE-051 (Japan Tobacco Inc), PRN1008 (Principia), CTP-730 (Concert Pharmaceuticals), or GDC-0853 (Genentech).

In some embodiments, the BTK inhibitor is 4-(tert-butyl)-N-(2-methyl-3-(4-methyl-6-((4-(morpholine-4-carbonyl)phenyl)amino)-5-oxo-4,5-dihydropyrazin-2-yl)phenyl)benzamide (CGI-1746); 7-benzyl-1-(3-(piperidin-1-yl)propyl)-2-(4-(pyridin-4-yl)phenyl)-1H-imidazo[4,5-g]quinoxalin-6(5H)-one (CTA-056); (R)—N-(3-(6-(4-(1,4-dimethyl-3-oxopiperazin-2-yl)phenylamino)-4-methyl-5-oxo-4,5-dihydropyrazin-2-yl)-2-methylphenyl)-4,5,6,7-tetrahydrobenzo[b]thiophene-2-carboxamide (GDC-0834); 6-cyclopropyl-8-fluoro-2-(2-hydroxymethyl-3-{1-methyl-5-[5-(4-methyl-piperazin-1-yl)-pyridin-2-ylamino]-6-oxo-1,6-dihydro-pyridin-3-yl}-phenyl)-2H-isoquinolin-1-one (RN-486); N-[5-[5-(4-acetylpiperazine-1-carbonyl)-4-methoxy-2-methylphenyl]sulfanyl-1,3-thiazol-2-yl]-4-[(3,3-dimethylbutan-2-ylamino)methyl]benzamide (BMS-509744, HY-11092); or N-(5-((5-(4-Acetylpiperazine-1-carbonyl)-4-methoxy-2-methylphenyl)thio)thiazol-2-yl)-4-(((3-methylbutan-2-yl)amino)methyl)benzamide (HY11066); or a pharmaceutically acceptable salt thereof.

In some embodiments, the BTK inhibitor is:

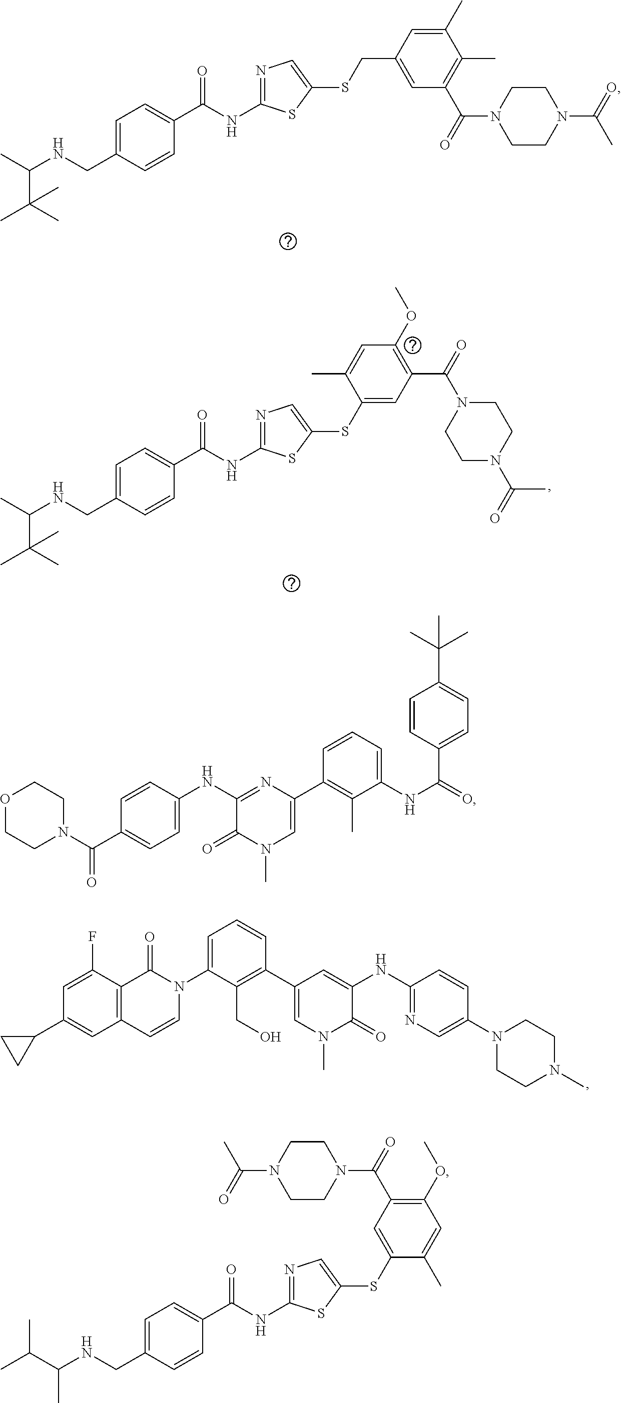

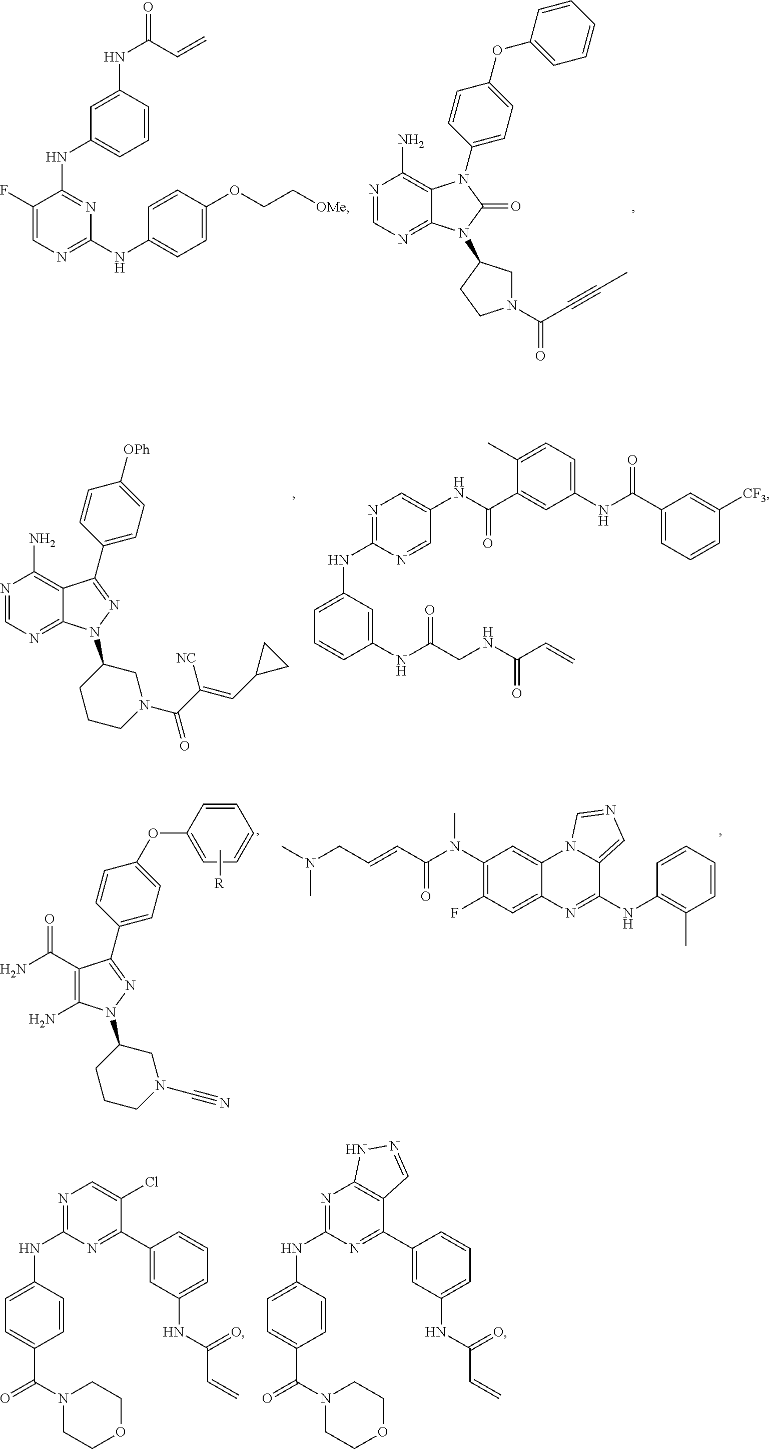

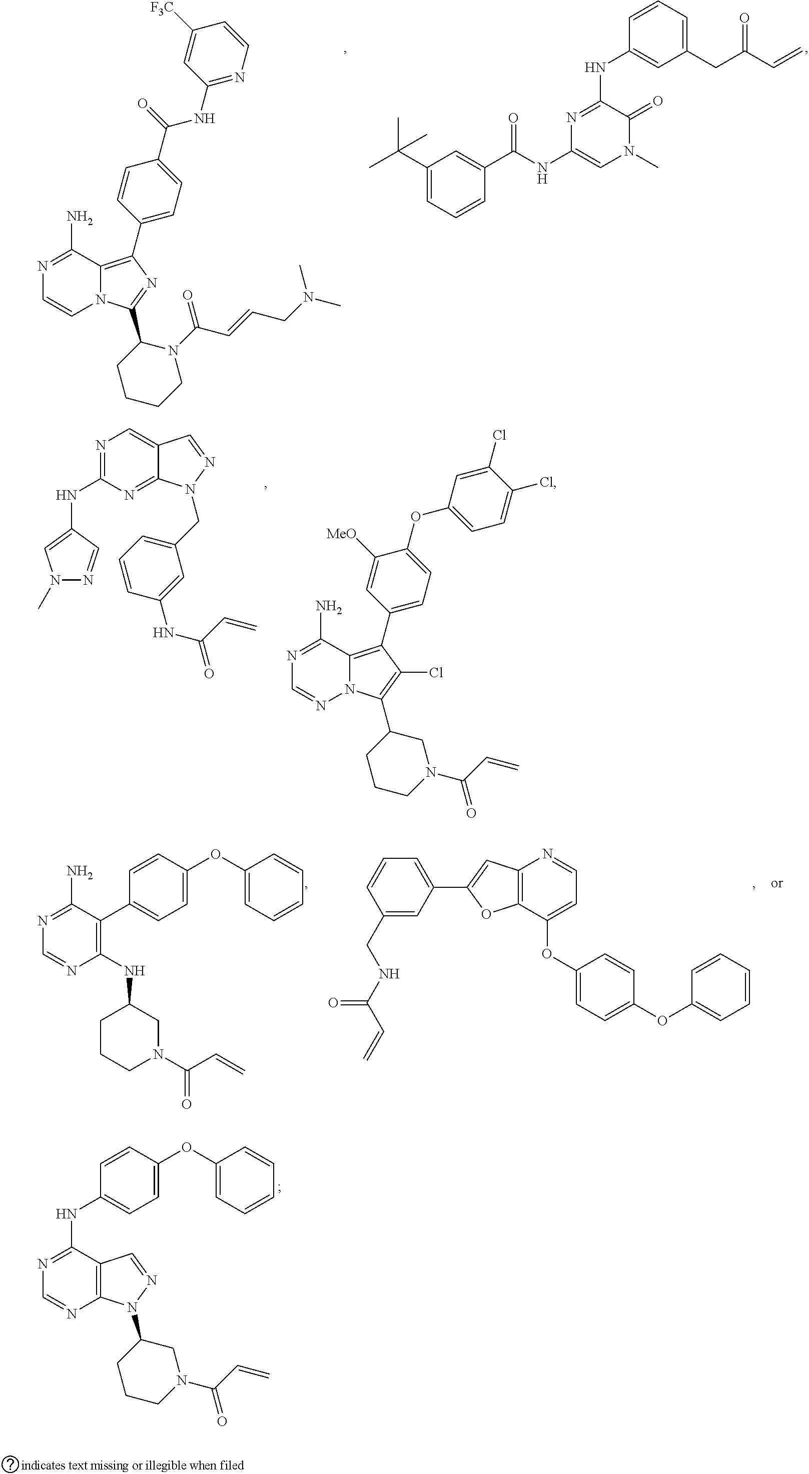

or a pharmaceutically acceptable salt thereof.

ITK Inhibitors

In some embodiments, the ITK inhibitor covalently binds to Cysteine 442 of ITK. In some embodiments, the ITK inhibitor is an ITK inhibitor compound described in WO 2002/0500071, which is incorporated by reference in its entirety. In some embodiments, the ITK inhibitor is an ITK inhibitor compound described in WO 2005/070420, which is incorporated by reference in its entirety. In some embodiments, the ITK inhibitor is an ITK inhibitor compound described in WO2005/079791, which is incorporated by reference in its entirety. In some embodiments, the ITK inhibitor is an ITK inhibitor compound described in WO 2007/076228, which is incorporated by reference in its entirety. In some embodiments, the ITK inhibitor is an ITK inhibitor compound described in WO 2007/058832, which is incorporated by reference in its entirety. In some embodiments, the ITK inhibitor is an ITK inhibitor compound described in WO 2004/016610, which is incorporated by reference in its entirety. In some embodiments, the ITK inhibitor is an ITK inhibitor compound described in WO 2004/016611, which is incorporated by reference in its entirety. In some embodiments, the ITK inhibitor is an ITK inhibitor compound described in WO 2004/016600, which is incorporated by reference in its entirety. In some embodiments, the ITK inhibitor is an ITK inhibitor compound described in WO 2004/016615, which is incorporated by reference in its entirety. In some embodiments, the ITK inhibitor is an ITK inhibitor compound described in WO 2005/026175, which is incorporated by reference in its entirety. In some embodiments, the ITK inhibitor is an ITK inhibitor compound described in WO 2006/065946, which is incorporated by reference in its entirety. In some embodiments, the ITK inhibitor is an ITK inhibitor compound described in WO 2007/027594, which is incorporated by reference in its entirety. In some embodiments, the ITK inhibitor is an ITK inhibitor compound described in WO 2007/017455, which is incorporated by reference in its entirety. In some embodiments, the ITK inhibitor is an ITK inhibitor compound described in WO 2008/025820, which is incorporated by reference in its entirety. In some embodiments, the ITK inhibitor is an ITK inhibitor compound described in WO 2008/025821, which is incorporated by reference in its entirety. In some embodiments, the ITK inhibitor is an ITK inhibitor compound described in WO 2008/025822, which is incorporated by reference in its entirety. In some embodiments, the ITK inhibitor is an ITK inhibitor compound described in WO 2011/017219, which is incorporated by reference in its entirety. In some embodiments, the ITK inhibitor is an ITK inhibitor compound described in WO 2011/090760, which is incorporated by reference in its entirety. In some embodiments, the ITK inhibitor is an ITK inhibitor compound described in WO 2009/158571, which is incorporated by reference in its entirety. In some embodiments, the ITK inhibitor is an ITK inhibitor compound described in WO 2009/051822, which is incorporated by reference in its entirety. In some embodiments, the Itk inhibitor is an Itk inhibitor compound described in US 20110281850, which is incorporated by reference in its entirety. In some embodiments, the Itk inhibitor is an Itk inhibitor compound described in WO 2014/082085, which is incorporated by reference in its entirety. In some embodiments, the Itk inhibitor is an Itk inhibitor compound described in WO 2014/093383, which is incorporated by reference in its entirety. In some embodiments, the Itk inhibitor is an Itk inhibitor compound described in U.S. Pat. No. 8,759,358, which is incorporated by reference in its entirety. In some embodiments, the Itk inhibitor is an Itk inhibitor compound described in WO 2014/105958, which is incorporated by reference in its entirety. In some embodiments, the Itk inhibitor is an Itk inhibitor compound described in US 20140256704, which is incorporated by reference in its entirety. In some embodiments, the Itk inhibitor is an Itk inhibitor compound described in US 20140315909, which is incorporated by reference in its entirety. In some embodiments, the Itk inhibitor is an Itk inhibitor compound described in US 20140303161, which is incorporated by reference in its entirety. In some embodiments, the Itk inhibitor is an Itk inhibitor compound described in WO 2014/145403, which is incorporated by reference in its entirety.

In some embodiments, the ITK inhibitor is selected from the group consisting of compounds of Formula (A), Formula (B), Formula (C), and Formula (D).

In some embodiments, the ITK inhibitor has a structure selected from the group consisting of:

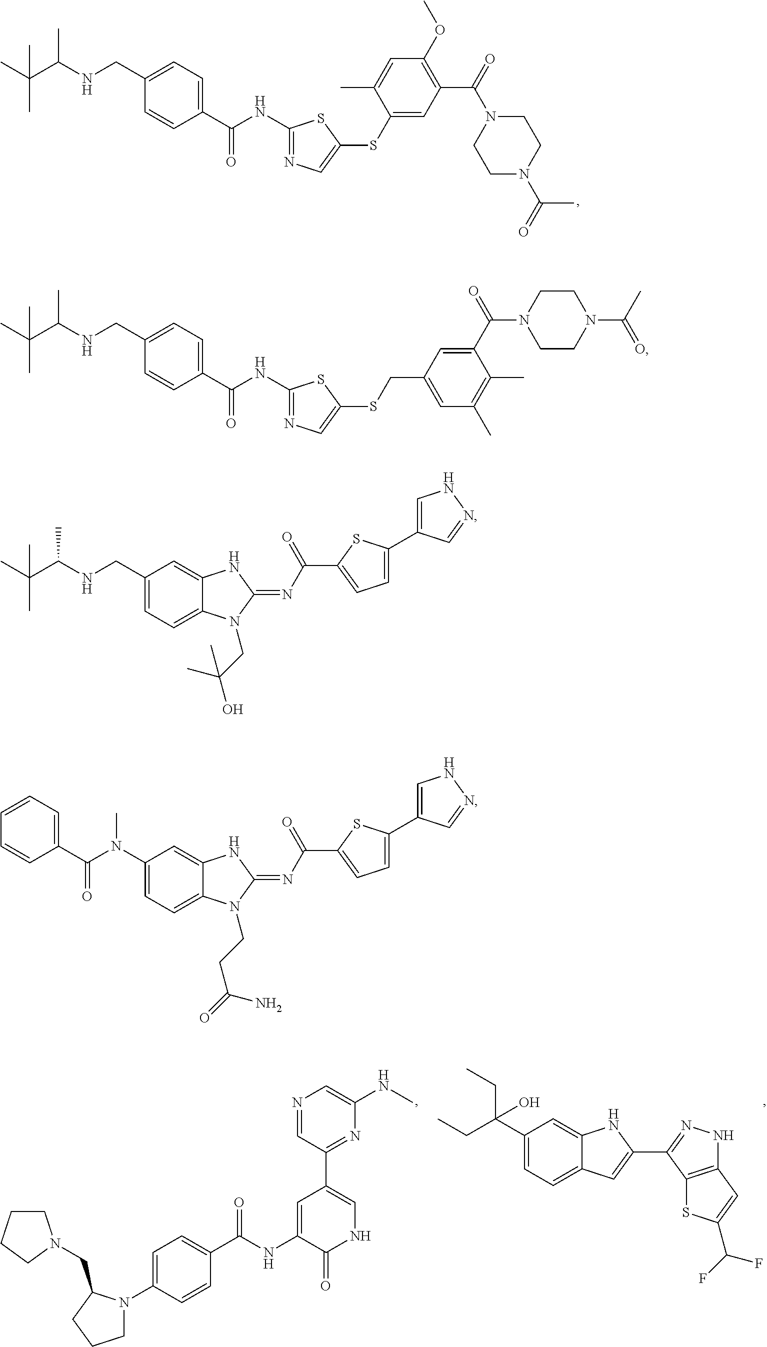

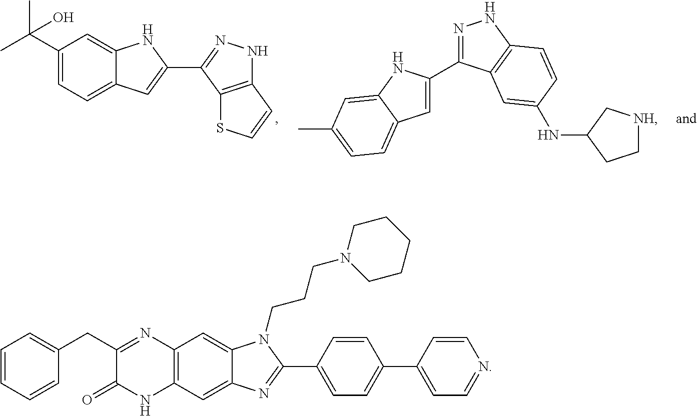

TLR Inhibitors

The TLR inhibitors or antagonists are compounds that target the members of the TLR family. TLR inhibitors include small molecule or biologic (antibodies, peptides, nucleic acids-antisense nucleic acids, ribozymes, siRNA nucleic acids) inhibitors. In some embodiments, the TLR inhibitors are non-specific TLR inhibitors, TLR6/7/8/9 antagonists, TLR7/8/9 antagonists, TLR7/9 antagonists, TLR7/8 antagonists, TLR6 antagonists, or TLR9 antagonists. In some embodiments, the TLR inhibitors are non-specific TLR inhibitors, TLR7/8/9 antagonists, TLR7/9 antagonists, TLR7/8 antagonists, or TLR9 antagonists. In some embodiments, the TLR inhibitors are non-specific or non-selective inhibitors that target all or most TLR proteins.

In some embodiments, TLR inhibitors include substituted quinoline compounds, substituted quinazole compounds, tricyclic TLR inhibitors (e.g., mianserin, desipramine, cyclobenzaprine, imiprimine, ketotifen, and amitriptyline), Vaccinia virus A52R protein (US 20050244430), Polymyxin-B (specific inhibitor of LPS-bioactivity), BX795, chloroquine, CLI-095, RDP58, ST2825, ML120B, PHA-408, insulin (Clinical trial NCT01151605), oligodeoxynucleotides (ODN) that suppress CpG-induced immune responses, G-rich ODN, and ODN with TTAGGG motifs. In some embodiments, TLR antagonists include those described in patents or patent applications US 20050119273, WO 2014052931, WO 2014108529, US 20140094504, US 20120083473, U.S. Pat. No. 8,729,088 and US 20090215908. In some embodiments, TLR inhibitors include ST2 antibody; sST2-Fc (functional murine soluble ST2-human IgG1 Fc fusion protein; see Biochemical and Biophysical Research Communications, 29 Dec. 2006, vol. 351, no. 4, 940-946); CRX-526 (Corixa); lipid IVA; RSLA (Rhodobacter sphaeroides lipid A); E5531 ((6-O-{2-deoxy-6-O-methyl-4-O-phosphono-3-O—[(R)-3-Z-dodec-5-endoyloxydecl]-2-[3-oxo-tetradecanoylamino]-β-O-phosphono-α-D-glucopyranose tetrasodium salt); E5564 (α-D-Glucopyranose,3-O-decyl-2-deoxy-6-O-[2-deoxy-3-O-[(3R)-3-methoxydecyl]-6-O-methyl-2-[[(11Z)-1-oxo-11-octadecenyl] amino]-4-O-phosphono-β-D-glucopyranosyl]-2-[(1,3-dioxotetradecyl)amino]-1-(dihydrogen phosphate), tetrasodium salt); compound 4a (hydrocinnamoyl-L-valyl pyrrolidine; see PNAS, Jun. 24, 2003, vol. 100, no. 13, 7971-7976); CPG 52364 (Coley Pharmaceutical Group); LY294002 (2-(4-Morpholinyl)-8-phenyl-4H-1-benzopyran-4-one); PD98059 (2-(2-amino-3-methoxyphenyl)-4H-1-Benzopyran-4-one); chloroquine; and an immune regulatory oligonucleotide (see U.S. Patent Application Publication No. 2008/0089883). In some embodiments, the TLR inhibitor is chloroquine, bafilomycin A, IMO-8400, ODN 4084-F, ODN INH-1, ODN INH-18, ODN TTAGGG, G-ODN, or ODN 2088. In some embodiments, the TLR inhibitor is chloroquine.

In some embodiments, the TLR inhibitor is a TLR9 antagonist. In some embodiments, TLR9 antagonists include chloroquine, quinacrine, monesin, bafilomycin A1, wortmannin, iODN as described in WO 2009089399, (+)-morphinans as described in US 20110015219, oligonucleotides as described in U.S. Pat. No. 8,853,375, oligodeoxynucleotide compounds containing unmethylated CpG dinucleotides as described in Yu et al (J. Med Chem, 2009, 52: 5108-5114), 9-aminoacridine, 4-aminoquinoline, 4-aminoquinolines such as 7,8,9,10-tetrahydro-6H-cyclohepta[b]quinolin-11-ylamine; 1-methyl-2,3-dihydro-1H-pyrrolo[2,3-b]quinolin-4-ylamine; 1,6-dimethyl-2,3-dihydro-1H-pyrrolo[2,3-b]quinolin-4-ylamine; 6-bromo-1-methyl-2,3-dihydro-1H-pyrrolo[2,3-b]quinolin-4-ylamine; 1-methyl-2,3,4,5-tetrahydro-1H-azepino[2,3-b]quinolin-6-ylamine; 3,3-dimethyl-3,4-dihydro-acridin-9-ylamine; 1-benzyl-2,3-dihydro-1H-pyrrolo[2,3-b]quinolin-4-ylamine; 6-methyl-1-phenyl-2,3-dihydro-1H-pyrrolo[2,3-b]quinolin-4-ylamine; N*2*,N*2*-Dimethyl-quinoline-2,4-diamine, 2,7-Dimethyl-dibenzo[b,g][1,8]naphthyridin-11-ylamine; 2,4-Dimethyl-benzo[b][1,8]naphthyridin-5-ylamine; 7-Fluoro-2,4-dimethyl-benzo[b][1,8]naphthyridin-5-ylamine; 1,2,3,4-Tetrahydro-acridin-9-ylamine Tacrine hydrochloridehydrate; 2,3-Dihydro-1H-cyclopenta[b]quinolin-9-ylamine; 2,4,9-Trimethyl-benzo[b][1,8]naphthyridin-5-ylamine; 9-Amino-3,3-dimethyl-1,2,3,4-tetrahydro-acridin-1-ol and 7-Ethoxy-N*3*-furan-2-ylmethyl-acridine-3,9-diamine; quinazolines such as N,N-dimethyl-N′-{2-[4-(4-methyl-piperazin-1-yl)-phenyl]-3,4-dihydro-quinazoline-4-yl}-ethane-1,2,-diamine; N′-[6,7-Dimethoxy-2-(4-phenyl-piperazin-1-yl)-quinazolin-4-yl]-N,N-dimethyl-ethane-1,2-diamine; N′-[6,7-Dimethoxy-2-(4-methyl-piperazin-1-yl)-quinazolin-4-yl]-N,N-dimethyl-ethane-1,2-diamine; N,N-Dimethyl-N′-(2-phenyl-quinazolin-4-yl)-ethane-1,2-diamine; Dimethyl-(2-{2-[4-(4-methyl-piperazin-1-yl)-phenyl]-quinazolin-4-yloxy}-ethyl)-amine; N′-(2-Biphenyl-4-yl-quinazolin-4-yl)-N,N-dimethyl-ethane-1,2-diamine and Dimethyl-[2-(2-phenyl-quinazolin-4-yloxy)-ethyl]-amine; ODN 2088, ODN with a TTAGGG sequence, G-ODN, statins such as atorvastatin (Clinical trial NCT00519363), IMO-2125 (Idera Pharmaceuticals), IRS 869, CMZ 203-84, CMZ 203-85, CMZ 203-88, CMZ 203-88-1, CMZ 203-89, CMZ 203-91, INH-ODN 2114, ODN A151, ODN INH-1, ODN INH-18, ODN 4084, ODN 4084-F, and ODN INH-47. In some embodiments, the TLR antagonist is selected from the group consisting of chloroquine, quinacrine, monesin, bafilomycin A1, wortmannin, iODN, (+)-morphinans, 9-aminoacridine, 4-aminoquinoline, 4-aminoquinolines, 7,8,9,10-tetrahydro-6H-cyclohepta[b]quinolin-11-ylamine; 1-methyl-2,3-dihydro-1H-pyrrolo[2,3-b]quinolin-4-ylamine; 1,6-dimethyl-2,3-dihydro-H-pyrrolo[2,3-b]quinolin-4-ylamine; 6-bromo-1-methyl-2,3-dihydro-1H-pyrrolo[2,3-b]quinolin-4-ylamine; 1-methyl-2,3,4,5-tetrahydro-1H-azepino[2,3-b]quinolin-6-ylamine; 3,3-dimethyl-3,4-dihydro-acridin-9-ylamine; 1-benzyl-2,3-dihydro-1H-pyrrolo[2,3-b]quinolin-4-ylamine; 6-methyl-1-phenyl-2,3-dihydro-1H-pyrrolo[2,3-b]quinolin-4-ylamine; N*2*,N*2*-Dimethyl-quinoline-2,4-diamine, 2,7-Dimethyl-dibenzo[b,g][1,8]naphthyridin-11-ylamine; 2,4-Dimethyl-benzo[b][1,8]naphthyridin-5-ylamine; 7-Fluoro-2,4-dimethyl-benzo[b][1,8]naphthyridin-5-ylamine; 1,2,3,4-Tetrahydro-acridin-9-ylamine Tacrine hydrochloridehydrate; 2,3-Dihydro-1H-cyclopenta[b]quinolin-9-ylamine; 2,4,9-Trimethyl-benzo[b][1,8]naphthyridin-5-ylamine; 9-Amino-3,3-dimethyl-1,2,3,4-tetrahydro-acridin-1-ol and 7-Ethoxy-N*3*-furan-2-ylmethyl-acridine-3,9-diamine; quinazolines, N,N-dimethyl-N′-{2-[4-(4-methyl-piperazin-1-yl)-phenyl]-3,4-dihydro-quinazoline-4-yl}-ethane-1,2,-diamine; N′-[6,7-Dimethoxy-2-(4-phenyl-piperazin-1-yl)-quinazolin-4-yl]-N,N-dimethyl-ethane-1,2-diamine; N′-[6,7-Dimethoxy-2-(4-methyl-piperazin-1-yl)-quinazolin-4-yl]-N,N-dimethyl-ethane-1,2-diamine; N,N-Dimethyl-N′-(2-phenyl-quinazolin-4-yl)-ethane-1,2-diamine; Dimethyl-(2-{2-[4-(4-methyl-piperazin-1-yl)-phenyl]-quinazolin-4-yloxy}-ethyl)-amine; N′-(2-Biphenyl-4-yl-quinazolin-4-yl)-N,N-dimethyl-ethane-1,2-diamine and Dimethyl-[2-(2-phenyl-quinazolin-4-yloxy)-ethyl]-amine; ODN 2088, ODN with a TTAGGG sequence, G-ODN, statins, atorvastatin, IMO-2125 (Idera Pharmaceuticals), IRS 869, CMZ 203-84, CMZ 203-85, CMZ 203-88, CMZ 203-88-1, CMZ 203-89, CMZ 203-91, INH-ODN 2114, ODN A151, ODN INH-1, ODN INH-18, ODN 4084, ODN 4084-F, and ODN INH-47.

In some embodiments, the TLR7/9 antagonists include IRS 954 (DV-1709, Dynavax), chloroquine, hydroxychloroquine, quinacrine and bafilomycin A, DV1079 (GlaxoSmithKline), IM03100 (Idera Pharmaceuticals), 9-substituted-8-oxo-adenine compounds as described in U.S. Pat. No. 8,063,051, and ODNs as disclosed in Oligodeoxyribonucleotide-Based Antagonists for Toll-Like Receptors 7 and 9, J. Med. Chem., 2009, 52 (2), pp 551-558.

In some embodiments, the TLR inhibitor is a TLR7/8/9 antagonist that targets TLR7, TLR8, and TLR9. In some embodiments, the TLR7/8/9 antagonist is CPG52364 (WO 2008152471), IMO 8400 (Clinical trial NCT01899729, Idera Pharmaceuticals), IMO-9200 (Idera Pharmaceuticals), small molecule antagonists as described in U.S. Pat. No. 7,410,975, 1H imidazoquinoline derived compounds as described in U.S. Pat. No. 8,728,486, oligonucleotides containing a 7-deaza-dG or arabino-G modification in the immune-stimulatory motif and 2′-O-methylribonucleotides (Design, synthesis and biological evaluation of novel antagonist compounds of Toll-like receptors 7, 8 and 9. Nucl. Acids Res. first published online Feb. 8, 2013 doi: 10.1093/nar/gkt078), and oligonucleotide-based antagonist compounds containing a (5-methyl-dC)p(7-deaza-dG) or (5-methyl-dC)p(arabino-G) motif and an immune-regulatory 2′-O-methylribonucleotide motif adjacent to the immune-stimulatory motif (Design, synthesis and biological evaluation of novel antagonist compounds of Toll-like receptors 7, 8 and 9, Nucleic Acids Res. April 2013; 41(6): 3947-3961).

In some embodiments, the TLR7/8 antagonists include IRS 661 and substituted benzoazepines as described in US 20140088085.

In some embodiments, the TLR6 antagonists include the monoclonal anti-hTLR6 IgG (C5C8) antibody.

TAK1 Inhibitors

TAK1 inhibitors are compounds that target transforming growth factor-β-activated kinase 1 (TAK1). In some embodiments, an inhibitor of TAK1 (MAP3K7) is a small molecule, a protein, an antibody or fragment thereof, or an RNAi molecule such as a siRNA or a shRNA molecule.

Exemplary TAK1 (MAP3K7) inhibitors include, but are not limited to: 5Z-7-oxozeaenol, LYTAK1, NG-25, celastrol, and epoxyquinol B (EPQB).

In some embodiments, a TAK1 (MAP3K7) inhibitor is a protein that serves as a negative regulator of TAK1 function. In some instances, an inhibitor of TAK1 includes nemo-like kinase (NLK), USP18, and VopZ.

In some embodiments, a TAK1 (MAP3K7) inhibitor is a biologically active diterpene triepoxide such as triptolide, which inhibits TAK1 kinase activity by interfering with the formation of the TAK1-TAB1 complex (Lu et al., “TAB1: a target of triptolide in macrophages,” Chem Biol. 21(2):246-256 (2014)).

In some embodiments, a TAK1 (MAP3K7) inhibitor is a TAK1 (MAP3K7) inhibitor disclosed in Tan et al. “Discovery of type II inhibitors of TFGβ-activated kinase 1 (TAK1) and mitogen-activated protein kinase kinase kinase kinase 2 (MAP4K2),” J Med Chem. (Jul. 30 2014); Hornberger et al., “Discovery of 7-aminofuro[2,3-c]pyridine inhibitors of TAK1,” Bioorg Med Chem Lett 23(16):4517-4522 (2013); Hornberger et al., “Discovery of 7-aminofuro[2,3-c]pyridine inhibitors of TAK1: optimization of kinase selectivity and pharmacokinetics,” Bioorg Med Chem Lett 23(16):4511-4516 (2013); Shao et al, “7b, a novel naphthalimide derivative, exhibited anti-inflammatory effects via targeted-inhibiting TAK1 following down-regulation of ERK1/2- and p38 MAPK-mediated activation of NF-κB in LPS-stimulated RAW264.7 macrophages,” Int Immunopharmacol 17(2):216-228 (2013); Kitty, et al., “TAK1 inhibition in the DFG-out conformation,” Chem Biol Drug Des 82(5):500-505 (2013); Urich et al., “De novo design of protein kinase inhibitors by in silico identification of hinge region-binding fragments,” ACS Chem Biol 8(5):1044-1052 (2013); and Lockman et al., “Oxindole derivatives as inhibitors of TAK1 kinase,” Bioorg Med Chem Lett 21(6):1724-1727 (2011).

In some embodiments, a TAK1 (MAP3K7) inhibitor is a TAK1 (MAP3K7) inhibitor disclosed in any of the following patent publications: WO2014018888; WO2014155300; WO2013012998; WO2012042091; WO2011100502; WO2008007072; WO2004083854; WO2002048135; and U.S. Pat. No. 8,378,104.

In some embodiments, the TAK1 inhibitor is selected from the group consisting of 5Z-7-oxozeaenol, LYTAK1, NG-25, celastrol, epoxyquinol B (EPQB), nemo-like kinase (NLK), USP18, VopZ, diterpene triepoxide, triptolide, 7-aminofuro[2,3-c]pyridines, naphthalimide derivatives, and oxindole derivatives. In some embodiments, the TAK1 inhibitor is selected from the group consisting of 5Z-7-oxozeaenol, LYTAK1, NG-25, celastrol, and epoxyquinol B (EPQB). In some embodiments, the TAK1 inhibitor is 5Z-7-oxozeaenol.

Hematological Malignancies

Hematological malignancies are a diverse group of cancer that affects the blood, bone marrow, and lymph nodes. In some embodiments, the hematological malignancy is a leukemia, a lymphoma, a myeloma, a non-Hodgkin's lymphoma, a Hodgkin's lymphoma, T-cell malignancy, or a B-cell malignancy.

In some embodiments, the hematological malignancy is a T-cell malignancy. In some embodiments, T-cell malignancies include peripheral T-cell lymphoma not otherwise specified (PTCL-NOS), anaplastic large cell lymphoma, angioimmunoblastic lymphoma, cutaneous T-cell lymphoma, adult T-cell leukemia/lymphoma (ATLL), blastic NK-cell lymphoma, enteropathy-type T-cell lymphoma, hematosplenic gamma-delta T-cell lymphoma, lymphoblastic lymphoma, nasal NK/T-cell lymphomas, or treatment-related T-cell lymphomas.

In some embodiments, the hematological malignancy is a B-cell malignancy. In some embodiments, B-cell malignancies is marginal zone lymphoma (MZL), acute lymphoblastic leukemia (ALL), acute myelogenous leukemia (AML), chronic myelogenous leukemia (CML), acute monocytic leukemia (AMoL), chronic lymphocytic leukemia (CLL), high-risk chronic lymphocytic leukemia (CLL), small lymphocytic lymphoma (SLL), high-risk small lymphocytic lymphoma (SLL), follicular lymphoma (FL), diffuse large B-cell lymphoma (DLBCL), mantle cell lymphoma (MCL), Waldenstrom's macroglobulinemia, multiple myeloma, extranodal marginal zone B cell lymphoma, nodal marginal zone B cell lymphoma, Burkitt's lymphoma, non-Burkitt high grade B cell lymphoma, primary mediastinal B-cell lymphoma (PMBL), immunoblastic large cell lymphoma, precursor B-lymphoblastic lymphoma, B cell prolymphocytic leukemia, lymphoplasmacytic lymphoma, splenic marginal zone lymphoma, plasma cell myeloma, plasmacytoma, mediastinal (thymic) large B cell lymphoma, intravascular large B cell lymphoma, primary effusion lymphoma, or lymphomatoid granulomatosis. In some embodiments, the B-cell malignancy is diffuse large B-cell lymphoma (DLBCL). In some embodiments, the hematological malignancy is diffuse large B-cell lymphoma (DLBCL). In some embodiments, the DLBCL is an activated B-cell DLBCL (ABC-DLBCL), a germinal center B-cell like DLBCL (GBC-DLBCL), a double hit DLBCL (DH-DLBCL), or a triple hit DLBCL (TH-DLBCL).

In some embodiments, the hematological malignancy is a relapsed or refractory hematological malignancy. In some embodiments, the relapsed or refractory hematological malignancy is a relapsed or refractory T-cell malignancy. In some embodiments, the relapsed or refractory hematological malignancy is a relapsed or refractory B-cell malignancy. In some embodiments, the B-cell malignancy is marginal zone lymphoma (MZL), acute lymphoblastic leukemia (ALL), acute myelogenous leukemia (AML), chronic myelogenous leukemia (CML), acute monocytic leukemia (AMoL), chronic lymphocytic leukemia (CLL), high-risk chronic lymphocytic leukemia (CLL), small lymphocytic lymphoma (SLL), high-risk small lymphocytic lymphoma (SLL), follicular lymphoma (FL), diffuse large B-cell lymphoma (DLBCL), mantle cell lymphoma (MCL), Waldenstrom's macroglobulinemia, multiple myeloma, extranodal marginal zone B cell lymphoma, nodal marginal zone B cell lymphoma, Burkitt's lymphoma, non-Burkitt high grade B cell lymphoma, primary mediastinal B-cell lymphoma (PMBL), immunoblastic large cell lymphoma, precursor B-lymphoblastic lymphoma, B cell prolymphocytic leukemia, lymphoplasmacytic lymphoma, splenic marginal zone lymphoma, plasma cell myeloma, plasmacytoma, mediastinal (thymic) large B cell lymphoma, intravascular large B cell lymphoma, primary effusion lymphoma, or lymphomatoid granulomatosis. In some embodiments, the relapsed or refractory B-cell malignancy is diffuse large B-cell lymphoma (DLBCL). In some embodiments, the hematological malignancy is diffuse large B-cell lymphoma (DLBCL). In some embodiments, the DLBCL is an activated B-cell DLBCL (ABC-DLBCL), a germinal center B-cell like DLBCL (GBC-DLBCL), a double hit DLBCL (DH-DLBCL), or a triple hit DLBCL (TH-DLBCL). In some embodiments, the relapsed or refractory hematological malignancy is diffuse large B-cell lymphoma (DLBCL).

In some embodiments, the hematological malignancy is a relapsed hematological malignancy. In some embodiments, the hematological malignancy is a refractory hematological malignancy.

In some embodiments, the hematological malignancy is a non-Hodgkin's lymphoma (NHL). In some embodiments, the NHL is selected from the group consisting of marginal zone lymphoma (MZL), extranodal marginal zone B-cell lymphoma (also known as mucosa-associated lymphoid tissue (MALT) lymphomas), nodal marginal zone B-cell lymphoma, splenic marginal zone B-cell lymphoma, lymphoplasmacytic lymphoma (Waldenstrom macroglobulinemia), hairy cell leukemia, primary central nervous system (CNS) lymphoma, Burkitt lymphoma, chronic lymphocytic leukemia/small lymphocytic lymphoma (CLL/SLL), diffuse large B-cell lymphoma (DLBCL), primary mediastinal B-cell lymphoma, Intravascular large B-cell lymphoma, follicular lymphoma, immunoblastic large cell lymphoma, precursor B-lymphoblastic lymphoma, and mantle cell lymphoma.

In some embodiments, the hematological malignancy is an ibrutinib-resistant hematological malignancy. In some embodiments, the ibrutinib-resistant hematological malignancy is an ibrutinib-resistant T-cell malignancy. In some embodiments, the ibrutinib-resistant hematological malignancy is an ibrutinib-resistant B-cell malignancy. In some embodiments, the ibrutinib-resistant B-cell malignancy is marginal zone lymphoma (MZL), acute lymphoblastic leukemia (ALL), acute myelogenous leukemia (AML), chronic myelogenous leukemia (CML), acute monocytic leukemia (AMoL), chronic lymphocytic leukemia (CLL), high-risk chronic lymphocytic leukemia (CLL), small lymphocytic lymphoma (SLL), high-risk small lymphocytic lymphoma (SLL), follicular lymphoma (FL), diffuse large B-cell lymphoma (DLBCL), mantle cell lymphoma (MCL), Waldenstrom's macroglobulinemia, multiple myeloma, extranodal marginal zone B cell lymphoma, nodal marginal zone B cell lymphoma, Burkitt's lymphoma, non-Burkitt high grade B cell lymphoma, primary mediastinal B-cell lymphoma (PMBL), immunoblastic large cell lymphoma, precursor B-lymphoblastic lymphoma, B cell prolymphocytic leukemia, lymphoplasmacytic lymphoma, splenic marginal zone lymphoma, plasma cell myeloma, plasmacytoma, mediastinal (thymic) large B cell lymphoma, intravascular large B cell lymphoma, primary effusion lymphoma, or lymphomatoid granulomatosis. In some embodiments, the ibrutinib-resistant B-cell malignancy is ibrutinib-resistant diffuse large B-cell lymphoma (DLBCL). In some embodiments, the ibrutinib-resistant hematological malignancy is ibrutinib-resistant diffuse large B-cell lymphoma (DLBCL). In some embodiments, the DLBCL is an activated B-cell DLBCL (ABC-DLBCL), a germinal center B-cell like DLBCL (GBC-DLBCL), a double hit DLBCL (DH-DLBCL), or a triple hit DLBCL (TH-DLBCL). In some embodiments, the hematological malignancy is ibrutinib-resistant diffuse large B-cell lymphoma (DLBCL).

In some embodiments, the ibrutinib-resistant hematological malignancy is an ibrutinib-resistant non-Hodgkin's lymphoma (NHL). In some embodiments, the ibrutinib-resistant NHL is selected from the group consisting of marginal zone lymphoma (MZL), extranodal marginal zone B-cell lymphoma (also known as mucosa-associated lymphoid tissue (MALT) lymphomas), nodal marginal zone B-cell lymphoma, splenic marginal zone B-cell lymphoma, lymphoplasmacytic lymphoma (Waldenstrom macroglobulinemia), hairy cell leukemia, primary central nervous system (CNS) lymphoma, Burkitt lymphoma, chronic lymphocytic leukemia/small lymphocytic lymphoma (CLL/SLL), diffuse large B-cell lymphoma (DLBCL), primary mediastinal B-cell lymphoma, Intravascular large B-cell lymphoma, follicular lymphoma, immunoblastic large cell lymphoma, precursor B-lymphoblastic lymphoma, and mantle cell lymphoma.

In some embodiments, the hematological malignancy is an ibrutinib-sensitive hematological malignancy. In some embodiments, the ibrutinib-sensitive hematological malignancy is an ibrutinib-sensitive T-cell malignancy. In some embodiments, the ibrutinib-sensitive hematological malignancy is an ibrutinib-sensitive B-cell malignancy.

DLBCL

Disclosed herein, in certain embodiments, is a method for treating a diffuse large B-cell lymphoma (DLBCL) comprising administering to a subject in need thereof a therapeutically effective amount of a combination comprising a BTK inhibitor and a TLR inhibitor. In certain embodiments, also disclosed herein, is a method for treating a diffuse large B-cell lymphoma (DLBCL) comprising administering to a subject in need thereof a therapeutically effective amount of a combination comprising a compound of Formula (A), Formula (B), Formula (C), or Formula (D); and a TLR inhibitor.

As used herein, the term “diffuse large B-cell lymphoma (DLBCL)” refers to a neoplasm of the germinal center B lymphocytes with a diffuse growth pattern and a high-intermediate proliferation index. DLBCLs represent approximately 30% of all lymphomas and may present with several morphological variants including the centroblastic, immunoblastic, T-cell/histiocyte rich, anaplastic and plasmoblastic subtypes. Genetic tests have shown that there are different subtypes of DLBCL. These subtypes seem to have different outlooks (prognoses) and responses to treatment. DLBCL can affect any age group but occurs mostly in older people (the average age is mid-60s).

Disclosed herein, in certain embodiments, is a method for treating diffuse large B-cell lymphoma, activated B cell-like subtype (ABC-DLBCL) comprising administering to a subject in need thereof a therapeutically effective amount of a combination comprising a BTK inhibitor and a TLR inhibitor. The ABC subtype of diffuse large B-cell lymphoma (ABC-DLBCL) is thought to arise from post germinal center B cells that are arrested during plasmatic differentiation. The ABC subtype of DLBCL (ABC-DLBCL) accounts for approximately 30% total DLBCL diagnoses. It is considered the least curable of the DLBCL molecular subtypes and, as such, patients diagnosed with the ABC-DLBCL typically display significantly reduced survival rates compared with individuals with other types of DLCBL. ABC-DLBCL is most commonly associated with chromosomal translocations deregulating the germinal center master regulator BCL6 and with mutations inactivating the PRDM1 gene, which encodes a transcriptional repressor required for plasma cell differentiation. In some embodiments, ABC-DLBCL is characterized by a mutation in MYD88. In some embodiments, the mutation is at amino acid position 198 or 265 of MYD88. In some embodiments, the mutation at amino acid position 198 of MYD88 is S198N. In some embodiments, the mutation is at position 265 of MYD88. In some embodiments, the mutation is an L265P mutation of MYD88.

Marginal Zone Lymphoma (MZL)

Marginal zone lymphomas are a group of indolent (slow-growing) NHL B-cell lymphomas, which account for approximately 12 percent of all B-cell lymphomas. The median age for diagnosis is 65 years old. There are three types of marginal zone lymphoma: extranodal marginal zone lymphoma or mucosa-associated lymphoid tissue (MALT), nodal marginal zone lymphoma (sometimes called monocytoid B-cell lymphoma), and splenic marginal zone lymphoma. Extranodal marginal zone lymphoma or mucosa-associated lymphoid tissue (MALT) is the most common form of marginal zone lymphoma. It occurs outside the lymph nodes, in places such as the stomach, small intestine, salivary gland, thyroid, eyes, and lungs. MALT lymphoma is divided into two categories: gastric, which develops in the stomach, and non-gastric, which develops outside of the stomach. This form of lymphoma makes up about nine percent of all B-cell lymphomas. In many cases of MALT lymphoma, there is a previous medical history of inflammation or autoimmune disorders. For example, Helicobacter pylori (H. pylori), a microbial pathogen linked to chronic gastritis, has been associated with a significant portion of patients with gastric MALT lymphoma. Nodal marginal zone lymphoma (sometimes called monocytoid B-cell lymphoma) occurs within the lymph nodes and accounts for about two percent of all B-cell lymphomas. Splenic marginal zone lymphoma occurs most often in the spleen and blood. It has been associated with Hepatitis C. This form of lymphoma makes up about one percent of all B-cell lymphomas.

PIM Inhibitors

Disclosed herein, in certain embodiments, are PIM inhibitors in combination with a BTK inhibitor for the treatment of a hematological malignancy. As used herein, “PIM inhibitor(s)” may be “pan-PIM inhibitor.” “PIM inhibitor(s) may also be “PIM1 inhibitors.” Accordingly, in some embodiments, a “PIM inhibitor” refers to an inhibitor of PIM1. In some embodiments, “PIM inhibitor” refers to a “pan-PIM inhibitor,” or an inhibitor of PIM1, PIM2, and PIM3. PIM inhibitors may also be referred to as PIM kinase inhibitors. Exemplary PIM inhibitors include, but are not limited to, mitoxantrone, SGI-1776, AZD1208, AZD1897, LGH447, JP_11646, Pim1 inhibitor 2, SKI-O-068, CX-6258, AR460770, AR00459339 (Array Biopharma Inc.), miR-33a, Pim-1 inhibitory p27 (Kip1) peptide, LY333′531, K00135, quercetagein (3,3′,4′,5,6,7-hydroxyflavone), and LY294002. In some embodiments, the PIM inhibitor is AZD1208.

In some embodiments, PIM1 inhibitors include rucaparib and veliparib as described in Antolin, et al., “Linking off-target kinase pharmacology to the differential cellular effects observed among PARP inhibitors,” Oncotarget 5(10):3023-3028 (2014); pyrrolo[1,2-a]pyrazinones as described in Casuscelli et al., “Discovery and optimization of pyrrolo[1,2-a]pyrazinones leads to novel and selective inhibitors of PIM kinases,” Bioorg Med Chem. 21(23):7364-7380 (2013); as described in Yoshida et al., “Synthesis, resolution, and biological evaluation of atropisomeric (aR)- and (aS)-16-methyllamellarins N: unique effects of the axial chirality on the selectivity of protein kinases inhibition,” J Med Chem 56(18):7289-7301 (2013); as described in Cozza et al., “Exploiting the repertoire of CK2 inhibitors to target DYRK and PIM kinases,” Biochim Biophys Acta 1834(7):1402-1409 (2013); triazolo[4,5-b]pyridines as described in Saluste et al., “Fragment-hopping-based discovery of a novel chemical series of proto-oncogene PIM-1 kinase inhibitors,” PLoS One 7(10:e45964 (2012); PJ34 as described in Antolin et al., “Identification of pim kinases as noel targets for PJ34 with confounding effects in PARP biology,” ACS Chem Biol. 7(12):1962-1967 (2012); as described in Ogawa et al., “Insights from Pim1 structure for anti-cancer drug design,” Expert Opin Drug Discov. 7(12): 1177-1192 (2012); as described in Brault et al., “PIM kinases are progression markers and emerging therapeutic targets in diffuse large B-cell lymphoma,” Br J Cancer 107(3):491-500 (2012); as described in Nakano et al., “Rational evolution of a novel type of potent and selective proviral integration site in Moloney murine leukemia virus kinase 1 (PIM1) inhibitor from a screening-hit compound,” 55(11):5151-5164 (2012); as described in Hill et al., “Targeting diverse signaling interaction sites allows the rapid generation of bivalent kinase inhibitors,” ACS Chem Biol 7(3):487-495 (2012); as described in Huber et al., “7,8-dichloro-1-oxo-β-carbolines as a versatile scaffold for the development of potent and selective kinase inhibitors with unusual binding modes,” J Med Chem 55(1):403-413 (2012); as described in Morishita et al., “Cell-permeable carboxyl-terminal p27(Kip1) peptide exhibits anti-tumor activity by inhibiting Pim-1 kinase,” J Biol Chem 286(4):2681-2688 (2011); Bullock et al., “Structural basis of inhibitor specificity of the human protooncogene proviral insertion site in moloney murine leukemia virus (PIM-1) kinase,” J. Med. Chem. 48:7604-7614 (2005); Debreczeni et al., “Ruthenium half-sandwich complexes bound to protein kinase Pim-1,” Angew. Chem. Int. Ed. Engl. 45:1580-1585 (2006); Bregman et al., “Ruthenium half-sandwich complexes as protein kinase inhibitors: an N-succinimidyl ester for rapid derivatizations of the cyclopentadienyl moiety,” Org. Lett. 8:5465-5468 (2006); Pogacic et al., “Structural analysis identifies imidazo[1,2-b] pyridazines as PIM kinase inhibitors with in vitro antileukemic activity,” Cancer Res. 67:6916-6924 (2007); Cheney et al., “Identification and structure-activity relationships of substituted pyridones as inhibitors of Pim-1 kinase,” Bioorg. Med. Chem. Lett. 17:1679-1683 (2007); Holder et al., “Comparative molecular field analysis of flavonoid inhibitors of the PIM-1 kinase,” Bioorg. Med. Chem. 15:6463-6473 (2007); Pierce et al., “Docking study yields four novel inhibitors of the protooncogene Pim-1 kinase,” J. Med. Chem. 51:1972-1975 (2008); Tong et al., “Isoxazolo[3,4-b]quinoline-3,4(1H,9H)-diones as unique, potent and selective inhibitors for Pim-1 and Pim-2 kinases: chemistry, biological activities, and molecular modeling,” Bioorg. Med. Chem. Lett. 18:5206-5208 (2008); Xia et al., “Synthesis and evaluation of novel inhibitors of Pim-1 and Pim-2 protein kinases,” J. Med. Chem. 52:74-86 (2009); Qian et al, “Hit to lead account of the discovery of a new class of inhibitors of Pim kinases and crystallographic studies revealing an unusual kinase binding mode,” J. Med. Chem. 52:1814-1827 (2009); Tao et al., “Discovery of 3H-benzo[4,5]thieno[3,2-d] pyrimidin-4-ones as potent, highly selective, and orally bioavailable inhibitors of the human protooncogene proviral insertion site in moloney murine leukemia virus (PIM) kinases,” J. Med. Chem. 52:6621-6636 (2009); Tong et al., “Isoxazolo[3,4-b]quinoline-3,4(1H,9H)-diones as unique, potent and selective inhibitors for Pim-1 and Pim-2 kinases: chemistry, biological activities, and molecular modeling,” Bioorg med Chem Lett. 18(19):5206-5208 (2008); and Pogacic et al., “Structural analysis identifies imidazo[1,2-b]pyridazines as PIM kinase inhibitors with in vitro antileukemic activity,” Cancer Res 67(14):6916-6924 (2007).

In some embodiments, PIM1 inhibitors are described in: U.S. Pat. No. 8,889,704; U.S. Pat. No. 8,822,497; U.S. Pat. No. 8,604,217; U.S. Pat. No. 8,557,809; U.S. Pat. No. 8,575,145; U.S. Pat. No. 8,541,576; U.S. Pat. No. 8,435,976; U.S. Pat. No. 8,242,129; U.S. Pat. No. 8,124,649; U.S. Pat. No. 8,138,181; U.S. Pat. No. 8,829,193; U.S. Pat. No. 8,710,057; U.S. Pat. No. 8,053,454; U.S. Pat. No. 7,268,136; US2014045835; US20140162999; US20140162998; US20110263664; US2011237600; US2011294789; US2010144751; WO2014048939; WO2014033630; WO2014022752; WO2014170403; WO2013175388; WO2013130660; WO2013066684; WO2013013188; WO2013004984; WO2013005041; WO2012156756; WO2012145617; WO2012129338; WO2012148775; WO2012120415; WO2012225062; WO2012098387; WO2012078777; WO2012020215; WO2011101644; WO2011080510; WO2011079274; WO2011035022; WO2011035019; WO2011031979; WO2011025859; WO2011057784; WO2010135571; and WO2009064486.

In some embodiments, disclosed herein are PIM1 inhibitors such as mitoxantrone, SGI-1776, AZD1208, AZD1897, LGH447, JP_11646, Pim1 inhibitor 2, SKI-O-068, CX-6258, AR460770, AR00459339 (Array Biopharma Inc.), miR-33a, Pim-1 inhibitory p27 (Kip1) peptide, LY333′531, K00135, quercetagein (3,3′,4′,5,6,7-hydroxyflavone), or LY294002 in combination with a BTK inhibitor for the treatment of a hematological malignancy. In some embodiments, the the Btk inhibitor is ibrutinib, PCI-45292, PCI-45466, AVL-101/CC-101 (Avila Therapeutics/Celgene Corporation), AVL-263/CC-263 (Avila Therapeutics/Celgene Corporation), AVL-292/CC-292 (Avila Therapeutics/Celgene Corporation), AVL-291/CC-291 (Avila Therapeutics/Celgene Corporation), CNX 774 (Avila Therapeutics), BMS-488516 (Bristol-Myers Squibb), BMS-509744 (Bristol-Myers Squibb), CGI-1746 (CGI Pharma/Gilead Sciences), CGI-560 (CGI Pharma/Gilead Sciences), CTA-056, GDC-0834 (Genentech), HY-11066 (also, CTK417891, HMS3265G21, HMS3265G22, HMS3265H21, HMS3265H22, 439574-61-5, AG-F-54930), ONO-4059 (Ono Pharmaceutical Co., Ltd.), ONO-WG37 (Ono Pharmaceutical Co., Ltd.), PLS-123 (Peking University), RN486 (Hoffmann-La Roche), HM71224 (Hanmi Pharmaceutical Company Limited), LFM-A13, BGB-3111 (Beigene), KBP-7536 (KBP BioSciences), ACP-196 (Acerta Pharma) or JTE-051 (Japan Tobacco Inc). In some embodiments, the BTK inhibitor is ibrutinib.

In some embodiments, disclosed herein are PIM1 inhibitors such as mitoxantrone, SGI-1776, AZD1208, AZD1897, LGH447, JP_11646, Pim1 inhibitor 2, SKI-O-068, CX-6258, AR460770, AR00459339 (Array Biopharma Inc.), miR-33a, Pim-1 inhibitory p27 (Kip1) peptide, LY333′531, K00135, quercetagein (3,3′,4′,5,6,7-hydroxyflavone), or LY294002 in combination with ibrutinib for the treatment of a hematological malignancy. In some embodiments, the hematological malignancy is MCL. In some embodiments, the MCL is a primary resistant MCL.

Diagnostic and Therapeutic Methods

Biomarker

Disclosed herein, in certain embodiments, are methods of treating a B-cell malignancy associated with over-activated TLR signaling, comprising: (a) detecting the presence or absence of a mutation in MYD88 in a sample from an individual; and (b) administering to the individual a therapeutically effective amount of a combination comprising a BTK inhibitor and a TLR inhibitor if the individual has a mutation in MYD88. Also disclosed herein, in certain embodiments, are methods of selecting an individual having a B-cell malignancy for therapy with a combination comprising a BTK inhibitor and a TLR inhibitor, comprising: (a) detecting the presence of absence of a mutation in MYD88 in a sample from an individual; and (b) characterizing the individual as a candidate for therapy with the combination comprising a BTK inhibitor and a TLR inhibitor if the individual has a mutation in MYD88. In some embodiments, an ITK inhibitor is used in combination with a TLR inhibitor. In some embodiments, a TEC inhibitor is used in combination with a TLR inhibitor. In some embodiments, a compound of Formula (A), Formula (B), Formula (C), or Formula (D); is used in combination with a TLR inhibitor.

In some instances, also comprised herein are biomarkers related to the presence, absence, or gene expression levels of TLRs, TLR interacting molecules, TLR downstream effectors, or TLR-related cytokines or chemokines. In some embodiments, exemplary TLRs include TLR1, TLR2, TLR3, TLR4, TLR5, TLR6, TLR7, TLR8, TLR9, TLR10, TLR11, TLR12, or TLR13. In some instances, TLR downstream effectors include CASP8, CHUK, EIF2AK2, IKBKB, IRAK2, IRF1, MAP2K4, NFKB2, NFKBIL1, NFRKB, PPARA, PTGS2, RELA, TAB1, or TRAF6. In some embodiments, TLR interacting molecules include CD14, HSPA1A, LY96, JIP3, RIPK2, or TIRAP. In some embodiments, TLR related cytokines or chemokines include CCL2, CSF2, CSF3, CXCL10, IFNA1, IFNB1, IFNG, IL12A, IL1A, IL1B, IL2, IL6, IL8, or LTA.

In some instances, the expression level of the TLR biomarker or the TLR-related biomarker increases by 0.5-fold, 1-fold, 1.5-fold, 2-fold, 2.5-fold, 3-fold, 3.5-fold, 4-fold, 4.5-fold, 5-fold, 5.5-fold, 6-fold, 6.5-fold, 7-fold, 7.5-fold, 8-fold, 8.5-fold, 9-fold, 9.5-fold, 10-fold, 15-fold, 20-fold, 50-fold, or more compared to the control.

In some instances, the control is the expression levels of the TLR biomarker or the TLR-related biomarker in an individual who is not insensitive toward the BTK inhibitor (e.g. ibrutinib).

In some instances, the control is the expression levels of the TLR biomarker or the TLR-related biomarker in an individual who has not been treated with the BTK inhibitor (e.g. ibrutinib).

Diagnostic Methods

Methods for determining presence of biomarker genes such as MYD88 mutations are well known in the art. Mutations or modifications and expression levels of biomarkers are measured by RT-PCR, Qt-PCR, microarray, Northern blot, or other similar technologies.

As disclosed herein, determining the presence, modifications, or expression of the biomarker of interest at the protein or nucleotide level are accomplished using any detection method known to those of skill in the art. As used herein, “modification” and “mutation” are used interchangeably. The term “biomarker” refers to in some cases the protein of interest. In some cases, “biomarker” refers to the gene of interest. In some cases, the terms “biomarker” and “biomarker gene” are used interchangeably.

In certain aspects of the method provided herein, one or more subpopulation of lymphocytes are isolated, detected or measured. In certain embodiments, the one or more subpopulation of lymphocytes are isolated, detected or measured using immunophenotyping techniques. In other embodiments, one or more subpopulation of lymphocytes are isolated, detected or measured using fluorescence activated cell sorting (FACS) techniques.

In certain aspects, the modifications, expression, or presence of these various biomarkers and any clinically useful prognostic markers in a biological sample are detected at the protein or nucleic acid level, using, for example, immunohistochemistry techniques or nucleic acid-based techniques such as in situ hybridization and RT-PCR. In one embodiments, the modifications, expression, or presence of one or more biomarkers is carried out by a means for nucleic acid amplification, a means for nucleic acid sequencing, a means utilizing a nucleic acid microarray (DNA and RNA), or a means for in situ hybridization using specifically labeled probes.

In some embodiments, the determining the modification, expression, or presence of one or more biomarkers is carried out through gel electrophoresis. In one embodiment, the determination is carried out through transfer to a membrane and hybridization with a specific probe.

In other embodiments, the determining the modification, expression, or presence of one or more biomarkers carried out by a diagnostic imaging technique.

In still other embodiments, the determining the modification, expression, or presence of one or more biomarkers carried out by a detectable solid substrate. In one embodiment, the detectable solid substrate is paramagnetic nanoparticles functionalized with antibodies.

Thus, in some embodiments, the detection of a biomarker or other protein of interest is assayed at the nucleic acid level using nucleic acid probes. The term “nucleic acid probe” refers to any molecule that is capable of selectively binding to a specifically intended target nucleic acid molecule, for example, a nucleotide transcript. Probes are synthesized by one of skill in the art, or derived from appropriate biological preparations. Probes are specifically designed to be labeled, for example, with a radioactive label, a fluorescent label, an enzyme, a chemiluminescent tag, a colorimetric tag, or other labels or tags that are discussed above or that are known in the art. Examples of molecules that are utilized as probes include, but are not limited to, RNA and DNA.

For example, isolated mRNA are used in hybridization or amplification assays that include, but are not limited to, Southern or Northern analyses, polymerase chain reaction analyses and probe arrays. One method for the detection of mRNA levels involves contacting the isolated mRNA with a nucleic acid molecule (probe) that hybridize to the mRNA encoded by the gene being detected. The nucleic acid probe comprises of, for example, a full-length cDNA, or a portion thereof, such as an oligonucleotide of at least 7, 15, 30, 50, 100, 250 or 500 nucleotides in length and sufficient to specifically hybridize under stringent conditions to an mRNA or genomic DNA encoding a biomarker, biomarker described herein above. Hybridization of an mRNA with the probe indicates that the biomarker or other target protein of interest is being expressed.

In one embodiment, the mRNA is immobilized on a solid surface and contacted with a probe, for example by running the isolated mRNA on an agarose gel and transferring the mRNA from the gel to a membrane, such as nitrocellulose. In an alternative embodiment, the probe(s) are immobilized on a solid surface and the mRNA is contacted with the probe(s), for example, in a gene chip array. A skilled artisan readily adapts known mRNA detection methods for use in detecting the level of mRNA encoding the biomarkers or other proteins of interest.

Modifications or expression levels of an RNA of interest are monitored using a membrane blot (such as used in hybridization analysis such as Northern, dot, and the like), or microwells, sample tubes, gels, beads or fibers (or any solid support comprising bound nucleic acids). See U.S. Pat. Nos. 5,770,722, 5,874,219, 5,744,305, 5,677,195 and 5,445,934, which are incorporated herein by reference. The detection of expression also comprises using nucleic acid probes in solution.

In some embodiments, microarrays are used to determine expression or presence of one or more biomarkers. Microarrays are particularly well suited for this purpose because of the reproducibility between different experiments. DNA microarrays provide one method for the simultaneous measurement of the expression levels of large numbers of genes. Each array consists of a reproducible pattern of capture probes attached to a solid support. Labeled RNA or DNA is hybridized to complementary probes on the array and then detected by laser scanning Hybridization intensities for each probe on the array are determined and converted to a quantitative value representing relative gene expression levels. See, U.S. Pat. Nos. 6,040,138, 5,800,992, 6,020,135, 6,033,860, 6,344,316, and U.S. Pat. Application 20120208706, each of which is hereby incorporated in its entirety for all purposes. High-density oligonucleotide arrays are particularly useful for determining the gene expression profile for a large number of RNA's in a sample. Exemplary microarray chips include FoundationOne and FoundationOne Heme from Foundation Medicine, Inc; GeneChip® Human Genome U133 Plus 2.0 array from Affymetrix; and Human DiscoveryMAP® 250+ v. 2.0 from Myraid RBM.

Techniques for the synthesis of these arrays using mechanical synthesis methods are described in, e.g., U.S. Pat. No. 5,384,261. In some embodiments, an array is fabricated on a surface of virtually any shape or even a multiplicity of surfaces. In some embodiments, an array is a planar array surface. In some embodiments, arrays include peptides or nucleic acids on beads, gels, polymeric surfaces, fibers such as fiber optics, glass or any other appropriate substrate, see U.S. Pat. Nos. 5,770,358, 5,789,162, 5,708,153, 6,040,193 and 5,800,992, each of which is hereby incorporated in its entirety for all purposes. In some embodiments, arrays are packaged in such a manner as to allow for diagnostics or other manipulation of an all-inclusive device.

In certain embodiments, expression or presence of one or more biomarkers or other proteins of interest within a biological sample, for example, a sample of bodily fluid, is determined by radioimmunoassays or enzyme-linked immunoassays (ELISAs), competitive binding enzyme-linked immunoassays, dot blot (see, for example, Promega Protocols and Applications Guide, Promega Corporation (1991), Western blot (see, for example, Sambrook et al. (1989) Molecular Cloning, A Laboratory Manual, Vol. 3, Chapter 18 (Cold Spring Harbor Laboratory Press, Plainview, N.Y.), chromatography such as high performance liquid chromatography (HPLC), or other assays known in the art. Thus, the detection assays involve steps such as, but not limited to, immunoblotting, immunodiffusion, immunoelectrophoresis, or immunoprecipitation.

In certain other embodiments, the methods disclosed herein are useful for identifying and treating a hematological malignancy, including those listed herein, that are refractory to (i.e., resistant to, or have become resistant to) first-line oncotherapeutic treatments.

Samples

In some embodiments, the sample for use in the methods is from any tissue or fluid containing nucleic acids from a patient. Samples include, but are not limited, to whole blood, dissociated bone marrow, bone marrow aspirate, pleural fluid, peritoneal fluid, central spinal fluid, abdominal fluid, pancreatic fluid, cerebrospinal fluid, brain fluid, ascites, pericardial fluid, urine, saliva, bronchial lavage, sweat, tears, ear flow, sputum, hydrocele fluid, semen, vaginal flow, milk, amniotic fluid, and secretions of respiratory, intestinal or genitourinary tract. In particular embodiments, the sample is a blood serum sample. In particular embodiments, the sample is from a fluid or tissue that is part of, or associated with, the lymphatic system or circulatory system. In some embodiments, the sample is a blood sample that is a venous, arterial, peripheral, tissue, cord blood sample. In particular embodiments, the sample is a blood cell sample containing one or more peripheral blood mononuclear cells (PBMCs). In some embodiments, the sample contains one or more circulating tumor cells (CTCs). In some embodiments, the sample contains one or more disseminated tumor cells (DTC, e.g., in a bone marrow aspirate sample). In some embodiments, the sample contains tumor cells.

In some embodiments, the samples are obtained from the individual by any suitable means of obtaining the sample using well-known and routine clinical methods. Procedures for obtaining fluid samples from an individual are well known. For example, procedures for drawing and processing whole blood and lymph are well-known and can be employed to obtain a sample for use in the methods provided. Typically, for collection of a blood sample, an anti-coagulation agent (e.g., EDTA, or citrate and heparin or CPD (citrate, phosphate, dextrose) or comparable substances) is added to the sample to prevent coagulation of the blood. In some examples, the blood sample is collected in a collection tube that contains an amount of EDTA to prevent coagulation of the blood sample.

In some embodiments, the sample for use in the methods is obtained from cells of a hematological malignant cell line. In some embodiments, the sample is obtained from cells of a acute lymphoblastic leukemia (ALL), acute myelogenous leukemia (AML), chronic myelogenous leukemia (CML), acute monocytic leukemia (AMoL), chronic lymphocytic leukemia (CLL), high risk CLL, small lymphocytic lymphoma (SLL), high risk SLL, follicular lymphoma (FL), diffuse large B-cell lymphoma (DLBCL), mantle cell lymphoma (MCL), Waldenstrom's macroglobulinemia, multiple myeloma, extranodal marginal zone B cell lymphoma, nodal marginal zone B cell lymphoma, Burkitt's lymphoma, non-Burkitt high grade B cell lymphoma, primary mediastinal B-cell lymphoma (PMBL), immunoblastic large cell lymphoma, precursor B-lymphoblastic lymphoma, B cell prolymphocytic leukemia, lymphoplasmacytic lymphoma, splenic marginal zone lymphoma, plasma cell myeloma, plasmacytoma, mediastinal (thymic) large B cell lymphoma, intravascular large B cell lymphoma, primary effusion lymphoma, or lymphomatoid granulomatosis cell line. In some embodiments, the sample is obtained from cells of a DLBCL cell line.

In some embodiments, the sample is a DLBCL cell or population of DLBCL cells. In some embodiments, the DLBCL cell line is an activated B-cell-like (ABC)-DLBCL cell line. In some embodiments, the DLBCL cell line is a germinal center B-cell-like (GCB)-DLBCL cell line. In some embodiments, the DLBCL cell line is OCI-Ly1, OCI-Ly2, OCI-Ly3, OCI-Ly4, OCI-Ly6, OCI-Ly7, OCI-Ly10, OCI-Ly18, OCI-Ly19, U2932, DB, HBL-1, RIVA, SUDHL2, or TMD8. In some embodiments, the DLBCL cell line that is sensitive to treatment with a BTK inhibitor is TMD8, HBL-1 or OCI-Ly10. In some embodiments, the DLBCL cell line that is resistant to treatment with a BTK inhibitor is OCI-Ly3, DB or OCI-Ly19.

Patient Identification

In some embodiments, the invention relates to a method of identifying a patient for combination therapy comprising a BTK inhibitor and a second agent. In some embodiments, the second agent is a PIM inhibitor. In some embodiments, the PIM inhibitor is a pan-PIM inhibitor. In some embodiments, the PIM inhibitor is a PIM-1 inhibitor. In some embodiments, the patient has non-Hodgkin's lymphoma. In some embodiments, the method of selection includes determining the if the patient is resistant to ibrutinib. In some embodiments, determining if the patient is resistant to ibrutinib comprises performing a drug resistance testing assay. In some embodiments, the drug resistance testing assay is a phenotypic resistance assay. In some embodiments, determining if the patient is resistant to ibrutinib comprises determining an overexpression of TLR4, ILR1 or both. In some embodiments, the overexpression of TLR4, ILR1 or both comprises comparing the expression level of TLR4, ILR1 or both to a reference level. In some embodiments, the patient is not completely resistant to ibrutinib.

In some embodiments, a reference level is level of expression of TLR4, ILR1 or both in a normal patient (e.g., a patient without a hematological malignancy). In some embodiments, a reference level is level of expression of TLR4, ILR1 or both in a sample (e.g., a serum sample) taken from the patient prior to administration of the therapeutically effective amount of the BTK inhibitor.

In some embodiments, the method further comprises administering a combination therapy of a BTK inhibitor and a PIM inhibitor if the patient is resistant to ibrutinib. In some embodiments, the method further comprises administering a combination therapy of ibrutinib and a PIM inhibitor if the patient is resistant to ibrutinib.

In some embodiments, the method further comprises administering a combination therapy of a BTK inhibitor and a PIM inhibitor if the patient's expression level of TLR4, ILR1 or both is higher than the reference level. In some embodiments, the method further comprises administering a combination therapy of ibrutinib and a PIM inhibitor if the patient's expression level of TLR4, ILR1 or both is higher than the reference level.

Additional Combination Therapies

In certain embodiments, a TEC inhibitor and TLR inhibitor are administered in combination with an additional therapeutic agent for the treatment of a hematological malignancy. In some embodiments, the TEC inhibitor is a BTK inhibitor, an ITK inhibitor, a TEC inhibitor, a RLK inhibitor, or a BMX inhibitor. In certain embodiments, an ITK inhibitor and a TLR inhibitor are administered in combination with an additional therapeutic agent for the treatment of a hematological malignancy. In certain embodiments, a BTK inhibitor (e.g., ibrutinib) and a TLR inhibitor are administered in combination with an additional therapeutic agent for the treatment of a hematological malignancy. In some embodiments, the additional therapeutic agent is selected from a chemotherapeutic agent, a biologic agent, radiation therapy, bone marrow transplant or surgery.

In some embodiments, the third therapeutic agent is selected from among a chemotherapeutic agent, a biologic agent, radiation therapy, bone marrow transplant or surgery. In some embodiments, the chemotherapeutic agent is selected from among chlorambucil, ifosfamide, doxorubicin, mesalazine, thalidomide, lenalidomide, temsirolimus, everolimus, fludarabine, fostamatinib, paclitaxel, docetaxel, ofatumumab, rituximab, dexamethasone, prednisone, CAL-101, ibritumomab, tositumomab, bortezomib, pentostatin, endostatin, bendamustine, cyclophosphamide, vincristine, or a combination thereof.

Pharmaceutical Compositions and Formulations

Disclosed herein, in certain embodiments, are pharmaceutical compositions and formulations comprising: a BTK inhibitor; and a TLR inhibitor. In some embodiments, the combination further comprises a pharmaceutically-acceptable excipient. In some embodiments, the TLR inhibitor is selected from a non-specific TLR inhibitor, a TLR7/8/9 antagonist, and a TLR9 antagonist. In some embodiments, the non-specific TLR inhibitor is selected from the group consisting of chloroquine and bafilomycin A. In some embodiments, the TLR7/8/9 antagonist is selected from the group consisting of CPG52364, IMO 8400, and IMO-9200. In some embodiments, the TLR9 antagonist is selected from the group consisting of chloroquine, quinacrine, monesin, bafilomycin A1, wortmannin, iODN, (+)-morphinans, 9-aminoacridine, 4-aminoquinoline, 4-aminoquinolines, 7,8,9,10-tetrahydro-6H-cyclohepta[b]quinolin-11-ylamine; 1-methyl-2,3-dihydro-1H-pyrrolo[2,3-b]quinolin-4-ylamine; 1,6-dimethyl-2,3-dihydro-1H-pyrrolo[2,3-b]quinolin-4-ylamine; 6-bromo-1-methyl-2,3-dihydro-1H-pyrrolo[2,3-b]quinolin-4-ylamine; 1-methyl-2,3,4,5-tetrahydro-H-azepino[2,3-b]quinolin-6-ylamine; 3,3-dimethyl-3,4-dihydro-acridin-9-ylamine; 1-benzyl-2,3-dihydro-1H-pyrrolo[2,3-b]quinolin-4-ylamine; 6-methyl-1-phenyl-2,3-dihydro-1H-pyrrolo[2,3-b]quinolin-4-ylamine; N*2*,N*2*-Dimethyl-quinoline-2,4-diamine, 2,7-Dimethyl-dibenzo[b,g][1,8]naphthyridin-11-ylamine; 2,4-Dimethyl-benzo[b][1,8]naphthyridin-5-ylamine; 7-Fluoro-2,4-dimethyl-benzo[b][1,8]naphthyridin-5-ylamine; 1,2,3,4-Tetrahydro-acridin-9-ylamine Tacrine hydrochloridehydrate; 2,3-Dihydro-1H-cyclopenta[b]quinolin-9-ylamine; 2,4,9-Trimethyl-benzo[b][1,8]naphthyridin-5-ylamine; 9-Amino-3,3-dimethyl-1,2,3,4-tetrahydro-acridin-1-ol and 7-Ethoxy-N*3*-furan-2-ylmethyl-acridine-3,9-diamine; quinazolines, N,N-dimethyl-N′-{2-[4-(4-methyl-piperazin-1-yl)-phenyl]-3,4-dihydro-quinazoline-4-yl}-ethane-1,2,-diamine; N′-[6,7-Dimethoxy-2-(4-phenyl-piperazin-1-yl)-quinazolin-4-yl]-N,N-dimethyl-ethane-1,2-diamine; N′-[6,7-Dimethoxy-2-(4-methyl-piperazin-1-yl)-quinazolin-4-yl]-N,N-dimethyl-ethane-1,2-diamine; N,N-Dimethyl-N′-(2-phenyl-quinazolin-4-yl)-ethane-1,2-diamine; Dimethyl-(2-{2-[4-(4-methyl-piperazin-1-yl)-phenyl]-quinazolin-4-yloxy}-ethyl)-amine; N′-(2-Biphenyl-4-yl-quinazolin-4-yl)-N,N-dimethyl-ethane-1,2-diamine and Dimethyl-[2-(2-phenyl-quinazolin-4-yloxy)-ethyl]-amine; ODN 2088, ODN with a TTAGGG sequence, G-ODN, statins, atorvastatin, IMO-2125 (Idera Pharmaceuticals), IRS 869, CMZ 203-84, CMZ 203-85, CMZ 203-88, CMZ 203-88-1, CMZ 203-89, CMZ 203-91, INH-ODN 2114, ODN A151, ODN INH-1, ODN INH-18, ODN 4084, ODN 4084-F, and ODN INH-47. In some embodiments, the BTK inhibitor is a compound of Formula (D)

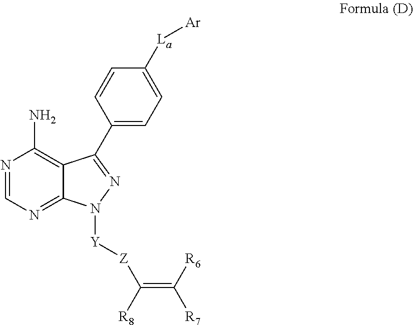

wherein

Lₐ is CH₂, O, NH or S;

Ar is an optionally substituted aromatic carbocycle or an aromatic heterocycle;

Y is an optionally substituted alkyl, heteroalkyl, carbocycle, heterocycle, or combination thereof;

Z is C(O), OC(O), NHC(O), C(S), S(O)ₓ, OS(O)ₓ, NHS(O)ₓ, where x is 1 or 2; and

R₆, R₇, and R₈ are independently selected from H, alkyl, heteroalkyl, carbocycle, heterocycle, or combinations thereof.

In some embodiments, the BTK inhibitor is ibrutinib. In some embodiments, the BTK inhibitor is ibrutinib and the TLR inhibitor is chloroquine.

In some embodiments, the combination provides a synergistic therapeutic effect compared to administration of the BTK inhibitor or the TLR inhibitor alone. In some embodiments, the combination of a BTK inhibitor and a TLR inhibitor exert a very strong synergistic effect, a strong synergistic effect, a synergistic effect, a moderate synergistic effect, a slight synergistic effect, or a combination thereof. In some embodiments, the combination of a BTK inhibitor and a TLR inhibitor exert a very strong synergistic effect. In some embodiments, the BTK inhibitor is ibrutinib.

In some embodiments, the combination of ibrutinib and a TLR inhibitor exert a synergistic effect. In some embodiments, the combination of ibrutinib and a TLR inhibitor sensitize cells to ibrutinib. In some embodiments, synergism is further subdivided into very strong synergism, strong synergism, synergism, moderate synergism, and slight synergism. In some embodiments, the combination of ibrutinib and a TLR inhibitor exert a very strong synergistic effect, a strong synergistic effect, a synergistic effect, a moderate synergistic effect, a slight synergistic effect, or a combination thereof. In some embodiments, the combination of ibrutinib and a TLR inhibitor exert a very strong synergistic effect.

In some embodiments, a combination index (CI) value is used to indicate the behavior of the combination of a BTK inhibitor (e.g. ibrutinib) and a TLR inhibitor. In some embodiments, CI<1 indicates a synergistic effect. In some embodiments, CI=1 indicates an addictive effect. In some embodiments, CI>1 indicates an antagonistic effect. In some embodiments, synergism is further subdivided into very strong synergism, strong synergism, synergism, moderate synergism, and slight synergism. In some embodiments, the CI value for a very strong synergism is at most 0.1, or less. In some embodiments, the CI value for a strong synergism is from about 0.1 to about 0.9, about 0.1 to about 0.5, or about 0.1 to about 0.3. In some embodiments, the CI value for a synergism is from about 0.1 to about 0.9, about 0.2 to about 0.8, or about 0.3 to about 0.7. In some embodiments, the CI value for a moderate synergism is from about 0.1 to about 0.9, about 0.3 to about 0.9, or about 0.7 to about 0.85. In some embodiments, the CI value for a slight synergism is from about 0.1 to about 0.9, about 0.5 to about 0.9, or about 0.85 to about 0.9.

In some embodiments, the combination of an ITK inhibitor and a TLR inhibitor exert a synergistic effect. In some embodiments, the combination of an ITK inhibitor and a TLR inhibitor sensitize cells to the ITK inhibitor. In some embodiments, the combination of a TEC inhibitor and a TLR inhibitor exert a synergistic effect. In some embodiments, the combination of a TEC inhibitor and a TLR inhibitor sensitize cells to the TEC inhibitor.

Pharmaceutical compositions may be formulated in a conventional manner using one or more physiologically acceptable carriers including excipients and auxiliaries which facilitate processing of the active compounds into preparations which can be used pharmaceutically. Proper formulation is dependent upon the route of administration chosen. Any of the well-known techniques, carriers, and excipients may be used as suitable and as understood in the art. A summary of pharmaceutical compositions described herein may be found, for example, in Remington: The Science and Practice of Pharmacy, Nineteenth Ed (Easton, Pa.: Mack Publishing Company, 1995); Hoover, John E., Remington's Pharmaceutical Sciences, Mack Publishing Co., Easton, Pa. 1975; Liberman, H. A. and Lachman, L., Eds., Pharmaceutical Dosage Forms, Marcel Decker, New York, N.Y., 1980; and Pharmaceutical Dosage Forms and Drug Delivery Systems, Seventh Ed. (Lippincott Williams & Wilkins 1999), herein incorporated by reference in their entirety.

A pharmaceutical composition, as used herein, refers to a mixture of a compound described herein, such as, for example, ibrutinib and a TLR inhibitor, with other chemical components, such as carriers, stabilizers, diluents, dispersing agents, suspending agents, thickening agents, and/or excipients. The pharmaceutical composition facilitates administration of the compound to an organism. In practicing the methods of treatment or use provided herein, therapeutically effective amounts of compounds described herein are administered in a pharmaceutical composition to a mammal having a disease, disorder, or condition to be treated. Preferably, the mammal is a human. A therapeutically effective amount can vary widely depending on the severity of the disease, the age and relative health of the subject, the potency of the compound used and other factors. The compounds can be used singly or in combination with one or more therapeutic agents as components of mixtures.

In certain embodiments, compositions may also include one or more pH adjusting agents or buffering agents, including acids such as acetic, boric, citric, lactic, phosphoric and hydrochloric acids; bases such as sodium hydroxide, sodium phosphate, sodium borate, sodium citrate, sodium acetate, sodium lactate and tris-hydroxymethylaminomethane; and buffers such as citrate/dextrose, sodium bicarbonate and ammonium chloride. Such acids, bases and buffers are included in an amount required to maintain pH of the composition in an acceptable range.

In other embodiments, compositions may also include one or more salts in an amount required to bring osmolality of the composition into an acceptable range. Such salts include those having sodium, potassium or ammonium cations and chloride, citrate, ascorbate, borate, phosphate, bicarbonate, sulfate, thiosulfate or bisulfite anions; suitable salts include sodium chloride, potassium chloride, sodium thiosulfate, sodium bisulfite and ammonium sulfate.

The term “pharmaceutical combination” as used herein, means a product that results from the mixing or combining of more than one active ingredient and includes both fixed and non-fixed combinations of the active ingredients. The term “fixed combination” means that the active ingredients, e.g. a compound described herein and a co-agent, are both administered to a patient simultaneously in the form of a single entity or dosage. The term “non-fixed combination” means that the active ingredients, e.g. a compound described herein and a co-agent, are administered to a patient as separate entities either simultaneously, concurrently or sequentially with no specific intervening time limits, wherein such administration provides effective levels of the two compounds in the body of the patient. The latter also applies to cocktail therapy, e.g. the administration of three or more active ingredients.

The pharmaceutical formulations described herein can be administered to a subject by multiple administration routes, including but not limited to, oral, parenteral (e.g., intravenous, subcutaneous, intramuscular), intranasal, buccal, topical, rectal, or transdermal administration routes. The pharmaceutical formulations described herein include, but are not limited to, aqueous liquid dispersions, self-emulsifying dispersions, solid solutions, liposomal dispersions, aerosols, solid dosage forms, powders, immediate release formulations, controlled release formulations, fast melt formulations, tablets, capsules, pills, delayed release formulations, extended release formulations, pulsatile release formulations, multiparticulate formulations, and mixed immediate and controlled release formulations.

Pharmaceutical compositions including a compound described herein may be manufactured in a conventional manner, such as, by way of example only, by means of conventional mixing, dissolving, granulating, dragee-making, levigating, emulsifying, encapsulating, entrapping or compression processes.

“Antifoaming agents” reduce foaming during processing which can result in coagulation of aqueous dispersions, bubbles in the finished film, or generally impair processing. Exemplary anti-foaming agents include silicon emulsions or sorbitan sesquoleate.

“Antioxidants” include, for example, butylated hydroxytoluene (BHT), sodium ascorbate, ascorbic acid, sodium metabisulfite and tocopherol. In certain embodiments, antioxidants enhance chemical stability where required.

In certain embodiments, compositions provided herein may also include one or more preservatives to inhibit microbial activity. Suitable preservatives include mercury-containing substances such as merfen and thiomersal; stabilized chlorine dioxide; and quaternary ammonium compounds such as benzalkonium chloride, cetyltrimethylammonium bromide and cetylpyridinium chloride.

Formulations described herein may benefit from antioxidants, metal chelating agents, thiol containing compounds and other general stabilizing agents. Examples of such stabilizing agents, include, but are not limited to: (a) about 0.5% to about 2% w/v glycerol, (b) about 0.1% to about 1% w/v methionine, (c) about 0.1% to about 2% w/v monothioglycerol, (d) about 1 mM to about 10 mM EDTA, (e) about 0.01% to about 2% w/v ascorbic acid, (f) 0.003% to about 0.02% w/v polysorbate 80, (g) 0.001% to about 0.05% w/v. polysorbate 20, (h) arginine, (i) heparin, (j) dextran sulfate, (k) cyclodextrins, (l) pentosan polysulfate and other heparinoids, (m) divalent cations such as magnesium and zinc; or (n) combinations thereof.

“Binders” impart cohesive qualities and include, e.g., alginic acid and salts thereof; cellulose derivatives such as carboxymethylcellulose, methylcellulose (e.g., Methocel®), hydroxypropylmethylcellulose, hydroxyethylcellulose, hydroxypropylcellulose (e.g., Klucel®), ethylcellulose (e.g., Ethocel®), and microcrystalline cellulose (e.g., Avicel®); microcrystalline dextrose; amylose; magnesium aluminum silicate; polysaccharide acids; bentonites; gelatin; polyvinylpyrrolidone/vinyl acetate copolymer; crospovidone; povidone; starch; pregelatinized starch; tragacanth, dextrin, a sugar, such as sucrose (e.g., Dipac®), glucose, dextrose, molasses, mannitol, sorbitol, xylitol (e.g., Xylitab®), and lactose; a natural or synthetic gum such as acacia, tragacanth, ghatti gum, mucilage of isapol husks, polyvinylpyrrolidone (e.g., Polyvidone® CL, Kollidon® CL, Polyplasdone® XL-10), larch arabogalactan, Veegum®, polyethylene glycol, waxes, sodium alginate, and the like.

A “carrier” or “carrier materials” include any commonly used excipients in pharmaceutics and should be selected on the basis of compatibility with compounds disclosed herein, such as, compounds of ibrutinib and a TLR inhibitor, and the release profile properties of the desired dosage form. Exemplary carrier materials include, e.g., binders, suspending agents, disintegration agents, filling agents, surfactants, solubilizers, stabilizers, lubricants, wetting agents, diluents, and the like. “Pharmaceutically compatible carrier materials” may include, but are not limited to, acacia, gelatin, colloidal silicon dioxide, calcium glycerophosphate, calcium lactate, maltodextrin, glycerine, magnesium silicate, polyvinylpyrrollidone (PVP), cholesterol, cholesterol esters, sodium caseinate, soy lecithin, taurocholic acid, phosphotidylcholine, sodium chloride, tricalcium phosphate, dipotassium phosphate, cellulose and cellulose conjugates, sugars sodium stearoyl lactylate, carrageenan, monoglyceride, diglyceride, pregelatinized starch, and the like. See, e.g., Remington: The Science and Practice of Pharmacy, Nineteenth Ed (Easton, Pa.: Mack Publishing Company, 1995); Hoover, John E., Remington's Pharmaceutical Sciences, Mack Publishing Co., Easton, Pa. 1975; Liberman, H. A. and Lachman, L., Eds., Pharmaceutical Dosage Forms, Marcel Decker, New York, N.Y., 1980; and Pharmaceutical Dosage Forms and Drug Delivery Systems, Seventh Ed. (Lippincott Williams & Wilkins 1999).

“Dispersing agents,” and/or “viscosity modulating agents” include materials that control the diffusion and homogeneity of a drug through liquid media or a granulation method or blend method. In some embodiments, these agents also facilitate the effectiveness of a coating or eroding matrix. Exemplary diffusion facilitators/dispersing agents include, e.g., hydrophilic polymers, electrolytes, Tween® 60 or 80, PEG, polyvinylpyrrolidone (PVP; commercially known as Plasdone®), and the carbohydrate-based dispersing agents such as, for example, hydroxypropyl celluloses (e.g., HPC, HPC-SL, and HPC-L), hydroxypropyl methylcelluloses (e.g., HPMC K100, HPMC K4M, HPMC K15M, and HPMC K100M), carboxymethylcellulose sodium, methylcellulose, hydroxyethylcellulose, hydroxypropylcellulose, hydroxypropylmethylcellulose phthalate, hydroxypropylmethylcellulose acetate stearate (HPMCAS), noncrystalline cellulose, magnesium aluminum silicate, triethanolamine, polyvinyl alcohol (PVA), vinyl pyrrolidone/vinyl acetate copolymer (S630), 4-(1,1,3,3-tetramethylbutyl)-phenol polymer with ethylene oxide and formaldehyde (also known as tyloxapol), poloxamers (e.g., Pluronics F68®, F88®, and F108®, which are block copolymers of ethylene oxide and propylene oxide); and poloxamines (e.g., Tetronic 908®, also known as Poloxamine 908®, which is a tetrafunctional block copolymer derived from sequential addition of propylene oxide and ethylene oxide to ethylenediamine (BASF Corporation, Parsippany, N.J.)), polyvinylpyrrolidone K12, polyvinylpyrrolidone K17, polyvinylpyrrolidone K25, or polyvinylpyrrolidone K30, polyvinylpyrrolidone/vinyl acetate copolymer (S-630), polyethylene glycol, e.g., the polyethylene glycol can have a molecular weight of about 300 to about 6000, or about 3350 to about 4000, or about 7000 to about 5400, sodium carboxymethylcellulose, methylcellulose, polysorbate-80, sodium alginate, gums, such as, e.g., gum tragacanth and gum acacia, guar gum, xanthans, including xanthan gum, sugars, cellulosics, such as, e.g., sodium carboxymethylcellulose, methylcellulose, sodium carboxymethylcellulose, polysorbate-80, sodium alginate, polyethoxylated sorbitan monolaurate, polyethoxylated sorbitan monolaurate, povidone, carbomers, polyvinyl alcohol (PVA), alginates, chitosans and combinations thereof. Plasticizers such as cellulose or triethyl cellulose can also be used as dispersing agents. Dispersing agents particularly useful in liposomal dispersions and self-emulsifying dispersions are dimyristoyl phosphatidyl choline, natural phosphatidyl choline from eggs, natural phosphatidyl glycerol from eggs, cholesterol and isopropyl myristate.

Combinations of one or more erosion facilitator with one or more diffusion facilitator can also be used in the present compositions.

The term “diluent” refers to chemical compounds that are used to dilute the compound of interest prior to delivery. Diluents can also be used to stabilize compounds because they can provide a more stable environment. Salts dissolved in buffered solutions (which also can provide pH control or maintenance) are utilized as diluents in the art, including, but not limited to a phosphate buffered saline solution. In certain embodiments, diluents increase bulk of the composition to facilitate compression or create sufficient bulk for homogenous blend for capsule filling. Such compounds include e.g., lactose, starch, mannitol, sorbitol, dextrose, microcrystalline cellulose such as Avicel®; dibasic calcium phosphate, dicalcium phosphate dihydrate; tricalcium phosphate, calcium phosphate; anhydrous lactose, spray-dried lactose; pregelatinized starch, compressible sugar, such as Di-Pac® (Amstar); mannitol, hydroxypropylmethylcellulose, hydroxypropylmethylcellulose acetate stearate, sucrose-based diluents, confectioner's sugar; monobasic calcium sulfate monohydrate, calcium sulfate dihydrate; calcium lactate trihydrate, dextrates; hydrolyzed cereal solids, amylose; powdered cellulose, calcium carbonate; glycine, kaolin; mannitol, sodium chloride; inositol, bentonite, and the like.

The term “disintegrate” includes both the dissolution and dispersion of the dosage form when contacted with gastrointestinal fluid. “Disintegration agents or disintegrants” facilitate the breakup or disintegration of a substance. Examples of disintegration agents include a starch, e.g., a natural starch such as corn starch or potato starch, a pregelatinized starch such as National 1551 or Amijel®, or sodium starch glycolate such as Promogel® or Explotab®, a cellulose such as a wood product, methylcrystalline cellulose, e.g., Avicel®, Avicel® PH101, Avicel® PH102, Avicel® PH105, Elcema P100, Emcocel®, Vivacel®, Ming Tia®, and Solka-Floc®, methylcellulose, croscarmellose, or a cross-linked cellulose, such as cross-linked sodium carboxymethylcellulose (Ac-Di-Sol®), cross-linked carboxymethylcellulose, or cross-linked croscarmellose, a cross-linked starch such as sodium starch glycolate, a cross-linked polymer such as crospovidone, a cross-linked polyvinylpyrrolidone, alginate such as alginic acid or a salt of alginic acid such as sodium alginate, a clay such as Veegum® HV (magnesium aluminum silicate), a gum such as agar, guar, locust bean, Karaya, pectin, or tragacanth, sodium starch glycolate, bentonite, a natural sponge, a surfactant, a resin such as a cation-exchange resin, citrus pulp, sodium lauryl sulfate, sodium lauryl sulfate in combination starch, and the like.

“Drug absorption” or “absorption” typically refers to the process of movement of drug from site of administration of a drug across a barrier into a blood vessel or the site of action, e.g., a drug moving from the gastrointestinal tract into the portal vein or lymphatic system.

An “enteric coating” is a substance that remains substantially intact in the stomach but dissolves and releases the drug in the small intestine or colon. Generally, the enteric coating comprises a polymeric material that prevents release in the low pH environment of the stomach but that ionizes at a higher pH, typically a pH of 6 to 7, and thus dissolves sufficiently in the small intestine or colon to release the active agent therein.

“Erosion facilitators” include materials that control the erosion of a particular material in gastrointestinal fluid. Erosion facilitators are generally known to those of ordinary skill in the art. Exemplary erosion facilitators include, e.g., hydrophilic polymers, electrolytes, proteins, peptides, and amino acids.

“Filling agents” include compounds such as lactose, calcium carbonate, calcium phosphate, dibasic calcium phosphate, calcium sulfate, microcrystalline cellulose, cellulose powder, dextrose, dextrates, dextran, starches, pregelatinized starch, sucrose, xylitol, lactitol, mannitol, sorbitol, sodium chloride, polyethylene glycol, and the like.

“Flavoring agents” and/or “sweeteners” useful in the formulations described herein, include, e.g., acacia syrup, acesulfame K, alitame, anise, apple, aspartame, banana, Bavarian cream, berry, black currant, butterscotch, calcium citrate, camphor, caramel, cherry, cherry cream, chocolate, cinnamon, bubble gum, citrus, citrus punch, citrus cream, cotton candy, cocoa, cola, cool cherry, cool citrus, cyclamate, cylamate, dextrose, eucalyptus, eugenol, fructose, fruit punch, ginger, glycyrrhetinate, glycyrrhiza (licorice) syrup, grape, grapefruit, honey, isomalt, lemon, lime, lemon cream, monoammonium glyrrhizinate (MagnaSweet®), maltol, mannitol, maple, marshmallow, menthol, mint cream, mixed berry, neohesperidine DC, neotame, orange, pear, peach, peppermint, peppermint cream, Prosweet® Powder, raspberry, root beer, rum, saccharin, safrole, sorbitol, spearmint, spearmint cream, strawberry, strawberry cream, stevia, sucralose, sucrose, sodium saccharin, saccharin, aspartame, acesulfame potassium, mannitol, talin, sylitol, sucralose, sorbitol, Swiss cream, tagatose, tangerine, thaumatin, tutti fruitti, vanilla, walnut, watermelon, wild cherry, wintergreen, xylitol, or any combination of these flavoring ingredients, e.g., anise-menthol, cherry-anise, cinnamon-orange, cherry-cinnamon, chocolate-mint, honey-lemon, lemon-lime, lemon-mint, menthol-eucalyptus, orange-cream, vanilla-mint, and mixtures thereof.

“Lubricants” and “glidants” are compounds that prevent, reduce or inhibit adhesion or friction of materials. Exemplary lubricants include, e.g., stearic acid, calcium hydroxide, talc, sodium stearyl fumerate, a hydrocarbon such as mineral oil, or hydrogenated vegetable oil such as hydrogenated soybean oil (Sterotex®), higher fatty acids and their alkali-metal and alkaline earth metal salts, such as aluminum, calcium, magnesium, zinc, stearic acid, sodium stearates, glycerol, talc, waxes, Stearowet®, boric acid, sodium benzoate, sodium acetate, sodium chloride, leucine, a polyethylene glycol (e.g., PEG-4000) or a methoxypolyethylene glycol such as Carbowax™, sodium oleate, sodium benzoate, glyceryl behenate, polyethylene glycol, magnesium or sodium lauryl sulfate, colloidal silica such as Syloid™, Cab-O-Sil®, a starch such as corn starch, silicone oil, a surfactant, and the like.

A “measurable serum concentration” or “measurable plasma concentration” describes the blood serum or blood plasma concentration, typically measured in mg, μg, or ng of therapeutic agent per mL, dL, or L of blood serum, absorbed into the bloodstream after administration. As used herein, measurable plasma concentrations are typically measured in ng/ml or μg/ml.

“Pharmacodynamics” refers to the factors which determine the biologic response observed relative to the concentration of drug at a site of action.

“Pharmacokinetics” refers to the factors which determine the attainment and maintenance of the appropriate concentration of drug at a site of action.

“Plasticizers” are compounds used to soften the microencapsulation material or film coatings to make them less brittle. Suitable plasticizers include, e.g., polyethylene glycols such as PEG 300, PEG 400, PEG 600, PEG 1450, PEG 3350, and PEG 800, stearic acid, propylene glycol, oleic acid, triethyl cellulose and triacetin. In some embodiments, plasticizers can also function as dispersing agents or wetting agents.

“Solubilizers” include compounds such as triacetin, triethylcitrate, ethyl oleate, ethyl caprylate, sodium lauryl sulfate, sodium doccusate, vitamin E TPGS, dimethylacetamide, N-methylpyrrolidone, N-hydroxyethylpyrrolidone, polyvinylpyrrolidone, hydroxypropylmethyl cellulose, hydroxypropyl cyclodextrins, ethanol, n-butanol, isopropyl alcohol, cholesterol, bile salts, polyethylene glycol 200-600, glycofurol, transcutol, propylene glycol, and dimethyl isosorbide and the like.

“Stabilizers” include compounds such as any antioxidation agents, buffers, acids, preservatives and the like.

“Steady state,” as used herein, is when the amount of drug administered is equal to the amount of drug eliminated within one dosing interval resulting in a plateau or constant plasma drug exposure.

“Suspending agents” include compounds such as polyvinylpyrrolidone, e.g., polyvinylpyrrolidone K12, polyvinylpyrrolidone K17, polyvinylpyrrolidone K25, or polyvinylpyrrolidone K30, vinyl pyrrolidone/vinyl acetate copolymer (S630), polyethylene glycol, e.g., the polyethylene glycol can have a molecular weight of about 300 to about 6000, or about 3350 to about 4000, or about 7000 to about 5400, sodium carboxymethylcellulose, methylcellulose, hydroxypropylmethylcellulose, hydroxymethylcellulose acetate stearate, polysorbate-80, hydroxyethylcellulose, sodium alginate, gums, such as, e.g., gum tragacanth and gum acacia, guar gum, xanthans, including xanthan gum, sugars, cellulosics, such as, e.g., sodium carboxymethylcellulose, methylcellulose, sodium carboxymethylcellulose, hydroxypropylmethylcellulose, hydroxyethylcellulose, polysorbate-80, sodium alginate, polyethoxylated sorbitan monolaurate, polyethoxylated sorbitan monolaurate, povidone and the like.

“Surfactants” include compounds such as sodium lauryl sulfate, sodium docusate, Tween 60 or 80, triacetin, vitamin E TPGS, sorbitan monooleate, polyoxyethylene sorbitan monooleate, polysorbates, polaxomers, bile salts, glyceryl monostearate, copolymers of ethylene oxide and propylene oxide, e.g., Pluronic® (BASF), and the like. Some other surfactants include polyoxyethylene fatty acid glycerides and vegetable oils, e.g., polyoxyethylene (60) hydrogenated castor oil; and polyoxyethylene alkylethers and alkylphenyl ethers, e.g., octoxynol 10, octoxynol 40. In some embodiments, surfactants may be included to enhance physical stability or for other purposes.

“Viscosity enhancing agents” include, e.g., methyl cellulose, xanthan gum, carboxymethyl cellulose, hydroxypropyl cellulose, hydroxypropylmethyl cellulose, hydroxypropylmethyl cellulose acetate stearate, hydroxypropylmethyl cellulose phthalate, carbomer, polyvinyl alcohol, alginates, acacia, chitosans and combinations thereof.

“Wetting agents” include compounds such as oleic acid, glyceryl monostearate, sorbitan monooleate, sorbitan monolaurate, triethanolamine oleate, polyoxyethylene sorbitan monooleate, polyoxyethylene sorbitan monolaurate, sodium docusate, sodium oleate, sodium lauryl sulfate, sodium doccusate, triacetin, Tween 80, vitamin E TPGS, ammonium salts and the like.

Dosage Forms

The compositions described herein can be formulated for administration to a subject via any conventional means including, but not limited to, oral, parenteral (e.g., intravenous, subcutaneous, or intramuscular), buccal, intranasal, rectal or transdermal administration routes. In some embodiments, the composition is formulated for administration in a combined dosage form. In some embodiments, the composition is formulated for administration in a separate dosage forms. As used herein, the term “subject” is used to mean an animal, preferably a mammal, including a human or non-human. The terms “individual(s)”, “subject(s)” and “patient(s)” are used interchangeably herein, and mean any mammal. In some embodiments, the mammal is a human. In some embodiments, the mammal is a non-human. None of the terms require or are limited to situations characterized by the supervision (e.g. constant or intermittent) of a health care worker (e.g. a doctor, a registered nurse, a nurse practitioner, a physician's assistant, an orderly or a hospice worker).

Moreover, the pharmaceutical compositions described herein, which include ibrutinib and/or a TLR inhibitor can be formulated into any suitable dosage form, including but not limited to, aqueous oral dispersions, liquids, gels, syrups, elixirs, slurries, suspensions and the like, for oral ingestion by a patient to be treated, solid oral dosage forms, aerosols, controlled release formulations, fast melt formulations, effervescent formulations, lyophilized formulations, tablets, powders, pills, dragees, capsules, delayed release formulations, extended release formulations, pulsatile release formulations, multiparticulate formulations, and mixed immediate release and controlled release formulations.

Pharmaceutical preparations for oral use can be obtained by mixing one or more solid excipient with one or more of the compounds described herein, optionally grinding the resulting mixture, and processing the mixture of granules, after adding suitable auxiliaries, if desired, to obtain tablets or dragee cores. Suitable excipients include, for example, fillers such as sugars, including lactose, sucrose, mannitol, or sorbitol; cellulose preparations such as, for example, maize starch, wheat starch, rice starch, potato starch, gelatin, gum tragacanth, methylcellulose, microcrystalline cellulose, hydroxypropylmethylcellulose, sodium carboxymethylcellulose; or others such as: polyvinylpyrrolidone (PVP or povidone) or calcium phosphate. If desired, disintegrating agents may be added, such as the cross-linked croscarmellose sodium, polyvinylpyrrolidone, agar, or alginic acid or a salt thereof such as sodium alginate.

Dragee cores are provided with suitable coatings. For this purpose, concentrated sugar solutions may be used, which may optionally contain gum arabic, talc, polyvinylpyrrolidone, carbopol gel, polyethylene glycol, and/or titanium dioxide, lacquer solutions, and suitable organic solvents or solvent mixtures. Dyestuffs or pigments may be added to the tablets or dragee coatings for identification or to characterize different combinations of active compound doses.

Pharmaceutical preparations which can be used orally include push-fit capsules made of gelatin, as well as soft, sealed capsules made of gelatin and a plasticizer, such as glycerol or sorbitol. The push-fit capsules can contain the active ingredients in admixture with filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols. In addition, stabilizers may be added. All formulations for oral administration should be in dosages suitable for such administration.

In some embodiments, the solid dosage forms disclosed herein may be in the form of a tablet, (including a suspension tablet, a fast-melt tablet, a bite-disintegration tablet, a rapid-disintegration tablet, an effervescent tablet, or a caplet), a pill, a powder (including a sterile packaged powder, a dispensable powder, or an effervescent powder) a capsule (including both soft or hard capsules, e.g., capsules made from animal-derived gelatin or plant-derived HPMC, or “sprinkle capsules”), solid dispersion, solid solution, bioerodible dosage form, controlled release formulations, pulsatile release dosage forms, multiparticulate dosage forms, pellets, granules, or an aerosol. In other embodiments, the pharmaceutical formulation is in the form of a powder. In still other embodiments, the pharmaceutical formulation is in the form of a tablet, including but not limited to, a fast-melt tablet. Additionally, pharmaceutical formulations described herein may be administered as a single capsule or in multiple capsule dosage form. In some embodiments, the pharmaceutical formulation is administered in two, or three, or four, capsules or tablets.

In some embodiments, solid dosage forms, e.g., tablets, effervescent tablets, and capsules, are prepared by mixing particles of ibrutinib and/or a TLR inhibitor, with one or more pharmaceutical excipients to form a bulk blend composition. When referring to these bulk blend compositions as homogeneous, it is meant that the particles of ibrutinib and/or a TLR inhibitor, are dispersed evenly throughout the composition so that the composition may be readily subdivided into equally effective unit dosage forms, such as tablets, pills, and capsules. The individual unit dosages may also include film coatings, which disintegrate upon oral ingestion or upon contact with diluent. These formulations can be manufactured by conventional pharmacological techniques.

Conventional pharmacological techniques include, e.g., one or a combination of methods: (1) dry mixing, (2) direct compression, (3) milling, (4) dry or non-aqueous granulation, (5) wet granulation, or (6) fusion. See, e.g., Lachman et al., The Theory and Practice of Industrial Pharmacy (1986). Other methods include, e.g., spray drying, pan coating, melt granulation, granulation, fluidized bed spray drying or coating (e.g., wurster coating), tangential coating, top spraying, tableting, extruding and the like.

The pharmaceutical solid dosage forms described herein can include a compound described herein and one or more pharmaceutically acceptable additives such as a compatible carrier, binder, filling agent, suspending agent, flavoring agent, sweetening agent, disintegrating agent, dispersing agent, surfactant, lubricant, colorant, diluent, solubilizer, moistening agent, plasticizer, stabilizer, penetration enhancer, wetting agent, anti-foaming agent, antioxidant, preservative, or one or more combination thereof. In still other aspects, using standard coating procedures, such as those described in Remington's Pharmaceutical Sciences, 20th Edition (2000), a film coating is provided around the formulation of ibrutinib and/or a TLR inhibitor. In another embodiment, some or all of the particles of ibrutinib and/or a TLR inhibitor, are not microencapsulated and are uncoated.

Suitable carriers for use in the solid dosage forms described herein include, but are not limited to, acacia, gelatin, colloidal silicon dioxide, calcium glycerophosphate, calcium lactate, maltodextrin, glycerine, magnesium silicate, sodium caseinate, soy lecithin, sodium chloride, tricalcium phosphate, dipotassium phosphate, sodium stearoyl lactylate, carrageenan, monoglyceride, diglyceride, pregelatinized starch, hydroxypropylmethylcellulose, hydroxypropylmethylcellulose acetate stearate, sucrose, microcrystalline cellulose, lactose, mannitol and the like.

Suitable filling agents for use in the solid dosage forms described herein include, but are not limited to, lactose, calcium carbonate, calcium phosphate, dibasic calcium phosphate, calcium sulfate, microcrystalline cellulose, cellulose powder, dextrose, dextrates, dextran, starches, pregelatinized starch, hydroxypropylmethycellulose (HPMC), hydroxypropylmethycellulose phthalate, hydroxypropylmethylcellulose acetate stearate (HPMCAS), sucrose, xylitol, lactitol, mannitol, sorbitol, sodium chloride, polyethylene glycol, and the like.

In order to release the compound of ibrutinib and/or a TLR inhibitor, from a solid dosage form matrix as efficiently as possible, disintegrants are often used in the formulation, especially when the dosage forms are compressed with binder. Disintegrants help rupturing the dosage form matrix by swelling or capillary action when moisture is absorbed into the dosage form. Suitable disintegrants for use in the solid dosage forms described herein include, but are not limited to, natural starch such as corn starch or potato starch, a pregelatinized starch such as National 1551 or Amijel®, or sodium starch glycolate such as Promogel® or Explotab®, a cellulose such as a wood product, methylcrystalline cellulose, e.g., Avicel®, Avicel® PH101, Avicel® PH102, Avicel® PH105, Elcema P100, Emcocel®, Vivacel®, Ming Tia®, and Solka-Floc®, methylcellulose, croscarmellose, or a cross-linked cellulose, such as cross-linked sodium carboxymethylcellulose (Ac-Di-Sol®), cross-linked carboxymethylcellulose, or cross-linked croscarmellose, a cross-linked starch such as sodium starch glycolate, a cross-linked polymer such as crospovidone, a cross-linked polyvinylpyrrolidone, alginate such as alginic acid or a salt of alginic acid such as sodium alginate, a clay such as Veegum® HV (magnesium aluminum silicate), a gum such as agar, guar, locust bean, Karaya, pectin, or tragacanth, sodium starch glycolate, bentonite, a natural sponge, a surfactant, a resin such as a cation-exchange resin, citrus pulp, sodium lauryl sulfate, sodium lauryl sulfate in combination starch, and the like.

Binders impart cohesiveness to solid oral dosage form formulations: for powder filled capsule formulation, they aid in plug formation that can be filled into soft or hard shell capsules and for tablet formulation, they ensure the tablet remaining intact after compression and help assure blend uniformity prior to a compression or fill step. Materials suitable for use as binders in the solid dosage forms described herein include, but are not limited to, carboxymethylcellulose, methylcellulose (e.g., Methocel®), hydroxypropylmethylcellulose (e.g. Hypromellose USP Pharmacoat-603, hydroxypropylmethylcellulose acetate stearate (Aqoate HS-LF and HS), hydroxyethylcellulose, hydroxypropylcellulose (e.g., Klucel®), ethylcellulose (e.g., Ethocel®), and microcrystalline cellulose (e.g., Avicel®), microcrystalline dextrose, amylose, magnesium aluminum silicate, polysaccharide acids, bentonites, gelatin, polyvinylpyrrolidone/vinyl acetate copolymer, crospovidone, povidone, starch, pregelatinized starch, tragacanth, dextrin, a sugar, such as sucrose (e.g., Dipac®), glucose, dextrose, molasses, mannitol, sorbitol, xylitol (e.g., Xylitab®), lactose, a natural or synthetic gum such as acacia, tragacanth, ghatti gum, mucilage of isapol husks, starch, polyvinylpyrrolidone (e.g., Povidone® CL, Kollidon® CL, Polyplasdone® XL-10, and Povidone® K-12), larch arabogalactan, Veegum®, polyethylene glycol, waxes, sodium alginate, and the like.

In general, binder levels of 20-70% are used in powder-filled gelatin capsule formulations. Binder usage level in tablet formulations varies whether direct compression, wet granulation, roller compaction, or usage of other excipients such as fillers which itself can act as moderate binder. Formulators skilled in art can determine the binder level for the formulations, but binder usage level of up to 70% in tablet formulations is common.

Suitable lubricants or glidants for use in the solid dosage forms described herein include, but are not limited to, stearic acid, calcium hydroxide, talc, corn starch, sodium stearyl fumerate, alkali-metal and alkaline earth metal salts, such as aluminum, calcium, magnesium, zinc, stearic acid, sodium stearates, magnesium stearate, zinc stearate, waxes, Stearowet®, boric acid, sodium benzoate, sodium acetate, sodium chloride, leucine, a polyethylene glycol or a methoxypolyethylene glycol such as Carbowax™, PEG 4000, PEG 5000, PEG 6000, propylene glycol, sodium oleate, glyceryl behenate, glyceryl palmitostearate, glyceryl benzoate, magnesium or sodium lauryl sulfate, and the like.

Suitable diluents for use in the solid dosage forms described herein include, but are not limited to, sugars (including lactose, sucrose, and dextrose), polysaccharides (including dextrates and maltodextrin), polyols (including mannitol, xylitol, and sorbitol), cyclodextrins and the like.

The term “non water-soluble diluent” represents compounds typically used in the formulation of pharmaceuticals, such as calcium phosphate, calcium sulfate, starches, modified starches and microcrystalline cellulose, and microcellulose (e.g., having a density of about 0.45 g/cm³, e.g. Avicel, powdered cellulose), and talc.

Suitable wetting agents for use in the solid dosage forms described herein include, for example, oleic acid, glyceryl monostearate, sorbitan monooleate, sorbitan monolaurate, triethanolamine oleate, polyoxyethylene sorbitan monooleate, polyoxyethylene sorbitan monolaurate, quaternary ammonium compounds (e.g., Polyquat 10®), sodium oleate, sodium lauryl sulfate, magnesium stearate, sodium docusate, triacetin, vitamin E TPGS and the like.

Suitable surfactants for use in the solid dosage forms described herein include, for example, sodium lauryl sulfate, sorbitan monooleate, polyoxyethylene sorbitan monooleate, polysorbates, polaxomers, bile salts, glyceryl monostearate, copolymers of ethylene oxide and propylene oxide, e.g., Pluronic® (BASF), and the like.

Suitable suspending agents for use in the solid dosage forms described here include, but are not limited to, polyvinylpyrrolidone, e.g., polyvinylpyrrolidone K12, polyvinylpyrrolidone K17, polyvinylpyrrolidone K25, or polyvinylpyrrolidone K30, polyethylene glycol, e.g., the polyethylene glycol can have a molecular weight of about 300 to about 6000, or about 3350 to about 4000, or about 7000 to about 5400, vinyl pyrrolidone/vinyl acetate copolymer (S630), sodium carboxymethylcellulose, methylcellulose, hydroxy-propylmethylcellulose, polysorbate-80, hydroxyethylcellulose, sodium alginate, gums, such as, e.g., gum tragacanth and gum acacia, guar gum, xanthans, including xanthan gum, sugars, cellulosics, such as, e.g., sodium carboxymethylcellulose, methylcellulose, sodium carboxymethylcellulose, hydroxypropylmethylcellulose, hydroxyethylcellulose, polysorbate-80, sodium alginate, polyethoxylated sorbitan monolaurate, polyethoxylated sorbitan monolaurate, povidone and the like.

Suitable antioxidants for use in the solid dosage forms described herein include, for example, e.g., butylated hydroxytoluene (BHT), sodium ascorbate, and tocopherol.

It should be appreciated that there is considerable overlap between additives used in the solid dosage forms described herein. Thus, the above-listed additives should be taken as merely exemplary, and not limiting, of the types of additives that can be included in solid dosage forms described herein. The amounts of such additives can be readily determined by one skilled in the art, according to the particular properties desired.

In other embodiments, one or more layers of the pharmaceutical formulation are plasticized. Illustratively, a plasticizer is generally a high boiling point solid or liquid. Suitable plasticizers can be added from about 0.01% to about 50% by weight (w/w) of the coating composition. Plasticizers include, but are not limited to, diethyl phthalate, citrate esters, polyethylene glycol, glycerol, acetylated glycerides, triacetin, polypropylene glycol, polyethylene glycol, triethyl citrate, dibutyl sebacate, stearic acid, stearol, stearate, and castor oil.

Compressed tablets are solid dosage forms prepared by compacting the bulk blend of the formulations described above. In various embodiments, compressed tablets which are designed to dissolve in the mouth will include one or more flavoring agents. In other embodiments, the compressed tablets will include a film surrounding the final compressed tablet. In some embodiments, the film coating can provide a delayed release of ibrutinib or the second agent, from the formulation. In other embodiments, the film coating aids in patient compliance (e.g., Opadry® coatings or sugar coating). Film coatings including Opadry® typically range from about 1% to about 3% of the tablet weight. In other embodiments, the compressed tablets include one or more excipients.

A capsule may be prepared, for example, by placing the bulk blend of the formulation of ibrutinib or the second agent, described above, inside of a capsule. In some embodiments, the formulations (non-aqueous suspensions and solutions) are placed in a soft gelatin capsule. In other embodiments, the formulations are placed in standard gelatin capsules or non-gelatin capsules such as capsules comprising HPMC. In other embodiments, the formulation is placed in a sprinkle capsule, wherein the capsule may be swallowed whole or the capsule may be opened and the contents sprinkled on food prior to eating. In some embodiments, the therapeutic dose is split into multiple (e.g., two, three, or four) capsules. In some embodiments, the entire dose of the formulation is delivered in a capsule form.

In various embodiments, the particles of ibrutinib and/or a TLR inhibitor, and one or more excipients are dry blended and compressed into a mass, such as a tablet, having a hardness sufficient to provide a pharmaceutical composition that substantially disintegrates within less than about 30 minutes, less than about 35 minutes, less than about 40 minutes, less than about 45 minutes, less than about 50 minutes, less than about 55 minutes, or less than about 60 minutes, after oral administration, thereby releasing the formulation into the gastrointestinal fluid.

In another aspect, dosage forms may include microencapsulated formulations. In some embodiments, one or more other compatible materials are present in the microencapsulation material. Exemplary materials include, but are not limited to, pH modifiers, erosion facilitators, anti-foaming agents, antioxidants, flavoring agents, and carrier materials such as binders, suspending agents, disintegration agents, filling agents, surfactants, solubilizers, stabilizers, lubricants, wetting agents, and diluents.

Materials useful for the microencapsulation described herein include materials compatible with ibrutinib and/or a TLR inhibitor, which sufficiently isolate the compound of any of ibrutinib or a TLR inhibitor, from other non-compatible excipients. Materials compatible with compounds of any of ibrutinib or a TLR inhibitor, are those that delay the release of the compounds of any of ibrutinib or a TLR inhibitor, in vivo.

Exemplary microencapsulation materials useful for delaying the release of the formulations including compounds described herein, include, but are not limited to, hydroxypropyl cellulose ethers (HPC) such as Klucel® or Nisso HPC, low-substituted hydroxypropyl cellulose ethers (L-HPC), hydroxypropyl methyl cellulose ethers (HPMC) such as Seppifilm-LC, Pharmacoat®, Metolose SR, Methocel®-E, Opadry YS, PrimaFlo, Benecel MP824, and Benecel MP843, methylcellulose polymers such as Methocel®-A, hydroxypropylmethylcellulose acetate stearate Aqoat (HF-LS, HF-LG, HF-MS) and Metolose®, Ethylcelluloses (EC) and mixtures thereof such as E461, Ethocel®, Aqualon®-EC, Surelease®, Polyvinyl alcohol (PVA) such as Opadry AMB, hydroxyethylcelluloses such as Natrosol®, carboxymethylcelluloses and salts of carboxymethylcelluloses (CMC) such as Aqualon®-CMC, polyvinyl alcohol and polyethylene glycol co-polymers such as Kollicoat IR®, monoglycerides (Myverol), triglycerides (KLX), polyethylene glycols, modified food starch, acrylic polymers and mixtures of acrylic polymers with cellulose ethers such as Eudragit® EPO, Eudragit® L30D-55, Eudragit® FS 30D Eudragit® L100-55, Eudragit® L100, Eudragit® S100, Eudragit® RD 100, Eudragit® E100, Eudragit® L12.5, Eudragit® S12.5, Eudragit® NE30D, and Eudragit® NE 40D, cellulose acetate phthalate, sepifilms such as mixtures of HPMC and stearic acid, cyclodextrins, and mixtures of these materials.

In still other embodiments, plasticizers such as polyethylene glycols, e.g., PEG 300, PEG 400, PEG 600, PEG 1450, PEG 3350, and PEG 800, stearic acid, propylene glycol, oleic acid, and triacetin are incorporated into the microencapsulation material. In other embodiments, the microencapsulating material useful for delaying the release of the pharmaceutical compositions is from the USP or the National Formulary (NF). In yet other embodiments, the microencapsulation material is Klucel. In still other embodiments, the microencapsulation material is methocel.

Microencapsulated compounds of any of ibrutinib or a TLR inhibitor, may be formulated by methods known by one of ordinary skill in the art. Such known methods include, e.g., spray drying processes, spinning disk-solvent processes, hot melt processes, spray chilling methods, fluidized bed, electrostatic deposition, centrifugal extrusion, rotational suspension separation, polymerization at liquid-gas or solid-gas interface, pressure extrusion, or spraying solvent extraction bath. In addition to these, several chemical techniques, e.g., complex coacervation, solvent evaporation, polymer-polymer incompatibility, interfacial polymerization in liquid media, in situ polymerization, in-liquid drying, and desolvation in liquid media could also be used. Furthermore, other methods such as roller compaction, extrusion/spheronization, coacervation, or nanoparticle coating may also be used.

In one embodiment, the particles of compounds of any of ibrutinib or a TLR inhibitor, are microencapsulated prior to being formulated into one of the above forms. In still another embodiment, some or most of the particles are coated prior to being further formulated by using standard coating procedures, such as those described in Remington's Pharmaceutical Sciences, 20th Edition (2000).

In other embodiments, the solid dosage formulations of the compounds of any of ibrutinib and/or a TLR inhibitor, are plasticized (coated) with one or more layers. Illustratively, a plasticizer is generally a high boiling point solid or liquid. Suitable plasticizers can be added from about 0.01% to about 50% by weight (w/w) of the coating composition. Plasticizers include, but are not limited to, diethyl phthalate, citrate esters, polyethylene glycol, glycerol, acetylated glycerides, triacetin, polypropylene glycol, polyethylene glycol, triethyl citrate, dibutyl sebacate, stearic acid, stearol, stearate, and castor oil.

In other embodiments, a powder including the formulations with a compound of any of ibrutinib and/or a TLR inhibitor, described herein, may be formulated to include one or more pharmaceutical excipients and flavors. Such a powder may be prepared, for example, by mixing the formulation and optional pharmaceutical excipients to form a bulk blend composition. Additional embodiments also include a suspending agent and/or a wetting agent. This bulk blend is uniformly subdivided into unit dosage packaging or multi-dosage packaging units.

In still other embodiments, effervescent powders are also prepared in accordance with the present disclosure. Effervescent salts have been used to disperse medicines in water for oral administration. Effervescent salts are granules or coarse powders containing a medicinal agent in a dry mixture, usually composed of sodium bicarbonate, citric acid and/or tartaric acid. When salts of the compositions described herein are added to water, the acids and the base react to liberate carbon dioxide gas, thereby causing “effervescence.” Examples of effervescent salts include, e.g., the following ingredients: sodium bicarbonate or a mixture of sodium bicarbonate and sodium carbonate, citric acid and/or tartaric acid. Any acid-base combination that results in the liberation of carbon dioxide can be used in place of the combination of sodium bicarbonate and citric and tartaric acids, as long as the ingredients were suitable for pharmaceutical use and result in a pH of about 6.0 or higher.

In some embodiments, the solid dosage forms described herein can be formulated as enteric coated delayed release oral dosage forms, i.e., as an oral dosage form of a pharmaceutical composition as described herein which utilizes an enteric coating to affect release in the small intestine of the gastrointestinal tract. The enteric coated dosage form may be a compressed or molded or extruded tablet/mold (coated or uncoated) containing granules, powder, pellets, beads or particles of the active ingredient and/or other composition components, which are themselves coated or uncoated. The enteric coated oral dosage form may also be a capsule (coated or uncoated) containing pellets, beads or granules of the solid carrier or the composition, which are themselves coated or uncoated.

The term “delayed release” as used herein refers to the delivery so that the release can be accomplished at some generally predictable location in the intestinal tract more distal to that which would have been accomplished if there had been no delayed release alterations. In some embodiments the method for delay of release is coating. Any coatings should be applied to a sufficient thickness such that the entire coating does not dissolve in the gastrointestinal fluids at pH below about 5, but does dissolve at pH about 5 and above. It is expected that any anionic polymer exhibiting a pH-dependent solubility profile can be used as an enteric coating in the methods and compositions described herein to achieve delivery to the lower gastrointestinal tract. In some embodiments the polymers described herein are anionic carboxylic polymers. In other embodiments, the polymers and compatible mixtures thereof, and some of their properties, include, but are not limited to:

Shellac, also called purified lac, a refined product obtained from the resinous secretion of an insect. This coating dissolves in media of pH >7;

Acrylic polymers. The performance of acrylic polymers (primarily their solubility in biological fluids) can vary based on the degree and type of substitution. Examples of suitable acrylic polymers include methacrylic acid copolymers and ammonium methacrylate copolymers. The Eudragit series E, L, S, RL, RS and NE (Rohm Pharma) are available as solubilized in organic solvent, aqueous dispersion, or dry powders. The Eudragit series RL, NE, and RS are insoluble in the gastrointestinal tract but are permeable and are used primarily for colonic targeting. The Eudragit series E dissolve in the stomach. The Eudragit series L, L-30D and S are insoluble in stomach and dissolve in the intestine;

Cellulose Derivatives. Examples of suitable cellulose derivatives are: ethyl cellulose; reaction mixtures of partial acetate esters of cellulose with phthalic anhydride. The performance can vary based on the degree and type of substitution. Cellulose acetate phthalate (CAP) dissolves in pH >6. Aquateric (FMC) is an aqueous based system and is a spray dried CAP psuedolatex with particles <1 μm. Other components in Aquateric can include pluronics, Tweens, and acetylated monoglycerides. Other suitable cellulose derivatives include: cellulose acetate trimellitate (Eastman); methylcellulose (Pharmacoat, Methocel); hydroxypropylmethyl cellulose phthalate (HPMCP); hydroxypropylmethyl cellulose succinate (HPMCS); and hydroxypropylmethylcellulose acetate succinate (e.g., AQOAT (Shin Etsu)). The performance can vary based on the degree and type of substitution. For example, HPMCP such as, HP-50, HP-55, HP-55S, HP-55F grades are suitable. The performance can vary based on the degree and type of substitution. For example, suitable grades of hydroxypropylmethylcellulose acetate succinate include, but are not limited to, AS-LG (LF), which dissolves at pH 5, AS-MG (MF), which dissolves at pH 5.5, and AS-HG (HF), which dissolves at higher pH. These polymers are offered as granules, or as fine powders for aqueous dispersions; Poly Vinyl Acetate Phthalate (PVAP). PVAP dissolves in pH >5, and it is much less permeable to water vapor and gastric fluids.

In some embodiments, the coating can, and usually does, contain a plasticizer and possibly other coating excipients such as colorants, talc, and/or magnesium stearate, which are well known in the art. Suitable plasticizers include triethyl citrate (Citroflex 2), triacetin (glyceryl triacetate), acetyl triethyl citrate (Citroflec A2), Carbowax 400 (polyethylene glycol 400), diethyl phthalate, tributyl citrate, acetylated monoglycerides, glycerol, fatty acid esters, propylene glycol, and dibutyl phthalate. In particular, anionic carboxylic acrylic polymers usually will contain 10-25% by weight of a plasticizer, especially dibutyl phthalate, polyethylene glycol, triethyl citrate and triacetin. Conventional coating techniques such as spray or pan coating are employed to apply coatings. The coating thickness must be sufficient to ensure that the oral dosage form remains intact until the desired site of topical delivery in the intestinal tract is reached.

Colorants, detackifiers, surfactants, antifoaming agents, lubricants (e.g., carnuba wax or PEG) may be added to the coatings besides plasticizers to solubilize or disperse the coating material, and to improve coating performance and the coated product.

In other embodiments, the formulations described herein, which include ibrutinib and/or a TLR inhibitor, are delivered using a pulsatile dosage form. A pulsatile dosage form is capable of providing one or more immediate release pulses at predetermined time points after a controlled lag time or at specific sites. Many other types of controlled release systems known to those of ordinary skill in the art and are suitable for use with the formulations described herein. Examples of such delivery systems include, e.g., polymer-based systems, such as polylactic and polyglycolic acid, plyanhydrides and polycaprolactone; porous matrices, nonpolymer-based systems that are lipids, including sterols, such as cholesterol, cholesterol esters and fatty acids, or neutral fats, such as mono-, di- and triglycerides; hydrogel release systems; silastic systems; peptide-based systems; wax coatings, bioerodible dosage forms, compressed tablets using conventional binders and the like. See, e.g., Liberman et al., Pharmaceutical Dosage Forms, 2 Ed., Vol. 1, pp. 209-214 (1990); Singh et al., Encyclopedia of Pharmaceutical Technology, 2^{nd} Ed., pp. 751-753 (2002); U.S. Pat. Nos. 4,327,725, 4,624,848, 4,968,509, 5,461,140, 5,456,923, 5,516,527, 5,622,721, 5,686,105, 5,700,410, 5,977,175, 6,465,014 and 6,932,983.

In some embodiments, pharmaceutical formulations are provided that include particles of ibrutinib and/or a TLR inhibitor, described herein and at least one dispersing agent or suspending agent for oral administration to a subject. The formulations may be a powder and/or granules for suspension, and upon admixture with water, a substantially uniform suspension is obtained.

Liquid formulation dosage forms for oral administration can be aqueous suspensions selected from the group including, but not limited to, pharmaceutically acceptable aqueous oral dispersions, emulsions, solutions, elixirs, gels, and syrups. See, e.g., Singh et al., Encyclopedia of Pharmaceutical Technology, 2nd Ed., pp. 754-757 (2002). In addition the liquid dosage forms may include additives, such as: (a) disintegrating agents; (b) dispersing agents; (c) wetting agents; (d) at least one preservative, (e) viscosity enhancing agents, (f) at least one sweetening agent, and (g) at least one flavoring agent. In some embodiments, the aqueous dispersions can further include a crystalline inhibitor.

The aqueous suspensions and dispersions described herein can remain in a homogenous state, as defined in The USP Pharmacists' Pharmacopeia (2005 edition, chapter 905), for at least 4 hours. The homogeneity should be determined by a sampling method consistent with regard to determining homogeneity of the entire composition. In one embodiment, an aqueous suspension can be re-suspended into a homogenous suspension by physical agitation lasting less than 1 minute. In another embodiment, an aqueous suspension can be re-suspended into a homogenous suspension by physical agitation lasting less than 45 seconds. In yet another embodiment, an aqueous suspension can be re-suspended into a homogenous suspension by physical agitation lasting less than 30 seconds. In still another embodiment, no agitation is necessary to maintain a homogeneous aqueous dispersion.

Examples of disintegrating agents for use in the aqueous suspensions and dispersions include, but are not limited to, a starch, e.g., a natural starch such as corn starch or potato starch, a pregelatinized starch such as National 1551 or Amijel®, or sodium starch glycolate such as Promogel® or Explotab®; a cellulose such as a wood product, methylcrystalline cellulose, e.g., Avicel®, Avicel® PH101, Avicel® PH102, Avicel® PH105, Elcema P100, Emcocel®, Vivacel®, Ming Tia®, and Solka-Floc®, methylcellulose, croscarmellose, or a cross-linked cellulose, such as cross-linked sodium carboxymethylcellulose (Ac-Di-Sol®), cross-linked carboxymethylcellulose, or cross-linked croscarmellose; a cross-linked starch such as sodium starch glycolate; a cross-linked polymer such as crospovidone; a cross-linked polyvinylpyrrolidone; alginate such as alginic acid or a salt of alginic acid such as sodium alginate; a clay such as Veegum® HV (magnesium aluminum silicate); a gum such as agar, guar, locust bean, Karaya, pectin, or tragacanth; sodium starch glycolate; bentonite; a natural sponge; a surfactant; a resin such as a cation-exchange resin; citrus pulp; sodium lauryl sulfate; sodium lauryl sulfate in combination starch; and the like.

In some embodiments, the dispersing agents suitable for the aqueous suspensions and dispersions described herein are known in the art and include, for example, hydrophilic polymers, electrolytes, Tween® 60 or 80, PEG, polyvinylpyrrolidone (PVP; commercially known as Plasdone®), and the carbohydrate-based dispersing agents such as, for example, hydroxypropylcellulose and hydroxypropyl cellulose ethers (e.g., HPC, HPC-SL, and HPC-L), hydroxypropyl methylcellulose and hydroxypropyl methylcellulose ethers (e.g. HPMC K100, HPMC K4M, HPMC K15M, and HPMC K100M), carboxymethylcellulose sodium, methylcellulose, hydroxyethylcellulose, hydroxypropylmethyl-cellulose phthalate, hydroxypropylmethyl-cellulose acetate stearate, noncrystalline cellulose, magnesium aluminum silicate, triethanolamine, polyvinyl alcohol (PVA), polyvinylpyrrolidone/vinyl acetate copolymer (Plasdone®, e.g., S-630), 4-(1,1,3,3-tetramethylbutyl)-phenol polymer with ethylene oxide and formaldehyde (also known as tyloxapol), poloxamers (e.g., Pluronics F68®, F88®, and F108®, which are block copolymers of ethylene oxide and propylene oxide); and poloxamines (e.g., Tetronic 908®, also known as Poloxamine 908®, which is a tetrafunctional block copolymer derived from sequential addition of propylene oxide and ethylene oxide to ethylenediamine (BASF Corporation, Parsippany, N.J.)). In other embodiments, the dispersing agent is selected from a group not comprising one of the following agents: hydrophilic polymers; electrolytes; Tween® 60 or 80; PEG; polyvinylpyrrolidone (PVP); hydroxypropylcellulose and hydroxypropyl cellulose ethers (e.g., HPC, HPC-SL, and HPC-L); hydroxypropyl methylcellulose and hydroxypropyl methylcellulose ethers (e.g. HPMC K100, HPMC K4M, HPMC K15M, HPMC K100M, and Pharmacoat® USP 2910 (Shin-Etsu)); carboxymethylcellulose sodium; methylcellulose; hydroxyethylcellulose; hydroxypropylmethyl-cellulose phthalate; hydroxypropylmethyl-cellulose acetate stearate; non-crystalline cellulose; magnesium aluminum silicate; triethanolamine; polyvinyl alcohol (PVA); 4-(1,1,3,3-tetramethylbutyl)-phenol polymer with ethylene oxide and formaldehyde; poloxamers (e.g., Pluronics F68®, F88®, and F108®, which are block copolymers of ethylene oxide and propylene oxide); or poloxamines (e.g., Tetronic 908®, also known as Poloxamine 908®).

Wetting agents suitable for the aqueous suspensions and dispersions described herein are known in the art and include, but are not limited to, cetyl alcohol, glycerol monostearate, polyoxyethylene sorbitan fatty acid esters (e.g., the commercially available Tweens® such as e.g., Tween 20® and Tween 80® (ICI Specialty Chemicals)), and polyethylene glycols (e.g., Carbowaxs 3350® and 1450®, and Carbopol 934® (Union Carbide)), oleic acid, glyceryl monostearate, sorbitan monooleate, sorbitan monolaurate, triethanolamine oleate, polyoxyethylene sorbitan monooleate, polyoxyethylene sorbitan monolaurate, sodium oleate, sodium lauryl sulfate, sodium docusate, triacetin, vitamin E TPGS, sodium taurocholate, simethicone, phosphotidylcholine and the like.

Suitable preservatives for the aqueous suspensions or dispersions described herein include, for example, potassium sorbate, parabens (e.g., methylparaben and propylparaben), benzoic acid and its salts, other esters of parahydroxybenzoic acid such as butylparaben, alcohols such as ethyl alcohol or benzyl alcohol, phenolic compounds such as phenol, or quaternary compounds such as benzalkonium chloride. Preservatives, as used herein, are incorporated into the dosage form at a concentration sufficient to inhibit microbial growth.

Suitable viscosity enhancing agents for the aqueous suspensions or dispersions described herein include, but are not limited to, methyl cellulose, xanthan gum, carboxymethyl cellulose, hydroxypropyl cellulose, hydroxypropylmethyl cellulose, Plasdon® S-630, carbomer, polyvinyl alcohol, alginates, acacia, chitosans and combinations thereof. The concentration of the viscosity enhancing agent will depend upon the agent selected and the viscosity desired.

Examples of sweetening agents suitable for the aqueous suspensions or dispersions described herein include, for example, acacia syrup, acesulfame K, alitame, anise, apple, aspartame, banana, Bavarian cream, berry, black currant, butterscotch, calcium citrate, camphor, caramel, cherry, cherry cream, chocolate, cinnamon, bubble gum, citrus, citrus punch, citrus cream, cotton candy, cocoa, cola, cool cherry, cool citrus, cyclamate, cylamate, dextrose, eucalyptus, eugenol, fructose, fruit punch, ginger, glycyrrhetinate, glycyrrhiza (licorice) syrup, grape, grapefruit, honey, isomalt, lemon, lime, lemon cream, monoammonium glyrrhizinate (MagnaSweet®), maltol, mannitol, maple, marshmallow, menthol, mint cream, mixed berry, neohesperidine DC, neotame, orange, pear, peach, peppermint, peppermint cream, Prosweet® Powder, raspberry, root beer, rum, saccharin, safrole, sorbitol, spearmint, spearmint cream, strawberry, strawberry cream, stevia, sucralose, sucrose, sodium saccharin, saccharin, aspartame, acesulfame potassium, mannitol, talin, sucralose, sorbitol, swiss cream, tagatose, tangerine, thaumatin, tutti fruitti, vanilla, walnut, watermelon, wild cherry, wintergreen, xylitol, or any combination of these flavoring ingredients, e.g., anise-menthol, cherry-anise, cinnamon-orange, cherry-cinnamon, chocolate-mint, honey-lemon, lemon-lime, lemon-mint, menthol-eucalyptus, orange-cream, vanilla-mint, and mixtures thereof. In one embodiment, the aqueous liquid dispersion can comprise a sweetening agent or flavoring agent in a concentration ranging from about 0.001% to about 1.0% the volume of the aqueous dispersion. In another embodiment, the aqueous liquid dispersion can comprise a sweetening agent or flavoring agent in a concentration ranging from about 0.005% to about 0.5% the volume of the aqueous dispersion. In yet another embodiment, the aqueous liquid dispersion can comprise a sweetening agent or flavoring agent in a concentration ranging from about 0.01% to about 1.0% the volume of the aqueous dispersion.

In addition to the additives listed above, the liquid formulations can also include inert diluents commonly used in the art, such as water or other solvents, solubilizing agents, and emulsifiers. Exemplary emulsifiers are ethyl alcohol, isopropyl alcohol, ethyl carbonate, ethyl acetate, benzyl alcohol, benzyl benzoate, propyleneglycol, 1,3-butyleneglycol, dimethylformamide, sodium lauryl sulfate, sodium doccusate, cholesterol, cholesterol esters, taurocholic acid, phosphotidylcholine, oils, such as cottonseed oil, groundnut oil, corn germ oil, olive oil, castor oil, and sesame oil, glycerol, tetrahydrofurfuryl alcohol, polyethylene glycols, fatty acid esters of sorbitan, or mixtures of these substances, and the like.

In some embodiments, the pharmaceutical formulations described herein can be self-emulsifying drug delivery systems (SEDDS). Emulsions are dispersions of one immiscible phase in another, usually in the form of droplets. Generally, emulsions are created by vigorous mechanical dispersion. SEDDS, as opposed to emulsions or microemulsions, spontaneously form emulsions when added to an excess of water without any external mechanical dispersion or agitation. An advantage of SEDDS is that only gentle mixing is required to distribute the droplets throughout the solution. Additionally, water or the aqueous phase can be added just prior to administration, which ensures stability of an unstable or hydrophobic active ingredient. Thus, the SEDDS provides an effective delivery system for oral and parenteral delivery of hydrophobic active ingredients. SEDDS may provide improvements in the bioavailability of hydrophobic active ingredients. Methods of producing self-emulsifying dosage forms are known in the art and include, but are not limited to, for example, U.S. Pat. Nos. 5,858,401, 6,667,048, and 6,960,563, each of which is specifically incorporated by reference.

It is to be appreciated that there is overlap between the above-listed additives used in the aqueous dispersions or suspensions described herein, since a given additive is often classified differently by different practitioners in the field, or is commonly used for any of several different functions. Thus, the above-listed additives should be taken as merely exemplary, and not limiting, of the types of additives that can be included in formulations described herein. The amounts of such additives can be readily determined by one skilled in the art, according to the particular properties desired.

Intranasal Formulations

Intranasal formulations are known in the art and are described in, for example, U.S. Pat. Nos. 4,476,116, 5,116,817 and 6,391,452, each of which is specifically incorporated by reference. Formulations that include ibrutinib and/or a TLR inhibitor, which are prepared according to these and other techniques well-known in the art are prepared as solutions in saline, employing benzyl alcohol or other suitable preservatives, fluorocarbons, and/or other solubilizing or dispersing agents known in the art. See, for example, Ansel, H. C. et al., Pharmaceutical Dosage Forms and Drug Delivery Systems, Sixth Ed. (1995). Preferably these compositions and formulations are prepared with suitable nontoxic pharmaceutically acceptable ingredients. These ingredients are known to those skilled in the preparation of nasal dosage forms and some of these can be found in REMINGTON: THE SCIENCE AND PRACTICE OF PHARMACY, 21st edition, 2005, a standard reference in the field. The choice of suitable carriers is highly dependent upon the exact nature of the nasal dosage form desired, e.g., solutions, suspensions, ointments, or gels. Nasal dosage forms generally contain large amounts of water in addition to the active ingredient. Minor amounts of other ingredients such as pH adjusters, emulsifiers or dispersing agents, preservatives, surfactants, gelling agents, or buffering and other stabilizing and solubilizing agents may also be present. The nasal dosage form should be isotonic with nasal secretions.

For administration by inhalation described herein may be in a form as an aerosol, a mist or a powder. Pharmaceutical compositions described herein are conveniently delivered in the form of an aerosol spray presentation from pressurized packs or a nebulizer, with the use of a suitable propellant, e.g., dichlorodifluoromethane, trichlorofluoromethane, dichlorotetrafluoroethane, carbon dioxide or other suitable gas. In the case of a pressurized aerosol, the dosage unit may be determined by providing a valve to deliver a metered amount. Capsules and cartridges of, such as, by way of example only, gelatin for use in an inhaler or insufflator may be formulated containing a powder mix of the compound described herein and a suitable powder base such as lactose or starch.

Buccal Formulations

Buccal formulations may be administered using a variety of formulations known in the art. For example, such formulations include, but are not limited to, U.S. Pat. Nos. 4,229,447, 4,596,795, 4,755,386, and 5,739,136, each of which is specifically incorporated by reference. In addition, the buccal dosage forms described herein can further include a bioerodible (hydrolysable) polymeric carrier that also serves to adhere the dosage form to the buccal mucosa. The buccal dosage form is fabricated so as to erode gradually over a predetermined time period, wherein the delivery is provided essentially throughout. Buccal drug delivery, as will be appreciated by those skilled in the art, avoids the disadvantages encountered with oral drug administration, e.g., slow absorption, degradation of the active agent by fluids present in the gastrointestinal tract and/or first-pass inactivation in the liver. With regard to the bioerodible (hydrolysable) polymeric carrier, it will be appreciated that virtually any such carrier can be used, so long as the desired drug release profile is not compromised, and the carrier is compatible with ibrutinib and/or a TLR inhibitor, and any other components that may be present in the buccal dosage unit. Generally, the polymeric carrier comprises hydrophilic (water-soluble and water-swellable) polymers that adhere to the wet surface of the buccal mucosa. Examples of polymeric carriers useful herein include acrylic acid polymers and co, e.g., those known as “carbomers” (Carbopol®, which may be obtained from B.F. Goodrich, is one such polymer). Other components may also be incorporated into the buccal dosage forms described herein include, but are not limited to, disintegrants, diluents, binders, lubricants, flavoring, colorants, preservatives, and the like. For buccal or sublingual administration, the compositions may take the form of tablets, lozenges, or gels formulated in a conventional manner.

Transdermal Formulations

Transdermal formulations described herein may be administered using a variety of devices which have been described in the art. For example, such devices include, but are not limited to, U.S. Pat. Nos. 3,598,122, 3,598,123, 3,710,795, 3,731,683, 3,742,951, 3,814,097, 3,921,636, 3,972,995, 3,993,072, 3,993,073, 3,996,934, 4,031,894, 4,060,084, 4,069,307, 4,077,407, 4,201,211, 4,230,105, 4,292,299, 4,292,303, 5,336,168, 5,665,378, 5,837,280, 5,869,090, 6,923,983, 6,929,801 and 6,946,144, each of which is specifically incorporated by reference in its entirety.

The transdermal dosage forms described herein may incorporate certain pharmaceutically acceptable excipients which are conventional in the art. In one embodiments, the transdermal formulations described herein include at least three components: (1) a formulation of a compound of ibrutinib and a TLR inhibitor; (2) a penetration enhancer; and (3) an aqueous adjuvant. In addition, transdermal formulations can include additional components such as, but not limited to, gelling agents, creams and ointment bases, and the like. In some embodiments, the transdermal formulation can further include a woven or non-woven backing material to enhance absorption and prevent the removal of the transdermal formulation from the skin. In other embodiments, the transdermal formulations described herein can maintain a saturated or supersaturated state to promote diffusion into the skin.

Formulations suitable for transdermal administration of compounds described herein may employ transdermal delivery devices and transdermal delivery patches and can be lipophilic emulsions or buffered, aqueous solutions, dissolved and/or dispersed in a polymer or an adhesive. Such patches may be constructed for continuous, pulsatile, or on demand delivery of pharmaceutical agents. Still further, transdermal delivery of the compounds described herein can be accomplished by means of iontophoretic patches and the like. Additionally, transdermal patches can provide controlled delivery of ibrutinib and a TLR inhibitor. The rate of absorption can be slowed by using rate-controlling membranes or by trapping the compound within a polymer matrix or gel. Conversely, absorption enhancers can be used to increase absorption. An absorption enhancer or carrier can include absorbable pharmaceutically acceptable solvents to assist passage through the skin. For example, transdermal devices are in the form of a bandage comprising a backing member, a reservoir containing the compound optionally with carriers, optionally a rate controlling barrier to deliver the compound to the skin of the host at a controlled and predetermined rate over a prolonged period of time, and means to secure the device to the skin.

Injectable Formulations

Formulations that include a compound of ibrutinib and/or a TLR inhibitor, suitable for intramuscular, subcutaneous, or intravenous injection may include physiologically acceptable sterile aqueous or non-aqueous solutions, dispersions, suspensions or emulsions, and sterile powders for reconstitution into sterile injectable solutions or dispersions. Examples of suitable aqueous and non-aqueous carriers, diluents, solvents, or vehicles including water, ethanol, polyols (propyleneglycol, polyethylene-glycol, glycerol, cremophor and the like), suitable mixtures thereof, vegetable oils (such as olive oil) and injectable organic esters such as ethyl oleate. Proper fluidity can be maintained, for example, by the use of a coating such as lecithin, by the maintenance of the required particle size in the case of dispersions, and by the use of surfactants. Formulations suitable for subcutaneous injection may also contain additives such as preserving, wetting, emulsifying, and dispensing agents. Prevention of the growth of microorganisms can be ensured by various antibacterial and antifungal agents, such as parabens, chlorobutanol, phenol, sorbic acid, and the like. It may also be desirable to include isotonic agents, such as sugars, sodium chloride, and the like. Prolonged absorption of the injectable pharmaceutical form can be brought about by the use of agents delaying absorption, such as aluminum monostearate and gelatin.

For intravenous injections, compounds described herein may be formulated in aqueous solutions, preferably in physiologically compatible buffers such as Hank's solution, Ringer's solution, or physiological saline buffer. For transmucosal administration, penetrants appropriate to the barrier to be permeated are used in the formulation. Such penetrants are generally known in the art. For other parenteral injections, appropriate formulations may include aqueous or nonaqueous solutions, preferably with physiologically compatible buffers or excipients. Such excipients are generally known in the art.

Parenteral injections may involve bolus injection or continuous infusion. Formulations for injection may be presented in unit dosage form, e.g., in ampoules or in multi-dose containers, with an added preservative. The pharmaceutical composition described herein may be in a form suitable for parenteral injection as a sterile suspensions, solutions or emulsions in oily or aqueous vehicles, and may contain formulatory agents such as suspending, stabilizing and/or dispersing agents. Pharmaceutical formulations for parenteral administration include aqueous solutions of the active compounds in water-soluble form. Additionally, suspensions of the active compounds may be prepared as appropriate oily injection suspensions. Suitable lipophilic solvents or vehicles include fatty oils such as sesame oil, or synthetic fatty acid esters, such as ethyl oleate or triglycerides, or liposomes. Aqueous injection suspensions may contain substances which increase the viscosity of the suspension, such as sodium carboxymethyl cellulose, sorbitol, or dextran. Optionally, the suspension may also contain suitable stabilizers or agents which increase the solubility of the compounds to allow for the preparation of highly concentrated solutions. Alternatively, the active ingredient may be in powder form for constitution with a suitable vehicle, e.g., sterile pyrogen-free water, before use.

Other Formulations

In certain embodiments, delivery systems for pharmaceutical compounds may be employed, such as, for example, liposomes and emulsions. In certain embodiments, compositions provided herein can also include an mucoadhesive polymer, selected from among, for example, carboxymethylcellulose, carbomer (acrylic acid polymer), poly(methylmethacrylate), polyacrylamide, polycarbophil, acrylic acid/butyl acrylate copolymer, sodium alginate and dextran.

In some embodiments, the compounds described herein may be administered topically and can be formulated into a variety of topically administrable compositions, such as solutions, suspensions, lotions, gels, pastes, medicated sticks, balms, creams or ointments. Such pharmaceutical compounds can contain solubilizers, stabilizers, tonicity enhancing agents, buffers and preservatives.

The compounds described herein may also be formulated in rectal compositions such as enemas, rectal gels, rectal foams, rectal aerosols, suppositories, jelly suppositories, or retention enemas, containing conventional suppository bases such as cocoa butter or other glycerides, as well as synthetic polymers such as polyvinylpyrrolidone, PEG, and the like. In suppository forms of the compositions, a low-melting wax such as, but not limited to, a mixture of fatty acid glycerides, optionally in combination with cocoa butter is first melted.

Dosing and Treatment Regiments

In some embodiments, the amount of ibrutinib that is administered in combination with a TLR inhibitor is from 10 mg/day up to, and including, 1000 mg/day. In some embodiments, the amount of ibrutinib that is administered is from about 40 mg/day to 70 mg/day. In some embodiments, the amount of Ibrutinib that is administered per day is about 10 mg, about 11 mg, about 12 mg, about 13 mg, about 14 mg, about 15 mg, about 16 mg, about 17 mg, about 18 mg, about 19 mg, about 20 mg, about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, about 100 mg, about 110 mg, about 120 mg, about 125 mg, about 130 mg, about 135 mg, or about 140 mg. In some embodiments, the amount of ibrutinib that is administered is about 40 mg/day. In some embodiments, the amount of ibrutinib that is administered is about 50 mg/day. In some embodiments, the amount of ibrutinib that is administered is about 60 mg/day. In some embodiments, the amount of ibrutinib that is administered is about 70 mg/day.

In some embodiments, the amount of a TLR inhibitor that is administered in combination with ibrutinib is from 0.01 μM to, and including, 100 μM. In some embodiments, the amount of a TLR inhibitor is from about 0.01 μM to about 100 μM.

In some embodiments, ibrutinib is administered once per day, twice per day, or three times per day. In some embodiments, ibrutinib is administered once per day. In some embodiments, a TLR inhibitor is administered once per day, twice per day, or three times per day. In some embodiments, a TLR inhibitor is administered once per day. In some embodiments, Ibrutinib and a TLR inhibitor are co-administered (e.g., in a single dosage form), once per day.

In some embodiments, the compositions disclosed herein are administered for prophylactic, therapeutic, or maintenance treatment. In some embodiments, the compositions disclosed herein are administered for therapeutic applications. In some embodiments, the compositions disclosed herein are administered for therapeutic applications. In some embodiments, the compositions disclosed herein are administered as a maintenance therapy, for example for a patient in remission.

In the case wherein the patient's status does improve, upon the doctor's discretion the administration of the compounds may be given continuously; alternatively, the dose of drug being administered may be temporarily reduced or temporarily suspended for a certain length of time (i.e., a “drug holiday”). The length of the drug holiday can vary between 2 days and 1 year, including by way of example only, 2 days, 3 days, 4 days, 5 days, 6 days, 7 days, 10 days, 12 days, 15 days, 20 days, 28 days, 35 days, 50 days, 70 days, 100 days, 120 days, 150 days, 180 days, 200 days, 250 days, 280 days, 300 days, 320 days, 350 days, or 365 days. The dose reduction during a drug holiday may be from 10%-100%, including, by way of example only, 10%, 15%, 20%, 25%, 30%, 35%, 40%, 45%, 50%, 55%, 60%, 65%, 70%, 75%, 80%, 85%, 90%, 95%, or 100%.

Once improvement of the patient's conditions has occurred, a maintenance dose is administered if necessary. Subsequently, the dosage or the frequency of administration, or both, can be reduced, as a function of the symptoms, to a level at which the improved disease, disorder or condition is retained. Patients can, however, require intermittent treatment on a long-term basis upon any recurrence of symptoms.

The amount of a given agent that will correspond to such an amount will vary depending upon factors such as the particular compound, the severity of the disease, the identity (e.g., weight) of the subject or host in need of treatment, but can nevertheless be routinely determined in a manner known in the art according to the particular circumstances surrounding the case, including, e.g., the specific agent being administered, the route of administration, and the subject or host being treated. In general, however, doses employed for adult human treatment will typically be in the range of 0.02-5000 mg per day, or from about 1-1500 mg per day. The desired dose may conveniently be presented in a single dose or as divided doses administered simultaneously (or over a short period of time) or at appropriate intervals, for example as two, three, four or more sub-doses per day.

The pharmaceutical composition described herein may be in unit dosage forms suitable for single administration of precise dosages. In unit dosage form, the formulation is divided into unit doses containing appropriate quantities of one or more compound. The unit dosage may be in the form of a package containing discrete quantities of the formulation. Non-limiting examples are packaged tablets or capsules, and powders in vials or ampoules. Aqueous suspension compositions can be packaged in single-dose non-reclosable containers. Alternatively, multiple-dose reclosable containers can be used, in which case it is typical to include a preservative in the composition. By way of example only, formulations for parenteral injection may be presented in unit dosage form, which include, but are not limited to ampoules, or in multi-dose containers, with an added preservative.

The foregoing ranges are merely suggestive, as the number of variables in regard to an individual treatment regime is large, and considerable excursions from these recommended values are not uncommon. Such dosages may be altered depending on a number of variables, not limited to the activity of the compound used, the disease or condition to be treated, the mode of administration, the requirements of the individual subject, the severity of the disease or condition being treated, and the judgment of the practitioner.

Toxicity and therapeutic efficacy of such therapeutic regimens can be determined by standard pharmaceutical procedures in cell cultures or experimental animals, including, but not limited to, the determination of the LD50 (the dose lethal to 50% of the population) and the ED50 (the dose therapeutically effective in 50% of the population). The dose ratio between the toxic and therapeutic effects is the therapeutic index and it can be expressed as the ratio between LD50 and ED50. Compounds exhibiting high therapeutic indices are preferred. The data obtained from cell culture assays and animal studies can be used in formulating a range of dosage for use in human. The dosage of such compounds lies preferably within a range of circulating concentrations that include the ED50 with minimal toxicity. The dosage may vary within this range depending upon the dosage form employed and the route of administration utilized.

Kits/Article of Manufacture

Disclosed herein, in certain embodiments, are kits and articles of manufacture for use with one or more methods described herein. Such kits include a carrier, package, or container that is compartmentalized to receive one or more containers such as vials, tubes, and the like, each of the container(s) comprising one of the separate elements to be used in a method described herein. Suitable containers include, for example, bottles, vials, syringes, and test tubes. In one embodiment, the containers are formed from a variety of materials such as glass or plastic.

The articles of manufacture provided herein contain packaging materials. Examples of pharmaceutical packaging materials include, but are not limited to, blister packs, bottles, tubes, bags, containers, bottles, and any packaging material suitable for a selected formulation and intended mode of administration and treatment.

For example, the container(s) include ibrutinib, optionally in a composition or in combination with a TLR inhibitor as disclosed herein. Such kits optionally include an identifying description or label or instructions relating to its use in the methods described herein.

A kit typically includes labels listing contents and/or instructions for use, and package inserts with instructions for use. A set of instructions will also typically be included.

In one embodiment, a label is on or associated with the container. In one embodiment, a label is on a container when letters, numbers or other characters forming the label are attached, molded or etched into the container itself; a label is associated with a container when it is present within a receptacle or carrier that also holds the container, e.g., as a package insert. In one embodiment, a label is used to indicate that the contents are to be used for a specific therapeutic application. The label also indicates directions for use of the contents, such as in the methods described herein.

In certain embodiments, the pharmaceutical compositions are presented in a pack or dispenser device which contains one or more unit dosage forms containing a compound provided herein. The pack, for example, contains metal or plastic foil, such as a blister pack. In one embodiment, the pack or dispenser device is accompanied by instructions for administration. In one embodiment, the pack or dispenser is also accompanied with a notice associated with the container in form prescribed by a governmental agency regulating the manufacture, use, or sale of pharmaceuticals, which notice is reflective of approval by the agency of the form of the drug for human or veterinary administration. Such notice, for example, is the labeling approved by the U.S. Food and Drug Administration for prescription drugs, or the approved product insert. In one embodiment, compositions containing a compound provided herein formulated in a compatible pharmaceutical carrier are also prepared, placed in an appropriate container, and labeled for treatment of an indicated condition.

### Examples

These examples are provided for illustrative purposes only and not to limit the scope of the claims provided herein.

### Example 1: Combined Drug Treatment for Cell Viability in TMD8 Cell Line

ABC-DLBCL cell line TMD8 wild-type (wt), which contains the MYD88 L265P mutation, was tested in vitro to determine the effect of ibrutinib in combination with TLR antagonists on cell viability.

200 μl of TMD8 wt cells (1.0×10⁴ cells) at 5.0×10⁴ cells/ml was plated into each well of a 96-well plate. The cells were grown in RPMI-10P medium.

The TLR9 antagonists used for this experiment included ODN 4084-F, ODN INH-1, ODN INH-18, and ODN TTAGGG. Neutral ODN was used as a negative control in this experiment as it does not contain agonistic or antagonistic TLR activities. The TLR9 agonists used for this experiment included ODN 2006, ODN 2216, and ODN 2395. The TLR9 agonists were used to stimulate TLR signaling. Chloroquine is a non-specific TLR antagonist.

Ibrutinib (Lot#131098) at 100, 20, 4, 0.8, 0.16, 0.032, 0.0064, 0.00128, 0.000256, 0 nM concentrations was used during the experiment. The concentrations of the TLR9 antagonists, chloroquine, and TLR9 agonists are shown in Table 1. The stock solution for ibrutinib was prepared at 20 mM concentration. The stock solutions for the TLR9 antagonists and the TLR9 agonists were each prepared at 500 μM concentration. The stock solution for chloroquine diphosphate was prepared at 50 mM concentration.

TABLE 1

| | |
|---|---|
| Final | |
| Concentration | |
| (μM) | Lot Number |

TLR9 agonist

| | | |
|---|---|---|
| ODN 2006 | 1 | InvivoGen #tlr1- |
| | | 2006: 14B19-MM |
| ODN 2216 | 1 | InvivoGen #tlr1- |
| | | 2216: 14B19-MM |
| ODN 2395 | 1 | InvivoGen #tlr1- |
| | | 2395: 14B20-MM |

TLR9 antagonist

| | | |
|---|---|---|
| ODN 4084-F | 1 | InvivoGen #tlr1- |
| | | kit9i: 14B30-MM |
| ODN INH-1 | 1 | InvivoGen #tlr1- |
| | | kit9i: 14B30-MM |
| ODN INH-18 | 1 | InvivoGen #tlr1- |
| | | kit9i: 14B30-MM |
| ODN | 1 | InvivoGen #tlr1- |
| TTAGGG | | kit9i: 14B30-MM |

Additional agents

| | | |
|---|---|---|
| Neutral ODN | 1 | InvivoGen #tlr1- |
| | | kit9i: 14B30-MM |
| Chloroquine | 10 | SIGMA |
| diphosphate | | #C6628: BCBM9716V |

To each well of a 96-W plate was added 100 μL ibrutinib (2× of target concentration; diluted using RPMI-10P medium), 25 μL TLR9 antagonist (8× of target concentration), 25 μL TLR9 agonist (8× of target concentration), and 50 μL of cells (4× target concentration). The 96-W plate was then incubated for 3 days. Cell viability was examined using a CellTiter-Glo® assay.

CellTiter-Glo® Assay

A 40 μL aliquot of CellTiter-Glo® reagent was added directly into each well of the 96-W plate. The plate was then shaken on a Shaker (Labsystem Wellmix) at speed 5 for 10-20 min at room temperature. Next, about 100 μL of the mixed medium was transferred to a white, non-transparent, flat bottom 96-W plate for assaying. A Flexstation 3 luminometer was used for detecting and measuring the luminescent signals. Measurements were taken at room temperature.

CellTiter-Glo® reagents were thawed prior to use. Cells pre-plated onto a second 96-W plate and incubated at room temperature for 30 minutes were used for calibration purposes.

Table 2 indicates the experimental design layout on the 96-W plate.

TABLE 2

| | | | | | | | | | | | | | |
|---|---|---|---|---|---|---|---|---|---|---|---|---|---|
| | 1 | 2 | 3 | 4 | 5 | 6 | 7 | 8 | 9 | 10 | 11 | 12 | |
| A | | | | | | | | | | | | | |
| B | | | | | | | | | | | | | ODN 4084-F |
| C | | | | | | | | | | | | | ODN INH-1 |
| D | | | | | | | | | | | | | ODN INH-18 |
| E | | | | | | | | | | | | | ODN TTAGGG |
| F | | | | | | | | | | | | | Neutral ODN |
| G | | | | | | | | | | | | | Chloroguine |
| H | | | | | | | | | | | | | |
| | | ibrutinib (nM) | | | | | | | | | | | |
| | | 100 | 20 | 4 | 0.8 | 0.16 | 0.032 | 0.0054 | 0.00128 | 0.000256 | 0 | | |

Tables 3-6 illustrate the luminescent signals for the control and the three agonists.

TABLE 3
Control

| 2 | 3 | 4 | 5 | 6 | 7 | 8 | 9 | 10 | 11 | |
|---|---|---|---|---|---|---|---|---|---|---|
| 78937 | 76955 | 93641 | 165649 | 239801 | 251339 | 238306 | 242864 | 236676 | 224715 | ODN 4084-F |
| 78498 | 77123 | 96649 | 171506 | 251899 | 258497 | 230362 | 235178 | 242501 | 224752 | ODN INH-1 |
| 77536 | 84162 | 101472 | 172336 | 240238 | 253712 | 229710 | 242404 | 223330 | 216156 | ODN INH-18 |
| 68737 | 73545 | 89717 | 168788 | 246050 | 267750 | 268756 | 264882 | 268617 | 256196 | ODN |
| | | | | | | | | | | TTAGGG |
| 93912 | 102111 | 125774 | 216440 | 277147 | 284145 | 279991 | 280412 | 273598 | 260852 | Neutral ODN |
| 61700 | 78916 | 86168 | 177291 | 250469 | 276679 | 271348 | 265058 | 264724 | 275515 | Chloroquine |
| ibrutinib | | | | | | | | | | |
| (nM) | | | | | | | | | | |
| 100 | 20 | 4 | 0.8 | 0.16 | 0.032 | 0.0064 | 0.0013 | 0.0003 | 0 | |

TABLE 4
ODN 2006

| 2 | 3 | 4 | 5 | 6 | 7 | 8 | 9 | 10 | 11 | |
|---|---|---|---|---|---|---|---|---|---|---|
| 109024 | 117281 | 134582 | 234140 | 272765 | 261515 | 283420 | 271786 | 277741 | 279589 | ODN 4084-F |
| 110444 | 114144 | 126789 | 230251 | 249615 | 266577 | 261819 | 282412 | 291581 | 302524 | ODN INH-1 |
| 103654 | 113645 | 121971 | 230343 | 265175 | 276604 | 278901 | 278408 | 279217 | 283278 | ODN INH-18 |
| 115882 | 119138 | 125933 | 235605 | 284699 | 276265 | 288335 | 292266 | 309185 | 269696 | ODN |
| | | | | | | | | | | TTAGGG |
| 113156 | 114264 | 132460 | 237787 | 271941 | 265902 | 283536 | 282512 | 291749 | 257466 | Neutral ODN |
| 62288 | 67694 | 79843 | 138546 | 247058 | 239360 | 249082 | 250534 | 258871 | 275829 | Chloroquine |
| ibrutinib | | | | | | | | | | |
| (nM) | | | | | | | | | | |
| 100 | 20 | 4 | 0.8 | 0.16 | 0.032 | 0.0064 | 0.0013 | 0.0003 | 0 | |

TABLE 5
ODN 2216

| 2 | 3 | 4 | 5 | 6 | 7 | 8 | 9 | 10 | 11 | |
|---|---|---|---|---|---|---|---|---|---|---|
| 95243 | 86472 | 105244 | 160265 | 205817 | 176494 | 194739 | 188051 | 168181 | 182913 | ODN 4084-F |
| 91251 | 92165 | 114797 | 175159 | 219691 | 196680 | 205272 | 193954 | 179216 | 193928 | ODN INH-1 |
| 84968 | 89977 | 104696 | 156281 | 216832 | 190289 | 186537 | 197417 | 176786 | 199676 | ODN INH-18 |
| 80869 | 84552 | 105060 | 168449 | 228268 | 213643 | 231380 | 210433 | 209517 | 204495 | ODN |
| | | | | | | | | | | TTAGGG |
| 110716 | 121910 | 134578 | 210628 | 243180 | 230198 | 228765 | 225508 | 211610 | 212953 | Neutral ODN |
| 44662 | 48838 | 65959 | 115898 | 207276 | 199458 | 200219 | 229545 | 193567 | 203015 | Chloroquine |
| ibrutinib | | | | | | | | | | |
| (nM) | | | | | | | | | | |
| 100 | 20 | 4 | 0.8 | 0.16 | 0.032 | 0.0064 | 0.0013 | 0.0003 | 0 | |

TABLE 6
ODN 2395

| 2 | 3 | 4 | 5 | 6 | 7 | 8 | 9 | 10 | 11 | |
|---|---|---|---|---|---|---|---|---|---|---|
| 94393 | 102447 | 120345 | 194749 | 240828 | 237612 | 237920 | 262181 | 255933 | 245353 | ODN 4084-F |
| 85740 | 94585 | 115814 | 188750 | 241953 | 258968 | 245009 | 257825 | 253421 | 229994 | ODN INH-1 |
| 77528 | 87653 | 104034 | 175913 | 224324 | 241911 | 223309 | 240978 | 235770 | 230302 | ODN INH-18 |
| 87272 | 94606 | 112117 | 170424 | 233991 | 251894 | 231101 | 250522 | 229043 | 253189 | ODN |
| | | | | | | | | | | TTAGGG |
| 125534 | 126233 | 157895 | 227516 | 256703 | 276234 | 253415 | 261877 | 255044 | 262846 | Neutral ODN |
| 55950 | 59032 | 69482 | 100658 | 156507 | 167073 | 170957 | 177301 | 168027 | 170080 | Chloroquine |
| ibrutinib | | | | | | | | | | |
| (nM) | | | | | | | | | | |
| 100 | 20 | 4 | 0.8 | 0.16 | 0.032 | 0.0064 | 0.0013 | 0.0003 | 0 | |

The luminescent measurements were subsequently processed and analyzed using CalcuSyn (CI) and Chalice Analyzer (synergy score). CalcuSyn performs the multiple drug dose-effect calculations using the Median Effect methods described by T-C Chou and P. Talalay in “Analysis of combined drug effects: a new look at a very old problem,” Trends Pharmacol. Sci. 4:450-454 (1983). In general, the resulting combination index (CI) obtained from the Chou-Talalay method defines quantitatively for additive effect (CI=1), synergism (CI<1), and antagonism (CI>1) in drug combinations. Chalice Analyzer utilizes the method described in Lehar et al. “Synergistic drug combinations improve therapeutic selectivity,” Nat. Biotechnol. 27(7):659-666 (2009). Synergy scores is higher than 1 indicate synergy between two compounds, with higher synergy scores indicating better synergy.

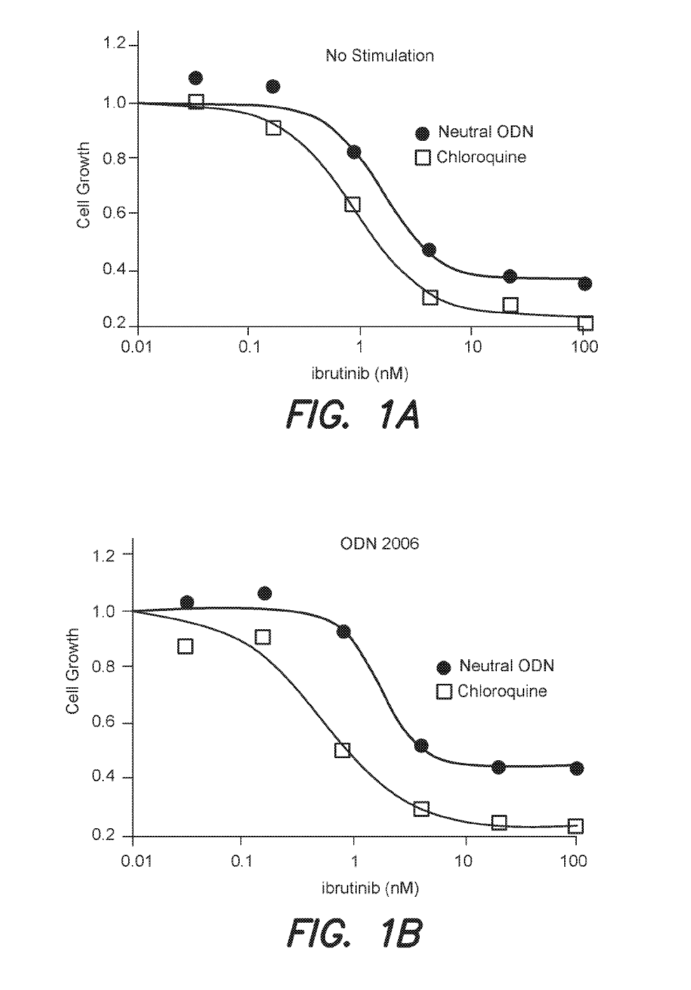

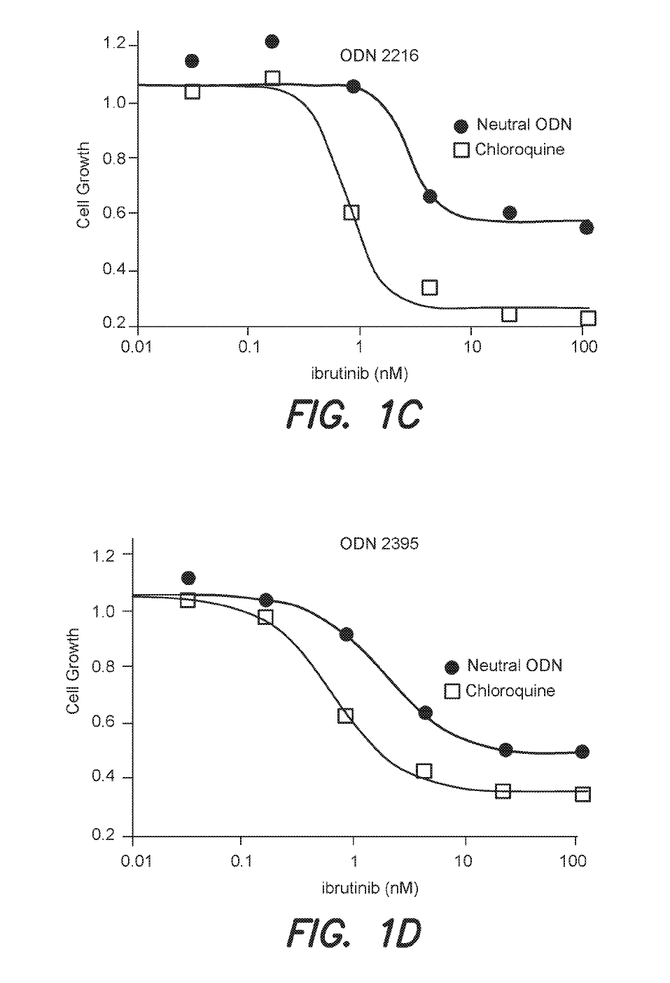

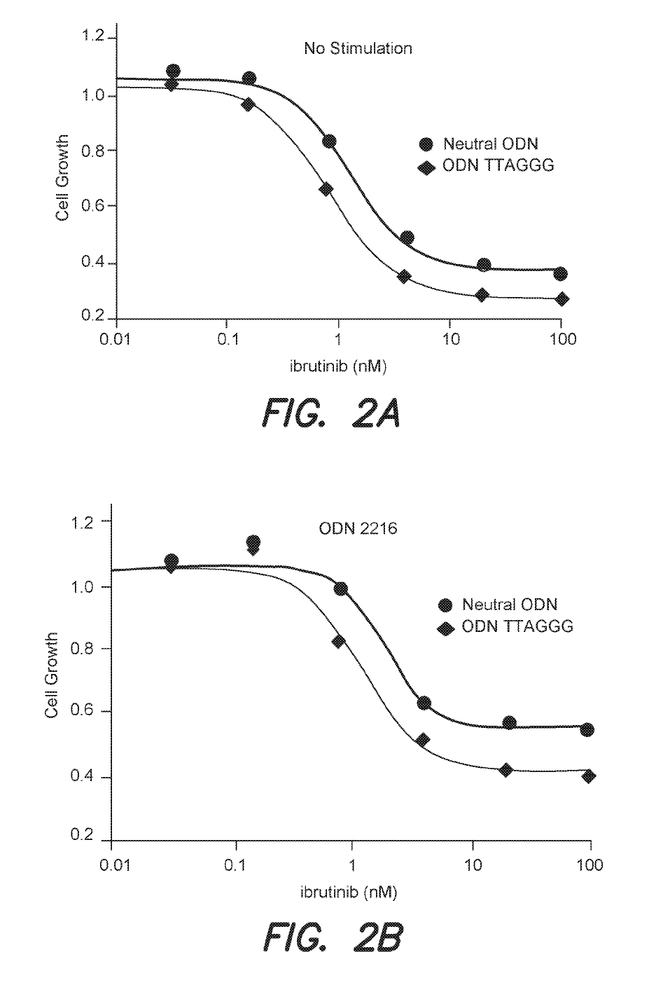

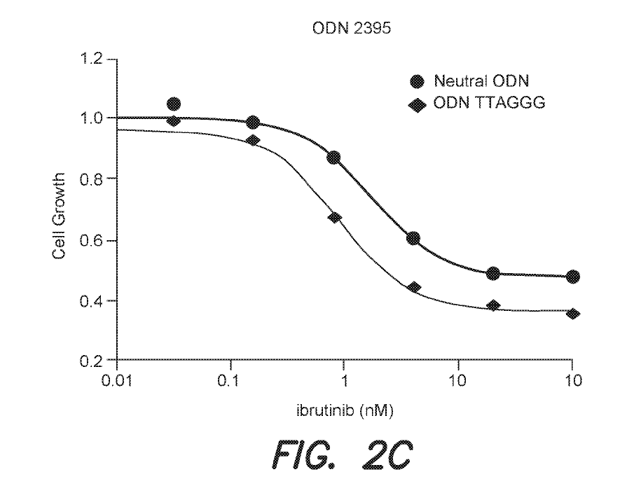

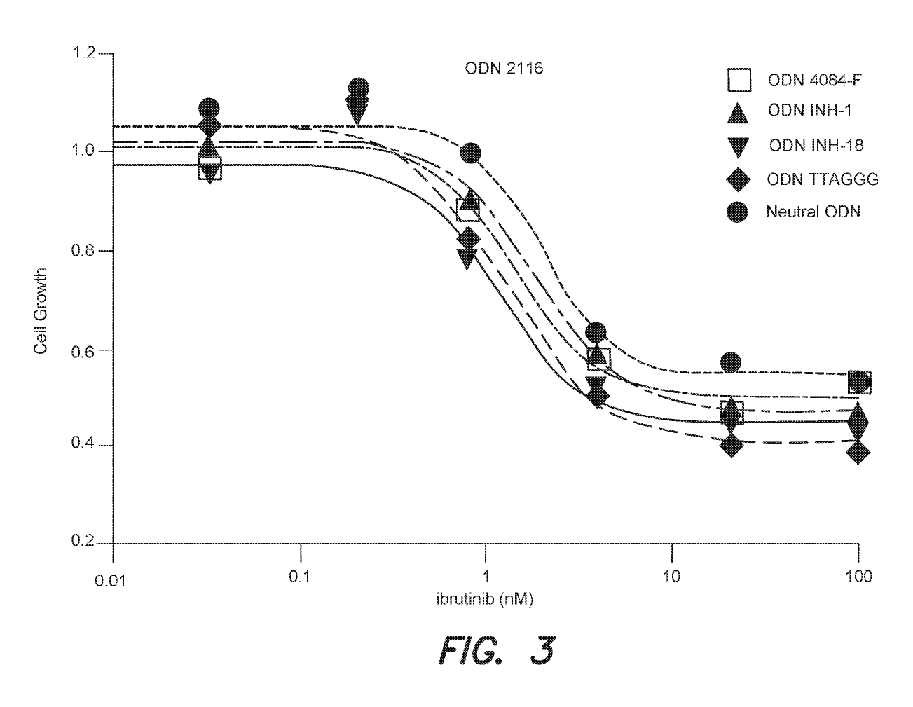

FIG. 1 illustrates the effect of the ibrutinib and chloroquine combination on TMD8 cells in the presence or absence (“no stimulation”) of TLR9 agonists (ODN 2006, ODN 2216, and ODN 2395). Neutral ODN was used as a negative control. FIG. 2 shows the effect of the combination of ibrutinib and TLR9 antagonist ODN TTAGGG on TMD8 cells in the presence or absence (“no stimulation”) of TLR9 agonists ODN2216 and ODN 2395. The TMD8 cells behaved similarly in the presence of ODN 2216 (FIG. 2B) or ODN 2395 (FIG. 2C). FIG. 3 shows the effect of the combination of ibrutinib and TLR antagonists on TMD8 cells in the presence of TLR9 agonist ODN 2116.

Synergy was observed between ibrutinib and chloroquine, a non-specific TLR antagonist; and was also observed between ibrutinib and the TLR9 antagonists tested, whether or not TLR agonist was present. The average CI values for the ibrutinib and chloroquine combination in the TMD8 cells with or without agonists were 0.11 and 0.40, respectively. The synergy score for the ibrutinib and chloroquine combination in TMD8 cells with or without agonists were 4.22 and 3.48, respectively. The CI values for ibrutinib in combination with ODN4084F, ODN INH-1, ODN INH-18, or ODN TTAGGG without agonist were 0.40, 0.47, 0.43, and 0.29, respectively. The CI values for ibrutinib in combination with ODN4084F, ODN INH-1, ODN INH-18, or ODN TTAGGG with agonist ODN 2216 were 0.25, 0.26, 0.19, and 0.20, respectively.

### Example 2: Combined Drug Treatment for Cell Viability in HBL1 and OCI-LY10 Cell Lines

ABC-DLBCL cell lines HBL1 and OCI-LY10, each of which cell lines contains the MYD88 L265P mutation, were tested in vitro to determine the effect of ibrutinib in combination with TLR antagonists on cell viability.

The experimental setup and the CellTiter-Glo® assay follow the protocols of Example 1.

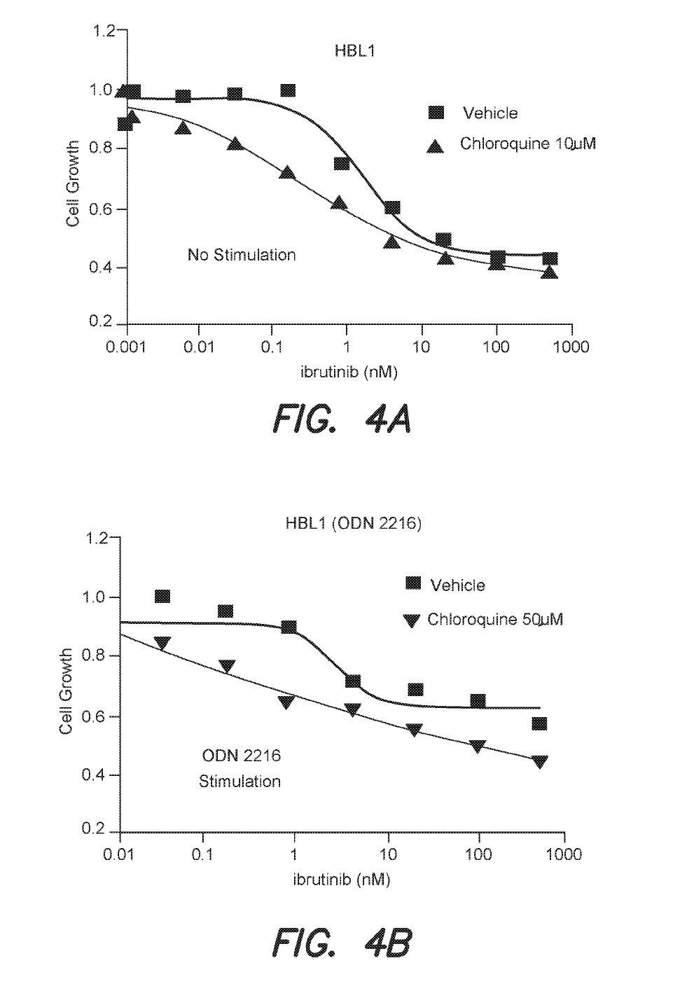

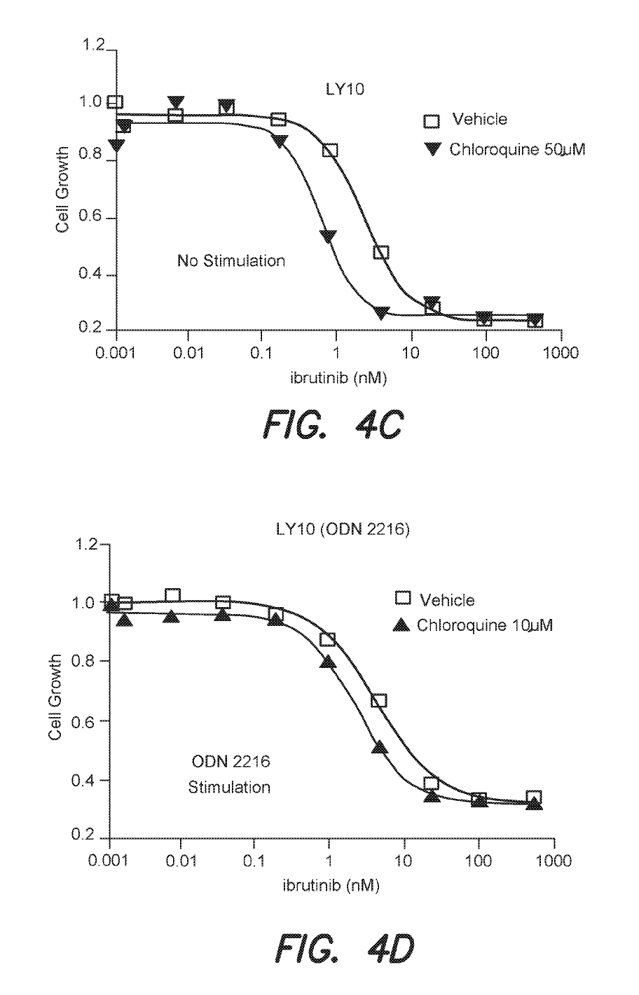

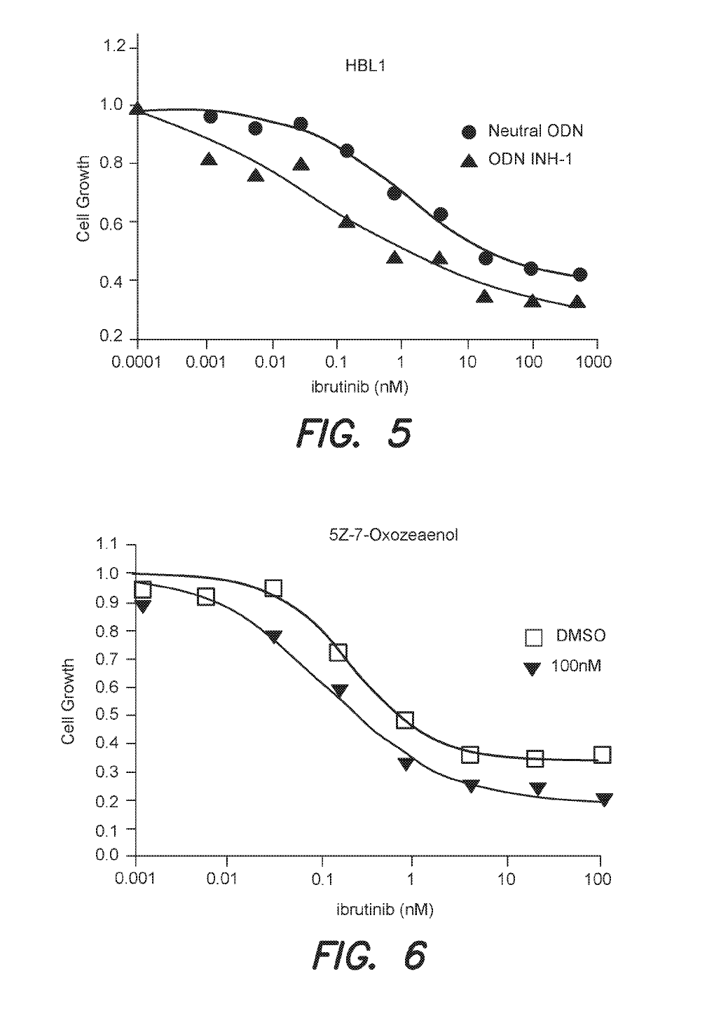

FIG. 4 shows the combination of chloroquine with ibrutinib in either HBL1 or OCI-LY 10 cell and with either ODN 2216 stimulation or without the stimulation of a TLR9 agonist. FIG. 5 shows the combination of ibrutinib with ODN INH-1 (TLR9 antagonist) in HBL1 cells. Neutral ODN was used as a negative control.

Synergy was observed between ibrutinib and chloroquine in both HLB1 and OCI-LY10 cell lines. The CI values for the chloroquine/ibrutinib combination in HBL1 cells, with or without agonist ODN2216, were 0.35 and 0.56, respectively. The CI values of LY10 with or without agonist ODN2216 were and 0.59 and 0.50, respectively. The synergy scores for the chloroquine/ibrutinib combination in HBL1 cells with or without agonist ODN2216 were and 3.5 and 3.03, respectively. The synergy scores in LY10 cells with or without agonist ODN2216 were and 2.63 and 2.44, respectively. Synergy was also observed between ibrutinib and the TLR9 antagonist ODN INH-1.

### Example 3: Combined Drug Treatment with 5Z-7-Oxozeaenol and Ibrutinib on Cell Viability in TMD8 Cell Line

The ABC-DLBCL cell line TMD8 was tested in vitro to determine the effect of ibrutinib in combination with the TAK1 inhibitor, 5Z-7-Oxozeaenol, on cell viability.

200 μl of TMD8 wt cells (1.0×10⁴ cells) at 5.0×10⁴ cells/ml was plated into each well of a 96-well plate. The cells were grown in RPMI-10P medium.

The TAK1 inhibitor used for this experiment was 5Z-7-oxozeaenol. Ibrutinib (Lot#131098) at 100, 20, 4, 0.8, 0.16, 0.032, 0.0064, 0.00128, 0.000256, 0 nM concentrations was used during the experiment. The concentrations of the TAK1 inhibitor were as shown in the table below. The stock solution for ibrutinib was prepared at 20 mM concentration. The stock solution for chloroquine diphosphate was prepared at 20 mM concentration. The CellTiter-Glo® assay follow the protocols of Example 1.

TABLE 7
5Z-7-Oxozeaenol

| | | | | | | | | | | 5Z-7- |
|---|---|---|---|---|---|---|---|---|---|---|
| | | | | | | | | | | OXO |
| | | | | | | | | | | (nM) |
| 6244.03 | 5957.34 | 5749.55 | 5441.83 | 5307.69 | 5299.8 | 4700.12 | 4731.68 | 4729.05 | 4563.35 | 10000 |
| 12982.5 | 13048.3 | 13032.5 | 12627.5 | 15015.6 | 20081.4 | 23976.6 | 25315.4 | 29868.2 | 27259.1 | 1000 |
| 34016 | 40678.2 | 42472 | 54512.9 | 98728.8 | 130054 | 151829 | 147600 | 168173 | 167090 | 100 |
| 86511.6 | 86196 | 96219.6 | 120925 | 175048 | 222768 | 223023 | 241442 | 260616 | 277804 | 10 |
| 78834.1 | 98536.7 | 98378.9 | 127363 | 181569 | 249545 | 236187 | 259290 | 249856 | 259932 | 1 |
| 94612.5 | 90633.1 | 96203.8 | 126175 | 190532 | 249480 | 240905 | 245879 | 268264 | 263270 | 0 |
| ibrutinib (nM) | | | | | | | | | | |
| 100 | 20 | 4 | 0.8 | 0.16 | 0.032 | 0.0064 | 0.00128 | 0.00026 | 0 | |

FIG. 6 shows the combination of 5Z-7-Oxozeaenol with ibrutinib in TMD8 cells. Synergy was observed between ibrutinib and 5Z-7-Oxozeaenol in TMD8 cells. The CI value for the 5Z-7-Oxozeaenol/ibrutinib combination in TMD8 cells was 0.17. The synergy score for the 5Z-7-Oxozeaenol/ibrutinib combination in TMD8 cells was 4.63.

### Example 4: Clinical Study of Ibrutinib and TLR9 Antagonist in ABC-DLBCL

The purpose of this study is to evaluate the safety and efficacy of ibrutinib in combination with a TLR9 antagonist (e.g., chloroquine) in activated B-cell (ABC) Diffuse Large B-cell Lymphoma (DLBCL) as compared to either drug alone.

Study Type: Interventional

Allocation: Eligible subjects will be randomized in a 1:1:1 ratio into 3 arms to receive: ibrutinib and TLR9 antagonist (Treatment Arm A); ibrutinib (Treatment Arm B); or TLR9 antagonist (Treatment Arm C).

Endpoint Classification: Safety Study

Intervention Model: Single Group Assignment

Masking: Open Label

Primary Purpose: Treatment

Intervention: 420 mg/day of ibrutinib, standard TLR9 antagonist regimen

Primary Outcome Measures:

To measure the number of patients with a response to study drug [Time Frame: 24 weeks from first dose]. Participants will be followed until progression of disease or start of another anti-cancer treatment.

Secondary Outcome Measures:

1. To measure the number of patients with adverse events as a measure of safety and tolerability. [Time Frame: For 30 days after the last dose of ibrutinib and/or TLR9 antagonist] Participants will be followed until progression of the disease or start of another anticancer treatment.

2. To measure a number of participants' pharmacokinetics to assist in determining how the body responses to the study drug combination. [Time Frame: Procedure will be performed during the first month of receiving study drug combination.]

Inclusion Criteria:

Men and women ≧18 years of age.

Eastern Cooperative Oncology Group (ECOG) performance status of ≦2.

Pathologically confirmed de novo DLBCL; subjects must have available archival tissue for central review to be eligible.

Subjects who have not received high dose chemotherapy/autologous stem cell transplant (HDT/ASCT) must be ineligible for HDT/ASCT as defined by meeting any of the following criteria:
Age ≧70 years
Diffuse lung capacity for carbon monoxide (DLCO)<50% by pulmonary function test (PFT)
Left ventricular ejection fraction (LVEF)<50% by multiple gated acquisition(MUGA)/echocardiograph (ECHO)
Other organ dysfunction or comorbidities precluding the use of HDT/ASCT on the basis of unacceptable risk of treatment-related morbidity
Subject refusal of HDT/ASCT

Subjects must have ≧1 measurable (>2 cm in longest dimension) disease sites on computed tomography (CT) scan.

Exclusion Criteria:

Transformed DLBCL or DLBCL with coexistent histologies (e.g., follicular or mucosa-associated lymphoid tissue [MALT] lymphoma)

Primary mediastinal (thymic) large B-cell lymphoma (PMBL)

Known central nervous system (CNS) lymphoma

Any chemotherapy, external beam radiation therapy, or anticancer antibodies within 3 weeks of the first dose of study drug

Radio- or toxin-immunoconjugates within 10 weeks of the first dose of study drug

Major surgery within 2 weeks of first dose of study drug

Any life-threatening illness, medical condition or organ system dysfunction which, in the investigator's opinion, could compromise the subject's safety, or put the study outcomes at undue risk

Clinically significant cardiovascular disease such as uncontrolled or symptomatic arrhythmias, congestive heart failure, or myocardial infarction within 6 months of screening, or any Class 3 or 4 cardiac disease as defined by the New York Heart Association Functional Classification

Unable to swallow capsules or malabsorption syndrome, disease significantly affecting gastrointestinal function, or resection of the stomach or small bowel or ulcerative colitis, symptomatic inflammatory bowel disease, or partial or complete bowel obstruction

Any of the following laboratory abnormalities:
Absolute neutrophil count (ANC)<750 cells/mm³ (0.75×109/L) unless there is documented bone marrow involvement;
Platelet count <50,000 cells/mm³ (50×109/L) independent of transfusion support unless there is documented bone marrow involvement; S
Serum aspartate transaminase (AST/SGOT) or alanine transaminase (ALT/SGPT) ≧3.0 upper limit of normal (ULN);
Creatinine >2.0×ULN

### Example 5: Clinical Study of Ibrutinib and TLR9 Antagonist in Marginal Zone Lymphoma

The purpose of this study is to evaluate the safety and efficacy of ibrutinib in combination with a TLR9 antagonist (e.g., chloroquine) in marginal zone lymphoma as compared to either drug alone.

Study Type: Interventional

Allocation: Eligible subjects will be randomized in a 1:1:1 ratio into 3 arms to receive ibrutinib and TLR9 antagonist (Treatment Arm A); ibrutinib (Treatment Arm B); or TLR9 antagonist (Treatment Arm C).

Endpoint Classification: Safety Study

Intervention Model: Single Group Assignment

Masking: Open Label

Primary Purpose: Treatment

Intervention: 420 mg/day of ibrutinib, standard TLR9 antagonist regimen

Primary Outcome Measures:

To measure the number of patients with a response to study drug [Time Frame: 24 weeks from first dose]. Participants will be followed until progression of disease or start of another anti-cancer treatment.

Secondary Outcome Measures:

1. To measure the number of patients with adverse events as a measure of safety and tolerability. [Time Frame: For 30 days after the last dose of ibrutinib and/or TLR9 antagonist] Participants will be followed until progression of the disease or start of another anticancer treatment.

2. To measure a number of participants' pharmacokinetics to assist in determining how the body responses to the study drug combination. [Time Frame: Procedure will be performed during the first month of receiving study drug combination.]

Inclusion Criteria:

Men and women ≧18 years of age.

Histologically confirmed marginal zone lymphoma (nodal, splenic, or extranodal) according to 2008 World Health Organization (WHO) criteria that is relapsed or refractory after at least 1 prior therapy

Patients with marginal zone lymphoma (MZL) are eligible after >=1 prior therapies

Body weight >=40 kg

Eastern Cooperative Oncology Group (ECOG) performance status of =<2

Agreement to use contraception during the study and for 30 days after the last dose of study drug if sexually active and able to bear children

Willing and able to participate in all required evaluations and procedures in this study protocol including swallowing capsules without difficulty

Ability to understand the purpose and risks of the study and provide signed and dated informed consent and authorization to use protected health information (in accordance with national and local patient privacy regulations)

Exclusion Criteria:

Prior malignancy, except for adequately treated basal cell or squamous cell skin cancer, in situ cervical cancer, or other cancer from which the patient has been disease free for at least 2 years or which will not limit survival to <2 years

Known central nervous system (CNS) lymphoma

Any chemotherapy, external beam radiation therapy, or anticancer antibodies within 3 weeks of the first dose of study drug

Radio- or toxin-immunoconjugates within 10 weeks of the first dose of study drug

Major surgery within 2 weeks of first dose of study drug

Any life-threatening illness, medical condition or organ system dysfunction which, in the investigator's opinion, could compromise the subject's safety, or put the study outcomes at undue risk

Clinically significant cardiovascular disease such as uncontrolled or symptomatic arrhythmias, congestive heart failure, or myocardial infarction within 6 months of screening, or any Class 3 or 4 cardiac disease as defined by the New York Heart Association Functional Classification

Unable to swallow capsules or malabsorption syndrome, disease significantly affecting gastrointestinal function, or resection of the stomach or small bowel or ulcerative colitis, symptomatic inflammatory bowel disease, or partial or complete bowel obstruction

Any of the following laboratory abnormalities:
Absolute neutrophil count (ANC)<750 cells/mm³ (0.75×109/L) unless there is documented bone marrow involvement;
Platelet count <50,000 cells/mm³ (50×109/L) independent of transfusion support unless there is documented bone marrow involvement; S
Serum aspartate transaminase (AST/SGOT) or alanine transaminase (ALT/SGPT) ≧3.0 upper limit of normal (ULN);
Creatinine >2.0×ULN

### Example 6: Clinical Study of Ibrutinib and TAK1 Inhibitor in ABC-DLBCL

The purpose of this study is to evaluate the safety and efficacy of ibrutinib in combination with a TAK1 inhibitor (e.g., 5Z-7-oxozeaenol) in activated B-cell (ABC) Diffuse Large B-cell Lymphoma (DLBCL) as compared to either drug alone.

Study Type: Interventional
Allocation: Eligible subjects will be randomized in a 1:1:1 ratio into 3 arms to receive ibrutinib and TAK1 inhibitor (Treatment Arm A); ibrutinib (Treatment Arm B); or TAK1 inhibitor (Treatment Arm C).

Endpoint Classification: Safety Study

Intervention Model: Single Group Assignment

Masking: Open Label

Primary Purpose: Treatment

Intervention: 420 mg/day of ibrutinib, standard TAK1 inhibitor regimen

Primary Outcome Measures:

To measure the number of patients with a response to study drug [Time Frame: 24 weeks from first dose]. Participants will be followed until progression of disease or start of another anti-cancer treatment.

Secondary Outcome Measures:

1. To measure the number of patients with adverse events as a measure of safety and tolerability. [Time Frame: For 30 days after the last dose of ibrutinib and/or TAK1 inhibitor] Participants will be followed until progression of the disease or start of another anticancer treatment.

2. To measure a number of participants' pharmacokinetics to assist in determining how the body responses to the study drug combination. [Time Frame: Procedure will be performed during the first month of receiving study drug combination.]

Inclusion Criteria:

Men and women ≧18 years of age.

Eastern Cooperative Oncology Group (ECOG) performance status of ≦2.

Pathologically confirmed de novo DLBCL; subjects must have available archival tissue for central review to be eligible.

Subjects who have not received high dose chemotherapy/autologous stem cell transplant (HDT/ASCT) must be ineligible for HDT/ASCT as defined by meeting any of the following criteria:
Age ≧70 years
Diffuse lung capacity for carbon monoxide (DLCO)<50% by pulmonary function test (PFT)
Left ventricular ejection fraction (LVEF)<50% by multiple gated acquisition(MUGA)/echocardiograph (ECHO)
Other organ dysfunction or comorbidities precluding the use of HDT/ASCT on the basis of unacceptable risk of treatment-related morbidity
Subject refusal of HDT/ASCT

Subjects must have ≧1 measurable (>2 cm in longest dimension) disease sites on computed tomography (CT) scan.

Exclusion Criteria:

Transformed DLBCL or DLBCL with coexistent histologies (e.g., follicular or mucosa-associated lymphoid tissue [MALT] lymphoma)

Primary mediastinal (thymic) large B-cell lymphoma (PMBL)

Known central nervous system (CNS) lymphoma

Any chemotherapy, external beam radiation therapy, or anticancer antibodies within 3 weeks of the first dose of study drug

Radio- or toxin-immunoconjugates within 10 weeks of the first dose of study drug

Major surgery within 2 weeks of first dose of study drug

Any life-threatening illness, medical condition or organ system dysfunction which, in the investigator's opinion, could compromise the subject's safety, or put the study outcomes at undue risk

Clinically significant cardiovascular disease such as uncontrolled or symptomatic arrhythmias, congestive heart failure, or myocardial infarction within 6 months of screening, or any Class 3 or 4 cardiac disease as defined by the New York Heart Association Functional Classification

Unable to swallow capsules or malabsorption syndrome, disease significantly affecting gastrointestinal function, or resection of the stomach or small bowel or ulcerative colitis, symptomatic inflammatory bowel disease, or partial or complete bowel obstruction

Any of the following laboratory abnormalities:
Absolute neutrophil count (ANC)<750 cells/mm³ (0.75×109/L) unless there is documented bone marrow involvement;
Platelet count <50,000 cells/mm³ (50×109/L) independent of transfusion support unless there is documented bone marrow involvement; S
Serum aspartate transaminase (AST/SGOT) or alanine transaminase (ALT/SGPT) ≧3.0 upper limit of normal (ULN);
Creatinine >2.0×ULN

### Example 7: Synergy Effect of Ibrutinib and Inhibitors Targeting TLR Signaling in ABC-DLBCL

The effects of ibrutinib in combination with TLR9 antagonists, a TAK1 inhibitor, or a TLR inhibitor were tested in ABC-DLBCL cell lines containing MYD88 mutations. TMD-8, HBL-1, and OCI-LY10 cell lines were treated with inhibitors or antagonists alone or in combination with ibrutinib for 3 days. Cell growth effects were determined by the CellTiter-Glo® luminescent cell viability assay (Promega). The combination index (C.I.), a drug interactivity measurement was calculated with Calcusyn. Synergy scores were calculated by the Chalice Analyzer (Horizon CombinatoRx). ApoDETECT Annexin V-FITC Kit was used to detect apoptotic cell population. LC3B antibody (Cell Signaling) was used for Western blot analysis to detect autophagic marker LC3B-II. HBL-1 cells were plated in MethoCult (StemCell Technologies) and number of colonies was counted 7 days after drug treatment to determine the effect on colony formation. TLR related gene expression was determined by using RT² Profiler PCR Array (Qiagen).

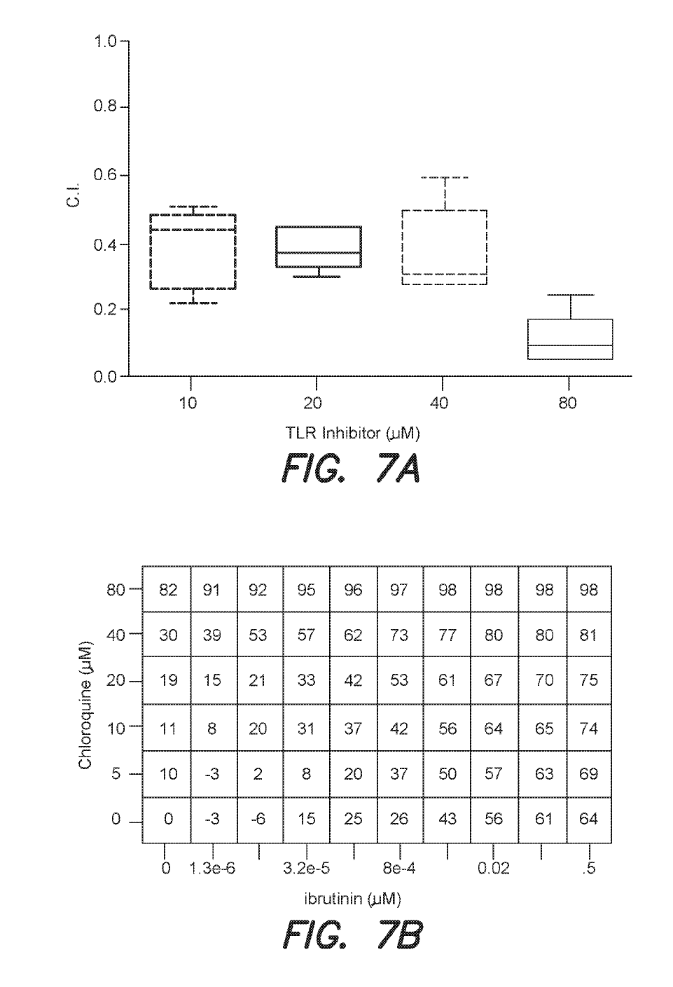

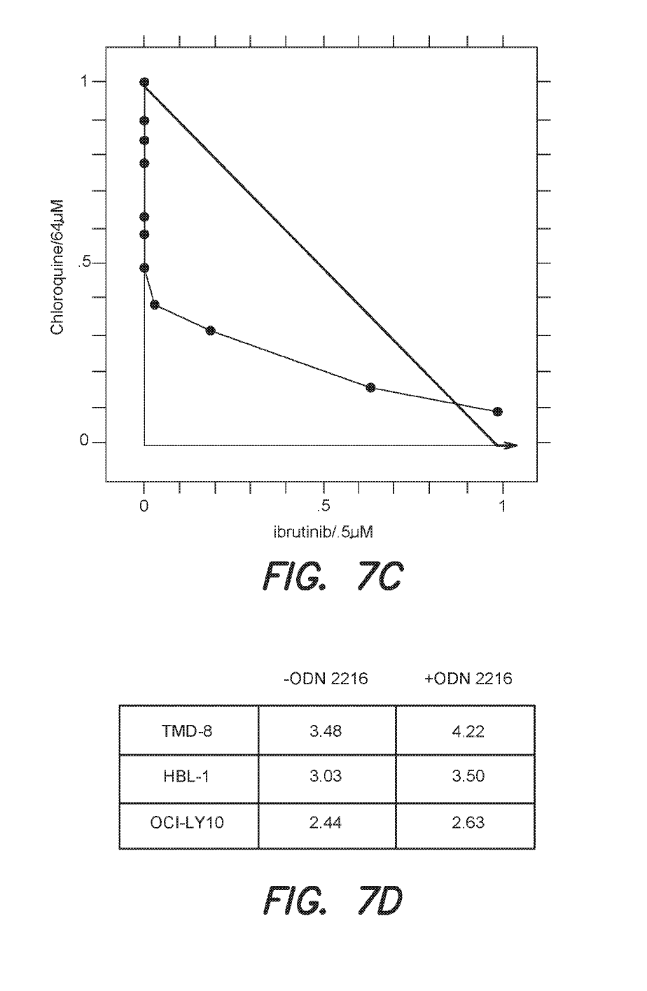

FIG. 7 illustrates the synergistic growth suppression effect of ibrutinib and TLR inhibitor in ABC-DLBCL cells. FIG. 7A shows the combination index (C.I.) of ibrutinib combination with TLR inhibitor at indicated concentrations in TMD-8 cells. FIG. 7B shows the drug dose matrix data of TMD-8 cell line. The numbers indicate the percentage of growth inhibition of cells treated for 3 days with the corresponding compound combination relative to vehicle control-treated cells. The data were visualized over matrix using a color scale. FIG. 7C exemplifies an isobologram analysis of the data in FIG. 7B. The analysis indicates strong synergy for the combination of ibrutinib and TLR inhibitor. FIG. 7D shows the synergy scores of ibrutinib combined with TLR inhibitor in ABC-DLBCL cell lines with or without the stimulation of TLR9 agonist ODN 2216.

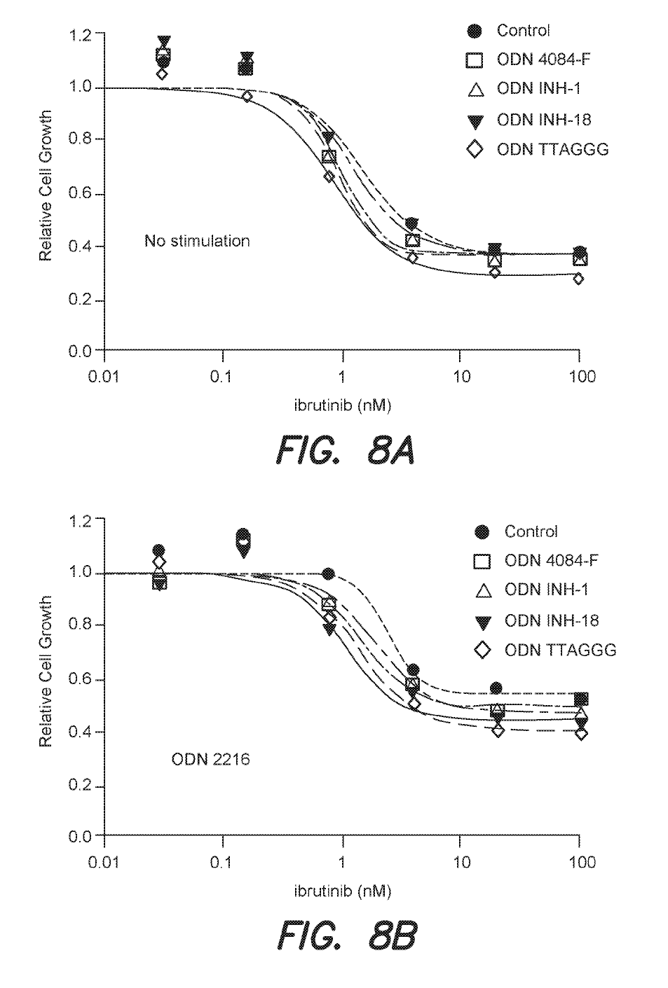

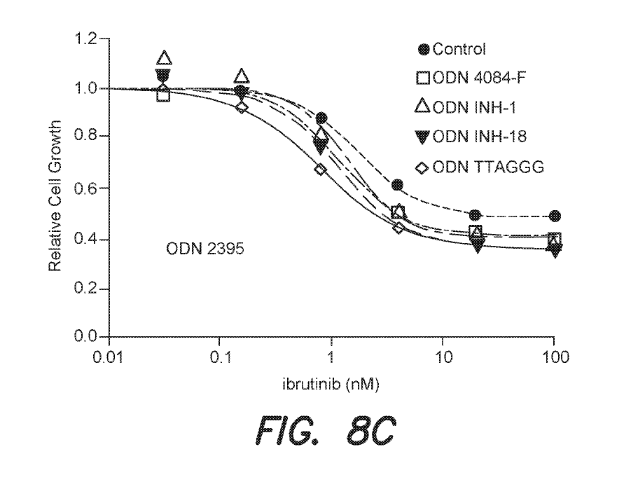

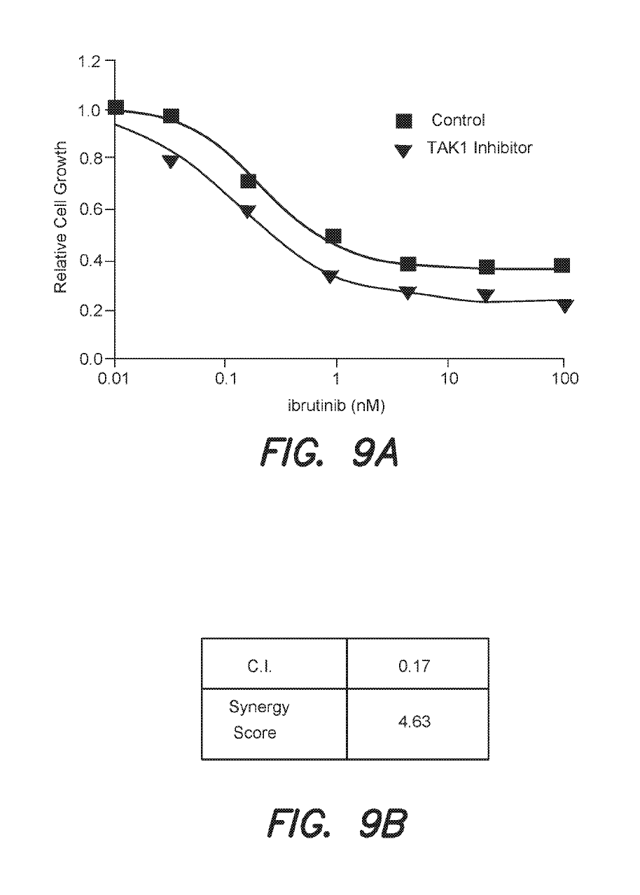

FIG. 8 illustrates increased ibrutinib sensitivity in TMD-8 cells by TLR9 antagonists in the presence or absence of TLR9 agonist stimulation. TMD-8 cells were treated with indicated concentrations of ibrutinib combined with TLR9 antagonists (ODN 4084-F, ODN INH-1, ODN INH-18, or ODN TTAGGG) or neutral ODN control in the absence (A) or presence of TLR9 agonists ODN 2216 (B) or ODN 2395 (C) for 3 days and the drug effect on cell growth was determined by CellTiter-Glo® luminescent cell viability assay.

FIG. 9 exemplifies increased ibrutinib sensitivity in TMD-8 cells by TAK1 inhibitor. In panel A, TMD-8 cells were treated with indicated concentrations of ibrutinib combined with TAK1 inhibitor (100 nM) or vehicle control for 3 days and the drug effect on cell growth was determined by CellTiter-Glo® luminescent cell viability assay. Panel B shows the combination index (C.I.) and synergy score of ibrutinib combined with TAK1 inhibitor in TMD-8 cells.

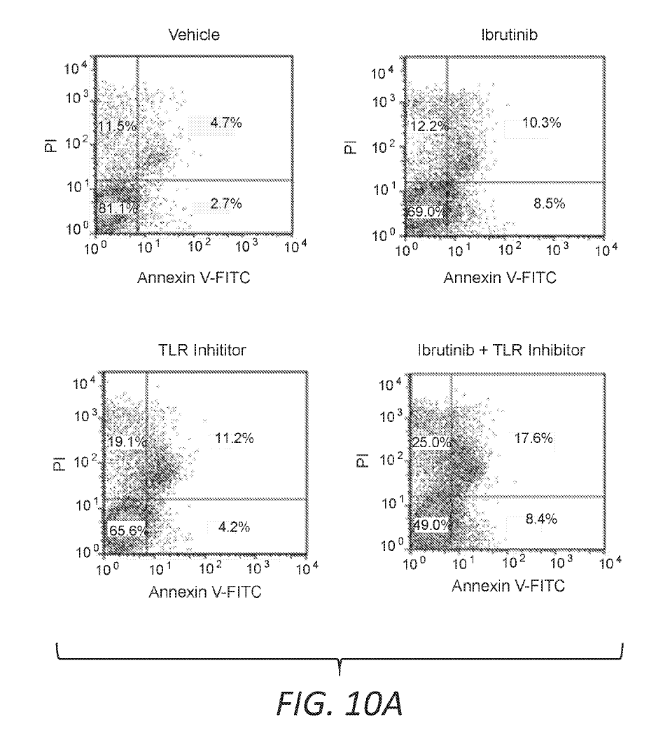

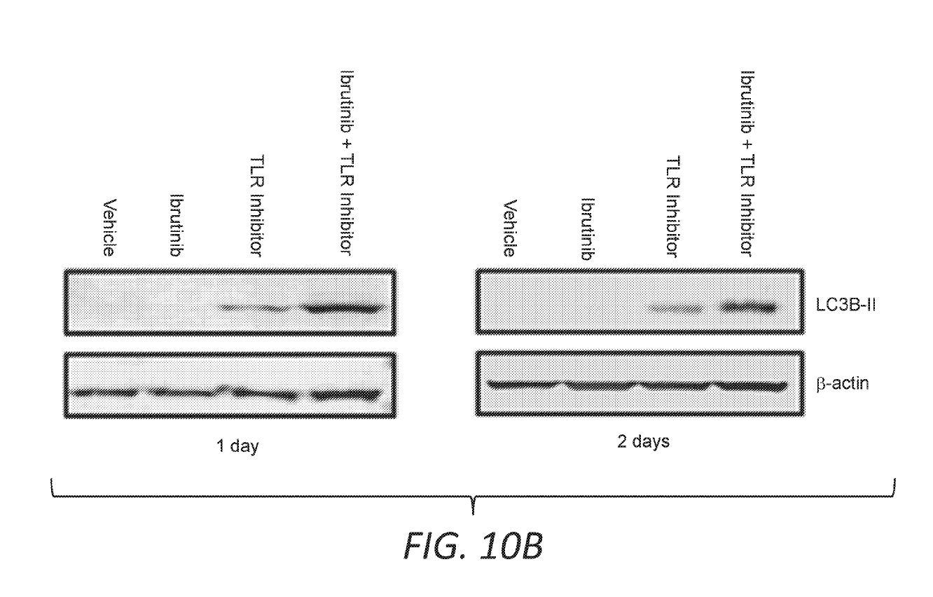

FIG. 10 illustrates the combination of ibrutinib and TLR inhibitor in increased autophagic cell death in TMD-8 cells. In panel A, TMD-8 cells were treated for 2 days with ibrutinib (100 nM), TLR inhibitor (40 μM), or a combination, and analyzed for annexin-V binding and for PI uptake. The percentage of cells as annexin V positive, PI positive or double positive for both annexin V and PI are indicated. In panel B, the autophagic marker LC3B-II analysis by Western Blot was performed 1 or 2 days after indicated drug treatment. B-actin was used as a loading control.

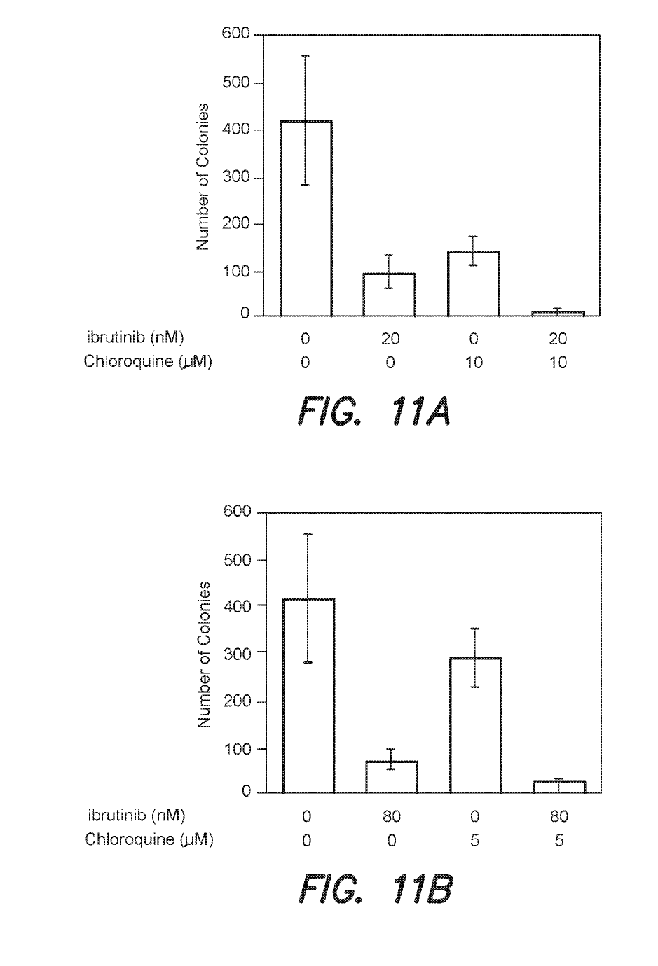

FIG. 11 shows the combination of ibrutinib and TLR inhibitor on colony formation in HBL-1 cells. The combination reduces colony formation. HBL-1 cells were plated in 0.9% MethoCult (1000 cells/well) with indicated drug treatment and colony formation was scored after 7 days. Each graph represents quantification of 3 wells, expressed as mean±SD.

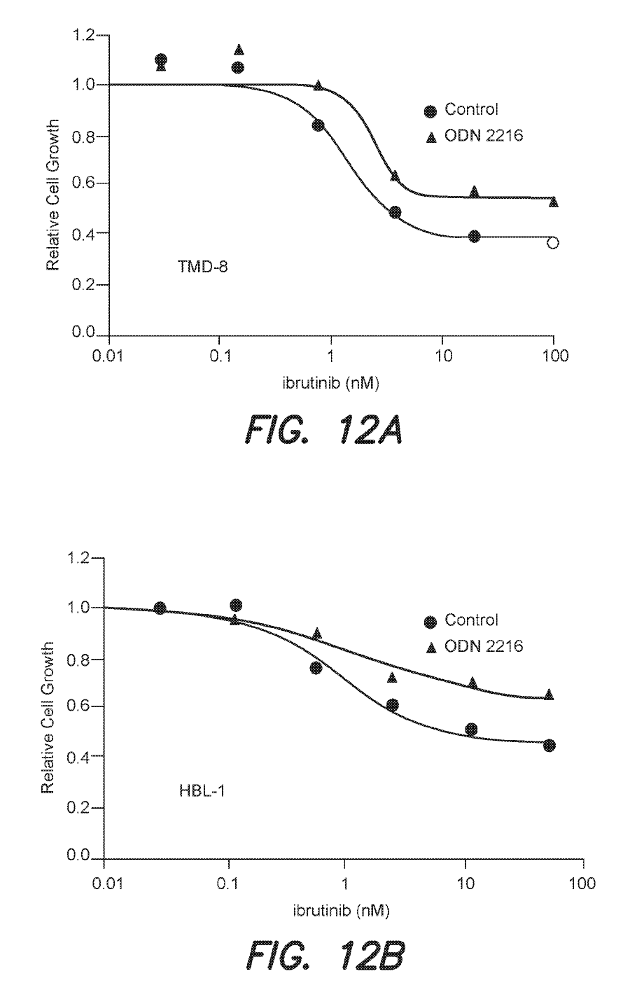

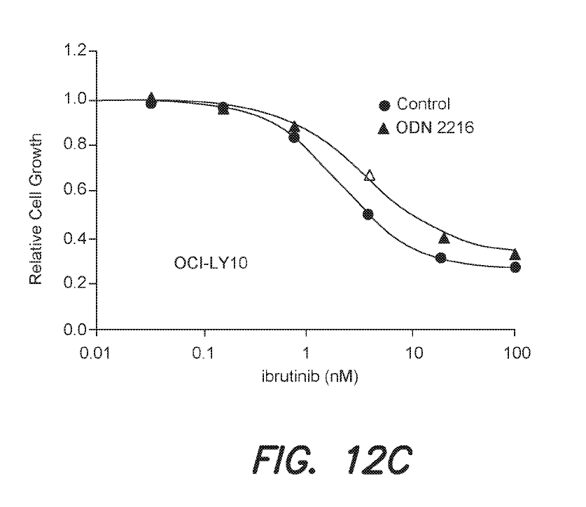

FIG. 12 exemplifies ibrutinib sensitivity in ABC-DLBCL cell lines in the presence of TLR9 agonist ODN2216. ODN2216 reduces ibrutinib sensitivity. ABC-DLBCL cell lines (A) TMD-8, (B) HBL-1, and (C) OCI-LY10 were treated with indicated concentrations of ibrutinib with or without the stimulation of TLR9 agonist ODN 2216 (1 μM) for 3 days and the drug effect on cell growth was determined by CellTiter-Glo® luminescent cell viability assay.

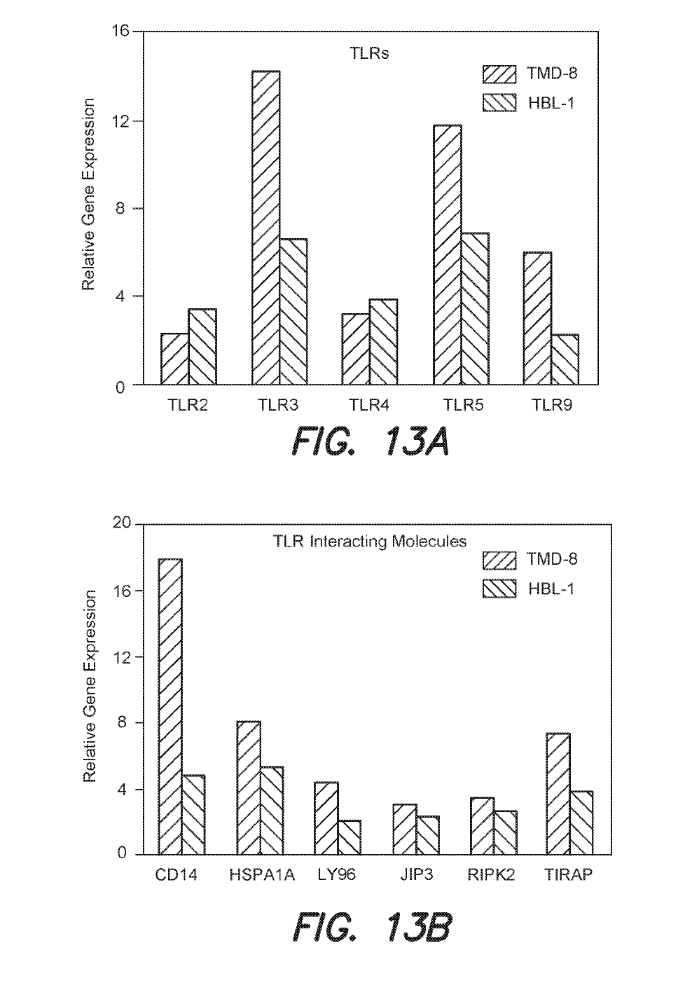

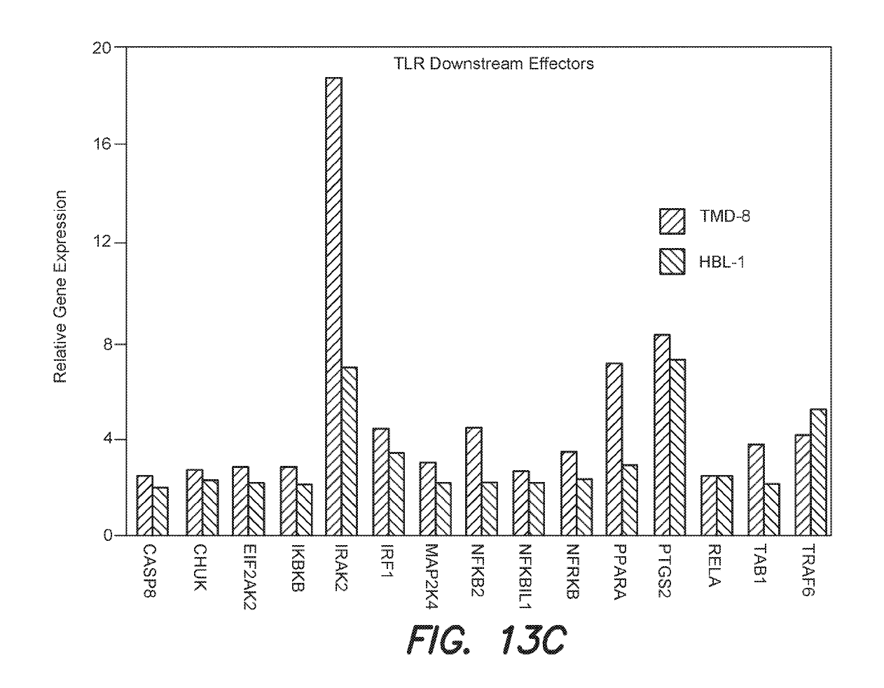

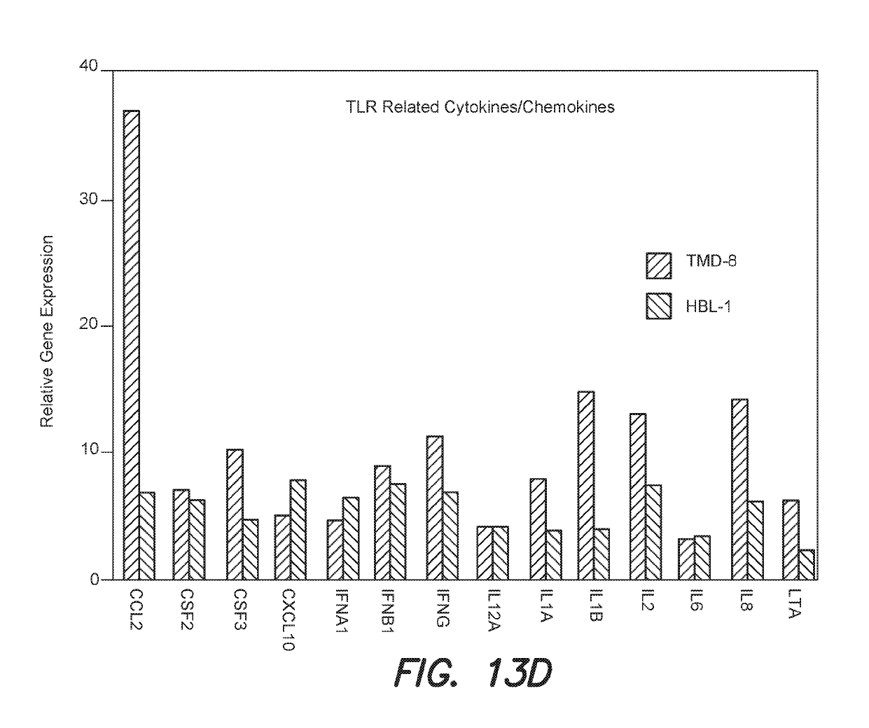

FIG. 13 shows the TLR gene expression in ibrutinib-resistant ABC-DLBCL cells. The gene expressions panels are illustrated as TLRs (A), TLR interacting molecules (B), TLR downstream effectors (C), and TLR related cytokines/chemokines (D) in TMD-8 and HBL-1 cells. The gene expressions were measured by qPCR. Expression data were normalized to microglobulin, GAPDH, and HPRT1 reference genes. All data were presented as gene expression fold change of ibrutinib-resistant samples relative to wild-type (WT) control samples.

### Example 8: PIM1 Mutations

PIM1 mutations were generated using the site-directed mutagenesis method as is known in the art. Wild-type (WT) or mutant (MUT) PIM1 cDNAs were inserted into a lentiviral vector pCDH. TMD8 cells were infected with pCDH contructs. After infection, the cells were selected with puormycin. These cell lines also referred to herein as “modified cell lines” or “modified TMD8 cells.”

In this manner, modified TMD8 cells expressing PIM1-WT, PIM1 L2V, PIM1 P81S, PIM1 S97N were generated. The expression levels of various genes were tested in these modified cell lines.

TABLE 8
PIM1-WT (SEQ. ID NO.: 1)

MLLSKINSLAHLRAAPCNDLHATKLAPGKEKEPLESQYQVGPLLGSGGFG
SVYSGIRVSDNLPVAIKHVEKDRISDWGELPNGTRVPMEVVLLKKVSSGF
SGVIRLLDWFERPDSFVLILERPEPVQDLFDFITERGALQEELARSFFWQ
VLEAVRHCHNCGVLHRDIKDENILIDLNRGELKLIDFGSGALLKDTVYTD
FDGTRVYSPPEWIRYHRYHGRSAAVWSLGILLYDMVCGDIPFEHDEEIIR
GQVFFRQRVSSECQHLIRWCLALRPSDRPTFEEIQNHPWMQDVLLPQETA
EIHLHSLSPGPSK

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

What is claimed is:

1. A method of treating a B-cell malignancy in a subject in need thereof, comprising administering to the subject a therapeutically effective amount of a combination comprising a BTK inhibitor and a TLR9 inhibitor selected from the group consisting of a non-specific TLR inhibitor, a TLR6/7/8/9 antagonist, and a TLR9 antagonist, wherein the TLR9 antagonist is selected from the group consisting of chloroquine, quinacrine, monesin, bafilomycin A1, wortmannin, iODN, (+)-morphinans, 9-aminoacridine, 4-aminoquinoline, 4-aminoquinolines, 7,8,9,10-tetrahydro-6H-cyclohepta[b]quinolin-11-ylamine; 1-methyl-2,3-dihydro-1H-pyrrolo[2,3-b]quinolin-4-ylamine; 1,6-dimethyl-2,3-dihydro-1H-pyrrolo[2,3-b]quinolin-4-ylamine; 6-bromo-1-methyl-2,3-dihydro-1H-pyrrolo[2,3-b]quinolin-4-ylamine; 1-methyl-2,3,4,5-tetrahydro-1H-azepino[2,3-b]quinolin-6-ylamine; 3,3-dimethyl-3,4-dihydro-acridin-9-ylamine; 1-benzyl-2,3-dihydro-1H-pyrrolo[2,3-b]quinolin-4-ylamine; 6-methyl-1-phenyl-2,3-dihydro-1H-pyrrolo[2,3-b]quinolin-4-ylamine; N*2*,N*2*-Dimethyl-quinoline-2,4-diamine, 2,7-Dimethyl-dibenzo[b,g][1,8]naphthyridin-11-ylamine; 2,4-Dimethyl-benzo[b][1,8]naphthyridin-5-ylamine; 7-Fluoro-2,4-dimethyl-benzo[b][1,8]naphthyridin-5-ylamine; 1,2,3,4-Tetrahydro-acridin-9-ylamine Tacrine hydrochloridehydrate; 2,3-Dihydro-1H-cyclopenta[b]quinolin-9-ylamine; 2,4,9-Trimethyl-benzo[b][1,8]naphthyridin-5-ylamine; 9-Amino-3,3-dimethyl-1,2,3,4-tetrahydro-acridin-1-ol and 7-Ethoxy-N*3*-furan-2-ylmethyl-acridine-3,9-diamine; quinazolines, N,N-dimethyl-N′-{2-[4-(4-methyl-piperazin-1-yl)-phenyl]-3,4-dihydro-quinazoline-4-yl}-ethane-1,2,-diamine; N′-[6,7-Dimethoxy-2-(4-phenyl-piperazin-1-yl)-quinazolin-4-yl]-N,N-dimethyl-ethane-1,2-diamine; N′-[6,7-Dimethoxy-2-(4-methyl-piperazin-1-yl)-quinazolin-4-yl]-N,N-dimethyl-ethane-1,2-diamine; N,N-Dimethyl-N′-(2-phenyl-quinazolin-4-yl)-ethane-1,2-diamine; Dimethyl-(2-{2-[4-(4-methyl-piperazin-1-yl)-phenyl]-quinazolin-4-yloxy}-ethyl)-amine; N′-(2-Biphenyl-4-yl-quinazolin-4-yl)-N,N-dimethyl-ethane-1,2-diamine and Dimethyl-[2-(2-phenyl-quinazolin-4-yloxy)-ethyl]-amine; ODN 2088, ODN with a TTAGGG sequence, G-ODN, statins, atorvastatin, IMO-2125 (Idera Pharmaceuticals), IRS 869, CMZ 203-84, CMZ 203-85, CMZ 203-88, CMZ 203-88-1, CMZ 203-89, CMZ 203-91, INH-ODN 2114, ODN A151, ODN INH-1, ODN INH-18, ODN 4084, ODN 4084-F, and ODN INH-47.

2. The method of claim 1, wherein the combination provides a synergistic therapeutic effect compared to administration of the BTK inhibitor or the TLR inhibitor alone.

3. The method of any one of claims 1-2, wherein the non-specific TLR inhibitor is selected from the group consisting of chloroquine and bafilomycin A.

4. The method of any one of claims 1-2 wherein the TLR7/8/9 antagonist is selected from the group consisting of CPG52364, IMO 8400, and IMO-9200.

5. The method of any one of claims 1-4, wherein the BTK inhibitor is ibrutinib.

6. The method of any one of claims 1-5, wherein the B-cell malignancy is diffuse large B-cell lymphoma (DLBCL), marginal zone lymphoma (MZL), acute lymphoblastic leukemia (ALL), acute myelogenous leukemia (AML), chronic myelogenous leukemia (CML), acute monocytic leukemia (AMoL), chronic lymphocytic leukemia (CLL), small lymphocytic lymphoma (SLL), high-risk small lymphocytic lymphoma (SLL), follicular lymphoma (FL), mantle cell lymphoma (MCL), Waldenstrom's macroglobulinemia, multiple myeloma, extranodal marginal zone B cell lymphoma, nodal marginal zone B cell lymphoma, Burkitt's lymphoma, non-Burkitt high grade B cell lymphoma, primary mediastinal B-cell lymphoma (PMBL), immunoblastic large cell lymphoma, precursor B-lymphoblastic lymphoma, B cell prolymphocytic leukemia, lymphoplasmacytic lymphoma, splenic marginal zone lymphoma, plasma cell myeloma, plasmacytoma, mediastinal (thymic) large B cell lymphoma, intravascular large B cell lymphoma, primary effusion lymphoma, or lymphomatoid granulomatosis.

7. The method of claim 6, wherein the DLBCL is activated B-cell diffuse large B-cell lymphoma (ABC-DLBCL).

8. The method of claim 7, wherein the ABC-DLBCL is characterized by a mutation in MYD88.

9. The method of claim 8, wherein the mutation is at position 265 of MYD88.

10. The method of claim 9, wherein the mutation is an L265P mutation.

11. The method of any one of claims 5-10, wherein ibrutinib is administered once a day, two times per day, three times per day, four times per day, or five times per day.

12. The method of claim 11, wherein ibrutinib is administered at a dosage of about 40 mg/day to about 1000 mg/day.

13. The method of claim 12, wherein ibrutinib is administered orally.

14. The method of any one of claims 5-13, wherein ibrutinib and the TLR inhibitor are administered simultaneously, sequentially or intermittently.

15. The method of any one of claims 1-14, wherein the method further comprises administering a third therapeutic agent.

16. The method of claim 15, wherein the third therapeutic agent is selected from among a chemotherapeutic agent or radiation therapeutic agent.

17. The method of claim 16, wherein the chemotherapeutic agent is selected from among chlorambucil, ifosfamide, doxorubicin, mesalazine, thalidomide, lenalidomide, temsirolimus, everolimus, fludarabine, fostamatinib, paclitaxel, docetaxel, ofatumumab, rituximab, dexamethasone, prednisone, CAL-101, ibritumomab, tositumomab, bortezomib, pentostatin, endostatin, or a combination thereof.

18. A method of treating a diffuse large B-cell lymphoma (DLBCL) or a marginal zone lymphoma (MZL) comprising administering to a subject in need thereof a therapeutically effective amount of a combination comprising a BTK inhibitor and a TLR inhibitor, wherein the TLR inhibitor is a non-specific TLR inhibitor, a TLR6/7/8/9 antagonist, or a TLR9 antagonist selected from the group consisting of chloroquine, quinacrine, monesin, bafilomycin A1, wortmannin, iODN, (+)-morphinans, 9-aminoacridine, 4-aminoquinoline, 4-aminoquinolines, 7,8,9,10-tetrahydro-6H-cyclohepta[b]quinolin-11-ylamine; 1-methyl-2,3-dihydro-1H-pyrrolo[2,3-b]quinolin-4-ylamine; 1,6-dimethyl-2,3-dihydro-1H-pyrrolo[2,3-b]quinolin-4-ylamine; 6-bromo-1-methyl-2,3-dihydro-1H-pyrrolo[2,3-b]quinolin-4-ylamine; 1-methyl-2,3,4,5-tetrahydro-1H-azepino[2,3-b]quinolin-6-ylamine; 3,3-dimethyl-3,4-dihydro-acridin-9-ylamine; 1-benzyl-2,3-dihydro-H-pyrrolo[2,3-b]quinolin-4-ylamine; 6-methyl-1-phenyl-2,3-dihydro-1H-pyrrolo[2,3-b]quinolin-4-ylamine; N*2*,N*2*-Dimethyl-quinoline-2,4-diamine, 2,7-Dimethyl-dibenzo[b,g][1,8]naphthyridin-11-ylamine; 2,4-Dimethyl-benzo[b][1,8]naphthyridin-5-ylamine; 7-Fluoro-2,4-dimethyl-benzo[b][1,8]naphthyridin-5-ylamine; 1,2,3,4-Tetrahydro-acridin-9-ylamine Tacrine hydrochloridehydrate; 2,3-Dihydro-1H-cyclopenta[b]quinolin-9-ylamine; 2,4,9-Trimethyl-benzo[b][1,8]naphthyridin-5-ylamine; 9-Amino-3,3-dimethyl-1,2,3,4-tetrahydro-acridin-1-ol and 7-Ethoxy-N*3*-furan-2-ylmethyl-acridine-3,9-diamine; quinazolines, N,N-dimethyl-N′-{2-[4-(4-methyl-piperazin-1-yl)-phenyl]-3,4-dihydro-quinazoline-4-yl}-ethane-1,2,-diamine; N′-[6,7-Dimethoxy-2-(4-phenyl-piperazin-1-yl)-quinazolin-4-yl]-N,N-dimethyl-ethane-1,2-diamine; N′-[6,7-Dimethoxy-2-(4-methyl-piperazin-1-yl)-quinazolin-4-yl]-N,N-dimethyl-ethane-1,2-diamine; N,N-Dimethyl-N′-(2-phenyl-quinazolin-4-yl)-ethane-1,2-diamine; Dimethyl-(2-{2-[4-(4-methyl-piperazin-1-yl)-phenyl]-quinazolin-4-yloxy}-ethyl)-amine; N′-(2-Biphenyl-4-yl-quinazolin-4-yl)-N,N-dimethyl-ethane-1,2-diamine and Dimethyl-[2-(2-phenyl-quinazolin-4-yloxy)-ethyl]-amine; ODN 2088, ODN with a TTAGGG sequence, G-ODN, statins, atorvastatin, IMO-2125 (Idera Pharmaceuticals), IRS 869, CMZ 203-84, CMZ 203-85, CMZ 203-88, CMZ 203-88-1, CMZ 203-89, CMZ 203-91, INH-ODN 2114, ODN A151, ODN INH-1, ODN INH-18, ODN 4084, ODN 4084-F, and ODN INH-47.

19. The method of claim 18, wherein the combination provides a synergistic therapeutic effect compared to administration of the BTK inhibitor or the TLR inhibitor alone.

20. The method of any one of claims 18-19, wherein the non-specific TLR inhibitor is selected from the group consisting of chloroquine and bafilomycin A.

21. The method of any one of claims 18-19, wherein the TLR7/8/9 antagonist is selected from the group consisting of CPG52364, IMO 8400, and IMO-9200.

22. The method of any one of claims 18-21, wherein the BTK inhibitor is ibrutinib.

23. The method of claim 18, wherein the DLBCL is activated B-cell diffuse large B-cell lymphoma (ABC-DLBCL).

24. The method of claim 23, wherein the ABC-DLBCL is characterized by a mutation in MYD88.

25. The method of claim 24, wherein the mutation is at position 265 of MYD88.

26. The method of claim 25, wherein the mutation is an L265P mutation.

27. The method of any one of claims 22-26, wherein ibrutinib is administered once a day, two times per day, three times per day, four times per day, or five times per day.

28. The method of claim 27, wherein ibrutinib is administered at a dosage of about 40 mg/day to about 1000 mg/day.

29. The method of claim 28, wherein ibrutinib is administered orally.

30. The method of claim 18, wherein ibrutinib and the TLR inhibitor are administered simultaneously, sequentially or intermittently.

31. The method of any one of claims 18-30, wherein the method further comprises administering a third therapeutic agent.

32. The method of claim 31, wherein the third therapeutic agent is a chemotherapeutic agent or radiation therapeutic agent.

33. The method of claim 32, wherein the chemotherapeutic agent is selected from among chlorambucil, ifosfamide, doxorubicin, mesalazine, thalidomide, lenalidomide, temsirolimus, everolimus, fludarabine, fostamatinib, paclitaxel, docetaxel, ofatumumab, rituximab, dexamethasone, prednisone, CAL-101, ibritumomab, tositumomab, bortezomib, pentostatin, endostatin, or a combination thereof.

34. A method of treating a B-cell malignancy associated with over-activated TLR signaling, comprising:
detecting the presence of absence of a mutation in MYD88 in a sample from an individual; and
administering to the individual a therapeutically effective amount of a combination comprising a BTK inhibitor and a TLR inhibitor if the individual has a mutation in MYD88, wherein the TLR inhibitor is selected from the group consisting of a non-specific TLR inhibitor; a TLR6/7/8/9 antagonist; and a TLR9 antagonist, wherein the TLR9 antagonist is selected from the group consisting of chloroquine, quinacrine, monesin, bafilomycin A1, wortmannin, iODN, (+)-morphinans, 9-aminoacridine, 4-aminoquinoline, 4-aminoquinolines, 7,8,9,10-tetrahydro-6H-cyclohepta[b]quinolin-11-ylamine; 1-methyl-2,3-dihydro-1H-pyrrolo[2,3-b]quinolin-4-ylamine; 1,6-dimethyl-2,3-dihydro-1H-pyrrolo[2,3-b]quinolin-4-ylamine; 6-bromo-1-methyl-2,3-dihydro-1H-pyrrolo[2,3-b]quinolin-4-ylamine; 1-methyl-2,3,4,5-tetrahydro-1H-azepino[2,3-b]quinolin-6-ylamine; 3,3-dimethyl-3,4-dihydro-acridin-9-ylamine; 1-benzyl-2,3-dihydro-1H-pyrrolo[2,3-b]quinolin-4-ylamine; 6-methyl-1-phenyl-2,3-dihydro-1H-pyrrolo[2,3-b]quinolin-4-ylamine; N*2*,N*2*-Dimethyl-quinoline-2,4-diamine, 2,7-Dimethyl-dibenzo[b,g][1,8]naphthyridin-11-ylamine; 2,4-Dimethyl-benzo[b][1,8]naphthyridin-5-ylamine; 7-Fluoro-2,4-dimethyl-benzo[b][1,8]naphthyridin-5-ylamine; 1,2,3,4-Tetrahydro-acridin-9-ylamine Tacrine hydrochloridehydrate; 2,3-Dihydro-1H-cyclopenta[b]quinolin-9-ylamine; 2,4,9-Trimethyl-benzo[b][1,8]naphthyridin-5-ylamine; 9-Amino-3,3-dimethyl-1,2,3,4-tetrahydro-acridin-1-ol and 7-Ethoxy-N*3*-furan-2-ylmethyl-acridine-3,9-diamine; quinazolines, N,N-dimethyl-N′-{2-[4-(4-methyl-piperazin-1-yl)-phenyl]-3,4-dihydro-quinazoline-4-yl}-ethane-1,2,-diamine; N′-[6,7-Dimethoxy-2-(4-phenyl-piperazin-1-yl)-quinazolin-4-yl]-N,N-dimethyl-ethane-1,2-diamine; N′-[6,7-Dimethoxy-2-(4-methyl-piperazin-1-yl)-quinazolin-4-yl]-N,N-dimethyl-ethane-1,2-diamine; N,N-Dimethyl-N′-(2-phenyl-quinazolin-4-yl)-ethane-1,2-diamine; Dimethyl-(2-{2-[4-(4-methyl-piperazin-1-yl)-phenyl]-quinazolin-4-yloxy}-ethyl)-amine; N′-(2-Biphenyl-4-yl-quinazolin-4-yl)-N,N-dimethyl-ethane-1,2-diamine and Dimethyl-[2-(2-phenyl-quinazolin-4-yloxy)-ethyl]-amine; ODN 2088, ODN with a TTAGGG sequence, G-ODN, statins, atorvastatin, IMO-2125 (Idera Pharmaceuticals), IRS 869, CMZ 203-84, CMZ 203-85, CMZ 203-88, CMZ 203-88-1, CMZ 203-89, CMZ 203-91, INH-ODN 2114, ODN A151, ODN INH-1, ODN INH-18, ODN 4084, ODN 4084-F, and ODN INH-47.

35. The method of claim 34, wherein the mutation is at amino acid position 198 or 265 of MYD88.

36. The method of claim 35, wherein the mutation at amino acid position 198 of MYD88 is S198N.

37. The method of claim 35, wherein the mutation at amino acid position 265 of MYD88 is L265P.

38. The method of any one of claims 34-37, wherein sample is a nucleic acid molecule containing sample encoding MYD88 from the individual, and the detecting comprises testing the nucleic acid molecule containing sample to determine whether the nucleic acid molecules encoding MYD88 contain the mutation.

39. The method of claim 38, wherein the nucleic acid molecule is RNA or DNA.

40. The method of claim 39, wherein the DNA is genomic DNA.

41. The method of any one of claims 38-40, wherein testing comprises amplifying the nucleic acid molecules encoding MYD88.

42. The method of claim 34, wherein the combination provides a synergistic therapeutic effect compared to administration of the BTK inhibitor or the TLR inhibitor alone.

43. The method of any one of claims 34-42, wherein the non-specific TLR inhibitor is selected from the group consisting of chloroquine and bafilomycin A.

44. The method of any one of claims 34-42, wherein the TLR7/8/9 antagonist is selected from the group consisting of CPG52364, IMO 8400, and IMO-9200.

45. The method of any one of claims 34-44, wherein the BTK inhibitor is ibrutinib.

46. The method of any one of claims 34-45, wherein the B-cell malignancy is diffuse large B-cell lymphoma (DLBCL), marginal zone lymphoma (MZL), acute lymphoblastic leukemia (ALL), acute myelogenous leukemia (AML), chronic myelogenous leukemia (CML), acute monocytic leukemia (AMoL), chronic lymphocytic leukemia (CLL), small lymphocytic lymphoma (SLL), high-risk small lymphocytic lymphoma (SLL), follicular lymphoma (FL), mantle cell lymphoma (MCL), Waldenstrom's macroglobulinemia, multiple myeloma, extranodal marginal zone B cell lymphoma, nodal marginal zone B cell lymphoma, Burkitt's lymphoma, non-Burkitt high grade B cell lymphoma, primary mediastinal B-cell lymphoma (PMBL), immunoblastic large cell lymphoma, precursor B-lymphoblastic lymphoma, B cell prolymphocytic leukemia, lymphoplasmacytic lymphoma, splenic marginal zone lymphoma, plasma cell myeloma, plasmacytoma, mediastinal (thymic) large B cell lymphoma, intravascular large B cell lymphoma, primary effusion lymphoma, or lymphomatoid granulomatosis.

47. The method of claim 46, wherein the DLBCL is activated B-cell diffuse large B-cell lymphoma (ABC-DLBCL).

48. The method of any one of claims 34-47, wherein the B-cell malignancy is relapsed or refractory B-cell malignancy.

49. The method of claim 34, wherein the sample comprises one or more tumor cells.

50. The method of any one of claims 45-49, wherein ibrutinib is administered once a day, two times per day, three times per day, four times per day, or five times per day.

51. The method of claim 50, wherein ibrutinib is administered at a dosage of about 40 mg/day to about 1000 mg/day.

52. The method of claim 51, wherein ibrutinib is administered orally.

53. The method of any one of claims 45-52, wherein ibrutinib and the TLR inhibitor are administered simultaneously, sequentially or intermittently.

54. The method of any one of claims 34-53, wherein the method further comprises administering a third therapeutic agent.

55. The method of claim 54, wherein the third therapeutic agent is selected from among a chemotherapeutic agent or radiation therapeutic agent.

56. The method of claim 55, wherein the chemotherapeutic agent is selected from among chlorambucil, ifosfamide, doxorubicin, mesalazine, thalidomide, lenalidomide, temsirolimus, everolimus, fludarabine, fostamatinib, paclitaxel, docetaxel, ofatumumab, rituximab, dexamethasone, prednisone, CAL-101, ibritumomab, tositumomab, bortezomib, pentostatin, endostatin, or a combination thereof.

57. A method of selecting an individual having a B-cell malignancy for therapy with a combination comprising a BTK inhibitor and a TLR inhibitor, wherein the TLR inhibitor is selected from the group consisting of a non-specific TLR inhibitor; a TLR6/7/8/9 antagonist; and a TLR9 antagonist, wherein the TLR9 antagonist is selected from the group consisting of chloroquine, quinacrine, monesin, bafilomycin A1, wortmannin, iODN, (+)-morphinans, 9-aminoacridine, 4-aminoquinoline, 4-aminoquinolines, 7,8,9,10-tetrahydro-6H-cyclohepta[b]quinolin-11-ylamine; 1-methyl-2,3-dihydro-1H-pyrrolo[2,3-b]quinolin-4-ylamine; 1,6-dimethyl-2,3-dihydro-H-pyrrolo[2,3-b]quinolin-4-ylamine; 6-bromo-1-methyl-2,3-dihydro-1H-pyrrolo[2,3-b]quinolin-4-ylamine; 1-methyl-2,3,4,5-tetrahydro-1H-azepino[2,3-b]quinolin-6-ylamine; 3,3-dimethyl-3,4-dihydro-acridin-9-ylamine; 1-benzyl-2,3-dihydro-1H-pyrrolo[2,3-b]quinolin-4-ylamine; 6-methyl-1-phenyl-2,3-dihydro-1H-pyrrolo[2,3-b]quinolin-4-ylamine; N*2*,N*2*-Dimethyl-quinoline-2,4-diamine, 2,7-Dimethyl-dibenzo[b,g][1,8]naphthyridin-11-ylamine; 2,4-Dimethyl-benzo[b][1,8]naphthyridin-5-ylamine; 7-Fluoro-2,4-dimethyl-benzo[b][1,8]naphthyridin-5-ylamine; 1,2,3,4-Tetrahydro-acridin-9-ylamine Tacrine hydrochloridehydrate; 2,3-Dihydro-1H-cyclopenta[b]quinolin-9-ylamine; 2,4,9-Trimethyl-benzo[b][1,8]naphthyridin-5-ylamine; 9-Amino-3,3-dimethyl-1,2,3,4-tetrahydro-acridin-1-ol and 7-Ethoxy-N*3*-furan-2-ylmethyl-acridine-3,9-diamine; quinazolines, N,N-dimethyl-N′-{2-[4-(4-methyl-piperazin-1-yl)-phenyl]-3,4-dihydro-quinazoline-4-yl}-ethane-1,2,-diamine; N′-[6,7-Dimethoxy-2-(4-phenyl-piperazin-1-yl)-quinazolin-4-yl]-N,N-dimethyl-ethane-1,2-diamine; N′-[6,7-Dimethoxy-2-(4-methyl-piperazin-1-yl)-quinazolin-4-yl]-N,N-dimethyl-ethane-1,2-diamine; N,N-Dimethyl-N′-(2-phenyl-quinazolin-4-yl)-ethane-1,2-diamine; Dimethyl-(2-{2-[4-(4-methyl-piperazin-1-yl)-phenyl]-quinazolin-4-yloxy}-ethyl)-amine; N′-(2-Biphenyl-4-yl-quinazolin-4-yl)-N,N-dimethyl-ethane-1,2-diamine and Dimethyl-[2-(2-phenyl-quinazolin-4-yloxy)-ethyl]-amine; ODN 2088, ODN with a TTAGGG sequence, G-ODN, statins, atorvastatin, IMO-2125 (Idera Pharmaceuticals), IRS 869, CMZ 203-84, CMZ 203-85, CMZ 203-88, CMZ 203-88-1, CMZ 203-89, CMZ 203-91, INH-ODN 2114, ODN A151, ODN INH-1, ODN INH-18, ODN 4084, ODN 4084-F, and ODN INH-47, comprising:
detecting the presence of absence of a mutation in MYD88 in a sample from an individual; and
characterizing the individual as a candidate for therapy with the combination comprising a BTK inhibitor and a TLR inhibitor if the individual has a mutation in MYD88.

58. The method of claim 57, wherein the mutation is at amino acid position 198 or 265 of MYD88.

59. The method of claim 58, wherein the mutation at amino acid position 198 of MYD88 is S198N.

60. The method of claim 58, wherein the mutation at amino acid position 265 of MYD88 is L265P.

61. The method of claim 57, wherein the combination provides a synergistic therapeutic effect compared to administration of the BTK inhibitor or the TLR inhibitor alone.

62. The method of any one of claims 57-61, wherein the non-specific TLR inhibitor is selected from the group consisting of chloroquine and bafilomycin A.

63. The method of claim 57-61, wherein the TLR7/8/9 antagonist is selected from the group consisting of CPG52364, IMO 8400, and IMO-9200.

64. The method of any one of claims 57-63, wherein the B-cell malignancy is diffuse large B-cell lymphoma (DLBCL), marginal zone lymphoma (MZL), acute lymphoblastic leukemia (ALL), acute myelogenous leukemia (AML), chronic myelogenous leukemia (CML), acute monocytic leukemia (AMoL), chronic lymphocytic leukemia (CLL), small lymphocytic lymphoma (SLL), high-risk small lymphocytic lymphoma (SLL), follicular lymphoma (FL), mantle cell lymphoma (MCL), Waldenstrom's macroglobulinemia, multiple myeloma, extranodal marginal zone B cell lymphoma, nodal marginal zone B cell lymphoma, Burkitt's lymphoma, non-Burkitt high grade B cell lymphoma, primary mediastinal B-cell lymphoma (PMBL), immunoblastic large cell lymphoma, precursor B-lymphoblastic lymphoma, B cell prolymphocytic leukemia, lymphoplasmacytic lymphoma, splenic marginal zone lymphoma, plasma cell myeloma, plasmacytoma, mediastinal (thymic) large B cell lymphoma, intravascular large B cell lymphoma, primary effusion lymphoma, or lymphomatoid granulomatosis.

65. The method of claim 64, wherein the DLBCL is activated B-cell diffuse large B-cell lymphoma (ABC-DLBCL).

66. The method of any one of claims 57-65, wherein the B-cell malignancy is a relapsed or refractory B-cell malignancy.

67. The method of claim 57, wherein the sample comprises one or more tumor cells.

68. The method of claim 57, wherein the method further comprises administering the combination of a BTK inhibitor and a TLR inhibitor.

69. The method of any one of claims 57-68, wherein the BTK inhibitor is ibrutinib.

70. The method of claim 69, wherein ibrutinib is administered at a dosage of about 40 mg/day to about 1000 mg/day.

71. The method of claim 70, wherein ibrutinib is administered orally.

72. The method of claim 69-71, wherein ibrutinib and the TLR inhibitor are administered simultaneously, sequentially or intermittently.

73. The method of any one of claims 57-72, wherein the method further comprises administering a third therapeutic agent.

74. The method of claim 73, wherein the third therapeutic agent is selected from among a chemotherapeutic agent or radiation therapeutic agent.

75. The method of claim 74, wherein the chemotherapeutic agent is selected from among chlorambucil, ifosfamide, doxorubicin, mesalazine, thalidomide, lenalidomide, temsirolimus, everolimus, fludarabine, fostamatinib, paclitaxel, docetaxel, ofatumumab, rituximab, dexamethasone, prednisone, CAL-101, ibritumomab, tositumomab, bortezomib, pentostatin, endostatin, or a combination thereof.

76. A pharmaceutical combination comprising:
a BTK inhibitor; and
a TLR inhibitor, wherein the TLR inhibitor is selected from the group consisting of a non-specific TLR inhibitor; a TLR7/8/9 antagonist; and a TLR9 antagonist, wherein the TLR9 antagonist is selected from the group consisting of is selected from the group consisting of a non-specific TLR inhibitor; a TLR6/7/8/9 antagonist; and a TLR9 antagonist, wherein the TLR9 antagonist is selected from the group consisting of chloroquine, quinacrine, monesin, bafilomycin A1, wortmannin, iODN, (+)-morphinans, 9-aminoacridine, 4-aminoquinoline, 4-aminoquinolines, 7,8,9,10-tetrahydro-6H-cyclohepta[b]quinolin-11-ylamine; 1-methyl-2,3-dihydro-1H-pyrrolo[2,3-b]quinolin-4-ylamine; 1,6-dimethyl-2,3-dihydro-1H-pyrrolo[2,3-b]quinolin-4-ylamine; 6-bromo-1-methyl-2,3-dihydro-1H-pyrrolo[2,3-b]quinolin-4-ylamine; 1-methyl-2,3,4,5-tetrahydro-1H-azepino[2,3-b]quinolin-6-ylamine; 3,3-dimethyl-3,4-dihydro-acridin-9-ylamine; 1-benzyl-2,3-dihydro-1H-pyrrolo[2,3-b]quinolin-4-ylamine; 6-methyl-1-phenyl-2,3-dihydro-1H-pyrrolo[2,3-b]quinolin-4-ylamine; N*2*,N*2*-Dimethyl-quinoline-2,4-diamine, 2,7-Dimethyl-dibenzo[b,g][1,8]naphthyridin-11-ylamine; 2,4-Dimethyl-benzo[b][1,8]naphthyridin-5-ylamine; 7-Fluoro-2,4-dimethyl-benzo[b][1,8]naphthyridin-5-ylamine; 1,2,3,4-Tetrahydro-acridin-9-ylamine Tacrine hydrochloridehydrate; 2,3-Dihydro-1H-cyclopenta[b]quinolin-9-ylamine; 2,4,9-Trimethyl-benzo[b][1,8]naphthyridin-5-ylamine; 9-Amino-3,3-dimethyl-1,2,3,4-tetrahydro-acridin-1-ol and 7-Ethoxy-N*3*-furan-2-ylmethyl-acridine-3,9-diamine; quinazolines, N,N-dimethyl-N′-{2-[4-(4-methyl-piperazin-1-yl)-phenyl]-3,4-dihydro-quinazoline-4-yl}-ethane-1,2,-diamine; N′-[6,7-Dimethoxy-2-(4-phenyl-piperazin-1-yl)-quinazolin-4-yl]-N,N-dimethyl-ethane-1,2-diamine; N′-[6,7-Dimethoxy-2-(4-methyl-piperazin-1-yl)-quinazolin-4-yl]-N,N-dimethyl-ethane-1,2-diamine; N,N-Dimethyl-N′-(2-phenyl-quinazolin-4-yl)-ethane-1,2-diamine; Dimethyl-(2-{2-[4-(4-methyl-piperazin-1-yl)-phenyl]-quinazolin-4-yloxy}-ethyl)-amine; N′-(2-Biphenyl-4-yl-quinazolin-4-yl)-N,N-dimethyl-ethane-1,2-diamine and Dimethyl-[2-(2-phenyl-quinazolin-4-yloxy)-ethyl]-amine; ODN 2088, ODN with a TTAGGG sequence, G-ODN, statins, atorvastatin, IMO-2125 (Idera Pharmaceuticals), IRS 869, CMZ 203-84, CMZ 203-85, CMZ 203-88, CMZ 203-88-1, CMZ 203-89, CMZ 203-91, INH-ODN 2114, ODN A151, ODN INH-1, ODN INH-18, ODN 4084, ODN 4084-F, and ODN INH-47.

77. The pharmaceutical combination of claim 76, further comprising a pharmaceutically-acceptable excipient.

78. The pharmaceutical combination of claim 76, wherein the combination provides a synergistic therapeutic effect compared to administration of the BTK inhibitor or the TLR inhibitor alone.

79. The pharmaceutical combination of any one of claims 76-78, wherein the non-specific TLR inhibitor is selected from the group consisting of chloroquine and bafilomycin A.

80. The pharmaceutical combination of any one of claims 76-78, wherein the TLR7/8/9 antagonist is selected from the group consisting of CPG52364, IMO 8400, and IMO-9200.

81. The pharmaceutical combination of any one of claims 76-80, wherein the BTK inhibitor is ibrutinib.

82. The pharmaceutical combination of any one of claims 76-81, wherein the combination is in a combined dosage form.

83. The pharmaceutical combination of any one of claims 76-82, wherein the combination is in separate dosage forms.

84. A method of treating an ibrutinib-resistant non-Hodgkin's lymphoma in a subject in need thereof, comprising administering to the subject a therapeutically effective amount of a combination comprising ibrutinib and a TLR inhibitor.

85. The method of claim 84, wherein the TLR inhibitor is selected from the group consisting of a non-specific TLR inhibitor, a TLR6/7/8/9 antagonist, and a TLR9 antagonist.

86. The method of claim 84, wherein the combination provides a synergistic therapeutic effect compared to administration of ibrutinib or the TLR inhibitor alone.

87. The method of claim 85, wherein the non-specific TLR inhibitor is selected from the group consisting of chloroquine and bafilomycin A.

88. The method of claim 85, wherein the TLR7/8/9 antagonist is selected from the group consisting of CPG52364, IMO 8400, and IMO-9200.

89. The method of claim 85, wherein the TLR9 antagonist is selected from the group consisting of chloroquine, quinacrine, monesin, bafilomycin A1, wortmannin, iODN, (+)-morphinans, 9-aminoacridine, 4-aminoquinoline, 4-aminoquinolines, 7,8,9,10-tetrahydro-6H-cyclohepta[b]quinolin-11-ylamine; 1-methyl-2,3-dihydro-1H-pyrrolo[2,3-b]quinolin-4-ylamine; 1,6-dimethyl-2,3-dihydro-1H-pyrrolo[2,3-b]quinolin-4-ylamine; 6-bromo-1-methyl-2,3-dihydro-1H-pyrrolo[2,3-b]quinolin-4-ylamine; 1-methyl-2,3,4,5-tetrahydro-1H-azepino[2,3-b]quinolin-6-ylamine; 3,3-dimethyl-3,4-dihydro-acridin-9-ylamine; 1-benzyl-2,3-dihydro-1H-pyrrolo[2,3-b]quinolin-4-ylamine; 6-methyl-1-phenyl-2,3-dihydro-1H-pyrrolo[2,3-b]quinolin-4-ylamine; N*2*,N*2*-Dimethyl-quinoline-2,4-diamine, 2,7-Dimethyl-dibenzo[b,g][1,8]naphthyridin-11-ylamine; 2,4-Dimethyl-benzo[b][1,8]naphthyridin-5-ylamine; 7-Fluoro-2,4-dimethyl-benzo[b][1,8]naphthyridin-5-ylamine; 1,2,3,4-Tetrahydro-acridin-9-ylamine Tacrine hydrochloridehydrate; 2,3-Dihydro-1H-cyclopenta[b]quinolin-9-ylamine; 2,4,9-Trimethyl-benzo[b][1,8]naphthyridin-5-ylamine; 9-Amino-3,3-dimethyl-1,2,3,4-tetrahydro-acridin-1-ol and 7-Ethoxy-N*3*-furan-2-ylmethyl-acridine-3,9-diamine; quinazolines, N,N-dimethyl-N′-{2-[4-(4-methyl-piperazin-1-yl)-phenyl]-3,4-dihydro-quinazoline-4-yl}-ethane-1,2,-diamine; N′-[6,7-Dimethoxy-2-(4-phenyl-piperazin-1-yl)-quinazolin-4-yl]-N,N-dimethyl-ethane-1,2-diamine; N′-[6,7-Dimethoxy-2-(4-methyl-piperazin-1-yl)-quinazolin-4-yl]-N,N-dimethyl-ethane-1,2-diamine; N,N-Dimethyl-N′-(2-phenyl-quinazolin-4-yl)-ethane-1,2-diamine; Dimethyl-(2-{2-[4-(4-methyl-piperazin-1-yl)-phenyl]-quinazolin-4-yloxy}-ethyl)-amine; N′-(2-Biphenyl-4-yl-quinazolin-4-yl)-N,N-dimethyl-ethane-1,2-diamine and Dimethyl-[2-(2-phenyl-quinazolin-4-yloxy)-ethyl]-amine; ODN 2088, ODN with a TTAGGG sequence, G-ODN, statins, atorvastatin, IMO-2125 (Idera Pharmaceuticals), IRS 869, CMZ 203-84, CMZ 203-85, CMZ 203-88, CMZ 203-88-1, CMZ 203-89, CMZ 203-91, INH-ODN 2114, ODN A151, ODN INH-1, ODN INH-18, ODN 4084, ODN 4084-F, and ODN INH-47.

90. The method of any one of claims 84-89, wherein the ibrutinib-resistant non-Hodgkin's lymphoma is marginal zone lymphoma (MZL), extranodal marginal zone B-cell lymphoma (also known as mucosa-associated lymphoid tissue (MALT) lymphomas), nodal marginal zone B-cell lymphoma, splenic marginal zone B-cell lymphoma, lymphoplasmacytic lymphoma (Waldenstrom macroglobulinemia), hairy cell leukemia, primary central nervous system (CNS) lymphoma, Burkitt lymphoma, chronic lymphocytic leukemia/small lymphocytic lymphoma (CLL/SLL), diffuse large B-cell lymphoma (DLBCL), primary mediastinal B-cell lymphoma, Intravascular large B-cell lymphoma, follicular lymphoma, immunoblastic large cell lymphoma, precursor B-lymphoblastic lymphoma, or mantle cell lymphoma.

91. The method of claim 90, wherein the ibrutinib-resistant DLBCL is ibrutinib-resistant activated B-cell diffuse large B-cell lymphoma (ABC-DLBCL).

92. The method of claim 91, wherein the ibrutinib-resistant ABC-DLBCL is characterized by a mutation in MYD88.

93. The method of claim 92, wherein the mutation is at position 265 of MYD88.

94. The method of claim 93, wherein the mutation is an L265P mutation.

95. A method of selecting a subject having a non-Hodgkin's lymphoma for treatment with a combination of a BTK inhibitor and a TLR inhibitor, comprising:
determining the expression level of a TLR biomarker or a TLR-related biomarker; and
administering to the individual a therapeutically effective amount of a combination of a BTK inhibitor and a TLR inhibitor if there is no increase in the expression level of the TLR biomarker or the TLR-related biomarker relative to a control.

96. A method of monitoring the disease progression in a subject having a non-Hodgkin's lymphoma, comprising:
determining the expression level of a TLR biomarker or a TLR-related biomarker, and
characterizing the subject as developed a resistance to a BTK inhibitor if the subject shows an increase in expression level of the TLR biomarker or the TLR-related biomarker relative to a control.

97. The method of any one of claims 95-96, wherein the expression level of the TLR biomarker or the TLR-related biomarker increases by 0.5-fold, 1-fold, 1.5-fold, 2-fold, 2.5-fold, 3-fold, 3.5-fold, 4-fold, 4.5-fold, 5-fold, 5.5-fold, 6-fold, 6.5-fold, 7-fold, 7.5-fold, 8-fold, 8.5-fold, 9-fold, 9.5-fold, 10-fold, 15-fold, 20-fold, 50-fold, or more compared to the control.

98. The method of any one of the claims 95-97, wherein the control is the expression levels of the TLR biomarker or the TLR-related biomarker in an individual who is not insensitive toward the BTK inhibitor.

99. The method of any one of the claims 95-97, wherein the control is the expression levels of the TLR biomarker or the TLR-related biomarker in an individual who has not been treated with the BTK inhibitor.

100. The method of any one of claims 95-99, wherein the TLR biomarker comprises TLR2, TLR3, TLR4, TLR5, or TLR9.

101. The method of any one of claims 95-100, wherein the TLR-related biomarker comprises a TLR interacting molecule, a TLR downstream effector, or a TLR-related cytokine or chemokine.

102. The method of claim 101, wherein the TLR interacting molecule comprises CD14, HSPA1A, LY96, JIP3, RIPK2, or TIRAP.

103. The method of claim 101, wherein the TLR downstream effector comprises CASP8, CHUK, EIF2AK2, IKBKB, IRAK2, IRF1, MAP2K4, NFKB2, NFKBIL1, NFRKB, PPARA, PTGS2, RELA, TAB1, or TRAF6.

104. The method of claim 101, wherein the TLR related cytokine or chemokine comprises CCL2, CSF2, CSF3, CXCL10, IFNA1, IFNB1, IFNG, IL12A, IL1A, IL1B, IL2, IL6, IL8, or LTA.

105. The method of any one of claims 94-104, wherein the TLR inhibitor is selected from a non-specific TLR inhibitor, a TLR6/7/8/9 antagonist, and a TLR9 antagonist.

106. The method of claim 105, wherein the non-specific TLR inhibitor is selected from the group consisting of chloroquine and bafilomycin A.

107. The method of claim 105, wherein the TLR7/8/9 antagonist is selected from the group consisting of CPG52364, IMO 8400, and IMO-9200.

108. The method of claim 105, wherein the TLR9 antagonist is selected from the group consisting of chloroquine, quinacrine, monesin, bafilomycin A1, wortmannin, iODN, (+)-morphinans, 9-aminoacridine, 4-aminoquinoline, 4-aminoquinolines, 7,8,9,10-tetrahydro-6H-cyclohepta[b]quinolin-11-ylamine; 1-methyl-2,3-dihydro-1H-pyrrolo[2,3-b]quinolin-4-ylamine; 1,6-dimethyl-2,3-dihydro-1H-pyrrolo[2,3-b]quinolin-4-ylamine; 6-bromo-1-methyl-2,3-dihydro-1H-pyrrolo[2,3-b]quinolin-4-ylamine; 1-methyl-2,3,4,5-tetrahydro-1H-azepino[2,3-b]quinolin-6-ylamine; 3,3-dimethyl-3,4-dihydro-acridin-9-ylamine; 1-benzyl-2,3-dihydro-1H-pyrrolo[2,3-b]quinolin-4-ylamine; 6-methyl-1-phenyl-2,3-dihydro-1H-pyrrolo[2,3-b]quinolin-4-ylamine; N*2*,N*2*-Dimethyl-quinoline-2,4-diamine, 2,7-Dimethyl-dibenzo[b,g][1,8]naphthyridin-11-ylamine; 2,4-Dimethyl-benzo[b][1,8]naphthyridin-5-ylamine; 7-Fluoro-2,4-dimethyl-benzo[b][1,8]naphthyridin-5-ylamine; 1,2,3,4-Tetrahydro-acridin-9-ylamine Tacrine hydrochloridehydrate; 2,3-Dihydro-1H-cyclopenta[b]quinolin-9-ylamine; 2,4,9-Trimethyl-benzo[b][1,8]naphthyridin-5-ylamine; 9-Amino-3,3-dimethyl-1,2,3,4-tetrahydro-acridin-1-ol and 7-Ethoxy-N*3*-furan-2-ylmethyl-acridine-3,9-diamine; quinazolines, N,N-dimethyl-N′-{2-[4-(4-methyl-piperazin-1-yl)-phenyl]-3,4-dihydro-quinazoline-4-yl}-ethane-1,2,-diamine; N′-[6,7-Dimethoxy-2-(4-phenyl-piperazin-1-yl)-quinazolin-4-yl]-N,N-dimethyl-ethane-1,2-diamine; N′-[6,7-Dimethoxy-2-(4-methyl-piperazin-1-yl)-quinazolin-4-yl]-N,N-dimethyl-ethane-1,2-diamine; N,N-Dimethyl-N′-(2-phenyl-quinazolin-4-yl)-ethane-1,2-diamine; Dimethyl-(2-{2-[4-(4-methyl-piperazin-1-yl)-phenyl]-quinazolin-4-yloxy}-ethyl)-amine; N′-(2-Biphenyl-4-yl-quinazolin-4-yl)-N,N-dimethyl-ethane-1,2-diamine and Dimethyl-[2-(2-phenyl-quinazolin-4-yloxy)-ethyl]-amine; ODN 2088, ODN with a TTAGGG sequence, G-ODN, statins, atorvastatin, IMO-2125 (Idera Pharmaceuticals), IRS 869, CMZ 203-84, CMZ 203-85, CMZ 203-88, CMZ 203-88-1, CMZ 203-89, CMZ 203-91, INH-ODN 2114, ODN A151, ODN INH-1, ODN INH-18, ODN 4084, ODN 4084-F, and ODN INH-47.

109. The method of any one of claims 95-108, wherein the BTK inhibitor is ibrutinib.

110. The method of any one of claims 95-109, wherein the non-Hodgkin's lymphoma is marginal zone lymphoma (MZL), extranodal marginal zone B-cell lymphoma (also known as mucosa-associated lymphoid tissue (MALT) lymphomas), nodal marginal zone B-cell lymphoma, splenic marginal zone B-cell lymphoma, lymphoplasmacytic lymphoma (Waldenstrom macroglobulinemia), hairy cell leukemia, primary central nervous system (CNS) lymphoma, Burkitt lymphoma, chronic lymphocytic leukemia/small lymphocytic lymphoma (CLL/SLL), diffuse large B-cell lymphoma (DLBCL), primary mediastinal B-cell lymphoma, Intravascular large B-cell lymphoma, follicular lymphoma, immunoblastic large cell lymphoma, precursor B-lymphoblastic lymphoma, or mantle cell lymphoma.

111. The method of claim 110, wherein DLBCL is activated B-cell diffuse large B-cell lymphoma (ABC-DLBCL).

112. The method of claim 111, wherein the ABC-DLBCL is characterized by a mutation in MYD88.

113. The method of claim 112, wherein the mutation is at position 265 of MYD88.

114. The method of claim 113, wherein the mutation is an L265P mutation.

115. The method of any one of claims 95-114, wherein the non-Hodgkin's lymphoma is a relapsed or refractory non-Hodgkin's lymphoma.

116. The method of any one of claims 95-115, wherein the non-Hodgkin's lymphoma is an ibrutinib-resistant non-Hodgkin's lymphoma.

117. The method of any one of claims 1-33 and 84-94, wherein the subject does not overexpress TLR4.

118. The method of any one of claims 1-33 and 84-94, wherein the subject does not overexpress ILR1.

119. The method of any one of claims 1-33 and 84-94, wherein the subject does not overexpress TLR4 and ILR1.

120. The method of any one of claims 1-33 and 84-94, further comprising co-administering a PIM ihibitor.

121. The method of claim 120, wherein the PIM inhibitor is a pan-PIM inhibitor.

122. The method of claim 120, wherein the PIM inhibitor is a PIM1 inhibitor.

123. The method of any one of claims 1-33 and 84-94, further comprising co-administering a compound or oligonucleotide that downregulates expression of PIM.

124. The method of claim 123, wherein the compound or oligonucleotide downregulates expression of PIM1.
